(19) United States
(12) Patent Application Publication
(10) Pub. No.: US 2026/0293898 A1
(43) Pub. Date: Oct. 1, 2026

(54) FUNGICIDAL COMPOSITIONS

(71) Applicant: SYNGENTA CROP PROTECTION AG, Basel (CH)

(72) Inventors: Stephane André Marie JEANMART, Stein (CH); Mathias BLUM, Stein (CH); Alexandre Franco Jean Camille LUMBROSO, Stein (CH); Nicolas GERMAIN, Stein (CH); Martin POULIOT, Stein (CH); Camille LE CHAPELAIN, Stein (CH)

(73) Assignee: SYNGENTA CROP PROTECTION AG, Basel (CH)

(21) Appl. No.: 19/473,366

(22) PCT Filed: Apr. 12, 2024
(86) PCT No.: PCT/EP2024/060005

§ 371 Date: Oct. 7, 2025

(30) Foreign Application Priority Data
Apr. 13, 2023 (EP) 23167798.0

(51) Int. Cl. A01N 43/90; A01P 3/00; C07D 471/04

(52) U.S. Cl. CPC A01N 43/90; A01P 3/00; C07D 471/04

(57) ABSTRACT

The current invention relates to a fungicidal composition comprising components (A) and (B), wherein components (A) and (B) are as defined in claim 1, and use of the compositions in agriculture or horticulture for controlling or preventing infestation of plants by phytopathogenic microorganisms, preferably fungi.

The present invention relates to novel fungicidal compositions, to their use in agriculture or horticulture for controlling diseases caused by phytopathogens, especially phytopathogenic fungi and more particularly oomycetes, and to methods of controlling diseases on useful plants.

Whilst many fungicidal compounds and compositions, belonging to various different chemical classes, have been/are being developed for use as fungicides in crops of useful plants, crop tolerance and activity against particular phytopathogenic fungi do not always satisfy the needs of agricultural practice in many respects. Therefore, there is a continuing need to find new compounds and compositions having superior biological properties for use in controlling or preventing infestation of plants by phytopathogenic fungi. For example, compounds possessing a greater biological activity, an advantageous spectrum of activity, an increased safety profile, improved physico-chemical properties, or increased biodegradability. Or else, compositions possessing a broader spectrum of activity, improved crop tolerance, synergistic interactions or potentiating properties, or compositions which display a more rapid onset of action or which have longer lasting residual activity or which enable a reduction in the number of applications and/or a reduction in the application rate of the compounds and compositions required for effective control of a phytopathogen, thereby enabling beneficial resistance-management practices, reduced environmental impact and reduced operator exposure.

The use of compositions comprising mixtures of different fungicidal compounds possessing different modes of action can address some of these needs (eg, by combining fungicides with differing spectrums of activity).

According to the present invention, there is provided a fungicidal composition comprising components (A) and (B) as active ingredients, wherein component (A) is a compound of formula (I):

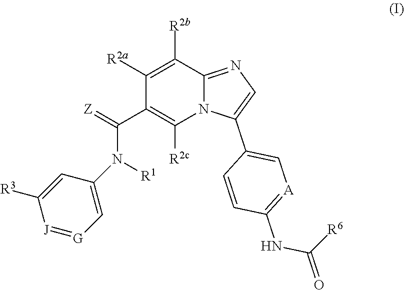

wherein Z is 0;
R¹ is selected from C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl, wherein each of the C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl groups is optionally substituted with CN;
R^{2a} is selected from H, C₁₋₆alkyl, C₃₋₆cycloalkyl, and C₁₋₆alkoxy-C₁₋₆alkyl;
R^{2b} is selected from H, CN, C₁₋₆alkyl, C₃₋₆cycloalkyl, C₁₋₆alkoxy-C₁₋₆alkyl, amino, and —NHC(O)C₁₋₆alkyl;
R^{2c} is H;
J is N or CR⁴;
G is CR⁵;
R³ is H;
R⁴ is halogen or CN;
R⁵ is selected from H, C₁₋₆alkyl, C₁₋₆alkoxy, and halogen;
A is CH or N; and
R⁶ is selected from C₁₋₆alkyl, C₁₋₆alkoxy, C₃₋₆cycloalkyl, C₁₋₆alkylamino, C₁₋₆alkoxyamino, and C₁₋₆alkylC₁₋₆alkoxyamino;
or a salt or N-oxide thereof;
and
component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide.

In a particular embodiment according to the present invention, the component (A) is a compound of formula (I):

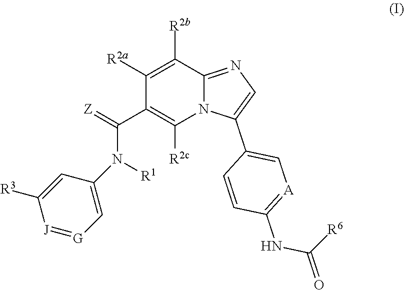

wherein Z is O;
R¹ is selected from C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl, wherein each of the C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl groups is optionally substituted with CN;
R^{2a} is selected from H, C₁₋₆alkyl, C₃₋₆cycloalkyl, and C₁₋₆alkoxy-C₁₋₆alkyl;
R^{2b} is selected from H, CN, C₁₋₆alkyl, C₃₋₆cycloalkyl, C₁₋₆alkoxy-C₁₋₆alkyl, amino, and —NHC(O)C₁₋₆alkyl;
R^{2c} is H;
J is CR⁴;
G is CR⁵;
R³ is H;
R⁴ is halogen or CN;
R⁵ is selected from H, C₁₋₆alkyl, C₁₋₆alkoxy, and halogen;
A is CH or N; and
R⁶ is selected from C₁₋₆alkyl, C₁₋₆alkoxy, C₃₋₆cycloalkyl, C₁₋₆alkylamino, C₁₋₆alkoxyamino, and C₁₋₆alkylC₁₋₆alkoxyamino;
or a salt or N-oxide thereof.

In another particular embodiment according to the present invention, the component (A) is a compound of formula (I):

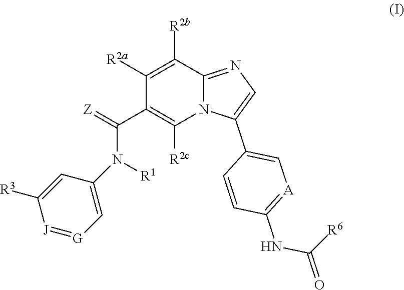

wherein Z is O;
R¹ is selected from C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl, wherein each of the C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl groups is optionally substituted with CN;
R^{2a} is selected from H, C₁₋₆alkyl, C₃₋₆cycloalkyl, and C₁₋₆alkoxy-C₁₋₆alkyl;
R^{2b} is selected from H, CN, C₁₋₆alkyl, C₃₋₆cycloalkyl, C₁₋₆alkoxy-C₁₋₆alkyl, amino, and —NHC(O)C₁₋₆alkyl;
R^{2c} is H;
J is N;
G is CR⁵;
R³ is H;
R⁵ is selected from H, C₁₋₆alkyl, C₁₋₆alkoxy, and halogen;
A is CH or N; and
R⁶ is selected from C₁₋₆alkyl, C₁₋₆alkoxy, C₃₋₆cycloalkyl, C₁₋₆alkylamino, C₁₋₆alkoxyamino, and C₁₋₆alkylC₁₋₆alkoxyamino;
or a salt or N-oxide thereof.

In the present invention, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide may be prepared from the methods described in WO2017/153380.

In the present invention, among the components selected from N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, the preferred compound (Compound Z) is the one having the following CAS number: 2132414-04-9 (feneptamidoquin). Compound Z is more particularly N-[(1RS)-1-benzyl-1,3-dimethyl-butyl]-8-fluoro-quinoline-3-carboxamide containing 80-100% of the R-enantiomer.

In the present invention, Streptomyces chrestomyceticus comprises a nucleotide sequence which has at least 99.8%, preferably at least 99.9%, preferably at least 99.91%, 99.92%, 99.93%, 99.94%, 99.95%, 99.96%, 99.97%, 99.98%, or 99.99% identity to SEQ ID NO: 1. In a particular embodiment, the Streptomyces chrestomyceticus comprises a nucleotide sequence which has 100% identity to SEQ ID NO:1. SEQ ID NO: 1 comprises the 16S RNA gene of Streptomyces chrestomyceticus Saigon413 deposited with the Westerdijk Institute under accession number CBS149411.

Throughout this document the expression “composition” stands for the various mixtures or combinations of components (A) and (B) (including the above-defined embodiments), for example in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the components (A) and (B) is not essential for working the present invention The term “halogen” refers to fluorine (fluoro or F), chlorine (chloro or CI), bromine (bromo or Br) or iodine (iodo or I), and preferably fluoro or chloro.

The term “amino” refers to a —NH₂ group.

The term “Alkyl” as used herein—in isolation or as part of a chemical group—represents straight-chain or branched hydrocarbons, preferably with 1 to 6 carbon atoms, for example methyl, ethyl, n-propyl, isopropyl, n-butyl, isobutyl, s-butyl, t-butyl, pentyl, 1-methylbutyl, 2-methylbutyl, 3-methylbutyl, 1,2-dimethylpropyl, 1,1-dimethylpropyl, 2,2-dimethylpropyl, 1-ethylpropyl, hexyl, 1-methylpentyl, 2-methylpentyl, 3-methylpentyl, 4-methylpentyl, 1,2-dimethylpropyl, 1,3-dimethylbutyl, 1,4-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl and 2-ethylbutyl. Alkyl groups with 1 to 4 carbon atoms are preferred, for example methyl, ethyl, n-propyl, isopropyl, n-butyl, isobutyl, s-butyl or t-butyl.

The term “cycloalkyl”—in isolation or as part of a chemical group—represents saturated or partially unsaturated mono-, bi- or tricyclic hydrocarbons, preferably with 3 to 10 carbon atoms, for example cyclopropyl, cyclobutyl, cyclopentyl, cyclohexyl, cycloheptyl, cyclooctyl, bicyclo[2.2.1]heptyl, bicyclo[2.2.2]octyl or adamantyl. Cycloalkyls with 3, 4, 5, 6 or 7 carbon atoms are preferred, for example cyclopropyl or cyclobutyl.

The term “alkoxy” refers to a radical of the formula —ORₐ wherein Rₐ is an alkyl radical as generally defined above. Examples of alkoxy include, but are not limited to methoxy, ethoxy, propoxy, iso-propoxy, and tert-butoxy. The term “alkoxyalkyl” refers to an alkyl radical (as mentioned above) substituted with said alkoxy group. Examples are methoxymethyl, methoxyethyl, ethoxymethyl and propoxymethyl.

The term “alkylamino” refers to a radical of the formula RₐNH— wherein Rₐ is an alkyl radical as generally defined above.

The term “alkoxyamino” refers to a radical of the formula RₐNH—, wherein Rₐ is an alkoxy radical as generally defined above.

The term “alkylsulfonyl” refers to a radical of the formula —S(O)₂Rₐ wherein Rₐ is an alkyl radical as generally defined above.

In the present invention, the weight ratio of component (A) to component (B) may preferably be from 100:1 to 1:100, from 50:1 to 1:50, from 20:1 to 1:40, from 15:1 to 1:30, from 12:1 to 1:25, from 10:1 to 1:20, from 5:1 to 1:15, from 3:1 to 1:10, or from 2:1 to 1:5.

Further according to the invention, there is provided a method of controlling or preventing phytopathogenic diseases, especially phytopathogenic fungi, on useful plants or on propagation material thereof, which comprises applying to the useful plants, the locus thereof or propagation material thereof a fungicidal composition according to the invention.

The benefits provided by certain fungicidal mixture compositions according to the invention may also include, inter alia, advantageous levels of biological activity for protecting plants against diseases that are caused by fungi or superior properties for use as agrochemical active ingredients (for example, greater biological activity, an advantageous spectrum of activity, an increased safety profile, improved physico-chemical properties, or increased biodegradability).

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

Preferred groups and values for the substituents R¹, R^{2a}, R^{2b}, R^{2c}, R³, R³, R⁴, R⁵, A, and R⁶ in the compounds of formula (I) are, in any combination thereof, as set out below:
R¹ is selected from C₁₋₆alkyl, C₁₋₆alkoxy-C₁₋₆alkyl, and C₁₋₆alkylsulfonyl-C₁₋₆alkyl, wherein each of the C₁₋₆alkyl, C₁₋₆alkoxy-C₁₋₆alkyl and C₁₋₆alkylsulfonyl-C₁₋₆alkyl groups is optionally substituted with CN, and preferably selected from CH₃, CH₂—CH₃, CH₂—O—CH₃, CH₂—SO₂—CH₃, and CH₂CN;
R^{2a} is selected from H and C₁₋₆alkyl, and preferably selected from H and CH₃;
R^{2b} is selected from H, C₁₋₆alkyl, C₁₋₆alkoxy-C₁₋₆alkyl, amino, and —NHC(O)C₁₋₆alkyl, and preferably selected from H, CH₃, —CH₂OCH₃, —NH₂, and —NHC(O)CH₃;
R^{2c} is H;
J is N or CR⁴;
G is CR⁵;
R³ is H;
R⁴ is halogen or CN, and preferably chloro, fluoro, or CN;
R⁵ is selected from H, C₁₋₆alkyl, C₁₋₆alkoxy, and halogen, and preferably selected from H, CH₃, —OCH₃, and fluoro;
A is CH or N; and
R⁶ is selected from C₁₋₆alkyl, C₁₋₆alkoxy, C₃₋₆cycloalkyl, C₁₋₆alkylamino, and C₁₋₆alkoxyamino, and preferably selected from cyclopropyl, —OCH₃, and —NH—O—CH₃;
   or a salt or N-oxide thereof.

In a most preferred embodiment according to the present invention, the compounds of formula (I) are, in any combination thereof, as set out below:
R¹ is selected from C₁₋₆alkyl, C₁₋₆alkoxy-C₁₋₆alkyl, and C₁₋₆alkylsulfonyl-C₁₋₆alkyl, wherein each of the C₁₋₆alkyl, C₁₋₆alkoxy-C₁₋₆alkyl and C₁₋₆alkylsulfonyl-C₁₋₆alkyl groups is optionally substituted with CN, and preferably selected from CH₃, CH₂—CH₃, CH₂—O—CH₃, CH₂—SO₂—CH₃, and CH₂CN;
R^{2a} is selected from H and C₁₋₆alkyl, and preferably selected from H and CH₃;
R^{2b} is selected from H, C₁₋₆alkyl, C₁₋₆alkoxy-C₁₋₆alkyl, amino, and —NHC(O)C₁₋₆alkyl, and preferably selected from H, CH₃, —CH₂OCH₃, —NH₂, and —NHC(O)CH₃;
R^{2c} is H;
J is CR⁴;
G is CR⁵;
R³ is H;
R⁴ is halogen or CN, and preferably chloro, fluoro, or CN;
R⁵ is selected from H, C₁₋₆alkyl, C₁₋₆alkoxy, and halogen, and preferably selected from H, CH₃, —OCH₃, and fluoro;
A is CH or N; and
R⁶ is selected from C₁₋₆alkyl, C₁₋₆alkoxy, C₃₋₆cycloalkyl, C₁₋₆alkylamino, and C₁₋₆alkoxyamino, and preferably selected from cyclopropyl, —OCH₃, and —NH—O—CH₃;
   or a salt or N-oxide thereof.

In another most preferred embodiment according to the present invention, the compounds of formula (I) are, in any combination thereof, as set out below:
R¹ is selected from C₁₋₆alkyl, C₁₋₆alkoxy-C₁₋₆alkyl, and C₁₋₆alkylsulfonyl-C₁₋₆alkyl, wherein each of the C₁₋₆alkyl, C₁₋₆alkoxy-C₁₋₆alkyl and C₁₋₆alkylsulfonyl-C₁₋₆alkyl groups is optionally substituted with CN, and preferably selected from CH₃, CH₂—CH₃, CH₂—O—CH₃, CH₂—SO₂—CH₃, and CH₂CN;
R^{2a} is selected from H and C₁₋₆alkyl, and preferably selected from H and CH₃;
R^{2b} is selected from H, C₁₋₆alkyl, C₁₋₆alkoxy-C₁₋₆alkyl, amino, and —NHC(O)C₁₋₆alkyl, and preferably selected from H, CH₃, —CH₂OCH₃, —NH₂, and —NHC(O)CH₃;
R^{2c} is H;
J is N;
G is CR⁵;
R³ is H;
R⁵ is selected from H, C₁₋₆alkyl, C₁₋₆alkoxy, and halogen, and preferably selected from H, CH₃, —OCH₃, and fluoro;
A is CH or N; and
R⁶ is selected from C₁₋₆alkyl, C₁₋₆alkoxy, C₃₋₆cycloalkyl, C₁₋₆alkylamino, and C₁₋₆alkoxyamino, and preferably selected from cyclopropyl, —OCH₃, and —NH—O—CH₃;
   or a salt or N-oxide thereof.

Most preferably, component (A) is a compound selected from compound no. X.1, X.02, X.03, X.04, X.05, X.06, X.07, X.08, X.09, X.10, X.11, X.12, X.13, X.14, X.15, X.16, X.17, X.18, X.19, X.20, X.21, X.22, and X.23, as defined in the Table X below. More preferably, component (A) is a compound selected from compound no. X.01, X.02, X.03, X.04, X.05, X.06, X.21, X.22, and X.23, as defined in the Table X below.

TABLE X

| Entry | Compound name | Molecule |
|---|---|---|
| X.01 | methyl N-[5-[6-[(4-fluoro- 3-methoxy-phenyl)-methyl- carbamoyl]imidazo[1,2-a] pyridin-3-yl]-2-pyridyl] carbamate | 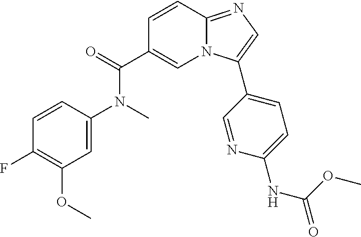 [SMILES: COC(=O)Nc1ccc(-c2cnc3ccc(C(=O)N(C)c4ccc(F)c(OC)c4)cn23)cn1] |
| X.02 | methyl N-[5-[6-[(4-fluoro- 3-methoxy-phenyl)-meth- yl-carbamoyl]-8-methyl- imidazo[1,2-a]pyridin-3- yl]-2-pyridyl]carbamate | 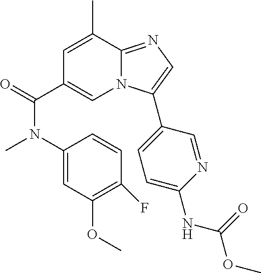 [SMILES: COC(=O)Nc1ccc(-c2cnc3c(C)cc(C(=O)N(C)c4ccc(F)c(OC)c4)cn23)cn1] |
| X.03 | methyl N-[5-[6-[(4-fluoro- 3-methoxy-phenyl)-(meth- oxymethyl)carbamoyl]-8- methyl-imidazo[1,2-a] pyridin-3-yl]-2-pyridyl] carbamate | 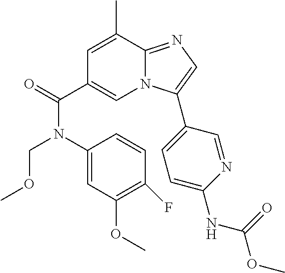 |
| X.04 | methyl N-[5-[6-[(4-fluoro- 3-methoxy-phenyl)-(meth- oxymethyl)carbamoyl] imidazo[1,2-a]pyridin-3- yl]-2-pyridyl]carbamate | 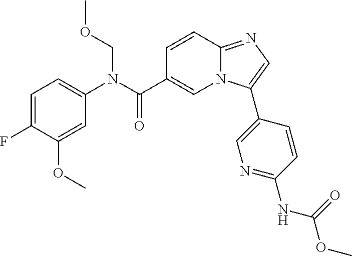 [SMILES: COCN(C(=O)c1ccc2ncc(-c3ccc(NC(=O)OC)nc3)n2c1)c1ccc(F)c(OC)c1] |
| X.05 | methyl N-[4-[6-[(4-fluoro- 3-methoxy-phenyl)-meth- ylcarbamoyl]imidazo[1,2- a]pyridin-3-yl]phenyl] carbamate | 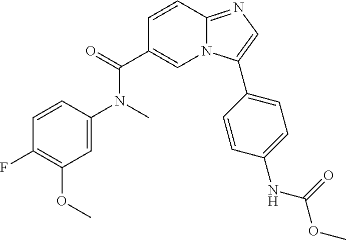 [SMILES: COC(=O)Nc1ccc(-c2cnc3ccc(C(=O)N(C)c4ccc(F)c(OC)c4)cn23)cc1] |
| X.06 | 3-[6-(cyclopropanecarbon- ylamino)-3-pyridyl]-N-(4- fluoro-3-methoxy-phenyl)- N-methyl-imidazo[1,2-a] pyridine-6-carboxamide | 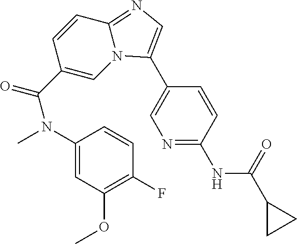 [SMILES: COc1cc(N(C)C(=O)c2ccc3ncc(-c4ccc(NC(=O)C5CC5)nc4)n3c2)ccc1F] |
| X.07 | methyl N-[4-[6-[(4-fluoro- phenyl)-methyl-carbamo- yl]imidazo[1,2-a]pyridin- 3-yl]phenyl]carbamate | 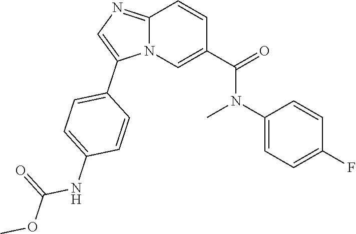 [SMILES: COC(=O)Nc1ccc(-c2cnc3ccc(C(=O)N(C)c4ccc(F)cc4)cn23)cc1] |
| X.08 | methyl N-[4-[6-[(4-chloro- phenyl)-methyl-carbamo- yl]-8-methyl-imidazo[1,2- a]pyridin-3-yl]phenyl] carbamate | 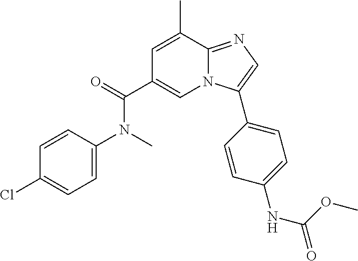 [SMILES: COC(=O)Nc1ccc(-c2cnc3c(C)cc(C(=O)N(C)c4ccc(Cl)cc4)cn23)cc1] |
| X.09 | methyl N-[5-[6-[(4-fluoro- 3-methyl-phenyl)-methyl- carbamoyl]imidazo[1,2-a] pyridin-3-yl]-2-pyridyl] carbamate | 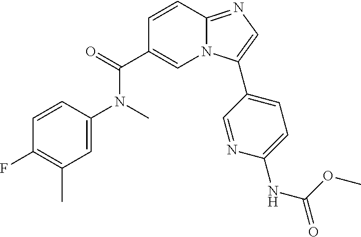 [SMILES: COC(=O)Nc1ccc(-c2cnc3ccc(C(=O)N(C)c4ccc(F)c(C)c4)cn23)cn1] |
| X.10 | methyl N-[5-[6-[(4-cyano- 3-methoxy-phenyl)-methyl- carbamoyl]imidazo[1,2-a] pyridin-3-yl]-2-pyridyl] carbamate | 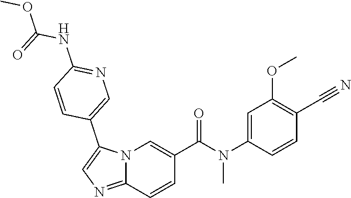 [SMILES: COC(=O)Nc1ccc(-c2cnc3ccc(C(=O)N(C)c4ccc(C#N)c(OC)c4)cn23)cn1] |
| X.11 | methyl N-[5-[6-[(4-chloro- 3-methoxy-phenyl)-methyl- carbamoyl]imidazo[1,2-a] pyridin-3-yl]-2-pyridyl] carbamate | 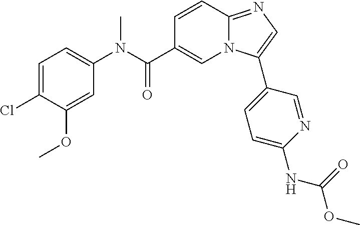 [SMILES: COC(=O)Nc1ccc(-c2cnc3ccc(C(=O)N(C)c4ccc(Cl)c(OC)c4)cn23)cn1] |
| X.12 | methyl N-[5-[6-[(4-fluoro- phenyl)-methyl-carbamo- yl]-8-methyl-imidazo[1,2- a]pyridin-3-yl]-2-pyridyl] carbamate | 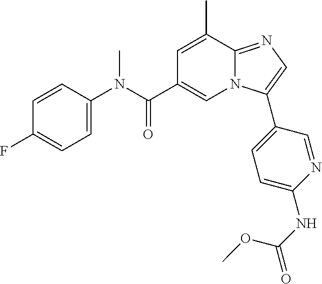 [SMILES: COC(=O)Nc1ccc(-c2cnc3c(C)cc(C(=O)N(C)c4ccc(F)cc4)cn23)cn1] |
| X.13 | methyl N-[5-[6-[(4-fluoro- phenyl)-methyl-carbamoyl]- 7-methyl-imidazo[1,2-a] pyridin-3-yl]-2-pyridyl] carbamate | 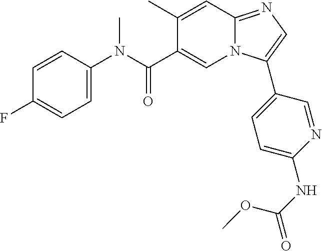 [SMILES: COC(=O)Nc1ccc(-c2cnc3cc(C)c(C(=O)N(C)c4ccc(F)cc4)cn23)cn1] |
| X.14 | methyl N-[5-[6-[cyano- methyl-(4-fluoro-3-meth- oxyphenyl)carbamoyl] imidazo[1,2-a]pyridin-3- yl]-2-pyridyl]carbamate | 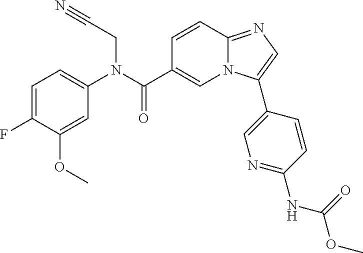 [SMILES: COC(=O)Nc1ccc(-c2cnc3ccc(C(=O)N(CC#N)c4ccc(F)c(OC)c4)cn23)cn1] |
| X.15 | methyl N-[5-[6-[(4-chloro- 3-methoxy-phenyl)(cyano- methyl)carbamoyl]imidazo [1,2-a]pyridin-3-yl]-2- pyridyl]carbamate | 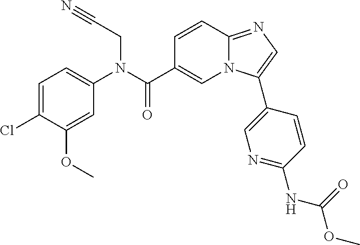 [SMILES: COC(=O)Nc1ccc(-c2cnc3ccc(C(=O)N(CC#N)c4ccc(Cl)c(OC)c4)cn23)cn1] |
| X.16 | methyl N-[5-[6-[(4-fluoro- 3-methoxy-phenyl)(methyl- sulfonylmethyl)carbamo- yl]imidazo[1,2-a]pyridin- 3-yl]-2-pyridyl]carbamate | 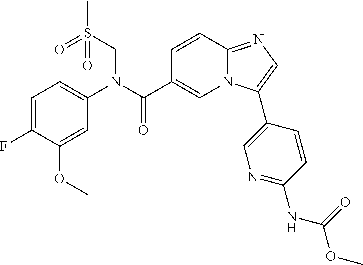 [SMILES: COC(=O)Nc1ccc(-c2cnc3ccc(C(=O)N(CS(C)(=O)=O)c4ccc(F)c(OC)c4)cn23)cn1] |
| X.17 | N-(4-fluoro-3-methoxy- phenyl)-3-[6-(methoxy- carbamoylamino)-3- pyridyl]-N-methyl- imidazo[1,2-a]pyridine- 6-carboxamide | 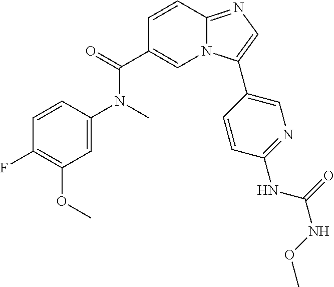 [SMILES: CONC(=O)Nc1ccc(-c2cnc3ccc(C(=O)N(C)c4ccc(F)c(OC)c4)cn23)cn1] |
| X.18 | methyl N-[5-[8-acetamido- 6-[(4-fluorophenyl)methyl- carbamoyl]imidazo[1,2-a] pyridin-3-yl]-2-pyridyl] carbamate | 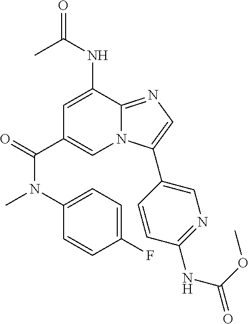 [SMILES: COC(=O)Nc1ccc(-c2cnc3c(NC(C)=O)cc(C(=O)N(C)c4ccc(F)cc4)cn23)cn1] |
| X.19 | methyl N-[5-[6-[ethyl-(4- fluorophenyl)carbamoyl] imidazo[1,2-a]pyridin- 3-yl]-2-pyridyl]carbamate | 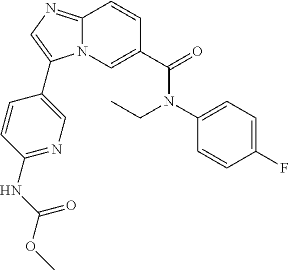 [SMILES: CCN(C(=O)c1ccc2ncc(-c3ccc(NC(=O)OC)nc3)n2c1)c1ccc(F)cc1] |
| X.20 | methyl N-[5-[8-acetamido- 6-[(4-fluoro-3-methoxy- phenyl)-methyl-carbamo- yl]imidazo[1,2-a]pyridin- 3-yl]-2-pyridyl]carbamate | 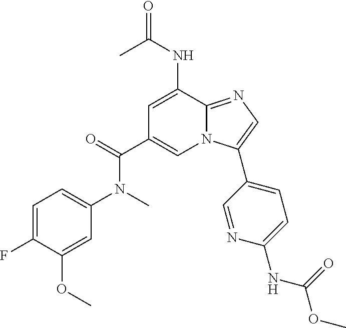 [SMILES: COC(=O)Nc1ccc(-c2cnc3c(NC(C)=O)cc(C(=O)N(C)c4ccc(F)c(OC)c4)cn23)cn1] |
| X.21 | methyl N-[5-[6-[(4-fluoro- 3-methoxy-phenyl)-meth- yl-carbamoyl]-8-(methoxy- methyl)imidazo[1,2-a] pyridin-3-yl]-2-pyridyl] carbamate | 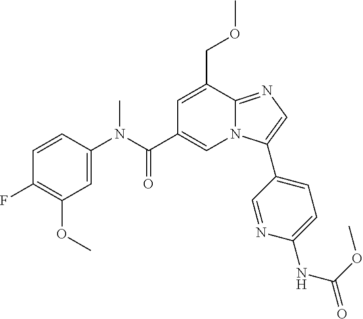 [SMILES: COCc1cc(C(=O)N(C)c2ccc(F)c(OC)c2)cn2c(-c3ccc(NC(=O)OC)nc3)cnc12] |
| X.22 | methyl N-[5-[6-[(2-meth- oxy-4-pyridyl)-methyl- carbamoyl]-8-methyl- imidazo[1,2-a]pyridin-3- yl]-2-pyridyl]carbamate | 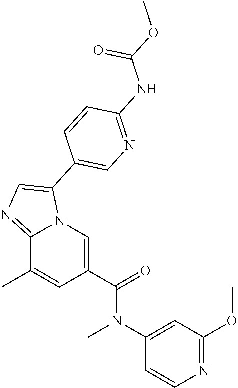 |
| X.23 | methyl N-[5-[6-[(4-cyano- 3-methoxy-phenyl)-meth- yl-carbamoyl]-8-methyl- imidazo[1,2-a]pyridin-3- yl]-2-pyridyl]carbamate | 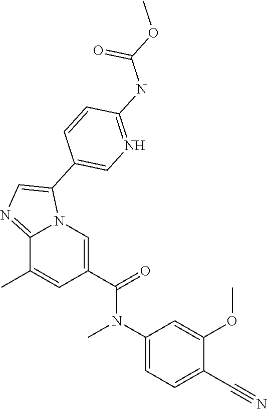 [SMILES: COC(=O)Nc1ccc(-c2cnc3c(C)cc(C(=O)N(C)c4ccc(C#N)c(OC)c4)cn23)cn1] |

Preferably, component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, N-[(1R)-1-benzyl-1,3-dimethyl-butyl]-8-fluoro-quinoline-3-carboxamide, and N-[(1 S)-1-benzyl-1,3-dimethyl-butyl]-8-fluoro-quinoline-3-carboxamide.

More preferably, component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin).

The component (B) compounds are referred to herein and above by a so-called “ISO common name” or another “common name” being used in individual cases or a trademark name. The component (B) compounds are known and are commercially available and/or can be prepared using procedures known in the art and/or procedures reported in the literature.

In a preferred composition according to the invention, component (A) is compound no. X.01 [methyl N-[5-[6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.02 [methyl N-[5-[6-[(4-fluoro-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.03 [methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.04 [methyl N-[5-[6-[(4-fluoro-3-methoxy-phenyl)-(methoxymethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.05 [methyl N-[4-[6-[(4-fluoro-3-methoxy-phenyl)-methyl-carbamoyl]imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.06 [3-[6-(cyclopropanecarbonylamino)-3-pyridyl]-N-(4-fluoro-3-methoxy-phenyl)-N-methyl-imidazo[1,2-a]pyridine-6-carboxamide] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.07 [methyl N-[4-[6-[(4-fluorophenyl)-methyl-carbamoyl]imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.08 [methyl N-[4-[6-[(4-chlorophenyl)-methyl-carbamoyl]-8-methyl-imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.09 [methyl N-[5-[6-[(4-fluoro-3-methyl-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.10 [methyl N-[5-[6-[(4-cyano-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.11 [methyl N-[5-[6-[(4-chloro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.12 [methyl N-[5-[6-[(4-fluorophenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.13 [methyl N-[5-[6-[(4-fluorophenyl)-methyl-carbamoyl]-7-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.14 [methyl N-[5-[6-[cyanomethyl-(4-fluoro-3-methoxy-phenyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.15 [methyl N-[5-[6-[(4-chloro-3-methoxy-phenyl)-(cyanomethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.16 [methyl N-[5-[6-[(4-fluoro-3-methoxy-phenyl)-(methylsulfonylmethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.17 [N-(4-fluoro-3-methoxy-phenyl)-3-[6-(methoxycarbamoylamino)-3-pyridyl]-N-methyl-imidazo[1,2-a]pyridine-6-carboxamide] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.18 [methyl N-[5-[8-acetamido-6-[(4-fluorophenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.19 [methyl N-[5-[6-[ethyl-(4-fluorophenyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.20 [methyl N-[5-[8-acetamido-6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.21 [methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.22 [methyl N-[5-[6-[(2-methoxy-4-pyridyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In another preferred composition according to the invention, component (A) is compound no. X.23 [methyl N-[5-[6-[(4-cyano-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 15:1 to 1:30.

In a more preferred composition according to the invention, component (A) is compound no. X.01 [methyl N-[5-[6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.02 [methyl N-[5-[6-[(4-fluoro-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.03 [methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.04 [methyl N-[5-[6-[(4-fluoro-3-methoxy-phenyl)-(methoxymethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.05 [methyl N-[4-[6-[(4-fluoro-3-methoxy-phenyl)-methyl-carbamoyl]imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.06 [3-[6-(cyclopropanecarbonylamino)-3-pyridyl]-N-(4-fluoro-3-methoxy-phenyl)-N-methyl-imidazo[1,2-a]pyridine-6-carboxamide] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.07 [methyl N-[4-[6-[(4-fluorophenyl)-methyl-carbamoyl]imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.08 [methyl N-[4-[6-[(4-chlorophenyl)-methyl-carbamoyl]-8-methyl-imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.09 [methyl N-[5-[6-[(4-fluoro-3-methyl-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.10 [methyl N-[5-[6-[(4-cyano-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.11 [methyl N-[5-[6-[(4-chloro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.12 [methyl N-[5-[6-[(4-fluorophenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.13 [methyl N-[5-[6-[(4-fluorophenyl)-methyl-carbamoyl]-7-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.14 [methyl N-[5-[6-[cyanomethyl-(4-fluoro-3-methoxy-phenyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.15 [methyl N-[5-[6-[(4-chloro-3-methoxy-phenyl)-(cyanomethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.16 [methyl N-[5-[6-[(4-fluoro-3-methoxy-phenyl)-(methylsulfonylmethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.17 [N-(4-fluoro-3-methoxy-phenyl)-3-[6-(methoxycarbamoylamino)-3-pyridyl]-N-methyl-imidazo[1,2-a]pyridine-6-carboxamide] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.18 [methyl N-[5-[8-acetamido-6-[(4-fluorophenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.19 [methyl N-[5-[6-[ethyl-(4-fluorophenyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.20 [methyl N-[5-[8-acetamido-6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.21 [methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.22 [methyl N-[5-[6-[(2-methoxy-4-pyridyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In another more preferred composition according to the invention, component (A) is compound no. X.23 [methyl N-[5-[6-[(4-cyano-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 15:1 to 1:30.

In a still more preferred composition according to the invention, component (A) is compound no. X.01 [methyl N-[5-[6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.02 [methyl N-[5-[6-[(4-fluoro-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.03 [methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.04 [methyl N-[5-[6-[(4-fluoro-3-methoxy-phenyl)-(methoxymethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no.

X.05 [methyl N-[4-[6-[(4-fluoro-3-methoxy-phenyl)-methyl-carbamoyl]imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.06 [3-[6-(cyclopropanecarbonylamino)-3-pyridyl]-N-(4-fluoro-3-methoxy-phenyl)-N-methyl-imidazo[1,2-a]pyridine-6-carboxamide] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.07 [methyl N-[4-[6-[(4-fluorophenyl)-methyl-carbamoyl]imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.08 [methyl N-[4-[6-[(4-chlorophenyl)-methyl-carbamoyl]-8-methyl-imidazo[1,2-a]pyridin-3-yl]phenyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.09 [methyl N-[5-[6-[(4-fluoro-3-methyl-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.10 [methyl N-[5-[6-[(4-cyano-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.11 [methyl N-[5-[6-[(4-chloro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.12 [methyl N-[5-[6-[(4-fluorophenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.13 [methyl N-[5-[6-[(4-fluorophenyl)-methyl-carbamoyl]-7-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.14 [methyl N-[5-[6-[cyanomethyl-(4-fluoro-3-methoxy-phenyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.15 [methyl N-[5-[6-[(4-chloro-3-methoxy-phenyl)-(cyanomethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.16 [methyl N-[5-[6-[(4-fluoro-3-methoxy-phenyl)-(methylsulfonylmethyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.17 [N-(4-fluoro-3-methoxy-phenyl)-3-[6-(methoxycarbamoylamino)-3-pyridyl]-N-methyl-imidazo[1,2-a]pyridine-6-carboxamide] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.18 [methyl N-[5-[8-acetamido-6-[(4-fluorophenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.19 [methyl N-[5-[6-[ethyl-(4-fluorophenyl)carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.20 [methyl N-[5-[8-acetamido-6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.21 [methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.22 [methyl N-[5-[6-[(2-methoxy-4-pyridyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In another still more preferred composition according to the invention, component (A) is compound no. X.23 [methyl N-[5-[6-[(4-cyano-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide, wherein the weight ratio of component (A) to component (B) is from 10:1 to 1:10 (or even more preferably, 5:1 to 1:5).

In a most preferred composition according to the invention, component (A) is compound no. X.01 [methyl N-[5-[6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.02 [methyl N-[5-[6-[(4-fluoro-3-methoxy-phenyl)-methyl-carbamoyl]-8-methyl-imidazo[1,2-a]pyridin-3-yl]-2-pyridyl]carbamate ] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.03 [methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.04 [methyl N-[5-[6-[(4-fluoro-3-methoxy-phenyl)-(methoxymethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.05 [methyl N-[4-[6-[(4-fluoro-3-methoxy-phenyl)-methyl-carbamoyl]imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.06 [3-[6-(cyclopropanecarbonylamino)-3-pyridyl]-N-(4-fluoro-3-methoxy-phenyl)-N-methyl-imidazo[1,2-a]pyridine-6-carboxamide] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.07 [methyl N-[4-[6-[(4-fluorophenyl)-methyl-carbamoyl]imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.08 [methyl N-[4-[6-[(4-chlorophenyl)-methyl-carbamoyl]-8-methyl-imidazo[1,2-a]pyridin-3-yl]phenyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.09 [methyl N-[5-[6-[(4-fluoro-3-methyl-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.10 [methyl N-[5-[6-[(4-cyano-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.11 [methyl N-[5-[6-[(4-chloro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.12 [methyl N-[5-[6-[(4-fluorophenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.13 [methyl N-[5-[6-[(4-fluorophenyl)-methyl-carbamoyl]-7-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.14 [methyl N-[5-[6-[cyanomethyl-(4-fluoro-3-methoxy-phenyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.15 [methyl N-[5-[6-[(4-chloro-3-methoxy-phenyl)-(cyanomethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.16 [methyl N-[5-[6-[(4-fluoro-3-methoxy-phenyl)-(methylsulfonylmethyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.17 [N-(4-fluoro-3-methoxy-phenyl)-3-[6-(methoxycarbamoylamino)-3-pyridyl]-N-methyl-imidazo[1,2-a]pyridine-6-carboxamide] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.18 [methyl N-[5-[8-acetamido-6-[(4-fluorophenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.19 [methyl N-[5-[6-[ethyl-(4-fluorophenyl)carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.20 [methyl N-[5-[8-acetamido-6-[(4-fluoro-3-methoxy-phenyl)-methyl-carbamoyl]imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.21 [methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.22 [methyl N-[5-[6-[(2-methoxy-4-pyridyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In another most preferred composition according to the invention, component (A) is compound no. X.23 [methyl N-[5-[6-[(4-cyano-3-methoxy-phenyl)-methyl-carbamoyl]-8-methyl-imidazo[1,2-a]pyridin-3-yl]-2-pyridyl]carbamate] or a salt, or N-oxide thereof, and component (B) is a compound selected from the group consisting of acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, and Compound Z (feneptamidoquin), wherein the weight ratio of component (A) to component (B) is from 10:1 to 1:10 (or even more preferably, 5:1 to 1:5).

In any of the preferred compositions according to the invention, the composition may comprise an additional active ingredient component (C), which is different to component (B), and is selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide.

The composition according to the present invention can be used in the agricultural sector and related fields of use for controlling plant pests or on non-living materials for control of spoilage microorganisms or organisms potentially harmful to man. The composition can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use the composition as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection. The composition according to the present invention can comprise a fungicidally effective amount of the component A and/or the component B.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

It is also possible to use the composition according to the present invention as dressing agents for the treatment of plant propagation material for the protection against fungal infections as well as against phytopathogenic fungi occurring in the soil. The propagation material can be treated with a composition according to the present invention before planting: seed, for example, can be dressed before being sown. The composition according to the present invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 19th Ed., British Crop Protection Council 2021.

Furthermore the composition according to the present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The composition according to the invention is effective against harmful microorganisms, such as microorganisms, that cause phytopathogenic diseases, in particular against phytopathogenic fungi and bacteria.

The composition of the invention may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes, and more preferably in the Oomycete classes.

The composition of the invention is effective in controlling a broad spectrum of plant diseases, such as foliar pathogens of ornamental, turf, vegetable, field, cereal, and fruit crops.

These pathogens may include:
Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare, Pythium sylvaticum and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola; Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f. sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae; Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f. sp. Hordei, Puccinia striiformis f. sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries;
Blastocladiomycetes, such as Physoderma maydis;
Mucoromycetes, such as Choanephora cucurbitarum.; Mucor spp.; Rhizopus arrhizus; as well as diseases caused by other species and genera closely related to those listed above.

In addition to their fungicidal activity, the composition according to the present invention may also have activity against bacteria such as Erwinia amylovora, Erwinia caratovora, Xanthomonas campestris, Pseudomonas syringae, Strptomyces scabies and other related species as well as certain protozoa.

Within the scope of the present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 6-endotoxins, vegetative insecticidal proteins (Vip), insecticidal proteins of bacteria colonising nematodes, and toxins produced by scorpions, arachnids, wasps and fungi. An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Toxins that can be expressed by transgenic plants include, for example, insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO03/018810).

More examples of such toxins or transgenic plants capable of synthesising such toxins are disclosed, for example, in EP-A-0 374 753, WO93/07278, WO95/34656, EP-A-0 427 529, EP-A-451 878 and WO03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard 1® (cotton variety that expresses a Cry1Ac toxin); Bollgard I1 (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cryl F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Compounds of formula (I) used in accordance with the present invention can be made as shown in the following schemes 1 to 19, in which, unless otherwise stated, the definition of each variable is as defined in the present invention.

Compounds of formula (I) can be prepared via Suzuki cross coupling of compounds of formula (II), wherein X is chloro (Cl), bromo (Br) or iodo (I), and a compound of formula (III), wherein either R⁷ is independently from each other hydrogen, C₁-C₆ alkyl or wherein two R⁷ together can form a C₃-C₈ cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. This transformation is depicted in Scheme 1.

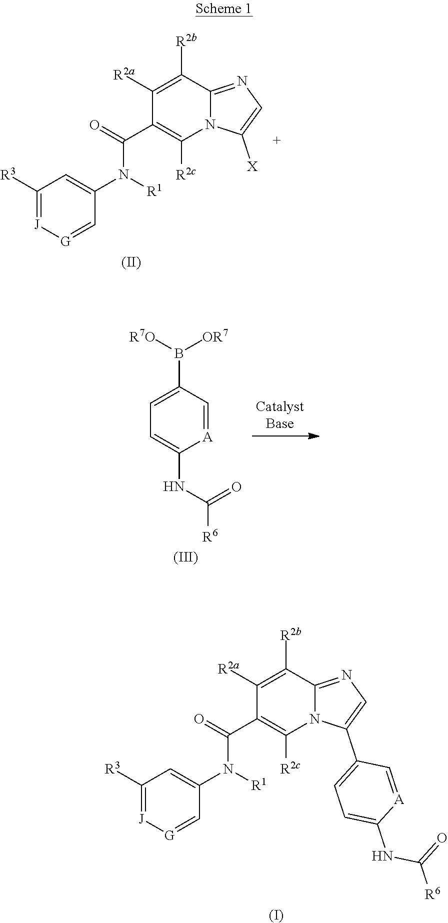

Compounds of formula (II), wherein X is Cl, Br or I, can be prepared by the reaction of a compound of formula (IV), wherein X is Cl, Br or I, with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such, as triethylamine, ethyldiisopropylamine or N-methylmorpholine in a suitable solvent such ethyl acetate, dimethylformamide, tetrahydrofuran or dichloromethane. This transformation is depicted in Scheme 2.

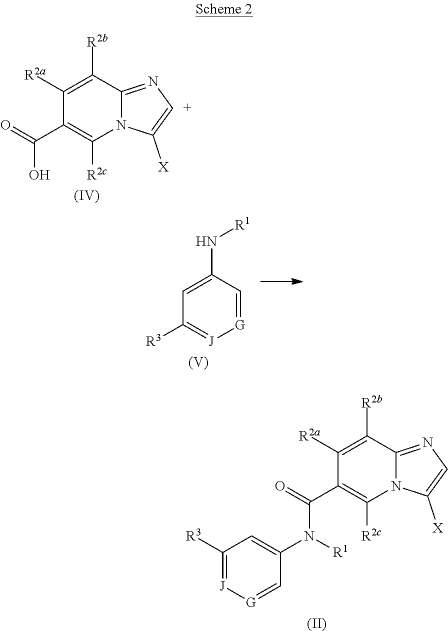

Compounds of formula (IV), wherein X is Cl, Br or I, are commercially available or, alternatively can be prepared by the saponification of compounds of formula (VI), wherein X is Cl, Br or I and R⁸ is a C₁-C₆ alkyl, using a base, such as NaOH or LiOH, in a suitable solvent such as methanol, ethanol or water at temperature between retention time (RT) and reflux. This transformation is depicted in Scheme 3.

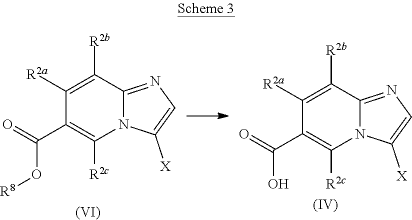

Alternatively, compounds of formula (II), wherein X is Cl, Br or I, can be prepared directly by the reaction of a compounds of formula (VI), wherein X is Cl, Br or I and R⁸ is a C₁-C₆ alkyl, and a compounds of formula (V) in the presence of (CH₃)₃Al or Bis(trimethylaluminum)-1,4-diazabicyclo[2.2.2]octane adduct in a suitable solvent, such as tetrahydrofuran or toluene. These transformations have been described in the literature (see for examples: Weinreb, S. M. et al. Tetrahedron Lett. 1977, 48, 4171; Woodward, S. et al. in Tetrahedron Letters 2006, 47, 5767; Woodward S. et al. in Org. Process Res. Dev. 2015, 19, 831). This transformation is depicted in scheme 4.

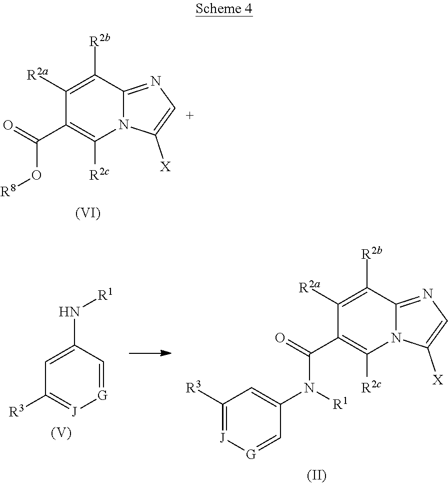

Compounds of formula (VI), wherein X is Cl, Br or I and R⁸ is a C₁-C₆ alkyl, are commercially available or, alternatively, can be prepared from the reaction of a compound of formula (VII), wherein R⁸ is a C₁-C₆ alkyl, and a halogenating agent, such as N-chlorosuccinimide, N-bromosuccinimide, N-iodosuccinimide or bromine in a suitable solvent, such as dichloromethane, chloroform, tetrahydrofuran or acetonitrile. This transformation is depicted in scheme 5.

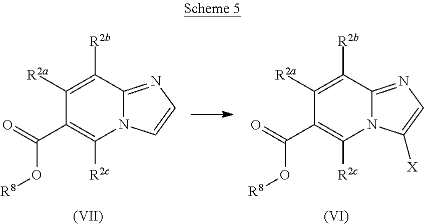

Compounds of formula (VII), wherein R⁸ is a C₁-C₆ alkyl, are commercially available or, alternatively, can be prepared by the reaction of a compound of formula (VIII), wherein X is Cl, Br or I, with carbon monoxide and an alcohol R⁸OH, wherein R⁸ is a C₁-C₆ alkyl, in the presence of a catalyst, such as bis(diphenylphosphino)ferrocene]dichloropalladium(II), and, optionally, a base such as triethylamine. This transformation is depicted in scheme 6.

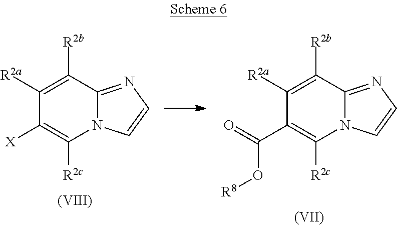

Compounds of formula (VIII) are commercially available or, alternatively, can be prepared by the reaction of a compound of formula (IX), wherein X is Cl, Br or I, and a compound of formula (X), wherein X is Cl, Br or I, or its corresponding acetal of formula (XI), wherein X is Cl, Br or I and either R⁹ is independently from each other C₁-C₆ alkyl or wherein two R⁹ together can form a C₃-C₈ cycloalkyl, in a solvent, such as water, ethanol, acetone or acetonitrile. In some instance, the outcome of the reaction can be improved by using a base, such as sodium bicarbonate or potassium carbonate, or by using an acid, such as p-toluenesulfonic acid or hydrogen bromide. Additionally, this transformation can be utilized to prepare compounds of formula (VII), wherein R⁸ is a C₁-C₆ alkyl, from a compound of formula (XII), wherein R⁸ is a C₁-C₆alkyl, and to prepare compounds of formula (XIII) from a compound of formula (XIV). These transformations are depicted in scheme 7

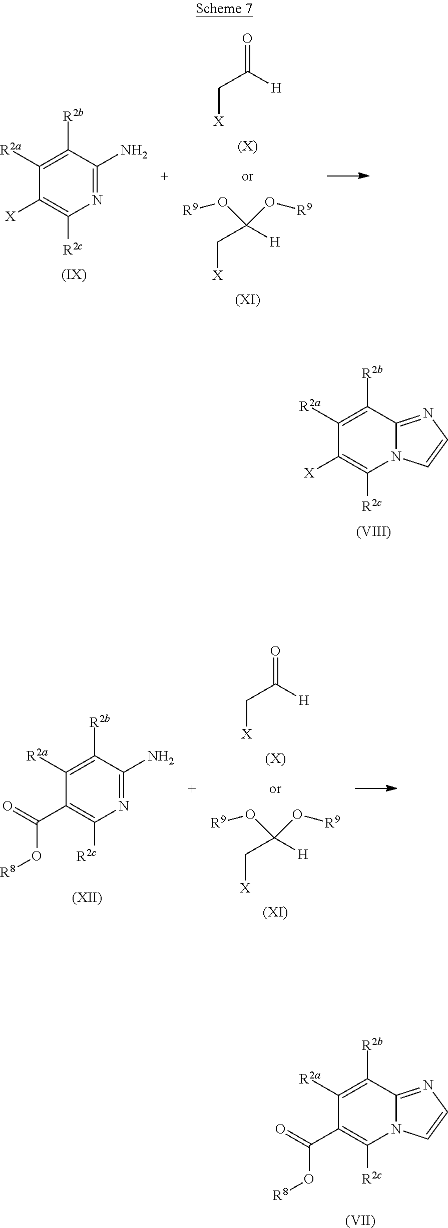

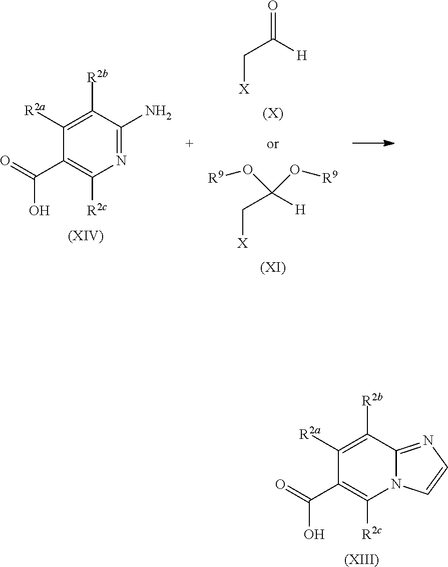

Compounds of formula (IX), wherein X is Cl, Br or I, compounds of formula (XII), wherein R⁸ is a C₁-C₆ alkyl, and compounds of formula (XIV) are prepared by known methods or are commercially available.

Alternatively, compounds of formula (I) can be prepared by the reaction of a compound of formula (XV), with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphophonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. This transformation is depicted in Scheme 8.

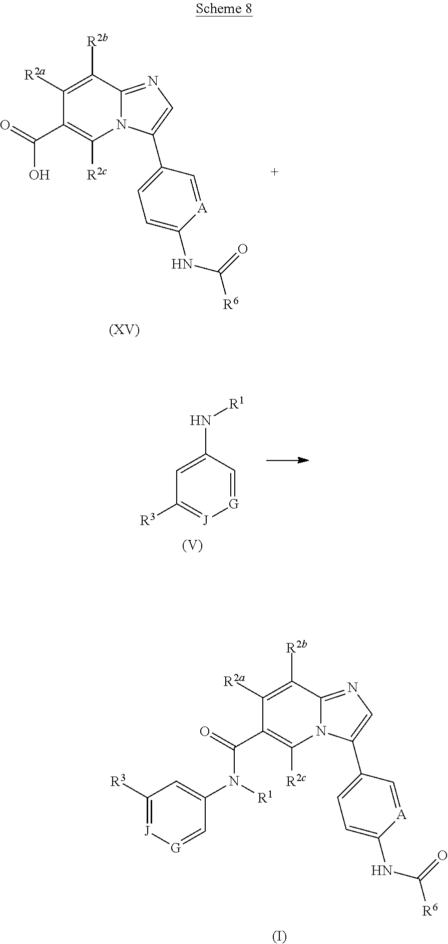

Compound of formula (XV) can be prepared by the saponification of compounds of formula (XVI), wherein R⁸ is a C₁-C₆ alkyl, using a base such as NaOH or LiOH, in a suitable solvent such as methanol, ethanol or water at temperature between room temperature and reflux. This transformation is depicted in Scheme 9.

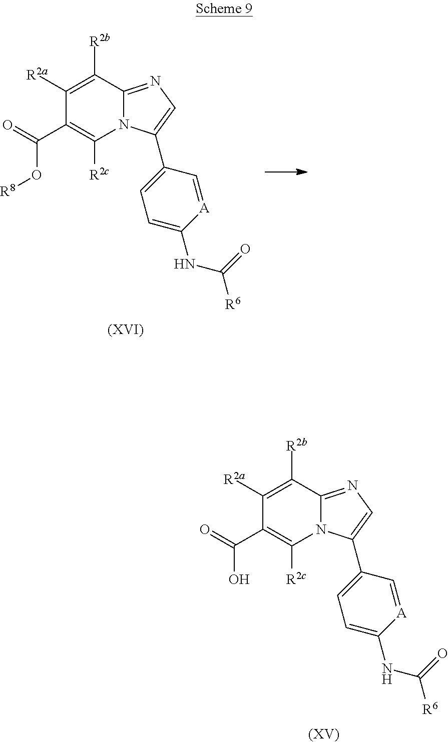

Compounds of formula (XVI), wherein R⁸ is a C₁-C₆ alkyl, can be prepared via Suzuki cross coupling of compounds of formula (VI), wherein X is Cl, Br or I, and R⁸ is C₁-C₆ alkyl, and a compound of formula (III), wherein either R⁷ is independently from each other hydrogen, C₁-C₆ alkyl or wherein two R⁷ together can form a C₃-C₈ cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. Additionally, this transformation can be utilized to prepare a compound of formula (XV) from a compound of formula (IV). These transformations are depicted in Scheme 10.

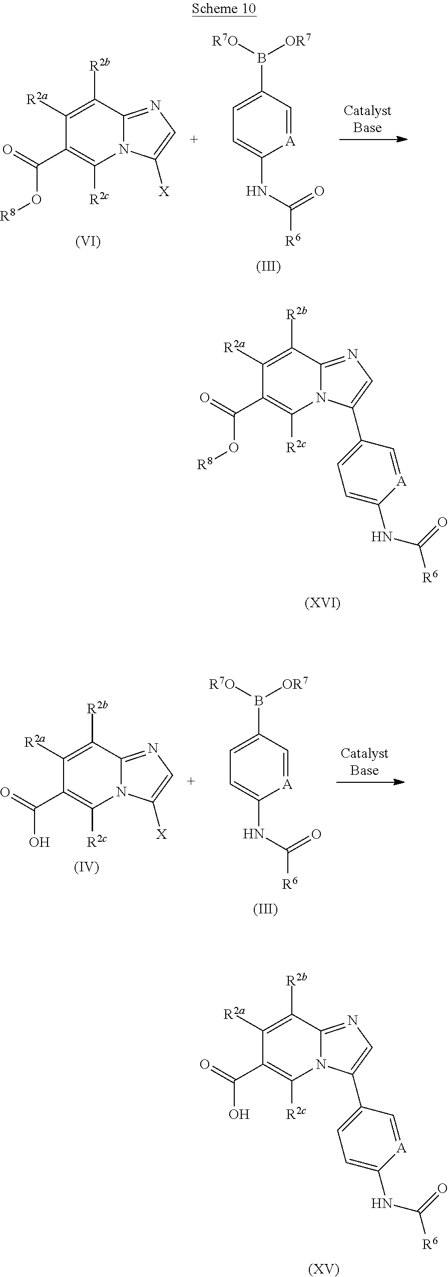

Alternatively, compounds of formula (II), wherein X is Cl, Br or I, can be prepared by the reaction of a compound of formula (XVII) and a halogenating agent, such as N-chlorosuccinimide, N-bromosuccinimide, N-iodosuccinimide or bromine in a suitable solvent, such as dichloromethane, chloroform, tetrahydrofuran or acetonitrile. This transformation is depicted in scheme 11.

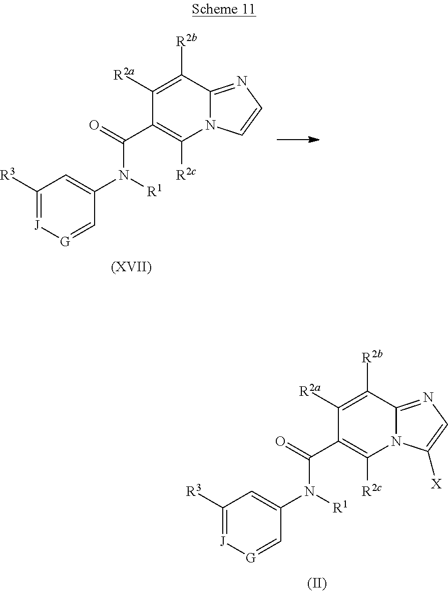

Compounds of formula (XVII) can be prepared by the reaction of a compound of formula (XIII), with a compounds of formula (V) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphophonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. This transformation is depicted in Scheme 12.

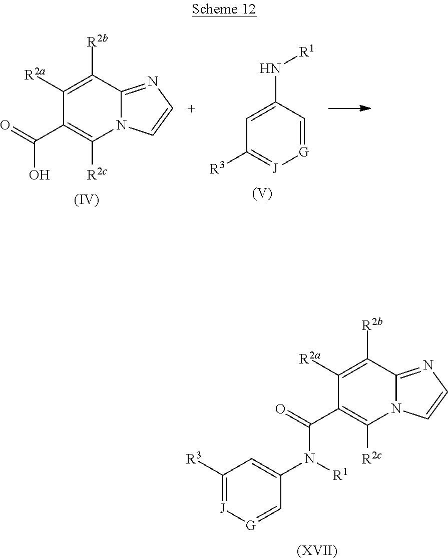

Alternatively, compounds of formula (XVII) can be synthesized by reacting compounds of formula (VIII), wherein X is Cl, Br or I, with amines of formula (V) and carbon monoxide in the presence of a catalyst such as [1,1-bis(diphenylphosphino)ferrocene]dichloropalladium(II), and, optionally, a base such as triethylamine or sodium carbonate. This transformation is described in Scheme 13.

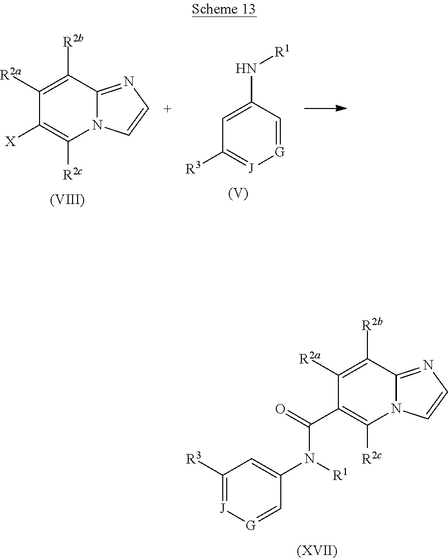

Alternatively, compounds of formula (II) can be prepared by the reaction of a compound of formula (XVIII), with a compounds of formula (XIX), wherein Y is Cl, Br, I, OSO₂CF₃, OSO₂C₆H₄CH₃ or OSO₂CH₃, in the presence of a base, such as Cs₂CO₃, K₂CO₃ or NaOtBu. This transformation is depicted in scheme 14.

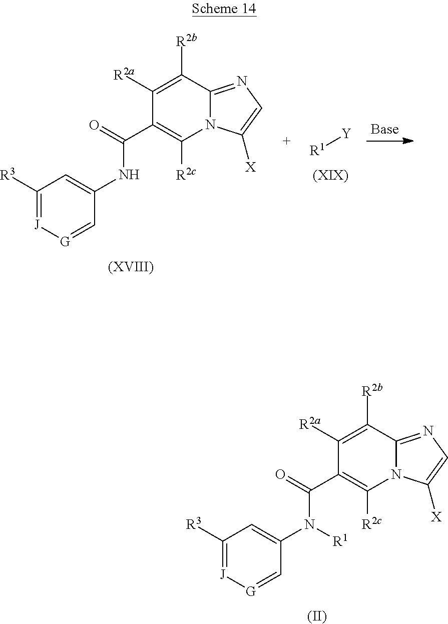

Compounds of formula (XVIII), wherein X is Cl, Br or I, can be prepared by the reaction of a compound of formula (IV), wherein X is Cl, Br or I, with a compounds of formula (XX) and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such, as triethylamine, ethyldiisopropylamine or N-methylmorpholine in a suitable solvent such ethyl acetate, dimethylformamide, tetrahydrofuran or dichloromethane. This transformation is depicted in Scheme 15.

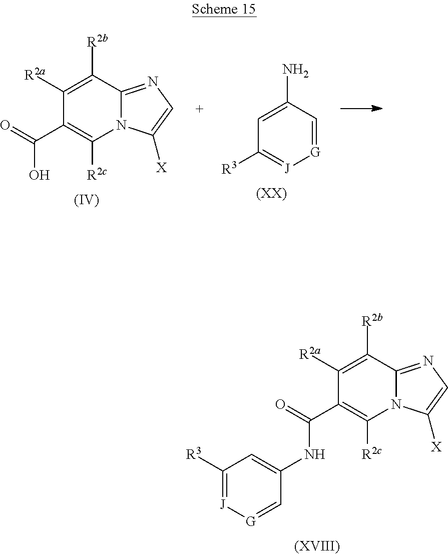

Alternatively, compounds of formula (I) can be synthesized by reacting compounds of formula (XXI), wherein X is Cl, Br or I, with amines of formula (V) and carbon monoxide in the presence of a catalyst such as [1,1-bis(diphenylphosphino)ferrocene]dichloropalladium(II), and, optionally, a base such as triethylamine or sodium carbonate. This transformation is described in Scheme 16.

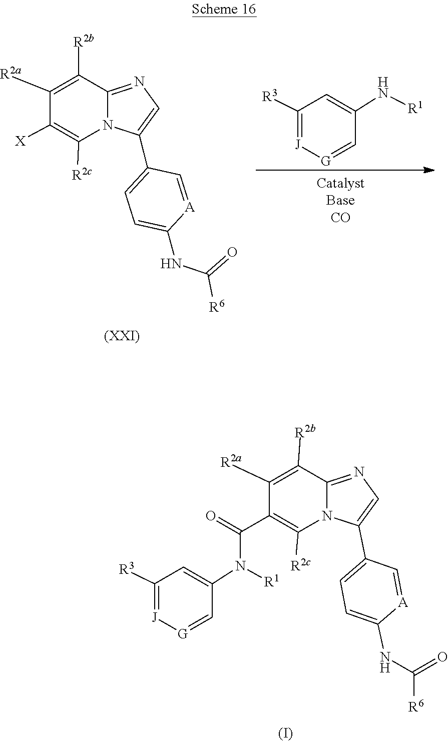

Compounds of formula (XXI), wherein X is Cl, Br or I, can be prepared from compounds of formula (XXII), wherein either X is independently from each other Cl, Br or I, through a Suzuki cross-coupling with compounds of formula (III), wherein either R⁷ is independently from each other hydrogen, C₁₋₆alkyl or wherein two R⁷ together can form a C₃₋₈cycloalkyl, in the presence of a base, such as Cs2CO₃, K₂CO₃or NaOtBu, and a suitable palladium catalyst, such as tetrakistriphenylphosphinepalladium, [1,1-bis(diphenylphosphino)ferrocene]dichloropalladium(II), bis(diphenylphosphine)palladium(II) chloride, palladium dichloride or palladium acetate, in a suitable solvent, such as dimethylformamide, dioxane, tetrahydrofuran, ethanol or water. Compounds of formula (XXII), wherein either X is independently from each other Cl, Br or I, can be prepared by halogenation of compounds of formula (VII), wherein X is Cl, Br or I, using a halogenating reagent such as N-chlorosuccinimide, N-bromosuccinimide, N-iodosuccinimide or bromine in a suitable solvent, such as dichloromethane, chloroform, tetrahydrofuran or acetonitrile. These transformations are depicted in Scheme 17.

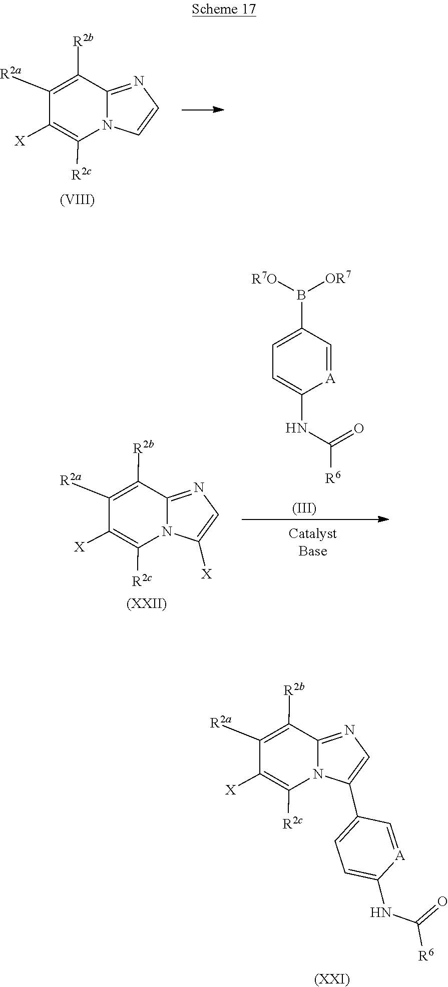

Alternatively, compounds of formula (I) can be prepared by the reaction of a compound of formula (XXIII) with a compound of formula (XXIV), wherein Y is OH, and a coupling agent, such as N,N′-dicyclohexylcarbodiimide, bis(2-oxo-3-oxazolidinyl)phosphinic chloride, 2-(1H-benzotriazole-1-yl)-1,1,3,3-tetramethyluronium hexafluorophosphate, 1-(3-dimethylaminopropyl)-3-ethylcarbodiimide hydrochloride, propylphosphonic anhydride or cyanuric chloride, and, optionally, a base such as triethylamine, ethyldiisopropylamine or N-methylmorpholine in a suitable solvent such ethyl acetate, dimethylformamide, tetrahydrofuran or dichloromethane. The transformation can also be accomplished by the reaction of a compound of formula (XXIII) with a compound of formula (XXIV), wherein Y is Cl, and, optionally, a base such as, triethylamine, ethyldiisopropylamine or pyridine in a suitable solvent such as ethyl acetate, pyridine or tetrahydrofuran. This transformation is depicted in Scheme 18.

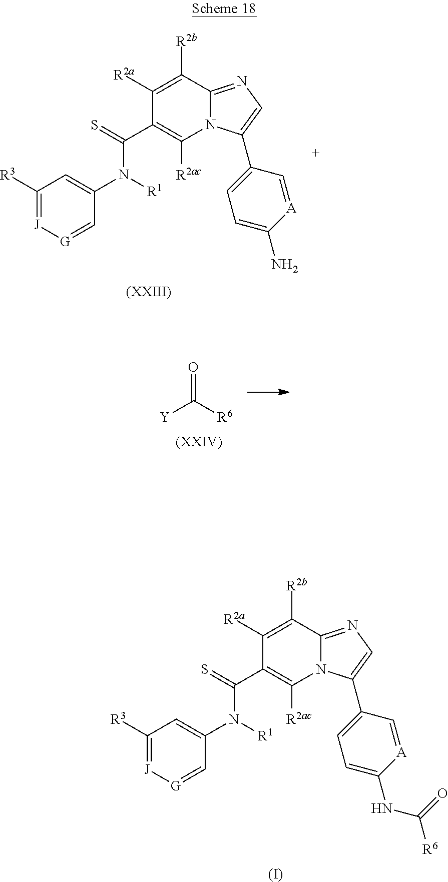

Compounds of formula (XXIII) can be prepared via Suzuki cross coupling of compounds of formula (II), wherein X is Cl, Br or I, and a compound of formula (XXV), wherein either R⁷ is independently from each other hydrogen, C₁-C₆ alkyl or wherein two R⁷ together can form a C₃—C cycloalkyl, in the presence of a base, such as Cs₂CO₃, K₂CO₃ or NaOtBu, and a suitable palladium catalyst, such as tetrakistriphenylphosphinepalladium, palladium dichloride, [1,1-bis(diphenylphosphino)ferrocene]dichloropalladium(II), palladium acetate or bis(diphenylphosphine)palladium(II) chloride), in a suitable solvent, such as dimethylformamide, dioxane, tetrahydrofuran, ethanol or water. This transformation is depicted in Scheme 19.

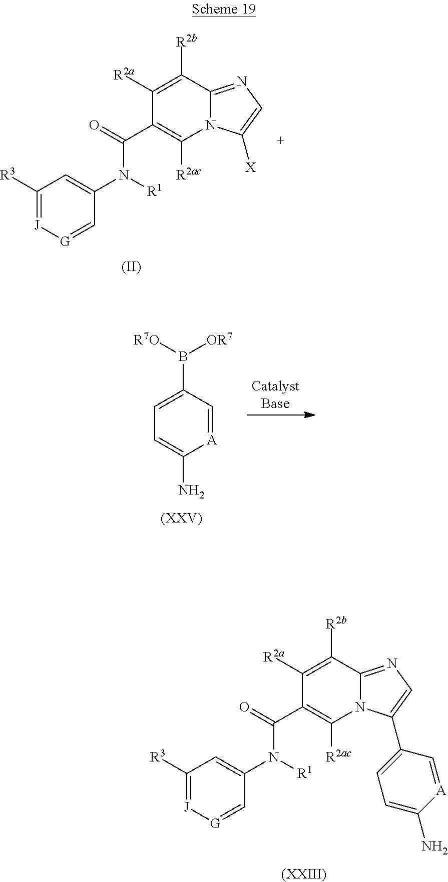

Compounds of formula (V) and of formula (XX) are prepared by known methods or are commercially available.

Compounds of formula (III), wherein either R⁷ is independently from each other hydrogen, C₁-C₆ alkyl or wherein two R⁷ together can form a C₃-C₈ cycloalkyl are prepared by known methods or are commercially available.

It is understood that a person skilled in the art would recognize that the amide coupling reactions described above between an acid, an amine and a coupling agent could also be performed using the corresponding acid chloride and amine. The transformation of an acid into its corresponding acid chloride is well known to the person skilled in the art.

Alternatively, the compounds of formula (I) according to the present invention can be obtained by using standard synthesis techniques known to the person skilled in the art. Non-exhaustive examples include oxidation reactions, reduction reactions, hydrolysis reactions, coupling reactions, aromatic nucleophilic or electrophilic substitution reactions, nucleophilic substitution reactions, nucleophilic addition reactions, olefination reactions, oxime formation, alkylation and halogenation reactions.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

The present invention also provides intermediates useful for the preparation of the compound of formula (I) according to the present invention.

The below intermediate forms a further aspect of the invention.

A compound of formula (XXI)

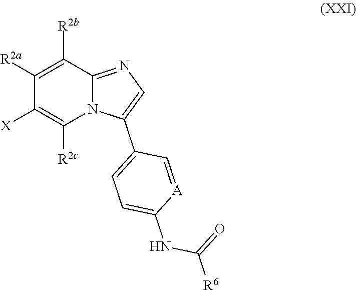

wherein A is CH or N;
R^{2a}, R^{2b} and R^{2c} independently selected from H, hydroxy, halogen, CN, C₁₋₆alkyl, C₃₋₆cycloalkyl, C₁₋₆alkoxy-C₁₋₆alkyl, C₃₋₆cycloalkyl-C₁₋₄alkyl, C₁₋₆alkylsulfanyl, C₁₋₆alkylsulfinyl, C₁₋₆alkylsulfonyl, C₁₋₆alkoxy, amino, and NHC(O)C₁₋₆alkyl; and preferably R², R^{2b} and R^{2c} are independently selected from H, halogen, CN, C₁₋₄alkyl and C₁₋₄alkoxy; and more preferably R^{2a} is selected from H, C₁₋₆alkyl, C₃₋₆cycloalkyl, and C₁₋₆alkoxy-C₁₋₆alkyl, R^{2b} is selected from H, CN, C₁₋₆alkyl, C₃₋₆cycloalkyl, C₁₋₆alkoxy-C₁₋₆alkyl, amino, and —NHC(O)C₁₋₆alkyl, and R^{2c} is H;
R⁶ is selected from C₁₋₆alkyl, C₁₋₆alkoxy, C₃₋₆cycloalkyl, C₃₋₆cycloalkyl-C₁,-alkyl, C₁₋₆alkoxyC₁₋₆alkyl, C₁₋₆alkylamino, diC₁₋₆alkylamino, C₁₋₆alkoxyamino, and C₁₋₆alkylC₁₋₆alkoxyamino, wherein each of said groups is optionally substituted with one to three substituents independently selected from halogen and CN; preferably R⁶ is selected from C₁₋₄alkyl, C₁₋₄alkoxy, C₃₋₆cycloalkyl and C₃₋₆cycloalkyl-C₁₋₃alkyl, wherein each of said groups is optionally substituted with one to three substituents independently selected from halogen; and preferably R⁶ is selected from C₁₋₆alkyl, C₁₋₆alkoxy, C₃₋₆cycloalkyl, C₁-6alkylamino, C₁₋₆alkoxyamino, and C₁₋₆alkylC₁₋₆alkoxyamino; and
X is chloro (CI), bromo (Br), or iodo (1);
or a salt or N-oxide thereof.

The composition according to the present invention may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and/or adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the components A and B in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and/or carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

Compositions of this invention, including all of the above disclosed embodiments and preferred examples thereof, can be mixed with one or more further pesticides including further fungicides, insecticides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection.

Examples of additional active ingredient component (C) are as follows, wherein the term “TX” represents a compound according to the definition of component (A) of the composition of the present invention selected from compound no. X.01, X.02, X.03, X.04, X.05, X.06, X.07, X.08, X.09, X.10, X.11, X.12, X.13, X.14, X.15, X.16, X.17, X.18, X.19, X.20, X.21, X.22, or X.23, as defined in the Table X above or Table T1 below):
(7E,9Z)-dodeca-7,9-dien-1-yl acetate+TX, (9Z,11 E)-tetradeca-9,11-dien-1-yl acetate+TX, (9Z,12E)-tetradeca-9,12-dien-1-yl acetate+TX, (E)-6-methylhept-2-en-4-ol+TX, (E)-dec-5-en-1-yl acetate with (E)-dec-5-en-1-ol+TX, (E)-tridec-4-en-1-yl acetate+TX, (E,Z)-tetradeca-4,10-dien-1-yl acetate+TX, (Z)-dodec-7-en-1-yl acetate+TX, (Z)-hexadec-11-en-1-yl acetate+TX, (Z)-hexadec-11-enal+TX, (Z)-hexadec-13-en-i 1-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, 1,2-dibromo-3-chloropropane+TX, 1,2-dichloropropane+TX, 1,2-dichloropropane with 1,3-dichloropropene+TX, 1,3-dichloropropene+TX, 14-methyloctadec-1-ene+TX, 1-hydroxy-1H-pyridine-2-thione+TX, 2-(octylthio)ethanol+TX, 2-chlorophenyl N-methylcarbamate (CPMC)+TX, 3-(4-chlorophenyl)-5-methylrhodanine+TX, 3,4-dichlorotetrahydrothiophene 1,1-dioxide+TX, 4-(quinoxalin-2-ylamino)benzenesulfonamide+TX, 4-methylnonan-5-ol with 4-methylnonan-5-one+TX, 5-methyl-6-thioxo-1,3,5-thiadiazinan-3-ylacetic acid+TX, 6-isopentenylaminopurine+TX, 8-hydroxyquinoline sulfate+TX, abamectin+TX, acequinocyl+TX, acetamiprid+TX, acetoprole+TX, acrinathrin+TX, acynonapyr+TX, Adoxophyes orana GV+TX, afidopyropen+TX, afoxolaner+TX, Agrobacterium radiobacter+TX, AKD-3088+TX, alanycarb+TX, aldicarb+TX, aldoxycarb+TX, allethrin+TX, alpha-cypermethrin+TX, alphamethrin+TX, alpha-multistriatin+TX, Amblyseius spp.+TX, amidoflumet+TX, amino acids+TX, aminocarb+TX, Anagrapha falcifera NPV+TX, Anagrus atomus+TX, Aphelinus abdominalis+TX, Aphidius colemani+TX, Aphidoletes aphidimyza+TX, apholate+TX, Autographa californica NPV+TX, AZ 60541+TX, azadirachtin+TX, azocyclotin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ175 (ATCC Accession No. 55608)+TX, Bacillus sp. AQ177 (ATCC Accession No. 55609)+TX, Bacillus sp. AQ178 (ATCC Accession No. 53522)+TX, Bacillus sphaericus Neide+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus subtilis unspecified+TX, Bacillus thuringiensis AQ52 (NRRL Accession No. B-21619)+TX, Bacillus thuringiensis BD #32 (NRRL Accession No B-21530)+TX, Bacillus thuringiensis Berliner+TX, Bacillus thuringiensis subsp. Aizawai+TX, Bacillus thuringiensis subsp. Israelensis+TX, Bacillus thuringiensis subsp. Japonensis+TX, Bacillus thuringiensis subsp. Kurstaki+TX, Bacillus thuringiensis subsp. Tenebrionis+TX, Bacillus thuringiensis subspec. kurstaki BMP 123+TX, Beauveria bassiana+TX, Beauveria brongniartii+TX, benclothiaz+TX, benomyl+TX, bensultap+TX, benzoximate+TX, benzpyrimoxan+TX, betacyfluthrin+TX, beta-cypermethrin+TX, bethoxazin+TX, bifenazate+TX, bifenthrin+TX, binapacryl+TX, bioallethrin+TX, bioresmethrin+TX, bis(tributyltin) oxide+TX, bisazir+TX, bistrifluron+TX, bisulflufen+TX, brevicomin+TX, broflanilide+TX, brofluthrinate+TX, bromoacetamide+TX, bromophos-ethyl+TX, bronopol+TX, busulfan+TX, butocarboxim+TX, butopyronoxyl+TX, butoxy(polypropylene glycol)+TX, butylpyridaben+TX, cadusafos+TX, calcium arsenate+TX, carbaryl+TX, carbofuran+TX, carbon disulfide+TX, carbosulfan+TX, cartap+TX, CAS number: 1594624-87-9+TX, CAS number: 1922957-47-8+TX, CAS number: 1255091-74-7+TX, CAS number: 1365070-72-9+TX, CAS number: 1445683-71-5+TX, CAS number: 1445684-82-1+TX, CAS number: 1594626-19-3+TX, CAS number: 1594637-65-6+TX, CAS number: 1632218-00-8+TX, CAS number: 1808115-49-2+TX, CAS number: 1922957-46-7+TX, CAS number: 1922957-48-9+TX, CAS number: 1956329-03-5+TX, CAS number: 1990457-52-7+TX, CAS number: 1990457-55-0+TX, CAS number: 1990457-57-2+TX, CAS number: 1990457-66-3+TX, CAS number: 1990457-77-6+TX, CAS number: 1990457-85-6+TX, CAS number: 2032403-97-5+TX, CAS number: 2044701-44-0+TX, CAS number: 2095470-94-1+TX, CAS Number: 2128706-04-5+TX, CAS number: 2128706-05-6+TX, CAS number: 2133042-31-4+TX, CAS number: 2133042-44-9+TX, CAS number: 2171099-09-3+TX, CAS number: 2220132-55-6+TX, CAS number: 2396747-83-2+TX, CAS number: 2408220-91-5+TX, CAS number: 2408220-94-8+TX, CAS number: 2415706-16-8+TX, Piperflanilide (CAS number: 2615135-05-0)+TX, CAS number: 2719848-60-7+TX, CAS number: RNA (Leptinotarsa decemlineata-specific recombinant double-stranded interfering GS2)+TX, chlorantraniliprole+TX, chlordane+TX, chlorfenapyr+TX, chloropicrin+TX, chloroprallethrin+TX, chlorpyrifos+TX, chromafenozide+TX, Chrysoperla carnea+TX, clenpirin+TX, cloethocarb+TX, clothianidin+TX, codlelure+TX, codlemone+TX, copper acetoarsenite+TX, copper dioctanoate+TX, copper hydroxide+TX, copper sulfate+TX, cresol+TX, crufomate+TX, Cryptolaemus montrouzieri+TX, cuelure+TX, cyanofenphos+TX, cyantraniliprole+TX, cybutryne+TX, cyclaniliprole+TX, cyclobutrifluram+TX, cycloprothrin+TX, cycloxaprid+TX, Cydia pomonella GV+TX, cyenopyrafen+TX, cyetpyrafen+TX, cyflumetofen+TX, cyfluthrin+TX, cyhalodiamide+TX, cylohalothrin+TX, cypermethrin+TX, cyphenothrin+TX, cyproflanilide+TX, cyromazine+TX, cytokinins+TX, Dacnusa sibirica+TX, dazomet+TX, DBCP+TX, DCIP+TX, deltamethrin+TX, diafenthiuron+TX, dialifos+TX, diamidafos+TX, dibrom+TX, dibutyl adipate+TX, dibutyl phthalate+TX, dibutyl succinate+TX, dichlofenthion+TX, dichlone+TX, dichlorophen+TX, dicliphos+TX, dicloromezotiaz+TX, diethyltoluamide+TX, diflubenzuron+TX, Diglyphus isaea+TX, dimatif+TX, dimethoate+TX, dimethyl carbate+TX, dimethyl phthalate+TX, dimpropyridaz+TX, dinactin+TX, dinocap+TX, dinotefuran+TX, dioxabenzofos+TX, dipyrithione+TX, disparlure+TX, D-limonene+TX, dodec-8-en-1-yl acetate+TX, dodec-9-en-1-yl acetate+TX, dodeca-8,10-dien-1-yl acetate+TX, dodicin+TX, dominicalure+TX, doramectin+TX, emamectin+TX, emamectin benzoate+TX, empenthrin+TX, Encarsia formosa+TX, endothal+TX, endrin+TX, eprinomectin+TX, epsilon—momfluorothrin+TX, epsilon-metofluthrin+TX, Eretmocerus eremicus+TX, esfenvalerate+TX, ethion+TX, ethiprole+TX, ethoprophos+TX, ethyl 4-methyloctanoate+TX, ethyl hexanediol+TX, ethylene dibromide+TX, etofenprox+TX, etoxazole+TX, etpyrafen+TX, eugenol+TX, Extract of seaweed and fermentation product derived from melasse+TX, Extract of seaweed and fermentation product derived from melasse comprising urea+TX, Extract of seaweed and fermented plant products+TX, Extract of seaweed and fermented plant products comprising phytohormones, vitamins, EDTA-chelated copper, zinc, and iron+TX, famphur+TX, fenaminosulf+TX, fenamiphos+TX, fenazaquin+TX, fenfluthrin+TX, fenitrothion+TX, fenmezoditiaz+TX, fenobucarb+TX, fenothiocarb+TX, fenoxycarb+TX, fenpropathrin+TX, fenpyrad+TX, fenpyroximate+TX, fensulfothion+TX, fenthion+TX, fentin+TX, fentinacetate+TX, fenvalerate+TX, ferric phosphate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone [318290-98-1]+TX, fluensulfone+TX, flufenerim+TX, flufenprox+TX, flufiprole+TX, fluhexafon+TX, flumethrin+TX, fluopyram+TX, flupyradifurone+TX, flupyrimin+TX, flupyroxystrobin+TX, fluralaner+TX, fluvalinate+TX, fluxametamide+TX, formaldehyde+TX, fosthiazate+TX, fosthietan+TX, frontalin+TX, furfural+TX, gamma-cyhalothrin+TX, Gossyplure® (1:1 mixture of the (Z,E) and (Z,Z) isomers of hexadeca-7,11-dien-1-yl-acetate)+TX, grandlure+TX, grandlure I+TX, grandlure II+TX, grandlure III+TX, grandlure IV+TX, Granulovirus+TX, guadipyr+TX, GY-81+TX, halfenprox+TX, halofenozide+TX, Harpin+TX, Helicoverpa armigera Nucleopolyhedrovirus+TX, Helicoverpa zea NPV+TX, Helicoverpa zea Nucleopolyhedrovirus+TX, Heliothis punctigera Nucleopolyhedrovirus+TX, Heliothis virescens Nucleopolyhedrovirus+TX, hemel+TX, hempa+TX, heptafluthrin+TX, heterophos+TX, Heterorhabditis bacteriophora and H. megidis+TX, hexalure+TX, hexamide+TX, hexythiazox+TX, Hippodamia convergens+TX, hydramethylnon+TX, hydrargaphen+TX, hydrated lime+TX, imicyafos+TX, imidacloprid+TX, imiprothrin+TX, Indazapyroxamet+TX, indoxacarb+TX, iodomethane+TX, iprodione+TX, ipsdienol+TX, ipsenol+TX, isamidofos+TX, isazofos+TX, isocycloseram+TX, Isoflualanam (CAS number: 2892524-05-7)+TX, isothioate+TX, ivermectin+TX, japonilure+TX, kappa-bifenthrin+TX, kappa-tefluthrin+TX, kasugamycin+TX, kasugamycin hydrochloride hydrate+TX, kinetin+TX, lambda-cyhalothrin+TX, ledprona+TX, lepimectin+TX, Leptomastix dactylopii+TX, lineatin+TX, litlure+TX, looplure+TX, lotilaner+TX, lufenuron+TX, Macrolophus caliginosus+TX, Mamestra brassicae NPV+TX, mecarphon+TX, medlure+TX, megatomoic acid+TX, metaflumizone+TX, metaldehyde+TX, metam+TX, metam-potassium+TX, metam-sodium+TX, Metaphycus helvolus+TX, Metarhizium anisopliae var. acridum+TX, Metarhizium anisopliae var. anisopliae+TX, Metarhizium spp.+TX, metepa+TX, methiocarb+TX, methiotepa+TX, methomyl+TX, methoquin-butyl+TX, methoxyfenozide+TX, methyl apholate+TX, methyl bromide+TX, methyl eugenol+TX, methyl isothiocyanate+TX, methylneodecanamide+TX, metofluthrin+TX, metolcarb+TX, mexacarbate+TX, milbemectin+TX, milbemycin oxime+TX, momfluorothrin+TX, morzid+TX, moxidectin+TX, muscalure+TX, Muscodor albus 620 (NRRL Accession No. 30547)+TX, Muscodor roseus A3-5 (NRRL Accession No. 30548)+TX, Myrothecium verrucaria composition+TX, nabam+TX, NC-184+TX, Neem tree based products+TX, Neodiprion sertifer NPV and N. Iecontei NPV+TX, nickel bis(dimethyldithiocarbamate)+TX, niclosamide+TX, niclosamide-olamine+TX, nicofluprole+TX, nitenpyram+TX, nithiazine+TX, nitrapyrin+TX, octadeca-2,13-dien-1-yl acetate+TX, octadeca-3,13-dien-1-yl acetate+TX, octhilinone+TX, omethoate+TX, orfralure+TX, Orius spp.+TX, oryctalure+TX, ostramone+TX, oxamate+TX, oxamyl+TX, oxazosulfyl+TX, oxolinic acid+TX, oxytetracycline+TX, Paecilomyces fumosoroseus+TX, Paecilomyces lilacinus+TX, parathion-ethyl+TX, Pasteuria nishizawae+TX, Pasteuria penetrans+TX, Pasteuria ramosa+TX, Pasteuria thornei+TX, Pasteuria usgae+TX, P-cymene+TX, penfluron+TX, pentachlorophenol+TX, permethrin+TX, phenothrin+TX, phorate+TX, phosphamidon+TX, phosphocarb+TX, Phytoseiulus persimilis+TX, picaridin+TX, pioxaniliprole+TX, piperazine+TX, piperonylbutoxide+TX, pirimicarb+TX, pirimiphos-ethyl+TX, pirimiphos-methyl+TX, Plutella xylostella Granulosis virus+TX, Plutella xylostella Nucleopolyhedrovirus+TX, Polyhedrosis virus+TX, potassium and molybdenum and EDTA-chelated manganese+TX, potassium ethylxanthate+TX, potassium hydroxyquinoline sulfate+TX, prallethrin+TX, probenazole+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyrethrum+TX, pyridaben+TX, pyridalyl+TX, pyridin-4-amine+TX, pyrifluquinazon+TX, pyrimidifen+TX, pyriminostrobin+TX, pyriprole [394730-71-3]+TX, pyriprole+TX, pyriproxyfen+TX, QRD 420 (a terpenoid blend)+TX, QRD 452 (a terpenoid blend)+TX, QRD 460 (a terpenoid blend)+TX, Quillaja saponaria+TX, quinoclamine+TX, quinonamid+TX, resmethrin+TX, Rhodococcus globerulus AQ719 (NRRL Accession No B-21663)+TX, sarolaner+TX, S-bioallethrin+TX, sebufos+TX, selamectin+TX, siglure+TX, silafluofen+TX, simazine+TX, sodium pentachlorophenoxide+TX, sordidin+TX, spidoxamat+TX, spinetoram+TX, spinosad+TX, spirobudifen+TX, spirodiclofen+TX, spiromesifen+TX, spiropidion+TX, spirotetramat+TX, Spodoptera exigua multicapsid nuclear polyhedrosis virus+TX, Spodoptera frugiperda Nucleopolyhedrovirus+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Streptomyces galbus (NRRL Accession No. 30232)+TX, Streptomyces sp. (NRRL Accession No. B-30145)+TX, streptomycin+TX, streptomycin sesquisulfate+TX, strychnine+TX, sulcatol+TX, sulfiflumin (CAS number: 2377084-09-6)+TX, sulfoxaflor+TX, tazimcarb+TX, tebufenozide+TX, tebufenpyrad+TX, tebupirimiphos+TX, tecloftalam+TX, tefluthrin+TX, temephos+TX, tepa+TX, terbam+TX, terbufos+TX, terpenoid blend+TX, tetrachlorantraniliprole+TX, tetrachlorothiophene+TX, tetradec-11-en-1-yl acetate+TX, tetradiphon+TX, tetramethrin+TX, tetramethylfluthrin+TX, tetranactin+TX, tetraniliprole+TX, theta-cypermethrin+TX, thiacloprid+TX, thiafenox+TX, thiamethoxam+TX, thiocyclam+TX, thiodicarb+TX, thiofanox+TX, thiohempa+TX, thiomersal+TX, thiometon+TX, thionazin+TX, thiophanate+TX, thiosultap+TX, thiotepa+TX, tigolaner+TX, tiorantraniliprole+TX, tioxazafen+TX, tolfenpyrad+TX, toxaphene+TX, tralomethrin+TX, transfluthrin+TX, tretamine+TX, triazamate+TX, triazophos+TX, triazuron+TX, tributyltin oxide+TX, trichlorfon+TX, trichloronate+TX, trichlorphon+TX, Trichogramma spp.+TX, trifenmorph+TX, trifluenfuronate+TX, triflumezopyrim+TX, trimedlure+TX, trimedlure A+TX, trimedlure B1+TX, trimedlure B2+TX, trimedlure C+TX, trimethacarb+TX, triphenyltin acetate+TX, triphenyltin hydroxide+TX, trunc-call+TX, tyclopyrazoflor+TX, Typhlodromus occidentalis+TX, uredepa+TX, Verticillium lecanii+TX, Verticillium spp.+TX, xylenols+TX, YI-5302+TX, zeatin+TX, zeta-Cypermethrin+TX,
N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427)+TX, (3′,4′,5′-trifluoro-biphenyl-2-yl)-amide+TX, (3-methylisoxazol-5-yl)-[4-[5-(trifluoromethyl)-1,2,4-oxadiazol-3-yl]phenyl]methanone (these compounds may be prepared from the methods described in WO 2017/220485)+TX, (4-phenoxyphenyl)methyl 2-amino-6-methyl-pyridine-3-carboxylate (this compound may be prepared from the methods described in WO 2014/006945)+TX, (5-methyl-2-pyridyl)-[4-[5-(trifluoromethyl)-1,2,4-oxadiazol-3-yl]phenyl]methanone+TX, (7E,9Z)-dodeca-7,9-dien-1-yl acetate+TX, (9Z,11 E)-tetradeca-9,11-dien-1-yl acetate+TX, (9Z,12E)-tetradeca-9,12-dien-1-yl acetate+TX, (E)-6-methylhept-2-en-4-ol+TX, (E)-dec-5-en-1-yl acetate with (E)-dec-5-en-1-ol+TX, (E)-tridec-4-en-1-yl acetate+TX, (E,Z)-tetradeca-4,10-dien-1-yl acetate, +TX, (R)-3-(difluoromethyl)-1-methyl-N-[1,1,3-trimethylindan-4-yl]pyrazole-4-carboxamide+TX, (Z)-dodec-7-en-1-yl acetate+TX, (Z)-hexadec-11-en-1-yl acetate+TX, (Z)-hexadec-11-enal+TX, (Z)-hexadec-13-en-i 1-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707)+TX, (Z,2E)-5-[1-(4-chlorophenyl)pyrazol-3-yl]oxy-2-methoxyimino-N,3-dimethyl-pent-3-enamide+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 1-(6-chloro-7-methyl-pyrazolo[1,5-a]pyridin-3-yl)-4,4-difluoro-3,3-dimethyl-isoquinoline (these compounds may be prepared from the methods described in WO2017/025510)+TX, 1,1-bis(4-chlorophenyl)-2-ethoxyethanol+TX, 1,1-dichloro-2,2-bis(4-ethylphenyl)ethane+TX, 1,2-dibromo-3-chloropropane+TX, 1,2-dichloropropane with 1,3-dichloropropene+TX, 1,3-dichloropropene+TX, 1,3-dimethoxy-1-[[4-[5-(trifluoromethyl)-1,2,4-oxadiazol-3-yl]phenyl]methyl]urea+TX, 1-[2-[[1-(4-chlorophenyl)pyrazol-3-yl]oxymethyl]-3-methyl-phenyl]-4-methyl-tetrazol-5-one+TX, 10-dien-1-yl acetate+TX, 14-methyloctadec-1-ene+TX, 1-bromo-2-chloroethane+TX, 1-dichloro-1-nitroethane+TX, 1-hydroxy-1H-pyridine-2-thione+TX, 1-methoxy-3-methyl-1-[[4-[5-(trifluoromethyl)-1,2,4-oxadiazol-3-yl]phenyl]methyl]urea+TX, 1-methyl-4-[3-methyl-2-[[2-methyl-4-(3,4,5-trimethylpyrazol-1-yl)phenoxy]methyl]phenyl]tetrazol-5-one+TX, 2-(difluoromethyl)—N-((3R)—1, 1, 3-trimethylindan-4-yl) pyridine-3-carboxamide+TX, 2-(difluoromethyl)—N-((3R)—1, 1, 3-trimethylindan-4-yl) pyridine-3-carboxamide+TX, 2-(1,3-dithiolan-2-yl)phenyl dimethylcarbamate+TX, 2-(2-butoxyethoxy)ethyl piperonylate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-(difluoromethyl)-N-(3-ethyl-1,1-dimethyl-indan-4-yl)pyridine-3-carboxamide+TX, 2-(difluoromethyl)-N-[(3R)-3-ethyl-1,1-dimethyl-indan-4-yl]pyridine-3-carboxamide+TX, 2-(difluoromethyl)-N-[(3S)-3-ethyl-1,1-dimethyl-indan-4-yl]pyridine-3-carboxamide (this compound may be prepared from the methods described in WO 2014/095675)+TX, 2-(difluoromethyl)-N-[3-ethyl-1,1-dimethyl-indan-4-yl]pyridine-3-carboxamide+TX, 2-(octylthio)ethanol+TX, 2,2,2-trichloro-1-(3,4-dichlorophenyl)ethyl acetate+TX, 2,2-dichlorovinyl 2-ethylsulfinylethyl methyl phosphate+TX, 2,2-difluoro-N-methyl-2-[4-[5-(trifluoromethyl)-1,2,4-oxadiazol-3-yl]phenyl]acetamide+TX, 2,4-dichlorophenyl benzenesulfonate+TX, 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281)+TX, 2-[2-fluoro-6-[(8-fluoro-2-methyl-3-quinolyl)oxy]phenyl]propan-2-ol+TX, 2-[6-(4-bromophenoxy)-2-(trifluoromethyl)-3-pyridyl]-1-(1,2,4-triazol-1-yl)propan-2-ol (this compound may be prepared from the methods described in WO 2017/029179)+TX, 2-[6-(4-chlorophenoxy)-2-(trifluoromethyl)-3-pyridyl]-1-(1,2,4-triazol-1-yl)propan-2-ol (this compound may be prepared from the methods described in WO 2017/029179)+TX, 2-chlorovinyl diethyl phosphate+TX, 2-fluoro-N-methyl-N-1-naphthylacetamide+TX, 2-imidazolidone+TX, 2-isovalerylindan-1,3-dione+TX, 2-methyl(prop-2-ynyl)aminophenyl methylcarbamate+TX, 2-oxo-N-propyl-2-[4-[5-(trifluoromethyl)-1,2,4-oxadiazol-3-yl]phenyl]acetamide (this compound may be prepared from the methods described in WO 2018/065414)+TX, 2-thiocyanatoethyl laurate+TX, 3-(4,4-difluoro-3,3-dimethyl-1-isoquinolyl)-7,8-dihydro-6H-cyclopenta[e]benzimidazole (these compounds may be prepared from the methods described in WO2016/156085)+TX, 3-(4,4-difluoro-3,4-dihydro-3,3-dimethylisoquinolin-1-yl)quinolone+TX, 3-(4-chlorophenyl)-5-methylrhodanine+TX, 3-(difluoromethyl)-1-methyl-N-[1,1,3-trimethylindan-4-yl]pyrazole-4-carboxamide+TX, 3,4-dichlorotetrahydrothiophene 1,1-dioxide+TX, 3-[2-(1-chlorocyclopropyl)-3-(2-fluorophenyl)-2-hydroxy-propyl]imidazole-4-carbonitrile (this compound may be prepared from the methods described in WO 2016/156290)+TX, 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290)+TX, 3-bromo-1-chloroprop-1-ene+TX, 3-chloro-6-methyl-5-phenyl-4-(2,4,6-trifluorophenyl)pyridazine+TX, 3-difluoromethyl-1-methyl-1H-pyrazole-4-carboxylic acid+TX, 3-ethyl-1-methoxy-1-[[4-[5-(trifluoromethyl)-1,2,4-oxadiazol-3-yl]phenyl]methyl]urea+TX, 3-methyl-1-phenylpyrazol-5-yl dimethylcarbamate+TX, 4-(2-bromo-4-fluorophenyl)—N-(2-chloro-6-fluorophenyl)—1, 3-dimethyl-1H-pyrazol-5-amine+TX, 4-(2,6-difluorophenyl)-6-methyl-5-phenyl-pyridazine-3-carbonitrile+TX, 4-(2-bromo-4-fluoro-phenyl)-N-(2-chloro-6-fluoro-phenyl)-2,5-dimethyl-pyrazol-3-amine+TX, 4-(quinoxalin-2-ylamino)benzenesulfonamide+TX, 4,4-difluoro-1-(5-fluoro-4-methyl-benzimidazol-1-yl)-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 4,4-dimethyl-2-[[4-[5-(trifluoromethyl)-1,2,4-oxadiazol-3-yl]phenyl]methyl]isoxazolidin-3-one+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, 4-chloro-2-(2-chloro-2-methyl-propyl)-5-[(6-iodo-3-pyridyl)methoxy]pyridazin-3-one+TX, 4-chlorophenyl phenyl sulfone+TX, 4-methyl(prop-2-ynyl)amino-3,5-xylyl methylcarbamate+TX, 4-methylnonan-5-ol with 4-methylnonan-5-one+TX, 5-(1,3-benzodioxol-5-yl)-3-hexylcyclohex-2-enone+TX, 5,5-dimethyl-2-[[4-[5-(trifluoromethyl)-1,2,4-oxadiazol-3-yl]phenyl]methyl]isoxazolidin-3-one+TX, 5,5-dimethyl-3-oxocyclohex-1-enyl dimethylcarbamate+TX, 5-amino-1,3,4-thiadiazole-2-thiol zinc salt (2:1)+TX, 5-methyl-6-thioxo-1,3,5-thiadiazinan-3-ylacetic acid+TX, 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391)+TX, 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391)+TX, 6-chloro-4,4-difluoro-3,3-dimethyl-1-(4-methylbenzimidazol-1-yl)isoquinoline+TX, 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391)+TX, 6-ethyl-5,7-dioxo-pyrrolo[4,5][1,4]dithiino[1,2-c]isothiazole-3-carbonitrile+TX, 6-isopentenylaminopurine+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, 8-hydroxyquinoline sulfate+TX, acethion+TX, acetoprole+TX, acibenzolar+TX, acibenzolar-S-methyl+TX, acrylonitrile+TX, Adoxophyes orana GV+TX, Agrobacterium radiobacter+TX, aldoxycarb+TX, aldrin+TX, allosamidin+TX, allyxycarb+TX, alpha-chlorohydrin+TX, alpha-ecdysone+TX, alpha-multistriatin+TX, aluminium phosphide+TX, Amblyseius spp.+TX, amectotractin+TX, ametoctradin+TX, amidithion+TX, amidothioate+TX, aminocarb+TX, aminopyrifen+TX, amisulbrom+TX, amiton+TX, amiton hydrogen oxalate+TX, amitraz+TX, anabasine+TX, Anagrapha falcifera NPV+TX, Anagrus atomus+TX, ancymidol+TX, anilazine+TX, anisiflupurin+TX, anthraquinone+TX, antu+TX, Aphelinus abdominalis+TX, Aphidius colemani+TX, Aphidoletes aphidimyza+TX, apholate+TX, aramite+TX, arsenous oxide+TX, athidathion+TX, Autographa californica NPV+TX, azaconazole+TX, azamethiphos+TX, azobenzene+TX, azothoate+TX, azoxystrobin+TX, Bacillus sphaericus Neide+TX, Bacillus thuringiensis delta endotoxins+TX, barium carbonate+TX, barium hexafluorosilicate+TX, barium polysulfide+TX, barthrin+TX, Bayer 22/190+TX, Bayer 22408+TX, Beauveria brongniartii+TX, benalaxyl+TX, benclothiaz+TX, benomyl+TX, benoxafos+TX, benthiavalicarb+TX, benzothiostrobin+TX, benzovindiflupyr+TX, benzyl benzoate+TX, beta-cyfluthrin+TX, beta-cypermethrin+TX, bethoxazin+TX, bioethanomethrin+TX, biopermethrin+TX, bis(2-chloroethyl) ether+TX, bis(tributyltin) oxide+TX, bisazir+TX, bisthiosemi+TX, bitertanol+TX, bixafen+TX, blasticidin-S+TX, borax+TX, bordeaux mixture+TX, boscalid+TX, brevicomin+TX, brodifacoum+TX, brofenvalerate+TX, bromadiolone+TX, bromethalin+TX, bromfenvinfos+TX, bromoacetamide+TX, bromocyclen+TX, bromo-DDT+TX, bromophos+TX, bromopropylate+TX, bromuconazole+TX, bronopol+TX, bufencarb+TX, bupirimate+TX, buprofezin+TX, busulfan+TX, but-3-ynyl N-[6-[[(Z)-[(1-methyltetrazol-5-yl)-phenyl-methylene]amino]oxymethyl]-2-pyridyl]carbamate+TX, butacarb+TX, butathiofos+TX, butocarboxim+TX, butonate+TX, butopyronoxyl+TX, butoxy(polypropylene glycol)+TX, butoxycarboxim+TX, butylpyridaben+TX, calcium arsenate+TX, calcium cyanide+TX, calcium polysulfide+TX, camphechlor+TX, captafol+TX, captan+TX, carbanolate+TX, carbendazim+TX, carbon disulfide+TX, carbon tetrachloride+TX, carbophenothion+TX, carboxin+TX, cartap hydrochloride+TX, CAS Number: 2132414-04-9+TX, CAS Number: 2344721-61-3+TX, cevadine+TX, chinomethionat+TX, chloralose+TX, chlorbenside+TX, chlorbicyclen+TX, chlordane+TX, chlordecone+TX, chlordimeform+TX, chlordimeform hydrochloride+TX, chlorfenethol+TX, chlorfenson+TX, chlorfensulfide+TX, chlorobenzilate+TX, chloroform+TX, chloroinconazide+TX, chloromebuform+TX, chloromethiuron+TX, chloroneb+TX, chlorophacinone+TX, chloropicrin+TX, chloropropylate+TX, chlorothalonil+TX, chlorphoxim+TX, chlorprazophos+TX, chlorthiophos+TX, chlozolinate+TX, cholecalciferol+TX, Chrysoperla carnea+TX, cinerin I+TX, cinerin II+TX, cinerins+TX, cismethrin+TX, cis-resmethrin+TX, clocythrin+TX, closantel+TX, codlelure+TX, codlemone+TX, copper acetoarsenite+TX, copper arsenate+TX, copper dioctanoate+TX, copper hydroxide+TX, copper naphthenate+TX, copper oleate+TX, copper oxide+TX, copper oxychloride+TX, copper sulfate+TX, coumachlor+TX, coumafuryl+TX, coumaphos+TX, coumatetralyl+TX, coumethoxystrobin (jiaxiangjunzhi)+TX, coumithoate+TX, coumoxystrobin+TX, cresol+TX, crimidine+TX, crotamiton+TX, crotoxyphos+TX, crufomate+TX, cryolite+TX, Cryptolaemus montrouzieri+TX, CS 708+TX, cuelure+TX, cufraneb+TX, cyanofenphos+TX, cyanophos+TX, cyanthoate+TX, cyazofamid+TX, cybutryne+TX, cyclethrin+TX, cyclobutrifluram+TX, Cydia pomonella GV+TX, cyflufenamid+TX, cymiazole+TX, cymoxanil+TX, cyproconazole+TX, cyprodinil+TX, cythioate+TX, cytokinins+TX, Dacnusa sibirica+TX, DAEP+TX, dazomet+TX, DCIP+TX, DCPM+TX, DDT+TX, debacarb+TX, decarbofuran+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, diamidafos+TX, dibutyl adipate+TX, dibutyl phthalate+TX, dibutyl succinate+TX, dicapthon+TX, dichlobentiazox+TX, dichlofenthion+TX, dichlofluanid+TX, dichlone+TX, dichlorophen+TX, dichlorvos+TX, dichlozoline+TX, dicliphos+TX, diclocymet+TX, diclomezine+TX, dicloran+TX, dicresyl+TX, dicyclanil+TX, dicyclopentadiene+TX, dieldrin+TX, dienochlor+TX, diethofencarb+TX, diethyl 5-methylpyrazol-3-yl phosphate+TX, diethyltoluamide+TX, difenacoum+TX, difenoconazole+TX, difethialone+TX, diflovidazin+TX, Diglyphus isaea+TX, dilor+TX, dimatif+TX, dimefluthrin+TX, dimefox+TX, dimetan+TX, dimethirimol+TX, dimethomorph+TX, dimethrin+TX, dimethyl carbate+TX, dimethyl phthalate+TX, dimethylvinphos+TX, dimetilan+TX, dimoxystrobin+TX, dinex+TX, dinex-diclexine+TX, diniconazole+TX, dinocap-4+TX, dinocap-6+TX, dinocton+TX, dinopenton+TX, dinoprop+TX, dinosam+TX, dinoseb+TX, dinosulfon+TX, dinoterbon+TX, diofenolan+TX, dioxabenzofos+TX, dioxathion+TX, diphacinone+TX, diphenyl sulfone+TX, dipymetitrone+TX, dipyrithione+TX, disparlure+TX, disulfiram+TX, dithianon+TX, dithicrofos+TX, DNOC+TX, dodec-8-en-1-yl acetate+TX, dodec-9-en-1-yl acetate+TX, dodeca-8+TX, dodemorph+TX, dodicin+TX, dodine+TX, dofenapyn+TX, dominicalure+TX, doramectin+TX, DSP+TX, d-tetramethrin+TX, ecdysterone+TX, edifenphos+TX, El 1642+TX, EMPC+TX, Encarsia formosa+TX, endothal+TX, endothion+TX, enestroburin+TX, enoxastrobin+TX, EPBP+TX, epoxiconazole+TX, eprinomectin+TX, Eretmocerus eremicus+TX, ergocalciferol+TX, etaphos+TX, ethaboxam+TX, ethiofencarb+TX, ethirimol+TX, ethoate-methyl+TX, ethyl 1-[[4-[(Z)-2-ethoxy-3,3,3-trifluoro-prop-1-enoxy]phenyl]methyl]pyrazole-3-carboxylate (may be prepared from the methods described in WO 2020/056090)+TX, ethyl 1-[[4-[[2-(trifluoromethyl)-1,3-dioxolan-2-yl]methoxy]phenyl]methyl]pyrazole-3-carboxylate (may be prepared from the methods described in WO 2020/056090)+TX, ethyl 1-[[4-[5-(trifluoromethyl)-1,2,4-oxadiazol-3-yl]phenyl]methyl]pyrazole-4-carboxylate+TX, ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365)+TX, ethyl 4-methyloctanoate+TX, ethyl formate+TX, ethyl hexanediol+TX, ethylene dibromide+TX, ethylene dichloride+TX, ethylene oxide+TX, etridiazole+TX, etrimfos+TX, eugenol+TX, EXD+TX, famoxadone+TX, farnesol+TX, farnesol with nerolidol+TX, fenamidone+TX, fenaminosulf+TX, fenaminstrobin+TX, fenarimol+TX, fenazaflor+TX, fenbuconazole+TX, fenbutatin oxide+TX, fenchlorphos+TX, fenethacarb+TX, fenfuram+TX, fenhexamid+TX, fenitrothion+TX, fenothiocarb+TX, fenoxacrim+TX, fenoxanil+TX, fenpiclonil+TX, fenpicoxamid+TX, fenpirithrin+TX, fenpropidin+TX, fenpropimorph+TX, fenpyrad+TX, fenpyrazamine+TX, fenpyroximate+TX, fenson+TX, fensulfothion+TX, fenthion+TX, fenthion-ethyl+TX, fentin+TX, fentrifanil+TX, ferbam+TX, ferimzone+TX, ferric phosphate+TX, flocoumafen+TX, florylpicoxamid+TX, fluazinam+TX, flubeneteram+TX, flubenzimine+TX, flucofuron+TX, flucycloxuron+TX, fludioxonil+TX, fluenetil+TX, flufenoxadiazam+TX, flufenoxystrobin+TX, fluindapyr+TX, flumetylsulforim+TX, flumorph+TX, fluopicolide+TX, fluopimomide+TX, fluopyram+TX, fluorbenside+TX, fluoroacetamide+TX, fluoroimide+TX, fluoxapiprolin+TX, fluoxastrobin+TX, fluoxytioconazole+TX, flupropadine+TX, flupropadine hydrochloride+TX, fluquinconazole+TX, flusilazole+TX, flusulfamide+TX, flutianil+TX, flutolanil+TX, flutriafol+TX, fluxapyroxad+TX, FMC 1137+TX, folpet+TX, formaldehyde+TX, formetanate+TX, formetanate hydrochloride+TX, formparanate+TX, fosetyl-aluminium+TX, fosmethilan+TX, fospirate+TX, fosthietan+TX, frontalin+TX, fuberidazole+TX, furalaxyl+TX, furametpyr+TX, furathiocarb+TX, furethrin+TX, furfural+TX, gamma-HCH+TX, glyodin+TX, grandlure+TX, grandlure I+TX, grandlure II+TX, grandlure III+TX, grandlure IV+TX, guazatine+TX, guazatine acetates+TX, halfenprox+TX, HCH+TX, hemel+TX, hempa+TX, HEOD+TX, heptachlor+TX, heterophos+TX, Heterorhabditis bacteriophora and H. megidis+TX, hexaconazole+TX, hexadecyl cyclopropanecarboxylate+TX, hexalure+TX, hexamide+TX, HHDN+TX, Hippodamia convergens+TX, hydrargaphen+TX, hydrated lime+TX, hydrogen cyanide+TX, hymexazol+TX, hyquincarb+TX, imanin+TX, imazalil+TX, imibenconazole+TX, iminoctadine+TX, inpyrfluxam+TX, ipconazole+TX, ipfentrifluconazole+TX, ipflufenoquin+TX, iprobenphos+TX, iprodione+TX, iprovalicarb+TX, ipsdienol+TX, ipsenol+TX, IPSP+TX, isamidofos+TX, isazofos+TX, isobenzan+TX, isocarbophos+TX, isodrin+TX, isofenphos+TX, isofetamid+TX, isoflucypram+TX, isolane+TX, isoprothiolane+TX, isopyrazam+TX, isotianil+TX, isoxathion+TX, japonilure+TX, jasmolin I+TX, jasmolin II+TX, jodfenphos+TX, juvenile hormone I+TX, juvenile hormone II+TX, juvenile hormone III+TX, kadethrin+TX, kasugamycin+TX, kasugamycin hydrochloride hydrate+TX, kelevan+TX, kinetin+TX, kinoprene+TX, kresoxim-methyl+TX, lead arsenate+TX, Leptomastix dactylopii+TX, leptophos+TX, lindane+TX, lineatin+TX, lirimfos+TX, litlure+TX, looplure+TX, Ivbenmixianan+TX, lythidathion+TX, Macrolophus caliginosus+TX, magnesium phosphide+TX, malonoben+TX, Mamestra brassicae NPV+TX, mancopper+TX, mancozeb+TX, mandestrobin+TX, mandipropamid+TX, maneb+TX, mazidox+TX, m-cumenyl methylcarbamate+TX, mecarbam+TX, mecarphon+TX, medlure+TX, mefentrifluconazole+TX, megatomoic acid+TX, menazon+TX, mepanipyrim+TX, meperfluthrin+TX, mephosfolan+TX, mepronil+TX, mercuric oxide+TX, mercurous chloride+TX, mesulfen+TX, mesulfenfos+TX, metalaxyl+TX, metam+TX, metam-potassium+TX, metam-sodium+TX, Metaphycus helvolus+TX, Metarhizium anisopliae var. acridum+TX, Metarhizium anisopliae var. anisopliae+TX, metarylpicoxamid+TX, metconazole+TX, metepa+TX, methacrifos+TX, methanesulfonyl fluoride+TX, methasulfocarb+TX, methiotepa+TX, methocrotophos+TX, methoprene+TX, methoquin-butyl+TX, methothrin+TX, methoxychlor+TX, methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate (these compounds may be prepared from the methods described in WO2020/193387)+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-(4-propyltriazol-2-yl)phenoxy]prop-2-enoate+TX, methyl (Z)-3-methoxy-2-[2-methyl-5-[3-(trifluoromethyl)pyrazol-1-yl]phenoxy]prop-2-enoate (these compounds may be prepared from the methods described in WO2020/079111)+TX, methyl (Z)-3-methoxy-2-[2-methyl-5-[4-(trifluoromethyl)triazol-2-yl]phenoxy]prop-2-enoate+TX, methyl apholate+TX, methyl bromide+TX, methyl eugenol+TX, methyl isothiocyanate+TX, methyl N-[[4-[1-(2,6-difluoro-4-isopropyl-phenyl)pyrazol-4-yl]-2-methyl-phenyl]methyl]carbamate (may be prepared from the methods described in WO 2020/097012)+TX, methyl N-[[4-[1-(4-cyclopropyl-2,6-difluoro-phenyl)pyrazol-4-yl]-2-methyl-phenyl]methyl]carbamate (may be prepared from the methods described in WO 2020/097012)+TX, methyl N-[[5-[4-(2,4-dimethylphenyl)triazol-2-yl]-2-methyl-phenyl]methyl]carbamate+TX, methylchloroform+TX, methylene chloride+TX, methylneodecanamide+TX, metiram+TX, metolcarb+TX, metominostrobin+TX, metoxadiazone+TX, metrafenone+TX, metyltetraprole+TX, MGK 264+TX, milbemycin oxime+TX, mipafox+TX, mirex+TX, monocrotophos+TX, morphothion+TX, morzid+TX, moxidectin+TX, muscalure+TX, myclobutanil+TX, myclozoline+TX, Myrothecium verrucaria composition+TX, N-((1R)-1-benzyl-3-chloro-1-methyl-but-3-enyl)-8-fluoro-quinoline-3-carboxamide (these compounds may be prepared from the methods described in WO2017/153380)+TX, N-((1S)-1-benzyl-3-chloro-1-methyl-but-3-enyl)-8-fluoro-quinoline-3-carboxamide (these compounds may be prepared from the methods described in WO2017/153380)+TX, N′-(2,5-dimethyl-4-phenoxy-phenyl)-N-ethyl-N-methyl-formamidine+TX, N′-(2-chloro-5-methyl-4-phenoxy-phenyl)-N-ethyl-N-methyl-formamidine+TX, N,2-dimethoxy-N-[[4-[5-(trifluoromethyl)-1,2,4-oxadiazol-3-yl]phenyl]methyl]propanamide+TX, N,N-dimethyl-1-[[4-[5-(trifluoromethyl)-1,2,4-oxadiazol-3-yl]phenyl]methyl]-1,2,4-triazol-3-amine (THESE COMPOUNDS may be prepared from the methods described in WO 2017/055473, WO 2017/055469, WO 2017/093348 and WO 2017/118689)+TX, N-[(1R)-1-benzyl-1,3-dimethyl-butyl]-7,8-difluoro-quinoline-3-carboxamide+TX, N-[(1R)-1-benzyl-1,3-dimethyl-butyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-1,3-dimethyl-butyl]-7,8-difluoro-quinoline-3-carboxamide+TX, N-[(1 S)-1-benzyl-1,3-dimethyl-butyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[[4-[5-(trifluoromethyl)-1,2,4-oxadiazol-3-yl]phenyl]methyl]propanamide+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, N′-[2-chloro-4-(2-fluorophenoxy)-5-methyl-phenyl]-N-ethyl-N-methyl-formamidine (this compound may be prepared from the methods described in WO 2016/202742)+TX, N′-[4-(4,5-dichlorothiazol-2-yl)oxy-2,5-dimethyl-phenyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075)+TX, N′-[5-bromo-2-methyl-6-(2-propoxypropoxy)-3-pyridyl]-N-ethyl-N-methyl-formamidine (this compound may be prepared from the methods described in IPCOM000249876D)+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-[(1S)-1-methyl-2-propoxy-ethoxy]-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N-[N-methoxy-C-methyl-carbonimidoyl]-4-[5-(trifluoromethyl)-1,2,4-oxadiazol-3-yl]benzamide (these compounds may be prepared from the methods described in WO 2018/202428)+TX, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896)+TX, nabam+TX, naftalofos+TX, naled+TX, naphthalene+TX, NC-170+TX, Neodiprion sertifer NPV and N. lecontei NPV+TX, nerolidol+TX, N-ethyl-2-methyl-N-[[4-[5-(trifluoromethyl)-1,2,4-oxadiazol-3-yl]phenyl]methyl]propanamide+TX, N-ethyl-N′-[5-methoxy-2-methyl-4-[(2-trifluoromethyl)oxetan-2-yl]phenyl]-N-methyl-formamidine+TX, nickel bis(dimethyldithiocarbamate)+TX, niclosamide-olamine+TX, nicotine+TX, nicotine sulfate+TX, nifluridide+TX, nikkomycins+TX, N-isopropyl-N′-[5-methoxy-2-methyl-4-(2,2,2-trifluoro-1-hydroxy-1-phenyl-ethyl)phenyl]-N-methyl-formamidine+TX, nithiazine+TX, nitrapyrin+TX, nitrilacarb+TX, nitrilacarb 1:1 zinc chloride complex+TX, nitrothal-isopropyl+TX, N-methoxy-N-[[4-[5-(trifluoromethyl)-1,2,4-oxadiazol-3-yl]phenyl]methyl]cyclopropanecarboxamide+TX, N-methyl-4-[5-(trifluoromethyl)-1,2,4-oxadiazol-3-yl]benzamide+TX, N-methyl-4-[5-(trifluoromethyl)-1,2,4-oxadiazol-3-yl]benzenecarbothioamide+TX, norbormide+TX, nuarimol+TX, O,O,O′,O′-tetrapropyl dithiopyrophosphate+TX, octadeca-2,13-dien-1-yl acetate+TX, octadeca-3,13-dien-1-yl acetate+TX, octhilinone+TX, ofurace+TX, oleic acid+TX, omethoate+TX, orfralure+TX, Orius spp.+TX, oryctalure+TX, orysastrobin+TX, ostramone+TX, oxadixyl+TX, oxamate+TX, oxathiapiprolin+TX, oxine-copper+TX, oxolinic acid+TX, oxycarboxin+TX, oxydeprofos+TX, oxydisulfoton+TX, oxytetracycline+TX, paclobutrazole+TX, Paecilomyces fumosoroseus+TX, para-dichlorobenzene+TX, parathion+TX, parathion-methyl+TX, pefurazoate+TX, penconazole+TX, pencycuron+TX, penflufen+TX, penfluron+TX, pentachlorophenol+TX, pentachlorophenyl laurate+TX, penthiopyrad+TX, permethrin+TX, PH 60-38+TX, phenamacril+TX, phenkapton+TX, phosacetim+TX, phosalone+TX, phosdiphen+TX, phosfolan+TX, phosglycin+TX, phosnichlor+TX, phosphamidon+TX, phosphine+TX, phosphorus+TX, phoxim-methyl+TX, phthalide+TX, Phytoseiulus persimilis+TX, picarbutrazox+TX, picaridin+TX, picoxystrobin+TX, pindone+TX, piperazine+TX, piperonyl butoxide+TX, piprotal+TX, pirimetaphos+TX, polychlorodicyclopentadiene isomers+TX, polychloroterpenes+TX, polynactins+TX, polyoxins+TX, potassium arsenite+TX, potassium ethylxanthate+TX, potassium hydroxyquinoline sulfate+TX, potassium thiocyanate+TX, pp′-DDT+TX, precocene I+TX, precocene II+TX, precocene III+TX, primidophos+TX, probenazole+TX, prochloraz+TX, proclonol+TX, procymidone+TX, profluthrin+TX, promacyl+TX, promecarb+TX, propamocarb+TX, propiconazole+TX, propineb+TX, propoxur+TX, propyl isomer+TX, proquinazid+TX, prothidathion+TX, prothioconazole+TX, prothiofos+TX, prothoate+TX, pydiflumetofen+TX, pyraclostrobin+TX, pyrametostrobin+TX, pyraoxystrobin+TX, pyrapropoyne+TX, pyraziflumid+TX, pyrazophos+TX, pyresmethrin+TX, pyrethrin I+TX, pyrethrin II+TX, pyrethrins+TX, pyribencarb+TX, pyridachlometyl+TX, pyridaphenthion+TX, pyridin-4-amine+TX, pyrifenox+TX, pyrimethanil+TX, pyrimitate+TX, pyrimorph+TX, pyrinuron+TX, pyriofenone+TX, pyrisoxazole+TX, pyroquilon+TX, quassia+TX, quinalphos+TX, quinalphos-methyl+TX, quinoclamine+TX, quinofumelin+TX, quinonamid+TX, quinothion+TX, quinoxyfen+TX, quintiofos+TX, quintozene+TX, R-1492+TX, rafoxanide+TX, resmethrin+TX, Reynoutria sachalinensis extract+TX, ribavirin+TX, Rmetalaxyl+TX, rotenone+TX, ryania+TX, ryanodine+TX, S421+TX, sabadilla+TX, schradan+TX, scilliroside+TX, seboctylamine+TX, sebufos+TX, sedaxane+TX, selamectin+TX, sesamex+TX, sesasmolin+TX, SI-0009+TX, siglure+TX, simazine+TX, simeconazole+TX, sodium arsenite+TX, sodium cyanide+TX, sodium fluoride+TX, sodium fluoroacetate+TX, sodium hexafluorosilicate+TX, sodium pentachlorophenoxide+TX, sodium selenate+TX, sodium tetrathiocarbonate+TX, sodium thiocyanate+TX, sophamide+TX, sordidin+TX, spiroxamine+TX, SSI-121+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, streptomycin+TX, streptomycin sesquisulfate+TX, strychnine+TX, sulcatol+TX, sulcofuron+TX, sulcofuron-sodium+TX, sulfiram+TX, sulfluramid+TX, sulfotep+TX, sulfoxide+TX, sulfur+TX, sulfuryl fluoride+TX, sulprofos+TX, tar oils+TX, tau-fluvalinate+TX, tazimcarb+TX, TDE+TX, tebuconazole+TX, tebufloquin+TX, tebupirimfos+TX, tecloftalam+TX, temephos+TX, tepa+TX, TEPP+TX, terallethrin+TX, terbam+TX, tert-butyl N-[6-[[[(1-methyltetrazol-5-yl)-phenyl-methylene]amino]oxymethyl]-2-pyridyl]carbamate+TX, tetrachloroethane+TX, tetrachlorothiophene+TX, tetraconazole+TX, tetradec-11-en-1-yl acetate+TX, tetradifon+TX, tetramethylfluthrin+TX, tetrasul+TX, thallium sulfate+TX, thiabendazole+TX, thiafenox+TX, thiapronil+TX, thicrofos+TX, thifluzamide+TX, thiocarboxime+TX, thiocyclam+TX, thiocyclam hydrogen oxalate+TX, thiodiazole copper+TX, thiofanox+TX, thiohempa+TX, thiomersal+TX, thiometon+TX, thionazin+TX, thiophanate+TX, thiophanate-methyl+TX, thioquinox+TX, thiosultap+TX, thiosultap-sodium+TX, thiotepa+TX, thiram+TX, thuringiensin+TX, tiadinil+TX, tolclofos-methyl+TX, tolprocarb+TX, tolylfluanid+TX, tralomethrin+TX, transpermethrin+TX, tretamine+TX, triadimefon+TX, triadimenol+TX, triamiphos+TX, triarathene+TX, triazamate+TX, triazophos+TX, triazoxide+TX, triazuron+TX, tributyltin oxide+TX, trichlormetaphos-3+TX, trichloronat+TX, Trichogramma spp.+TX, triclopyricarb+TX, tricyclazole+TX, tridemorph+TX, trifenmorph+TX, trifenofos+TX, trifloxystrobin+TX, triflumizole+TX, triforine+TX, trimedlure+TX, trimedlure A+TX, trimedlure B1+TX, trimedlure B2+TX, trimedlure C+TX, trimethacarb+TX, trinactin+TX, trinexapac+TX, triphenyltin acetate+TX, triphenyltin hydroxide+TX, triprene+TX, triticonazole+TX, trunc-call+TX, Typhlodromus occidentalis+TX, uredepa+TX, validamycin+TX, valifenalate+TX, vamidothion+TX, vaniliprole+TX, veratridine+TX, veratrine+TX, verbutin+TX, Verticillium lecanii+TX, vinclozoline+TX, warfarin+TX, XMC+TX, xylenols+TX, zeatin+TX, zetamethrin+TX, zhongshengmycin+TX, zinc naphthenate+TX, zinc phosphide+TX, zinc thiazole+TX, zineb+TX, ziram+TX, zolaprofos+TX; Acinetobacter lwoffii+TX, Acremonium alternatum+TX, Acremonium cephalosporium+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum (MicroAZ®, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain AQ746+TX, Bacillus chitinosporus strain CM-1+TX, Bacillus circulans+TX, Bacillus firmus (BioSafe®, BioNem-WP®) in particular strain CNMC 1-1582 (e.g. VOTIVO® from BASF SE)+TX, Bacillus licheniformis strain 3086 (EcoGuard®, Green Releaf®)+TX, Bacillus licheniformis strain HB-2 (Biostart™ formerly Rhizoboost®)+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain AQ717+TX, Bacillus pumilus strain GB34 (Yield Shield®)+TX, Bacillus pumilus strain QST 2808 (Sonata®, Ballad Plus®)+TX, Bacillus sphaericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain AQ153+TX, Bacillus subtilis strain AQ743+TX, Bacillus subtilis strain QST 713 (CEASE®, Serenade®, Rhapsody®)+TX, Bacillus subtilis strain QST 714 (JAZZ®)+TX, Bacillus subtilis strain QST3002+TX, Bacillus subtilis strain QST3004+TX, Bacillus subtilis var. amyloliquefaciens strain FZB24 (Taegro®, Rhizopro®)+TX, Bacillus thuringiensis aizawai GC 91 (Agree®)+TX, Bacillus thuringiensis Cry 2Ae+TX, Bacillus thuringiensis Cry1Ab+TX, Bacillus thuringiensis israelensis (BMP123®, Aquabac®, VectoBac®)+TX, Bacillus thuringiensis kurstaki (Javelin®, Deliver®, CryMax®, Bonide®, Scutella WP®, Turilav WP®, Astuto®, Dipel VP®, Biobit®, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain AQ52+TX, Bacillus thuringiensis strain BD #32+TX, Bacillus thuringiensis tenebrionis (Novodor®, BtBooster)+TX, Bacillus thuringiensis var. aizawai (XenTari®, DiPel®)+TX, bacteria spp. (GROWMEND®, GROWSWEET®, Shootup®)+TX, bacteriophage of Clavipacter michiganensis (AgriPhage®, Bakflor®)+TX, Beauveria bassiana (Beaugenic®, Brocaril WP®)+TX, Beauveria bassiana GHA (Mycotrol ES®, Mycotrol O®, BotaniGuard®)+TX, Beauveria brongniartii (Engerlingspilz®, Schweizer Beauveria®, Melocont®)+TX, Beauveria spp.+TX, Botrytis cineria+TX, Bradyrhizobium japonicum (TerraMax®)+TX, Brevibacillus brevis+TX, Burkholderia cepacia (Deny®, Intercept®, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®, Biocure®)+TX, Candida sake+TX, Candida spp.+TX, Candida tenius+TX, Cedecea davisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium chlorocephalum+TX, Cladosporium cladosporioides+TX, Cladosporium oxysporum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®, Madex®, Madex® Plus, Madex Max, Carpovirusine®+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®, Biofox C®)+TX, Fusarium proliferatum+TX, Fusarium spp.+TX, Galactomyces geotrichum+TX, Gliocladium catenulatum (Primastop®, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isaria fumosorosea (previously known as Paecilomyces fumosoroseus strain, PFR-97®, PreFeRal®)+TX, Isoflavone formononetin (Myconate®)+TX, Kloeckera apiculata+TX, Kloeckera spp.+TX, Lagenidium giganteum (Laginex®)+TX, Lecanicillium lecanii (formerly known as Verticillium lecanii (Mycotal®) conidia of strain KV01 (e.g. Vertalec® by Koppert/Arysta)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Destruxin WP®)+TX, Metarhizium anisopliae (Met52®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus in particular strain A3-5 (Accession No. NRRL 30548)+TX, Mycorrhizae spp. (AMykor®, Root Maximizer®)+TX, Myrothecium verrucaria strain AARC-0255 (DiTera®, BROS PLUS®)+TX, Ophiostoma piliferum strain D97 (Sylvanex®)+TX, Paecilomyces farinosus+TX, Paecilomyces lilacinus strain 251 (MeloCon WG®)+TX, Paecilomyces linacinus (Biostat WP®)+TX, Paenibacillus polymyxa+TX, Pantoea agglomerans (BlightBan C9-1®)+TX, Pantoea spp.+TX, Pasteuria nishizawae in particular strain Pn1 (CLARIVA from Syngenta/ChemChina); +TX, Pasteuria spp. (Econem®)+TX, Penicillium aurantiogriseum+TX, Penicillium billai (Jumpstart®, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilliermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens (Zequanox®)+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®, Polyversum®)+TX, Pythium periplocum+TX, Rhanella aquatilis+TX, Rhanella spp.+TX, Rhizobia (Dormal®, Vault®)+TX, Rhizoctonia+TX, Rhodococcus globerulus strain AQ719+TX, Rhodosporidium diobovatum+TX, Rhodosporidium toruloides+TX, Rhodotorula glutinis+TX, Rhodotorula graminis+TX, Rhodotorula mucilagnosa+TX, Rhodotorula rubra+TX, Rhodotorula spp.+TX, Saccharomyces cerevisiae+TX, Salinococcus roseus+TX, Sclerotinia minor (SARRITOR®)+TX, Sclerotinia minor+TX, Scytalidium spp.+TX, Scytalidium uredinicola+TX, Serratia marcescens+TX, Serratia plymuthica+TX, Serratia spp.+TX, Sordaria fimicola+TX, Spodoptera exigua nuclear polyhedrosis virus (Spod-X®, Spexit®)+TX, Spodoptera littoralis nucleopolyhedrovirus (Littovir®)+TX, Sporobolomyces roseus+TX, Stenotrophomonas maltophilia+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces hygroscopicus+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma atroviride (Plantmate®)+TX, Trichoderma gamsii (Tenet®)+TX, Trichoderma hamatum TH 382+TX, Trichoderma harzianum rifai (Mycostar®)+TX, Trichoderma harzianum T-22 (Trianum-P®, PlantShield HCO, RootShield®, Trianum-G®+TX, Trichoderma harzianum T-39 (Trichodex®)+TX, Trichoderma inhamatum+TX, Trichoderma koningii+TX, Trichoderma lignorum+TX, Trichoderma longibrachiatum+TX, Trichoderma polysporum (Binab T®)+TX, Trichoderma spp. LC 52 (Sentinel®)+TX, Trichoderma taxi+TX, Trichoderma virens (formerly Gliocladium virens GL-21) (SoilGuard®)+TX, Trichoderma virens+TX, Trichoderma viride+TX, Trichoderma viride strain ICC 080 (Remedier®)+TX, Trichosporon pullulans+TX, Trichosporon spp.+TX, Trichothecium roseum+TX, Trichothecium spp.+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus+TX,
AGNIQUE® MMF+TX, azadirachtin (Plasma Neem Oil®, AzaGuard®, MeemAzal®, Molt-X® e.g. AZATIN XL from Certis, US)+TX, Botanical IGR (Neemazad®, Neemix®)+TX, BugOil®+TX, canola oil (Lilly Miller Vegol®)+TX, Chenopodium ambrosioides near ambrosioides (Requiem®)+TX, Chrysanthemum extract (Crisant®)+TX, essentials oils of Labiatae (Botania®)+TX, extract of neem oil (Trilogy®)+TX, extracts of clove rosemary peppermint and thyme oil (Garden Insect Killer®)+TX, garlic+TX, Glycinebetaine (Greenstim®)+TX, kaolin (Screen®)+TX, lemongrass oil (GreenMatch®)+TX, Melaleuca alternifolia extract (also called tea tree oil) (Timorex Gold®)+TX, mixture of clove pepermint garlic oil and mint (Soil Shot®)+TX, mixture of clove rosemary and peppermint extract (EF 400®)+TX, mixture of rosemary sesame pepermint thyme and cinnamon extracts (EF 300®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pine oil (Retenol®)+TX, pyrethrum+TX, Quillaja saponaria (NemaQ®)+TX, Reynoutria sachalinensis (Regalia®, Sakalia®)+TX, rotenone (Eco Roten®)+TX, Rutaceae plant extract (Soleo®)+TX, soybean oil (Ortho Ecosense®)+TX, storage glucam of brown algae (Laminarin®)+TX, thyme oil+TX;
(E,Z)-7,9-Dodecadien-1-yl acetate+TX, (E,Z,Z)-3,8,11 Tetradecatrienyl acetate+TX, (Z,Z,E)-7,11,13-Hexadecatrienal+TX, 2-Methyl-1-butanol+TX, Biolure®+TX, blackheaded fireworm pheromone (3M Sprayable Blackheaded Fireworm Pheromone®)+TX, Calcium acetate+TX, Check-Mate®+TX, Codling Moth Pheromone (Paramount dispenser-(CM)/Isomate C-Plus®)+TX, Entostat powder (extract from palm tree) (Exosex CM®)+TX, Grape Berry Moth Pheromone (3M MEC-GBM Sprayable Pheromone®)+TX, Lavandulyl senecioate+TX, Leafroller pheromone (3M MEC—LR Sprayable Pheromone®)+TX, Muscamone (Snip7 Fly Bait®)+TX, Oriental Fruit Moth Pheromone (3M oriental fruit moth sprayable Pheromone®)+TX, Peachtree Borer Pheromone (Isomate-P®)+TX, Scenturion®+TX, Starbar Premium Fly Bait®+TX, Tomato Pinworm Pheromone (3M Sprayable Pheromone®)+TX,
Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®, Andersoni-System®)+TX, Amblyseius californicus (Amblyline®, Spical®)+TX, Amblyseius cucumeris (Thripex®, Bugline cucumeris®)+TX, Amblyseius fallacis (Fallacis®)+TX, Amblyseius swirskii (Bugline Swirskii®, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®, Aphiline®), +TX, Aphelinus asychis+TX, Aphidius colemani (Aphipar®)+TX, Aphidius ervi (Aphelinus-System®)+TX, Aphidius ervi (Ervipar®)+TX, Aphidius gifuensis+TX, Aphidius matricariae (Aphipar-M®)+TX, Aphidoletes aphidimyza (Aphidend®, Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Beeline®, Tripol®)+TX, Bombus terrestris (Natupol Beehive®)+TX, Cephalonomia stephanoderis+TX, Chilocorus nigritus+TX, Chrysoperla carnea (Chrysoline®, Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®, Cryptoline®)+TX, Cybocephalus nipponicus+TX, Dacnusa sibirica (Minusa®, DacDigline®, Minex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea (Diminex®, Miglyphus®, Digline®)+TX, Diversinervus spp.+TX, Encarsia citrina+TX, Encarsia formosa (Encarsia Max®, Encarline®, En-Strip®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Enermix®, Ercal®, Eretline e®, Bemimix®)+TX, Eretmocerus hayati+TX, Eretmocerus mundus (Bemipar®, Eretline m®)+TX, Eretmocerus siphonini+TX, Exochomus quadripustulatus+TX, Feltiella acarisuga (Feltiline®)+TX, Feltiella acarisuga (Spidend®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis bacteriophora (NemaShield HB®, Nemaseek®, Terranem-Nam®, Terranem®, Larvanem®, B-Green®, NemAttack®, Nematop®)+TX, Heterorhabditis megidis (Nemasys H®, BioNem H®, Exhibitline hm®, Larvanem-M®)+TX, Heterorhabditis spp. (Lawn Patrol®)+TX, Hippodamia convergens+TX, Hypoaspis aculeifer (Aculeifer-System®, Entomite-A®)+TX, Hypoaspis miles (Hypoline m®, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®, Macroline c®, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®, Nesibug®)+TX, Ophyra aenescens (Biofly®)+TX, Orius insidiosus (Thripor-I®, Oriline i®)+TX, Orius laevigatus (Thripor-L®, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®, Millenium®, BioNem C®, NemAttack®, Nemastar®, Capsanem®)+TX, Steinernema feltiae (NemaShield®, Nemasys F®, BioNem F®, Steinernema-System®, NemAttack®, Nemaplus®, Exhibitline sf®, Scia-rid®, Entonem®)+TX, Steinernema kraussei (Nemasys L®, BioNem L®, Exhibitline srb®)+TX, Steinernema riobrave (BioVector®, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator+TX;
abscisic acid+TX, Aminomite®+TX, BioGain®+TX, bioSea®+TX, CAS Number: 2643947-26-4+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, Nosema locustae (Semaspore Organic Grasshopper Control®)+TX, Pheromone trap (Thripline ams®)+TX, potassium bicarbonate (MilStop®)+TX, potassium iodide+potassiumthiocyanate (Enzicur®)+TX, potassium salts of fatty acids (Sanova®)+TX, potassium silicate solution (Sil-Matrix®)+TX, Spider venom+TX, Sticky traps (Trapline YF®, Rebell Amarillo®)+TX, SuffOil-X®+TX, Traps (Takitrapline y+b®)+TX; Bacillus mojavensis strain R3B (Accession No. NCAIM (P) B001389) (WO 2013/034938) from Certis USA LLC+TX, Bacillus pumilus, in particular strain BU F-33, having NRRL Accession No. 50185 (CARTISSA® from BASF, EPA Reg. No. 71840-19)+TX, Bacillus subtilis CX-9060 from Certis USA LLC, Bacillus sp., in particular strain D747 (available as DOUBLE NICKEL® from Kumiai Chemical Industry Co., Ltd.), having Accession No. FERM BP-8234, U.S. Pat. No. 7,094,592+TX, Bacillus subtilis strain BU1814, (VELONDIS® PLUS, VELONDIS® FLEX and VELONDIS® EXTRA from BASF SE)+TX, Bacillus subtilis var. amyloliquefaciens strain FZB24 having Accession No. DSM 10271 (available from Novozymes as TAEGRO® or TAEGRO® ECO (EPA Registration No. 70127-5))+TX, Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX, Paenibacillus polymyxa, in particular strain AC-1 (e.g. TOPSEED® from Green Biotech Company Ltd.)+TX, Paenibacillus sp. strain having Accession No. NRRL B-50972 or Accession No. NRRL B-67129, WO 2016/154297+TX, Pantoea agglomerans, in particular strain E325 (Accession No. NRRL B-21856) (available as BLOOMTIME BIOLOGICAL™ FD BIOPESTICIDE from Northwest Agri Products)+TX, Pseudomonas proradix (e.g. PRORADIX® from Sourcon Padena)+TX; Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX, Pseudozyma aphidis (as disclosed in WO2011/151819 by Yissum Research Development Company of the Hebrew University of Jerusalem)+TX, Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR+TX; Agrobacterium radiobacter strain K84 (e.g. GALLTROL-A® from AgBioChem, CA)+TX, Bacillus amyloliquefaciens isolate B246 (e.g. AVOGREEN™ from University of Pretoria)+TX, Bacillus amyloliquefaciens strain F727 (also known as strain MB1110) (NRRL Accession No. B-50768, WO 2014/028521) (STARGUS® from Marrone Bio Innovations)+TX, Bacillus amyloliquefaciens strain FZB42, Accession No. DSM 23117 (available as RHIZOVITAL® from ABiTEP, DE)+TX, Bacillus amyloliquefaciens, in particular strain D747 (available as Double Nickel™ from Kumiai Chemical Industry Co., Ltd., having accession number FERM BP-8234, U.S. Pat. No. 7,094,592)+TX, Bacillus licheniformis FMCH001 and Bacillus subtilis FMCH002 (QUARTZO® (WG) and PRESENCE® (WP) from FMC Corporation)+TX, Bacillus licheniformis, in particular strain SB3086, having Accession No. ATCC 55406, WO 2003/000051 (available as ECOGUARD® Biofungicide and GREEN RELEAF™ from Novozymes)+TX, Bacillus methylotrophicus strain BAC-9912 (from Chinese Academy of Sciences' Institute of Applied Ecology)+TX, Bacillus mycoides, isolate, having Accession No. B-30890 (available as BMJ TGAI® or WG and LifeGard™ from Certis USA LLC)+TX, Bacillus pumilus, in particular strain GB34 (available as Yield Shield® from Bayer AG, DE)+TX, Bacillus pumilus, in particular strain QST2808 (available as SONATA® from Bayer CropScience LP, US, having Accession No. NRRL B-30087 and described in U.S. Pat. No. 6,245,551)+TX, Bacillus subtilis CX-9060 from Certis USA LLC+TX, Bacillus subtilis IAB/BS03 (AVIV™ from STK Bio-Ag Technologies, PORTENTO® from Idai Nature)+TX, Bacillus subtilis KTSB strain (FOLIACTIVE® from Donaghys)+TX, Bacillus subtilis strain BU1814, (available as VELONDIS® PLUS, VELONDIS® FLEX and VELONDIS® EXTRA from BASF SE)+TX, Bacillus subtilis strain GB03 (available as Kodiak® from Bayer AG, DE)+TX, Bacillus subtilis strain MBI 600 (available as SUBTILEX from BASF SE), having Accession Number NRRL B-50595, U.S. Pat. No. 5,061,495+TX, Bacillus subtilis strain Y1336 (available as BIOBAC® WP from Bion-Tech, Taiwan, registered as a biological fungicide in Taiwan under Registration Nos. 4764, 5454, 5096 and 5277)+TX, Bacillus subtilis var. amyloliquefaciens strain FZB24 having Accession No. DSM 10271 (available from Novozymes as TAEGRO® or TAEGRO® ECO (EPA Registration No. 70127-5))+TX, Bacillus subtilis Y1336 (available as BIOBAC® WP from Bion-Tech, Taiwan, registered as a biological fungicide in Taiwan under Registration Nos. 4764, 5454, 5096 and 5277)+TX, Paenibacillus epiphyticus (WO 2016/020371) from BASF SE+TX, Paenibacillus polymyxa ssp. plantarum (WO 2016/020371) from BASF SE+TX, Paenibacillus sp. strain having Accession No. NRRL B-50972 or Accession No. NRRL B-67129, WO 2016/154297+TX, Pseudomonas chlororaphis strain AFS009, having Accession No. NRRL B-50897, WO 2017/019448 (e.g., HOWLER™ and ZIO® from AgBiome Innovations, US)+TX, Pseudomonas chlororaphis, in particular strain MA342 (e.g. CEDOMON®, CERALL®, and CEDRESS® by Bioagri and Koppert)+TX, Pseudomonas fluorescens strain A506 (e.g. BLIGHTBAN® A506 by NuFarm)+TX, Pseudomonas proradix (e.g. PRORADIX® from Sourcon Padena)+TX, Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX, Streptomyces lydicus strain WYEC108 (also known as Streptomyces lydicus strain WYCD108US) (ACTINO-IRON® and ACTINOVATE® from Novozymes)+TX, Trichoderma atroviride strain T11 (IM1352941/CECT20498)+TX, Ampelomyces quisqualis strain AQ10, having Accession No. CNCM 1-807 (e.g., AQ 10® by IntrachemBio Italia)+TX, Ampelomyces quisqualis, in particular strain AQ 10 (e.g. AQ 10® by IntrachemBio Italia)+TX, Aspergillus flavus strain NRRL 21882 (products known as AFLA-GUARD® from Syngenta/ChemChina)+TX, Aureobasidium pullulans, in particular blastospores of strain DSM 14941+TX, Aureobasidium pullulans, in particular blastospores of strain DSM14940+TX, Aureobasidium pullulans, in particular mixtures of blastospores of strains DSM14940 and DSM 14941 (e.g. Botector® by bio-ferm, CH)+TX, Chaetomium cupreum (Accession No. CABI 353812) (e.g. BIOKUPRUM™ by AgriLife)+TX, Chaetomium globosum (available as RIVADIOM® by Rivale)+TX, Cladosporium cladosporioides, strain H39, having Accession No. CBS122244, US 2010/0291039 (by Stichting Dienst Landbouwkundig Onderzoek)+TX, Coniothyrium minitans, in particular strain CON/M/91-8 (Accession No. DSM9660, e.g. Contans® from Bayer CropScience Biologics GmbH)+TX, Cryptococcus flavescens, strain 3C (NRRL Y-50378), +TX, Dactylaria candida, Dilophosphora alopecuri (available as TWIST FUNGUS®), Fusarium oxysporum, strain Fo47 (available as FUSACLEAN® by Natural Plant Protection)+TX, Gliocladium catenulatum (Synonym: Clonostachys rosea f. catenulate) strain J1446 (e.g. Prestop® by Lallemand)+TX, Gliocladium roseum (also known as Clonostachys rosea f rosea) strain IK726 (Jensen D F, et al. Development of a biocontrol agent for plant disease control with special emphasis on the near commercial fungal antagonist Clonostachys rosea strain ‘IK726’, Australasian Plant Pathol. 2007,36(2):95-101)+TX, Gliocladium roseum (also known as Clonostachys rosea f rosea), in particular strain 321U from Adjuvants Plus, strain ACM941 as disclosed in Xue A.G. (Efficacy of Clonostachys rosea strain ACM941 and fungicide seed treatments for controlling the root tot complex of field pea, Can Jour Plant Sci 2003, 83(3): 519-524)+TX, Metschnikowia fructicola, in particular strain NRRL Y-30752+TX, Microsphaeropsis ochracea, Penicillium steckii (DSM 27859, WO 2015/067800) from BASF SE+TX, mixtures of Trichoderma asperellum strain ICC 012 (also known as Trichoderma harzianum I00012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.)+TX, Penicillium vermiculatum+TX, Phlebiopsis gigantea strain VRA 1992 (ROTSTOP® C from Danstar Ferment)+TX, Pseudozyma flocculosa, strain PF-A22 UL (available as SPORODEX® L by Plant Products Co., CA)+TX, Saccharomyces cerevisiae strain LAS117 cell walls (CEREVISANE® from Lesaffre, ROMEO® from BASF SE)+TX, Saccharomyces cerevisiae strains CNCM No. 1-3936, CNCM No. 1-3937, CNCM No. 1-3938, CNCM No. 1-3939 (WO 2010/086790) from Lesaffre et Compagnie, FR+TX, Saccharomyces cerevisiae, in particular strain LASO2 (from Agro-Levures et Derives)+TX, Simplicillium lanosoniveum+TX, strain T34 (e.g. T34 Biocontrol by Biocontrol Technologies S.L., ES) or strain ICC 012 from Isagro+TX, strain WRL-076 (NRRL Y-30842), U.S. Pat. No. 7,579,183+TX, Talaromyces flavus, strain V117b+TX, Trichoderma asperelloides JM41R (Accession No. NRRL B-50759) (TRICHO PLUS® from BASF SE)+TX, Trichoderma asperellum, in particular strain SKT-1, having Accession No. FERM P-16510 (e.g. ECO-HOPE® from Kumiai Chemical Industry)+TX, Trichoderma asperellum, in particular, strain kd (e.g. T-Gro from Andermatt Biocontrol)+TX, Trichoderma atroviride strain 77B (T77 from Andermatt Biocontrol)+TX, Trichoderma atroviride strain ATCC 20476 (IMI 206040)+TX, Trichoderma atroviride strain LC52 (e.g. Tenet by Agrimm Technologies Limited)+TX, Trichoderma atroviride strain LU132 (e.g. Sentinel from Agrimm Technologies Limited)+TX, Trichoderma atroviride strain NMI no. V08/002388+TX, Trichoderma atroviride strain NMI no. V08/002389+TX, Trichoderma atroviride strain NMI no. V08/002390+TX, Trichoderma atroviride strain no. V08/002387+TX, Trichoderma atroviride strain SKT-1 (FERM P-16510), JP Patent Publication (Kokai) 11-253151 A+TX, Trichoderma atroviride strain SKT-2 (FERM P-16511), JP Patent Publication (Kokai) 11-253151 A+TX, Trichoderma atroviride strain SKT-3 (FERM P-17021), JP Patent Publication (Kokai) 11-253151 A+TX, Trichoderma atroviride, in particular strain SC1 (Accession No. CBS 122089, WO 2009/116106 and U.S. Pat. No. 8,431,120 (from Bi-PA))+TX, Trichoderma atroviride, strain CNCM 1-1237 (e.g. Esquive® WP from Agrauxine, FR)+TX, Trichoderma fertile (e.g. product TrichoPlus from BASF)+TX, Trichoderma gamsii (formerly T. viride)+TX, Trichoderma gamsii (formerly T. viride) strain ICC 080 (IMI CC 392151 CABI) (available as BIODERMA® by AGROBIOSOL DE MEXICO, S.A. DE C.V.), +TX, Trichoderma gamsii strain ICCO80 (IMI CC 392151 CABI, e.g. BioDerma by AGROBIOSOL DE MEXICO, S.A. DE C.V.), +TX, Trichoderma harmatum+TX, Trichoderma harmatum, having Accession No. ATCC 28012+TX, Trichoderma harzianum+TX, Trichoderma harzianum rifai T39 (e.g. Trichodex® from Makhteshim, US)+TX, Trichoderma harzianum strain Cepa SimbT5 (from Simbiose Agro), +TX, Trichoderma harzianum strain DB 103 (available as T-GRO®7456 by Dagutat Biolab)+TX, Trichoderma harzianum strain ITEM 908 (e.g. Trianum-P from Koppert)+TX, Trichoderma harzianum strain T-22 (e.g. Trianum-P from Andermatt Biocontrol or Koppert)+TX, Trichoderma harzianum strain TH35 (e.g. Root-Pro by Mycontrol)+TX, Trichoderma polysporum strain IMI 206039 (e.g. Binab TF WP by BINAB Bio-Innovation AB, Sweden)+TX, Trichoderma stromaticum having Accession No. Ts3550 (e.g. Tricovab by CEPLAC, Brazil)+TX, Trichoderma virens (also known as Gliocladium virens) in particular strain GL-21 (e.g. SoilGard by Certis, US)+TX, Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX, Trichoderma viride in particular strain B35 (Pietr et al., 1993, Zesz. Nauk. A R w Szczecinie 161: 125-137)+TX, Trichoderma viride strain TV1(e.g. Trianum-P by Koppert)+TX, Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX, Verticillium albo-atrum (formerly V. dahliae) strain WCS850 having Accession No. WCS850, deposited at the Central Bureau for Fungi Cultures (e.g., DUTCH TRIG® by Tree Care Innovations)+TX, Verticillium chlamydosporium+TX;
a mixture of Azotobacter vinelandii and Clostridium pasteurianum (available as INVIGORATE® from Agrinos)+TX, a mixture of Bacillus licheniformis FMCH001 and Bacillus subtilis FMCH002 (available as QUARTZO® (WG), PRESENCE® (WP) from FMC Corporation)+TX, Azorhizobium caulinodans, in particular strain ZB-SK-5+TX, Azospirillum brasilense (e.g., VIGOR® from KALO, Inc.)+TX, Azospirillum lipoferum (e.g., VERTEX-IF™ from TerraMax, Inc.)+TX, Azotobacter chroococcum, in particular strain H23+TX, Azotobacter vinelandii, in particular strain ATCC 12837+TX, Bacillus amyloliquefaciens BS27 (Accession No. NRRL B-5015)+TX, Bacillus amyloliquefaciens in particular strain FZB42 (e.g. RHIZOVITAL® from ABiTEP, DE)+TX, Bacillus amyloliquefaciens in particular strain IN937a+TX, Bacillus amyloliquefaciens pm414 (LOLI-PEPTA® from Biofilm Crop Protection)+TX, Bacillus amyloliquefaciens SB3281 (ATCC #PTA-7542, WO 2017/205258)+TX, Bacillus amyloliquefaciens TJ1000 (available as QUIKROOTS® from Novozymes)+TX, Bacillus cereus family member EE128 (NRRL No. B-50917)+TX, Bacillus cereus family member EE349 (NRRL No. B-50928)+TX, Bacillus cereus in particular strain BPO1 (ATCC 55675, e.g. MEPICHLOR® from Arysta Lifescience, US)+TX, Bacillus mycoides BT155 (NRRL No. B-50921)+TX, Bacillus mycoides BT46-3 (NRRL No. B-50922)+TX, Bacillus mycoides EE118 (NRRL No. B-50918)+TX, Bacillus mycoides EE141 (NRRL No. B-50916)+TX, Bacillus pumilus in particular strain GB34 (e.g. YIELD SHIELD® from Bayer Crop Science, DE), +TX, Bacillus pumilus in particular strain QST2808 (Accession No. NRRL No. B-30087)+TX, Bacillus siamensis in particular strain KCTC 13613T+TX, Bacillus subtilis in particular strain AQ30002 (Accession No. NRRL No. B-50421 and described in U.S. patent application Ser. No. 13/330,576)+TX, Bacillus subtilis in particular strain AQ30004 (NRRL No. B-50455 and described in U.S. patent application Ser. No. 13/330,576)+TX, Bacillus subtilis in particular strain MBI 600 (e.g. SUBTILEX® from BASF SE)+TX, Bacillus subtilis rm303 (RHIZOMAX® from Biofilm Crop Protection)+TX, Bacillus subtilis strain BU1814 (available as TEQUALIS® from BASF SE)+TX, Bacillus tequilensis in particular strain NII-0943+TX, Bacillus thuringiensis BT013A (NRRL No. B-50924) also known as Bacillus thuringiensis 4Q7+TX, Bradyrhizobium japonicum (e.g. OPTIMIZE® from Novozymes)+TX, Delftia acidovorans in particular strain RAY209 (e.g. BIOBOOST® from Brett Young Seeds)+TX, Lactobacillus sp. (e.g. LACTOPLANT® from LactoPAFI)+TX, Mesorhizobium cicer (e.g., NODULATOR from BASF SE)+TX, Paenibacillus polymyxa in particular strain AC-1 (e.g. TOPSEED® from Green Biotech Company Ltd.)+TX, Pseudomonas aeruginosa in particular strain PN1+TX, Pseudomonas proradix (e.g. PRORADIX® from Sourcon Padena)+TX, Rhizobium leguminosarium biovar viciae (e.g., NODULATOR from BASF SE)+TX, Rhizobium leguminosarum in particular bv. viceae strain Z25 (Accession No. CECT 4585)+TX, Serratia marcescens in particular strain SRM (Accession No. MTCC 8708), +TX, Sinorhizobium meliloti strain NRG-185-1 (NITRAGIN® GOLD from Bayer CropScience)+TX, Thiobacillus sp. (e.g. CROPAID® from Cropaid Ltd UK)+TX; Myrothecium verrucaria strain AARC-0255 (e.g. DiTera™ from Valent Biosciences)+TX, Penicillium bilaii strain ATCC 22348 (e.g. JumpStart® from Acceleron BioAg)+TX, Penicillium bilaii strain ATCC ATCC20851+TX, Purpureocillium lilacinum (previously known as Paecilomyces lilacinus) strain 251 (AGAL 89/030550, e.g. BioAct from Bayer CropScience Biologics GmbH)+TX, Pythium oligandrum strain DV74+TX, Pythium oligandrum strain M1 (ATCC 38472 e.g. Polyversum from Bioprepraty, CZ)+TX, Rhizopogon amylopogon (Myco-Sol from Agri-Enterprise, LLC, formerly Helena Chemical Company)+TX, Rhizopogon fulvigleba (e.g. Myco-Sol from Agri-Enterprise, LLC, formerly Helena Chemical Company)+TX, Talaromyces flavus strain V117b+TX, Trichoderma asperellum strain (Eco-T from Plant Health Products, ZA)+TX, Trichoderma asperellum strain kd (e.g. T-Gro from Andermatt Biocontrol)+TX, Trichoderma atroviride in particular strain no. V08/002387+TX, Trichoderma atroviride strain CNCM 1-1237 (e.g. Esquive® WP from Agrauxine, FR)+TX, Trichoderma atroviride strain LC52 (also known as Trichoderma atroviride strain LU132, e.g. Sentinel from Agrimm Technologies Limited)+TX, Trichoderma atroviride strain no. NMI No. V08/002388+TX, Trichoderma atroviride strain no. NMI No. V08/002389+TX, Trichoderma atroviride strain no. NMI No. V08/002390+TX, Trichoderma atroviride strain SC1 (described in WO2009/116106)+TX, Trichoderma harzianum strain 1295-22+TX, Trichoderma harzianum strain ITEM 908+TX, Trichoderma harzianum strain T-22 (e.g. Trianum-P from Andermatt Biocontrol or Koppert)+TX, Trichoderma harzianum strain TSTh20, +TX, Trichoderma virens strain GI-3+TX, Trichoderma virens strain GL-21 (e.g. SoilGard® from Certis, USA)+TX, Trichoderma viride strain B35 (Pietr et al., 1993, Zesz. Nauk. A R w Szczecinie 161: 125-137)+TX, Verticillium albo-atrum (formerly V. dahliae) strain WCS850 (CBS 276.92, e.g. Dutch Trig from Tree Care Innovations)+TX;
Agrobacterium radiobacter strain K84 (Galltrol from AgBiochem Inc.), +TX, Bacillus amyloliquefaciens in particular strain PTS-4838 (e.g. AVEO from Valent Biosciences, US), +TX, Bacillus mycoides, isolate J. (e.g. BmJ from Certis USA LLC), +TX, Bacillus sphaericus in particular Serotype H5a5b strain 2362 (strain ABTS-1743) (e.g. VECTOLEX® from Valent BioSciences, US), +TX, Bacillus thuringiensis israelensis strain BMP 144 (e.g. AQUABAC® by Becker Microbial Products IL)+TX, Bacillus thuringiensis subsp. aizawai strain GC-91+TX, Bacillus thuringiensis subsp. aizawai, in particular serotype H-7 (e.g. FLORBAC® WG from Valent BioSciences, US)+TX, Bacillus thuringiensis subsp. aizawai, in particular strain ABTS-1857 (SD-1372, e.g. XENTARI® from Valent BioSciences)+TX, Bacillus thuringiensis subsp. israelensis (serotype H-14) strain AM65-52 (Accession No. ATCC 1276) (e.g. VECTOBAC® by Valent BioSciences, US)+TX, Bacillus thuringiensis subsp. kurstaki strain ABTS 351+TX, Bacillus thuringiensis subsp. kurstaki strain BMP 123 (from Becker Microbial Products, IL, BARITONE from Bayer CropScience)+TX, Bacillus thuringiensis subsp. kurstaki strain EG 2348 (LEPINOX from Certis, US)+TX, Bacillus thuringiensis subsp. kurstaki strain EG 7841 (CRYMAX from Certis, US)+TX, Bacillus thuringiensis subsp. kurstaki strain HD-1 (e.g. DIPEL® ES from Valent BioSciences, US)+TX, Bacillus thuringiensis subsp. kurstaki strain PB 54+TX, Bacillus thuringiensis subsp. kurstaki strain SA 11 (JAVELIN from Certis, US)+TX, Bacillus thuringiensis subsp. kurstaki strain SA 12 (THURICIDE from Certis, US)+TX, Bacillus thuringiensis subsp. tenebrionis strain NB 176 (SD-5428, e.g. NOVODOR® FC from BioFa DE)+TX, Bacillus thuringiensis var. Colmeri (e.g. TIANBAOBTC by Changzhou Jianghai Chemical Factory)+TX, Bacillus thuringiensis var. japonensis strain Buibui+TX, Bacillus thuringiensis var. kurstaki strain EVB-113-19 (e.g., BIOPROTEC® from AEF Global)+TX, Brevibacillus laterosporus+TX, Burkholderia spp. in particular Burkholderia rinojensis strain A396 (also known as Burkholderia rinojensis strain MBI 305) (Accession No. NRRL B-50319, WO 2011/106491 and WO 2013/032693, e.g. MB1206 TGAI and ZELTO® from Marrone Bio Innovations),+TX, Chromobacterium subtsugae in particular strain PRAA4-1T (e.g. MBI-203, e.g. GRANDEVO® from Marrone Bio Innovations)+TX, Lecanicillium muscarium Ve6 (MYCOTAL from Koppert)+TX, Paenibacillus popilliae (formerly Bacillus popilliae, e.g. MILKY SPORE POWDER™ or MILKY SPORE GRANULAR™ from St. Gabriel Laboratories)+TX, Serratia entomophila (e.g. INVADE® by Wrightson Seeds)+TX, Serratia marcescens in particular strain SRM (Accession No. MTCC 8708)+TX, Trichoderma asperellum (TRICHODERMAX from Novozymes)+TX, Wolbachia pipientis ZAP strain (e.g., ZAP MALES® from MosquitoMate)+TX; Beauveria bassiana strain ATCC 74040 (e.g. NATURALIS® from Intrachem Bio Italia)+TX, Beauveria bassiana strain ATPO2 (Accession No. DSM 24665), Apopka 97 (PREFERAL from SePRO)+TX, Beauveria bassiana strain GHA (Accession No. ATCC74250, e.g. BOTANIGUARD® ES and MYCONTROL-O® from Laverlam International Corporation)+TX, Metarhizium anisopliae 3213-1 (deposited under NRRL accession number 67074 disclosed in WO 2017/066094, Pioneer Hi-Bred International)+TX, Metarhizium robertsii 15013-1 (deposited under NRRL accession number 67073)+TX, Metarhizium robertsii 23013-3 (deposited under NRRL accession number 67075)+TX, Paecilomyces lilacinus strain 251 (MELOCON from Certis, US)+TX; Cydia pomonella (codling moth) granulosis virus (GV)+TX, Helicoverpa armigera (cotton bollworm) nuclear polyhedrosis virus (NPV)+TX, of Adoxophyes orana (summer fruit tortrix) granulosis virus (GV)+TX, Spodoptera exigua (beet armyworm) mNPV+TX, Spodoptera frugiperda (fall armyworm) mNPV+TX;
Burkholderia spp. in particular Burkholderia cepacia (formerly known as Pseudomonas cepacia)+TX, Gigaspora spp.+TX, Glomus spp.+TX, Laccaria spp.+TX, LactoBacillus buchneri+TX, Paraglomus spp.+TX, Pisolithus tinctorus+TX, Pseudomonas spp.+TX, Rhizobium spp. in particular Rhizobium trifolii+TX, Rhizopogon spp.+TX, Scleroderma spp.+TX, Streptomyces spp.+TX, Suillus spp.+TX, Agrobacterium spp.+TX, Azorhizobium caulinodans+TX, Azospirillum spp.+TX, Azotobacter spp.+TX, Bradyrhizobium spp.+TX, Gigaspora monosporum+TX;
Allium sativum (NEMGUARD from Eco-Spray, BRALIC from ADAMA)+TX, Armour-Zen+TX, Artemisia absinthium+TX, Biokeeper WP+TX, Brassicaceae extract in particular oilseed rape powder or mustard powder+TX, Cassia nigricans+TX, Celastrus angulatus+TX, Chenopodium anthelminticum+TX, Chenopodium quinoa saponin extract from quinoa seeds (e.g. Heads Up® (Saponins of Quinoa) from Heads Up plant Protectants, CA)+TX, Chitin+TX, Dryopteris filix-mas+TX, Equisetum arvense+TX, Fortune Aza+TX, Fungastop+TX, Melaleuca alternifolia extract (TIMOREX GOLD from STK)+TX, naturally occurring Blad polypeptide extracted from Lupin seeds (FRACTURE® from FMC)+TX, naturally occurring Blad polypeptide extracted from Lupin seeds (PROBLAD® from Certis EU)+TX, Pyrethrins+TX, Quassia amara+TX, Quercus+TX, Quillaja extract (QL AGRI 35 from BASF)+TX, REGALIA MAXX from Marrone Bio)+TX, Requiem™ Insecticide+TX, Reynoutria sachalinensis extract (REGALLIA+TX, ryania/ryanodine+TX, Symphytum officinale+TX, Tanacetum vulgare+TX, Thymol+TX, Thymol mixed with Geraniol (CEDROZ from Eden Research)+TX, Thymol mixed with Geraniol and Eugenol (MEVALONE from Eden Research)+TX, Triact 70+TX, TriCon+TX, Tropaeulum majus+TX, Urtica dioica+TX, Veratrin+TX, Viscum album+TX;
mercuric oxide+TX, octhilinone+TX, thiophanate-methyl+TX;
MGK 264+TX, 2-(2-butoxyethoxy)ethyl piperonylate+TX, 2-isovalerylindan-1,3-dione+TX, 4-(quinoxalin-2-ylamino)benzenesulfonamide+TX, 5-(1,3-benzodioxol-5-yl)-3-hexylcyclohex-2-enone+TX, acibenzolar+TX, acibenzolar-S-methyl+TX, alpha-bromadiolone+TX, alpha-chlorohydrin+TX, aluminium phosphide+TX, anthraquinone+TX, antu+TX, arsenous oxide+TX, barium carbonate+TX, benoxacor+TX, bisthiosemi+TX, brodifacoum+TX, bromadiolone+TX, bromethalin+TX, calcium cyanide+TX, chloralose+TX, chlorophacinone+TX, cholecalciferol+TX, cloquintocet (including cloquintocet-mexyl)+TX, copper naphthenate+TX, copper oxychloride+TX, coumachlor+TX, coumafuryl+TX, coumatetralyl+TX, crimidine+TX, cyprosulfamide+TX, diazinon+TX, dichlormid+TX, dicyclopentadiene+TX, difenacoum+TX, difethialone+TX, diphacinone+TX, ergocalciferol+TX, farnesol+TX, farnesol with nerolidol+TX, fenchlorazole (including fenchlorazole-ethyl)+TX, fenclorim+TX, flocoumafen+TX, fluoroacetamide+TX, flupropadine+TX, flupropadine hydrochloride+TX, fluxofenim+TX, furilazole+TX, gamma-HCH+TX, guazatine+TX, guazatine acetates+TX, HCH+TX, hydrogen cyanide+TX, imanin+TX, iodomethane+TX, isoxadifen (including isoxadifen-ethyl)+TX, lindane+TX, magnesium phosphide+TX, MB-599+TX, mefenpyr (including mefenpyr-diethyl)+TX, metcamifen+TX, methiocarb+TX, methyl bromide+TX, nerolidol+TX, norbormide+TX, petroleum oils+TX, phosacetim+TX, phosphine+TX, phosphorus+TX, pindone+TX, piperonyl butoxide+TX, piprotal+TX, potassium arsenite+TX, probenazole+TX, propyl isomer+TX, pyridin-4-amine+TX, pyrinuron+TX, Reynoutria sachalinensis extract+TX, ribavirin+TX, S421+TX, scilliroside+TX, sesamex+TX, sesasmolin+TX, sodium arsenite+TX, sodium cyanide+TX, sodium fluoroacetate+TX, strychnine+TX, sulfoxide+TX, thallium sulfate+TX, thiram+TX, trimethacarb+TX, warfarin+TX, zinc naphthenate+TX, zinc phosphide+TX, ziram+TX.

The weight ratio of component (A) to component (C) may preferably be from 100:1 to 1:100, from 50:1 to 1:50, from 20:1 to 1:40, from 15:1 to 1:30, from 12:1 to 1:25, from 10:1 to 1:20, from 5:1 yo 1:15, from 3:1 to 1:10, or from 2:1 to 1:5.

A further aspect of invention is related to a method of controlling or preventing an infestation of plants, e.g. useful plants such as crop plants, propagation material thereof, e.g. seeds, harvested crops, e.g. harvested food crops, or of non-living materials by phytopathogenic or spoilage microorganisms or organisms potentially harmful to man, especially fungal organisms, which comprises the application of a composition according to the present invention to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a composition according to the present invention is foliar application. The frequency of application and the rate of application will depend on the risk of infestation by the corresponding pathogen or insect. However, the compounds A and/or B of the composition according to the present invention can also penetrate the plant through the roots via the soil (systemic action) by drenching the locus of the plant with a liquid formulation, or by applying said compounds in solid form to the soil, e.g. in granular form (soil application). In crops of water rice such granulates can be applied to the flooded rice field.

The compounds A and B as defined in the present invention may also be applied to seeds (coating) by impregnating the seeds or tubers either with a liquid formulation of the fungicide or coating them with a solid formulation.

The composition according to the present invention may be prepared in a known manner, typically by intimately mixing and/or grinding the compound with extenders, for example solvents, solid carriers and, optionally, surface active compounds (surfactants).

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the composition for controlling pests of the abovementioned type are other subjects of the invention. Typical rates of concentration are between 0.1 and 1000 ppm, preferably between 0.1 and 500 ppm, of active ingredient. The rate of application per hectare is preferably 1 g to 2000 g of active ingredient per hectare, more preferably 10 to 1000 g/ha, most preferably 10 to 600 g/ha. When used as seed drenching agent, convenient dosages are from 10 mg to 1 g of active substance per kg of seeds.

When the combinations of the present invention are used for treating seed, rates of 0.001 to 50 g of a compound of formula (I) per kg of seed, preferably from 0.01 to 10 g per kg of seed are generally sufficient. Suitably, the composition according to the present invention can be applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active ingredients, from 0 to 20% agriculturally acceptable surfactant and 10 to 99.99% solid or liquid formulation inerts and adjuvant(s), the active ingredients consisting of at least the components A and B as defined in the present invention, and optionally other active agents, particularly microbiocides or conservatives or the like. Concentrated forms of compositions generally contain in between about 2 and 80%, preferably between about 5 and 70% by weight of active ingredients. Application forms of formulation may for example contain from 0.01 to 20% by weight, preferably from 0.01 to 5% by weight of active ingredients. Whereas commercial products will preferably be formulated as concentrates, the end user will normally employ diluted formulations.

Whereas it is preferred to formulate commercial products as concentrates, the end user will normally use dilute formulations.

### EXAMPLES

The Examples which follow serve to illustrate the invention. The compounds (and compositions) of the invention may be distinguished from known compounds (and compositions) by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm or 0.2 ppm of active ingredient(s).

### FORMULATION EXAMPLES

| Wettable powders | a) | b) | c) |
|---|---|---|---|
| active ingredients [components (A) and (B)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients [components (A) and (B)] | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The active ingredient is thoroughly mixed with the adjuvants and the mixture is thoroughly ground in a suitable mill, affording powders that can be used directly for seed treatment.

| Emulsifiable concentrate | |
|---|---|
| active ingredients [components (A) and (B)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| active ingredients [components (A) and (B)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

| Extruder granules | |
|---|---|
| active ingredients [components (A) and (B)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

| Coated granules | |
|---|---|
| active ingredients [components (A) and (B)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredients [components (A) and (B)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients [components (A) and (B)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in water) | 0.5% |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the active ingredients [components (A) and (B)] is mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

### PREPARATION EXAMPLES

### Example 1: This example illustrates the preparation of methyl N-[5-[6-[(4-fluoro-3-methoxy-phenyl)-methyl-carbamoyl]imidazo[1,2-a]pyridin-3-yl]-2-pyridyl]carbamate (compound X.01 of Table T1)

### Step 1: Preparation of 3-bromo-N-(4-fluoro-3-methoxy-phenyl)-N-methyl-imidazo[1,2-a]pyridine-6-carboxamide

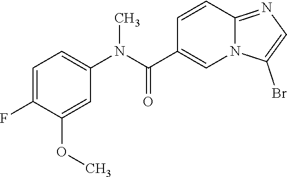 [SMILES: COc1cc(N(C)C(=O)c2ccc3ncc(Br)n3c2)ccc1F]

To a mixture of 3-bromoimidazo[1,2-a]pyridine-6-carboxylic acid (0.700 g, 2.90 mmol) in N,N-dimethylacetamide (17.5 mL) was added 4-fluoro-3-methoxy-N-methyl-aniline (0.712 g, 4.36 mmol) followed by N-ethyl-N-isopropyl-propan-2-amine (1.88 g, 2.49 mL, 14.5 mmol) and propanephosphonic acid anhydride (50% in ethyl acetate, 4.32 mL, 7.26 mmol). The reaction mixture was stirred for 1 hour at 50° C. The mixture was then poured into ice water, and then extracted with ethyl acetate. The combined organic layers were washed with saturated NaHCO₃ aq. and brine, dried over magnesium sulfate, filtered, and concentrated under reduced pressure. The crude residue was treated with diisopropylether and a precipitation occurred. The precipitate was filtered and washed with diisopropylether to afford 3-bromo-N-(4-fluoro-3-methoxy-phenyl)-N-methyl-imidazo[1,2-a]pyridine-6-carboxamide as a white solid.

LC/MS (Method A) retention time=0.81 min; [M+H]⁺=378

¹H NMR (400 MHz, CDCl₃) δ ppm 8.29 (s, 1H), 7.62 (s, 1H), 7.39 (dd, J=9.5, 0.7 Hz, 1H), 7.11 (dd, J=9.5, 1.8 Hz, 1H), 7.00 (dd, J=10.9, 8.7 Hz, 1H), 6.78 (dd, J=7.3, 2.5 Hz, 1H), 6.62-6.70 (m, 1H), 3.81 (s, 3H), 3.52 (s, 3H).

### Step 2: Preparation of N-[5-[6-((4-fluoro-3-methoxy-phenyl)-methyl-carbamoyl]imidazo[1,2-a]pyridin-3-yl]-2-pyridyl]carbamate

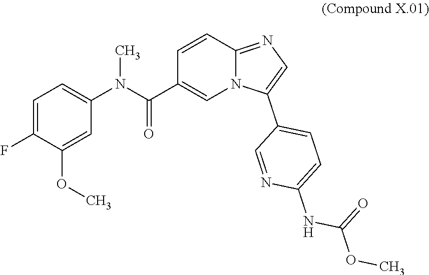 [SMILES: COC(=O)Nc1ccc(-c2cnc3ccc(C(=O)N(C)c4ccc(F)c(OC)c4)cn23)cn1]

A mixture of 3-bromo-N-(4-fluoro-3-methoxy-phenyl)-N-methyl-imidazo[1,2-a]pyridine-6-carboxamide (0.809 g, 2.14 mmol, 1equiv.), methyl N-[5-[6-[(4-fluoro-3-methoxy-phenyl)-methyl-carbamoyl]imidazo[1,2-a]pyridin-3-yl]-2-pyridyl]carbamate (0.877 g, 3.00 mmol, 1.4 equiv.) and cesium carbonate (1.045 g, 3.21 mmol, 1.5 equiv.) in water (4.3) and 1,4-dioxane (12.8 mL) was flushed with argon for 5 min. Tetrakis(triphenylphosphine)palladium(0) (0.127 g, 0.107 mmol, 0.05 equiv.) was then added and the reaction mixture was heated under microwave irradiation at 100° C. for 1 hour. The reaction mixture was cooled down to room temperature and then water was added. The aqueous layer was extracted with ethyl acetate and the combined organic layers were washed with brine, dried over sodium sulfate, filtered, and concentrated under reduced pressure. The crude residue was purified over a silica gel cartridge (cyclohexane/[ethyl acetate/EtOH 3:1]) to afford methyl N-[5-[6-[(4-fluoro-3-methoxy-phenyl)-methyl-carbamoyl]imidazo[1,2-a]pyridin-3-yl]-2-pyridyl]carbamate as a white solid. LC/MS (Method A) retention time=0.70; [M+H]⁺=450

¹H NMR (400 MHz, CDCl₃) δ ppm 8.28-8.26 (dd, J=0.7, 2.2 Hz, 1H), 8.20 (s, 2H), 8.15 (d, J=8.4 Hz, 1H), 7.68 (s, 1H), 7.55 (dd, J=2.4, 8.5 Hz, 1H), 7.51 (dd, J=0.7, 9.4 Hz, 1H), 7.23 (dd, J=1.6, 9.3 Hz, 1H), 7.00 (dd, J=8.5, 10.7 Hz, 1H), 6.74 (dd, J=2.5, 7.3 Hz, 1H), 6.64-6.58 (m, 1H), 3.87 (s, 3H), 3.80 (s, 3H), 3.47 (s, 3H).

### Example 2: This example illustrates the preparation of methyl N-[5-[6-[(4-fluoro-3-methoxy-phenyl)-methyl-carbamoyl]-8-methyl-imidazo[1,2-a]pyridin-3-yl]-2-pyridyl]carbamate (compound X.02 of Table T1)

### Step 1: Preparation of methyl 6-amino-5-chloro-pyridine-3-carboxylate

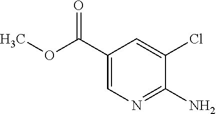

To an ice-cooled solution of methyl 6-aminopyridine-3-carboxylate (5.00 g, 32.9 mmol) in tetrahydrofuran (20.0 mL) was added N-chlorosuccinimide (4.39 g, 32.9 mmol, 1.00 equiv.). The reaction mixture was stirred at room temperature for 16 hours. The reaction mixture was then poured into water and extracted with ethyl acetate. The combined organic layers were washed with brine, dried over sodium sulfate, filtered, and concentrated under reduced pressure. The crude residue was purified over silica gel cartridge (cyclohexane/ethyl acetate) to afford methyl 6-amino-5-chloro-pyridine-3-carboxylate as an off-white solid.

¹H NMR (400 MHz, DMSO-d₆) δ ppm 8.47 (d, J=2.0 Hz, 1H), 7.93 (d, J=2.0 Hz, 1H), 7.73 (br s, 2H), 2.79 (s, 3H).

### Step 2: Preparation of methyl 8-chloroimidazo[1,2-a]pyridine-6-carboxylate

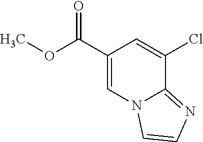

To a stirred solution of methyl 6-amino-5-chloro-pyridine-3-carboxylate (4.50 g, 21.7 mmol) in ethanol (90.0 mL) was added 2-chloroacetaldehyde (17.0 g, 109 mmol, 5.00 equiv.) at room temperature. The reaction mixture was stirred at 80° C. for 16 hours. The reaction mixture was then cooled, concentrated under reduced pressure, and the resulting concentrated solution was poured into a cold solution of saturated sodium carbonate and stirred for an additional 5 min. The aqueous layer was extracted with dichloromethane, the combined organic layers were washed with brine, dried over sodium sulfate, filtered, and concentrated under reduced pressure. The crude residue was purified over silica gel cartridge (cyclohexane/ethyl acetate) to afford methyl 8-chloroimidazo[1,2-a]pyridine-6-carboxylate as an off-white solid.

¹H NMR (400 MHz, DMSO-d₆) δ ppm 8.86 (d, J=1.2 Hz, 1H), 7.82 (d, J=1.6 Hz, 1H), 7.76 (d, J=1.2 Hz, 1H), 7.73 (d, J=1.2 Hz, 1H), 2.96 (s, 3H).

### Step 3: Preparation of 8-chloro-N-(4-fluoro-3-methoxy-phenyl)imidazo[1,2-a]pyridine-6-carboxamide

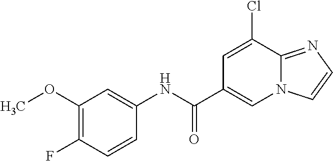 [SMILES: COc1cc(NC(=O)c2cc(Cl)c3nccn3c2)ccc1F]

To a stirred solution of methyl 8-chloroimidazo[1,2-a]pyridine-6-carboxylate (0.500 g, 2.14 mmol) and 4-fluoro-3-methoxy-aniline (0.377 g, 2.67 mmol, 1.25 equiv.) in dry toluene (20.0 mL) under a nitrogen atmosphere was added trimethylaluminium (2 mol/L, 3.20 mL, 6.41 mmol, 3.00 equiv.). The resulting mixture was stirred at 100° C. for 6 hours and then cooled to 0° C. The reaction mixture was quenched with a saturated aqueous solution of sodium sulfite, methanol was added, and the mixture further diluted with ethyl acetate. The reaction mixture was filtered through a pad of celite, the filtrate was washed with water and brine, dried over sodium sulfate, filtered, and concentrated under reduced pressure. The crude residue was purified over silica gel cartridge (cyclohexane/ethyl acetate) to afford 8-chloro-N-(4-fluoro-3-methoxy-phenyl)imidazo[1,2-a]pyridine-6-carboxamide as an off white solid.

¹H NMR (400 MHz, DMSO-d₆) δ ppm 10.38 (s, 1H), 9.26 (d, 1H), 8.21 (s, 1H), 7.95 (d, 1H), 7.72 (s, 1H), 7.55-7.62 (m, 1H), 7.37-7.33 (m, 1H), 7.24-7.16 (m, 1H), 2.85 (s, 3H).

### Step 4: Preparation of N-(4-fluoro-3-methoxy-phenyl)-8-methyl-imidazo[1,2-a]pyridine-6-carboxamide

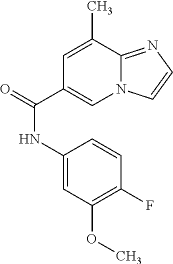 [SMILES: COc1cc(NC(=O)c2cc(C)c3nccn3c2)ccc1F]

To a stirred solution of 8-chloro-N-(4-fluoro-3-methoxy-phenyl)imidazo[1,2-a]pyridine-6-carboxamide (2.8 g, 7.88 mmol, 1.00 equiv.) and methylboronic acid (0.963 g, 15.8 mmol, 2.00 equiv.), in tetrahydrofuran (50.0 mL) in a seal tube, wase added potassium carbonate (2.78 g, 19.7 mmol, 2.50 equiv.).The reaction mixture was flushed with argon for 5 min, and chloro(2-dicyclohexylphosphino-2′,4′,6′-triisopropyl-1,1′-biphenyl)[2-(2′-amino-1,1′-biphenyl)]palladium(II) (0.632 g, 0.788 mmol, 0.10 equiv.) was added. The reaction mixture was stirred at 85° C. for 16 hours. The mixture was then poured into water, and then extracted with ethyl acetate. The combined organic layers were washed with brine, dried over MgSO4, filtered, and concentrated under reduced pressure. The crude residue was purified over silica gel cartridge (dichloromethane/methanol) to afford N-(4-fluoro-3-methoxy-phenyl)-8-methyl-imidazo[1,2-a]pyridine-6-carboxamide as off white solid.

¹H NMR (400 MHz, DMSO-d₆) δ ppm 10.31 (s, 1H), 9.09 (s, 1H), 8.08 (d, J=1.2 Hz, 1H), 7.62 (m, 2H), 7.55 (s, 1H), 7.33-7.37 (m, 1H), 7.18-7.23 (m, 1H), 3.84 (s, 3H), 2.59 (s, 3H)

### Step 5: Preparation of N-(4-fluoro-3-methoxy-phenyl)-N,8-dimethyl-imidazo[1,2-a]pyridine-6-carboxamide

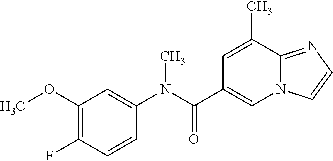 [SMILES: COc1cc(N(C)C(=O)c2cc(C)c3nccn3c2)ccc1F]

To an ice-cooled mixture of N-(4-fluoro-3-methoxy-phenyl)-8-methyl-imidazo[1,2-a]pyridine-6-carboxamide (95.0%, 1.00 g, 3.17 mmol, 1.00 equiv.) dissolved in tetrahydrofuran (15.0 mL) under argon, was added sodium hydride (60 mass % in oil (0.229 g, 9.52 mmol, 3.00 equiv.) portion wise, and the resulting mixture was stirred for 20 minutes at 0° C. Iodomethane (0.395 mL, 6.35 mmol, 2.00 equiv.) was then added dropwise and the resulting mixture was stirred at room temperature for 3 hours. The mixture was diluted with ethyl acetate and treated with a saturated solution of ammonium chloride. The desired material was extracted with ethyl acetate, the combined organic layers were washed with water, brine, dried over sodium sulfate, filtered, and concentrated under reduced pressure. The crude residue was purified over silica gel cartridge (dichloromethane/methanol) to afford N-(4-fluoro-3-methoxy-phenyl)-N,8-dimethyl-imidazo[1,2-a]pyridine-6-carboxamide as an off-white solid.

¹H NMR (400 MHz, DMSO-d₆) δ ppm 8.45 (s, 1H), 7.88 (d, J=1.2 Hz, 1H), 7.51 (d, J=1.2 Hz, 1H), 7.21-7.19 (dd, J=10.0, 2.4 Hz, 1H), 7.06-7.11 (m, 1H), 6.90 (s, 1H), 6.72-6.76 (m, 1H), 3.76 (s, 3H), 3.37 (s, 3H), 2.34 (s, 3H).

### Step 6: Preparation of N-(4-fluoro-3-methoxy-phenyl)-3-iodo-N,8-dimethyl-imidazo[1,2-a]pyridine-6-carboxamide

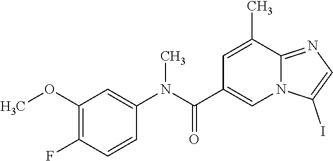 [SMILES: COc1cc(N(C)C(=O)c2cc(C)c3ncc(I)n3c2)ccc1F]

To a stirred solution of N-(4-fluoro-3-methoxy-phenyl)-N,8-dimethyl-imidazo[1,2-a]pyridine-6-carboxamide (95.0%, 80.0 mg, 0.243 mmol, 1.00equiv.) in dimethylformamide (2.00 mL) was added N-iodosuccinimide (66 mg, 0.29 mmol, 1.2 equiv.) and the resulting mixture was stirred at room temperature for 3 hours. The mixture was then poured into water, and the aqueous layer was extracted with ethyl acetate. The combined organic layers were washed with brine, dried over magnesium sulfate, filtered, and concentrated under reduced pressure. The crude residue was purified over silica gel cartridge (dichloromethane/methanol) to afford N-(4-fluoro-3-methoxy-phenyl)-3-iodo-N,8-dimethyl-imidazo[1,2-a]pyridine-6-carboxamide as an off-white solid.

¹H NMR (400 MHz, DMSO-d₆) δ ppm 7.99 (s, 1H), 7.66 (s, 1H), 7.39-7.36 (dd, J=10.4, 2.4 Hz, 1H), 7.13-7.05 (m, 2H), 6.78-6.75 (m, 1H), 3.79 (s, 3H), 3.39 (s, 3H), 2.40 (s, 3H).

### Step 7: Preparation of methyl N-[5-[6-((4-fluoro-3-methoxy-phenyl)-methyl-carbamoyl]-8-methyl-imidazo(1,2-a]pyridin-3-yl]-2-pyridyl]carbamate

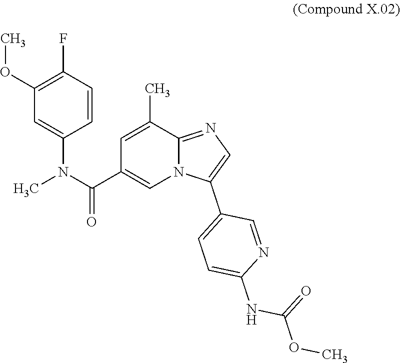 [SMILES: COC(=O)Nc1ccc(-c2cnc3c(C)cc(C(=O)N(C)c4ccc(F)c(OC)c4)cn23)cn1]

To a stirred mixture of N-(4-fluoro-3-methoxy-phenyl)-3-iodo-N,8-dimethyl-imidazo[1,2-a]pyridine-6-carboxamide (0.600 g, 1.30 mmol, 1.00 equiv.), methyl N-[5-(4,4,5,5-tetramethyl-1,3,2-dioxaborolan-2-yl)-2-pyridyl]carbamate (442 mg, 1.56 mmol, 1.20 equiv.) in 1,4-dioxane/water (3/1, 16.0 mL) was added cesium carbonate (863 mg, 2.60 mmol, 2.00 equiv.). The mixture was purged with a stream of argon for 2 minutes after which cataCXium® A Pd G3 (48.2 mg, 0.0649 mmol, 0.05 equiv.) was added. The resulting reaction mixture was irradiated under microwave irradiation at 100° C. for 1 hour. After cooling down to room temperature, the reaction mixture was filtered through a pad of celite. The filtrate was washed with water and brine, dried over sodium sulfate, filtered, and concentrated under reduced pressure. The crude residue was purified over silica gel cartridge (dichloromethane/methanol) to afford methyl N-[5-[6-[(4-fluoro-3-methoxy-phenyl)-methyl-carbamoyl]-8-methyl-imidazo[1,2-a]pyridin-3-yl]-2-pyridyl]carbamate as an off white solid.

¹H NMR (400 MHz, DMSO-d₆): δ ppm 10.46 (s, 1H), 8.33 (d, J=2.0 Hz, 1H), 8.09 (s, 1H), 7.99 (d, J=9.2 Hz, 1H), 7.73 (s, 1H), 7.63 (dd, J=8.8, 2.0 Hz, 1H), 7.30 (dd, J=7.6, 2.4 Hz, 1H), 7.14 (s, 1H), 7.03-7.08 (m, 1H), 6.70-6.73 (m, 1H), 3.73 (s, 3H), 3.72 (s, 3H), 3.36 (s, 3H), 2.44 (s, 3H).

### Example 3: This example illustrates the preparation of methyl N-[5-[6-[(4-fluoro-3-methoxy-phenyl)-(methoxymethyl)carbamoyl-8-methyl-imidazo[1,2-a]pyridin-3-yl]-2-pyridyl]carbamate (compound X.03 of Table T1)

### Step 1: Preparation of N-(4-fluoro-3-methoxy-phenyl)-N-(methoxymethyl)-8-methyl-imidazo(1,2-a]pyridine-6-carboxamide

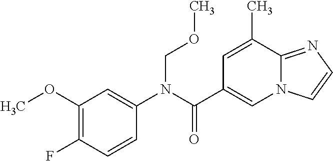 [SMILES: COCN(C(=O)c1cc(C)c2nccn2c1)c1ccc(F)c(OC)c1]

To an ice-cooled solution of N-(4-fluoro-3-methoxy-phenyl)-8-methyl-imidazo[1,2-a]pyridine-6-carboxamide (1.00 g, 3.01 mmol, 1.00 equiv.) in dry tetrahydrofuran (20.0 mL) was added sodium hydride (60% in oil, 0.361 g, 15.0 mmol, 5.00 equiv.) under a nitrogen atmosphere. The mixture was stirred for 30 minutes and then chloro(methoxy)methane (0.484 g, 6.01 mmol, 2.00 equiv.) was added. The resulting reaction mixture was warmed to room temperature and stirred for an additional 16 hours. The mixture was then diluted with ethyl acetate, and treated with crushed ice. The aqueous phase was extracted with ethyl acetate, the combined organic layers were washed with water and brine, dried over sodium sulfate, filtered, and concentrated under reduced pressure. The crude residue was purified over a silica gel cartridge (cyclohexane/ethyl acetate) to afford N-(4-fluoro-3-methoxy-phenyl)-N-(methoxymethyl)-8-methyl-imidazo[1,2-a]pyridine-6-carboxamide as an off white solid.

LC/MS (Method C) retention time=1.59 min; [M+H]⁺=344

¹H NMR (400 MHz, DMSO-d₆) δ 8.57 (s, 1H), 7.91 (d, J=1.2 Hz, 1H), 7.54 (d, J=1.2 Hz, 1H), 7.20 (dd, J=7.6, 2.4 Hz, 1H), 7.09-7.14 (m, 1H), 6.97 (s, 1H), 6.75-6.79 (m, 1H), 5.16 (s, 2H), 3.77 (s, 3H), 3.35 (s, 3H), 2.36 (s, 3H).

### Step 2: Preparation of N-(4-fluoro-3-methoxy-phenyl)-3-iodo-N-(methoxymethyl)-8-methyl-imidazo[1,2-a]pyridine-6-carboxamide

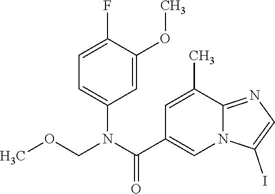 [SMILES: COCN(C(=O)c1cc(C)c2ncc(I)n2c1)c1ccc(F)c(OC)c1]

To a solution of N-(4-fluoro-3-methoxy-phenyl)-N-(methoxymethyl)-8-methyl-imidazo[1,2-a]pyridine-6-carboxamide (300 mg, 0.786 mmol, 1.00equiv.) in dimethylformamide (2.00 mL) was added N-iodosuccinimide (195 mg, 0.865 mmol, 1.10 equiv.) and the resulting mixture was stirred at room temperature for 16 hours. The mixture was then poured into water, and then extracted with ethyl acetate. The combined organic layers were washed with brine, dried over magnesium sulfate, filtered, and concentrated under reduced pressure. The crude residue was purified over silica gel cartridge (cyclohexane/ethyl acetate) to afford N-(4-fluoro-3-methoxy-phenyl)-3-iodo-N-(methoxymethyl)-8-methyl-imidazo[1,2-a]pyridine-6-carboxamide as an off-white solid.

¹H NMR (400 MHz, DMSO-d₆) δ ppm 8.15 (s, 1H), 7.69 (s, 1H), 7.36 (dd, J=8.00, 2.4 Hz, 1H), 7.19 (s, 1H), 7.08-7.13 (m, 1H), 6.79-6.81 (m, 1H), 5.18 (s, 2H), 3.80 (s, 3H), 3.37 (s, 3H), 2.42 (s, 3H).

### Step 3: Preparation of methyl N-[5-[6-((4-fluoro-3-methoxy-phenyl)-(methoxymethyl)carbamoyl]-8-methyl-imidazo[1,2-a]pyridin-3-yl]-2-pyridyl]carbamate

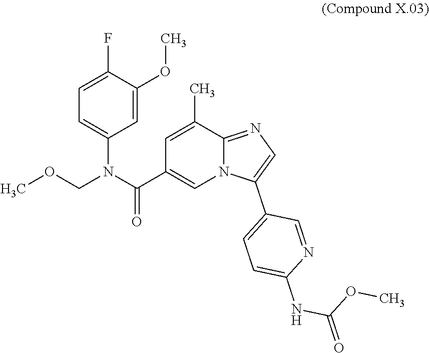

To a solution of N-(4-fluoro-3-methoxy-phenyl)-3-iodo-N-(methoxymethyl)-8-methyl-imidazo[1,2-a]pyridine-6-carboxamide (2.00 g, 3.84 mmol, 1.00 equiv.) and methyl N-[5-(4,4,5,5-tetramethyl-1,3,2-dioxaborolan-2-yl)-2-pyridyl]carbamate (1.31 g, 4.60 mmol, 1.20 equiv.) in 1,4-dioxane/water (3/1, 20.0 5 mL) was added cesium carbonate (2.50 g, 7.67 mmol, 2.00 equiv.). The mixture was purged with a stream of argon for 2 minutes after which cataCXium® A Pd G3 (0.140 g, 0.192 mmol, 0.05 equiv.) was added. The resulting mixture was heated under microwave irradiation at 100° C. for 1 hour. After cooling down to room temperature, the reaction mixture was filtered through a Celite pad and washed with ethyl acetate. The filtrate was washed with water and brine, dried over sodium sulfate, filtered, and concentrated under reduced pressure. The crude residue was purified over silica gel cartridge (dichloromethane/methanol) to afford methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate as an off white solid.

LC/MS (Method C) retention time=2.98 min; [M+H]⁺=494

¹H NMR (400 MHz, DMSO-d₆) δ ppm 10.44 (s, 1H), 8.36 (d, J=2.0 Hz, 1H), 8.25 (bs, 1H), 8.01 (d, J=8.8 Hz, 1H), 7.75 (s, 1H), 7.72 (br d, J=8.0 Hz, 1H), 7.27 (dd, J=8.0, 2.4 Hz, 1H), 7.19 (s, 1H), 7.08 (dd, J=11.2, 8.4 Hz, 1H), 6.78-6.75 (m, 1H), 5.15 (s, 2H), 3.73 (app s, 6H), 3.32 (s, 3H), 2.46 (s, 3H).

### Example 4: This example illustrates the preparation of methyl N-[5-[6-[(4-fluoro-3-methoxy-phenyl)-(methoxymethyl)carbamoyl]imidazo[1,2-a]pyridin-3-yl]-2-pyridyl]carbamate (compound X.04 of Table T1)

### Step 1: Preparation of N-(4-fluoro-3-methoxy-phenyl)imidazo[1,2-a]pyridine-6-carboxamide

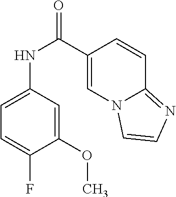 [SMILES: COc1cc(NC(=O)c2ccc3nccn3c2)ccc1F]

To a solution of methyl imidazo[1,2-a]pyridine-6-carboxylate (3.00 g, 16.2 mmol, 1.00 equiv.) in dry toluene (2.00 mL) under a nitrogen was added 4-fluoro-3-methoxy-aniline (2.74 g, 19.4 mmol, 1.20 equiv.) and trimethylaluminum (2M in toluene, 32.4 mmol, 2.00 equiv.) at room temperature. The resulting reaction mixture was stirred at 80° C. for 3 hours. The reaction mixture was cooled to 0° C., quenched with aqueous Rochelle's salt, filtered through a Celite pad and washed with ethyl acetate. The filtrate was washed with water and brine, dried over sodium sulfate, filtered, and concentrated under reduced pressure. The crude residue was purified over a silica gel cartridge (cyclohexane/ethyl acetate) to afford N-(4-fluoro-3-methoxy-phenyl)imidazo[1,2-a]pyridine-6-carboxamide as a white solid. LC/MS (Method C) retention time=0.11 min; [M+H]⁻=286

¹H NMR (400 MHz, DMSO-d₆) δ ppm 10.38 (s, 1H), 9.26 (s, 1H), 8.11 (s, 1H), 7.74 (dd, J=8.8, 1.6 Hz, 1H), 7.70-7.67 (m, 2H), 7.64 (dd, J=8.4, 2.4 Hz, 1H), 7.37-7.33 (m, 1H), 7.24-7.19 (m, 1H), 3.85 (s, 3H).

### Step 2: Preparation of N-(4-fluoro-3-methoxy-phenyl)-N-(methoxymethyl)imidazo(1,2-a]pyridine-6-carboxamide

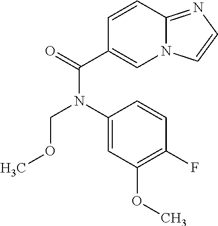 [SMILES: COCN(C(=O)c1ccc2nccn2c1)c1ccc(F)c(OC)c1]

To an ice-cooled solution of N-(4-fluoro-3-methoxy-phenyl)imidazo[1,2-a]pyridine-6-carboxamide (2.3 g, 7.66 mmol, 1.00 equiv.) in dry tetrahydrofuran (50.0 mL), under nitrogen was added sodium hydride (60% in oil, 0.919 g, 23.0 mmol, 3.00 eq). After 10 minutes of stirring, chloro(methoxy)methane (1.3 g, 15.3 mmol, 2.00 equiv.) was added and the reaction mixture was stirred for 30 minutes at 0° C. and then warmed up. After completion, the mixture was cooled down, diluted with ethyl acetate, and treated with crushed ice. The aqueous phase was extracted with ethyl acetate, the combined organic layers were washed with water and brine, dried over sodium sulfate, filtered, and concentrated under reduced pressure. The crude residue was purified over silica gel cartridge (cyclohexane/ethyl acetate) to afford N-(4-fluoro-3-methoxy-phenyl)-N-(methoxymethyl)imidazo[1,2-a]pyridine-6-carboxamide as a white solid.

LC/MS (Method C) retention time=1.29; [M+H]⁻=330

¹H NMR (400 MHz, DMSO-d₆) δ ppm 8.79 (s, 1H), 7.95 (s, 1H), 7.59 (s, 1H), 7.35-7.40 (d, 1H), 7.30 (dd, J=8.8, 1.6 Hz, 1H), 7.00-7.12 (m, 2H), 6.72-6.80 (m, 1H), 5.19 (s, 2H), 3.75 (s, 3H), 3.34 (s, 3H).

### Step 3: Preparation of N-(4-fluoro-3-methoxy-phenyl)-3-iodo-N-(methoxymethyl)imidazo[1,2-a]pyridine-6-carboxamide

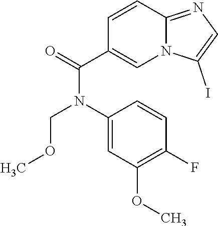 [SMILES: COCN(C(=O)c1ccc2ncc(I)n2c1)c1ccc(F)c(OC)c1]

To a solution of N-(4-fluoro-3-methoxy-phenyl)-N-(methoxymethyl)imidazo[1,2-a]pyridine-6-carboxamide (2.4 g, 6.9 mmol, 1.0 equiv.) in dimethylformamide (40.0 mL) at room temperature was added N-iodosuccinimide (1.75 g, 7.62 mmol, 1.10 equiv.) and the resulting mixture was stirred for 2 hours. The mixture was then poured into water and extracted with ethyl acetate. The combined organic layers were washed with brine, dried over sodium sulfate, filtered, and concentrated under reduced pressure. The crude residue was purified over silica gel cartridge (cyclohexane/ethyl acetate) to afford N-(4-fluoro-3-methoxy-phenyl)-3-iodo-N-(methoxymethyl)imidazo[1,2-a]pyridine-6-carboxamide as a brown solid.

LC/MS (Method C) retention time=1.61; [M+H]⁻=456

¹H NMR (400 MHz, CDCl₃) δ ppm 8.25 (s, 1H), 7.63 (s, 1H), 7.35 (d, J=9.6 Hz, 1H), 7.16 (d, J=8.8 Hz, 1H), 6.90-6.95 (m, 1H), 6.83 (dd, J=10.0, 2.4 Hz, 1H), 6.62-6.66 (m, 1H), 5.15 (s, 2H), 3.76 (s, 3H), 3.44 (s, 3H).

### Step 4: Preparation of methyl N-[5-[6-T(4-fluoro-3-methoxy-phenyl)-(methoxymethyl)carbamoyl]imidazo(1,2-a]pyridin-3-yl]-2-pyridyl]carbamate

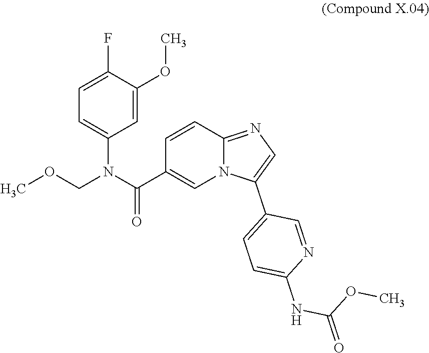

To a solution of N-(4-fluoro-3-methoxy-phenyl)-3-iodo-N-(methoxymethyl)imidazo[1,2-a]pyridine-6-carboxamide (0.600 g, 1.25 mmol, 1.00 equiv.) and methyl N-[5-(4,4,5,5-tetramethyl-1,3,2-dioxaborolan-2-yl)-2-pyridyl]carbamate (0.426 g, 1.50 mmol, 1.20 equiv.) in 1,4-dioxane/water (3/1, 16.0 mL) was added cesium carbonate (0.833 g, 2.50 mmol, 2.00 equiv.). The mixture was then purged with a stream of argon for 2 minutes and cataCXium® A Pd G3 (46.5 mg, 0.0626 mmol, 0.05 equiv.) was added. The resulting reaction mixture was heated under microwave irradiation at 100° C. for 1 hour.

After cooling down to room temperature, the mixture was filtered through a celite pad and washed with ethyl acetate. The filtrate was washed with water and brine, dried over sodium sulfate, filtered, and concentrated under reduced pressure. The crude residue was purified over silica gel cartridge (dichloromethane/methanol) to afford methyl N-[5-[6-[(4-fluoro-3-methoxy-phenyl)-(methoxymethyl)carbamoyl]imidazo[1,2-a]pyridin-3-yl]-2-pyridyl]carbamate as a white solid.

LC/MS (Method A) retention time=0.74; [M+H]⁺=480

¹H NMR (400 MHz, CDCl₃) δ ppm 8.30 (s, 1H), 8.24 (d, J=2.0 Hz, 1H), 8.14 (d, J=8.8 Hz, 1H), 7.96 (br s, 1H), 7.69 (s, 1H), 7.52-7.58 (m, 2H), 7.28-7.30 (m, 1H), 1) 6.98-7.03 (m, 1H), 6.84-6.86 (m, 1H), 6.62-6.68 (m, 1H), 5.18 (s, 2H), 3.86 (s, 3H), 3.81 (s, 3H), 3.45 (s, 3H).

### Example 5: This example illustrates the preparation of methyl N-[4-[6-[(4-fluoro-3-methoxy-phenyl)-methyl-carbamoyl]imidazo[1,2-a]pyridin-3-yl]phenyl]carbamate (compound X.05 of Table T1)

### Step 1: Preparation of methyl N-[4-[6-((4-fluoro-3-methoxy-phenyl)-methyl-carbamoyl]imidazo[1,2-a]pyridin-3-yl]phenyl]carbamate

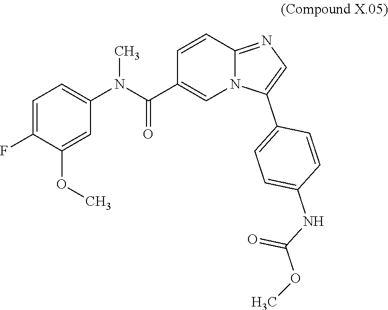 [SMILES: COC(=O)Nc1ccc(-c2cnc3ccc(C(=O)N(C)c4ccc(F)c(OC)c4)cn23)cc1]

To a mixture of 3-bromo-N-(4-fluoro-3-methoxy-phenyl)-N-methyl-imidazo[1,2-a]pyridine-6-carboxamide (0.425 g, 1.12 mmol, 1.00 equiv.), methyl (4-(4,4,5,5-tetramethyl-1,3,2-dioxaborolan-2-yl)phenyl)carbamate (0.445 g, 1.57 mmol, 1.40 equiv.), cesium carbonate (0.549 g, 1.69 mmol, 1.50 equiv.) and tetrakis(triphenylphosphine)palladium(0) (0.0669 g, 0.0562 mmol, 0.050 equiv.) under an atmosphere of nitrogen was added water (2.25 mL) and 2-methyltetrahydrofuran (6.74 mL). The suspension was purged with a stream of argon by three freeze-pump-thaw cycles and heated under microwave irritation at 100° C. for 60 minutes. After cooling down to room temperature, the mixture was filtered through a celite pad. The filtrate was washed with water and brine, dried over sodium sulfate, filtered, and concentrated under reduced pressure. The crude residue was purified over a silica gel cartridge (cyclohexane/ethyl acetate) to afford methyl N-[4-[6-[(4-fluoro-3-methoxy-phenyl)-methyl-carbamoyl]imidazo[1,2-a]pyridin-3-yl]phenyl]carbamate.

LC/MS (Method A) retention time=0.70; [M+H]⁻=449

¹H NMR (400 MHz, CDCl₃) δ ppm 8.22 (s, 1H), 7.62 (s, 1H), 7.53 (br d, J=8.4 Hz, 2H), 7.50 (dd, J=9.5, 0.7 Hz, 1H), 7.25 (dd, J=9.5, 1.8 Hz, 1H), 7.18 (d, J=8.4 Hz, 2H), 7.00 (dd, J=10.7, 8.5 Hz, 1H), 6.95 (s, 1H), 6.74 (dd, J=7.3, 2.5 Hz, 1H), 6.58-6.64 (m, 1H), 3.84 (s, 3H), 3.78 (s, 3H), 3.47 (s, 3H).

### Example 6: This example illustrates the preparation of 3-[6-(cyclopropanecarbonylamino)-3-pyridyl]-N-(4-fluoro-3-methoxy-phenyl)-N-methyl-imidazo[1,2-a]pyridine-6-carboxamide (compound X.06 of Table T1)

### Step 1: Preparation of N-[5-(4,4,5,5-tetramethyl-1,3,2-dioxaborolan-2-yl)-2-pyridyl]cyclopropanecarboxamide

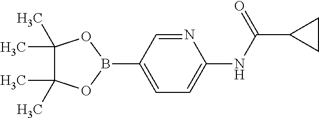 [SMILES: CC1(C)OB(c2ccc(NC(=O)C3CC3)nc2)OC1(C)C]

To a solution of 2-aminopyridine-5-boronic acid pinacolester (1.00 g, 4.32 mmol, 1.00 equiv.) and pyridine (1.42 mL, 17.3 mmol, 4.00 equiv.) in ethyl acetate (40 mL) at 10° C. was added, dropwise, cyclopropanecarbonyl chloride (1.40 g, 13.0 mmol, 3.00 equiv.). The reaction mixture was slowly warmed to room temperature and stirred until full conversion. The mixture was then diluted with ethyl acetate and treated with a saturated solution of sodium bicarbonate. The aqueous phase was extracted with ethyl acetate, the combined organic layers were washed with water and brine, dried over sodium sulfate, filtered, and concentrated under reduced pressure to afford N-[5-(4,4,5,5-tetramethyl-1,3,2-dioxaborolan-2-yl)-2-pyridyl]cyclopropanecarboxamide as a white solid. The crude material was used in the next step without further purification.

This compound can also be purchased (1201644-41-8)

### Step 2: Preparation of 3-[6-(cyclopropanecarbonylamino)-3-pyridyl]-N-(4-fluoro-3-methoxy-phenyl)-N-methyl-imidazo(1,2-a]pyridine-6-carboxamide

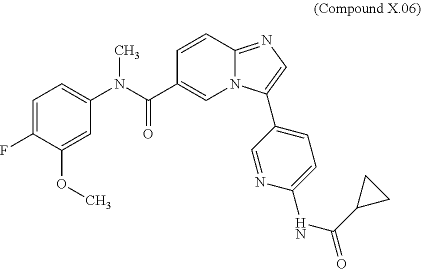 [SMILES: COc1cc(N(C)C(=O)c2ccc3ncc(-c4ccc(NC(=O)C5CC5)nc4)n3c2)ccc1F]

A mixture of 3-bromo-N-(4-fluoro-3-methoxy-phenyl)-N-methyl-imidazo[1,2-a]pyridine-6-carboxamide (0.200 g, 0.513 mmol, 1.00 equiv.), N-[5-(4,4,5,5-tetramethyl-1,3,2-dioxaborolan-2-yl)-2-pyridyl]cyclopropanecarboxamide (0,222 g, 0,769 mmol, 1.50 equiv.) and sodium carbonate (0.163 g, 1.54 mmol, 3.00 equiv.) in a mixture of acetonitrile (4 mL) and water (2 mL). was purged with a stream of argon for 5 minutes after which chloro(2-dicyclohexylphosphino-2′,4′,6′-triisopropyl-1,1′-biphenyl)[2-(2′-amino-1,1′-biphenyl)]palladium(II) (0.041 g, 0.051 mmol, 0.10 equiv.) was added. The resulting reaction mixture was heated under microwave irradiation at 100° C. for 1.5 hours. After cooling down to room temperature, the mixture was filtered through a celite pad and washed with ethyl acetate. The filtrate was washed with water and brine, dried over sodium sulfate, filtered, and concentrated under reduced pressure. The crude residue was purified by preparative HPLC (acetonitrile/water) to afford 3-[6-(cyclopropanecarbonylamino)-3-pyridyl]-N-(4-fluoro-3-methoxy-phenyl)-N-methyl-imidazo[1,2-a]pyridine-6-carboxamide.

LC/MS (Method C) retention time=0.94 min; [M+H]⁺=460

¹H NMR (400 MHz, CDCl₃) δ ppm 11.07 (s, 1H), 8.47-8.51 (m, 1H), 8.39 (s, 1H), 8.30 (d, J=8.7 Hz, 1H), 7.86 (s, 1H), 7.81 (dd, J=8.7, 2.4 Hz, 1H), 7.58 (d, J=9.3 Hz, 1H), 7.21-7.36 (m, 2H), 7.12 (dd, J=11.3, 8.5 Hz, 1H), 6.78-6.87 (m, 1H), 3.76 (s, 3H), 3.39 (s, 3H), 2.09-2.19 (m, 1H), 0.87-0.99 (m, 4H).

### Example 7: This example illustrates the preparation of methyl N-[5-[6-[ethyl-(4-fluorophenyl)carbamoyl]imidazo[1,2-a]pyridin-3-yl]-2-pyridyl]carbamate (compound X.19 of Table T1)

### Step 1: Preparation of 6-bromo-3-iodo-imidazo(1,2-a]pyridine

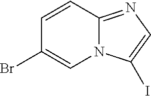 [SMILES: Brc1ccc2ncc(I)n2c1]

To a solution of 6-bromoimidazo[1,2-a]pyridine (2.00 g, 10.0 mmol) in acetonitrile (41 mL) was added N-iodosuccinimide (2.3 g, 10 mmol, 1.0 equiv.). The reaction was stirred at room temperature for 16 hours. The reaction mixture was poured into water and extracted with ethyl acetate. The combined organic layers were washed with sodium thiosulphate pentahydrate then brine, dried over sodium sulfate, filtered and concentrated under reduced pressure to afford 6-bromo-3-iodo-imidazo[1,2-a]pyridine as a white solid.

LC/MS (Method A) retention time=0.75 min; [M+H]⁺=325

### Step 2: Preparation of methyl N-[5-(6-bromoimidazo[1,2-a]pyridin-3-yl)-2-pyridyl]carbamate

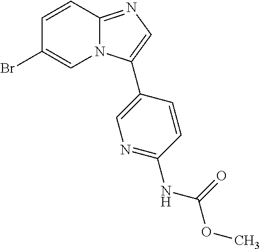 [SMILES: COC(=O)Nc1ccc(-c2cnc3ccc(Br)cn23)cn1]

To a suspension of 6-bromo-3-iodo-imidazo[1,2-a]pyridine (0.450 g, 1.40 mmol) in 2-methyltetrahydrofuran (9.1 mL) and water (2.3 mL) under argon were added methyl N-[5-(4,4,5,5-tetramethyl-1,3,2-dioxaborolan-2-yl)-2-pyridyl]carbamate (0.410 g, 1.40 mmol, 1.05 equiv.), potassium carbonate (0.260 g, 1.90 mmol, 1.40 equiv.) and [1,1′-bis(diphenylphosphino)ferrocene]dichloropalladium(II) (0.053 g, 0.068 mmol, 0.050 equiv.). The reaction mixture was stirred at 80° C. for 48 hours. The reaction mixture was filtered off. The solid was washed with 2-methyltetrahydrofuran and water, then dried under reduced pressure to afford methyl N-[5-(6-bromoimidazo[1,2-a]pyridin-3-yl)-2-pyridyl]carbamate.

LC/MS (Method A) retention time=0.63 min; [M+H]⁺=349

¹H NMR (400 MHz, CDCl₃) δ ppm 10.57-10.16 (m, 1H), 8.66 (s, 1H), 8.58-8.50 (m, 1H), 8.12 (br d, J=8.7 Hz, 1H), 8.01 (d, J=8.4 Hz, 1H), 7.83 (s, 1H), 7.66 (d, J=9.4 Hz, 1H), 7.43 (br d, J=9.4 Hz, 1H), 3.72 (s, 3H).

### Step 3: Preparation of methyl N-[5-[6-[ethyl-(4-fluorophenyl)carbamoyl]imidazo[1,2-a]pyridin-3-yl]-2-pyridyl]carbamate (compound X.19)

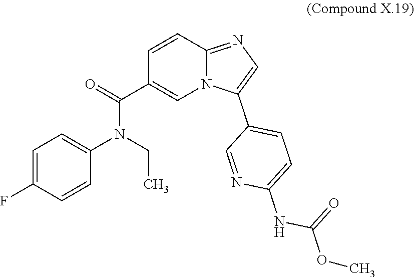 [SMILES: CCN(C(=O)c1ccc2ncc(-c3ccc(NC(=O)OC)nc3)n2c1)c1ccc(F)cc1]

To a solution of methyl N-[5-(6-bromoimidazo[1,2-a]pyridin-3-yl)-2-pyridyl]carbamate (0.175 g, 0.479 mmol) in toluene (1.20 mL) under argon were added N-ethyl-4-fluoro-aniline (0.078 mL, 0.60 mmol, 1.3 equiv.) followed by XantPhos Pd G3 (CAS 1445085-97-1) (0.0239 g, 0.0239 mmol, 0.0500 equiv.) and triethylamine (0.0974 g, 0.958 mmol, 2.00 equiv.). The vial was flushed with nitrogen, then filled with CO gas and pressurized with CO gas to 10 bar. The vessel was heated to 110° C. for 18 hours. The reaction mixture was then concentrated under reduced pressure. The residue was purified by flash chromatography over silica gel (eluting with cyclohexane/ethyl acetate/ethanol) to afford methyl N-[5-[6-[ethyl-(4-fluorophenyl)carbamoyl]imidazo[1,2-a]pyridin-3-yl]-2-pyridyl]carbamate as a brown oil.

LC/MS (Method A) retention time=0.73 min; [M+H]⁺=434

¹H NMR (400 MHz, CDCl₃) δ ppm 8.26 (s, 1H), 8.22 (s, 1H), 8.18 (br. d, J=8.72 Hz, 1H), 8.04-7.96 (m, 1H), 7.74-7.66 (m, 2H), 7.60 (br. d, J=8.36 Hz, 1H), 7.29 (br. d, J=9.08 Hz, 1H), 7.10-6.99 (m, 4H), 3.94 (q, J=7.0 Hz, 2H), 3.88 n(s, 3H), 1.21 (t, J=7.0 Hz, 3H).

### Example 8: This example illustrates the preparation of methyl N-[5-[8-acetamido-6-[(4-fluoro-3-methoxy-phenyl)-methyl-carbamoyl]imidazo[1,2-a]pyridin-3-yl]-2-pyridyl]carbamate (compound X.20 of Table T1)

### Step 1: Preparation of 6-bromoimidazo(1,2-a]pyridin-8-amine

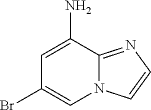 [SMILES: Nc1cc(Br)cn2ccnc12]

Chloroacetaldehyde (50% in water) (0.84 mL, 6.6 mmol, 1.25 equiv.) was added to a solution of 5-bromopyridine-2,3-diamine (1.00 g, 5.32 mmol) in ethanol (21 mL). The resulting solution was stirred at 100° C. for 3 hours. The reaction mixture was concentrated under reduced pressure to afford methyl 6-bromoimidazo[1,2-a]pyridine-8-carboxylate.

LC/MS (Method C) retention time=0.18 min; [M+H]⁺=213

### Step 2: Preparation of N-(6-bromoimidazo[1,2-a]pyridin-8-yl)acetamide

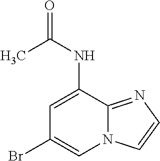

To a solution of 6-bromoimidazo[1,2-a]pyridin-8-amine (1.16 g, 4.38 mmol) in pyridine (11.6 mL) was added acetic anhydride (0.43 mL, 1.1 equiv.) dropwise at 0° C. The resulting mixture was stirred at room temperature for 12 hours. The reaction mixture was diluted with cold water, then extracted with ethyl acetate. The combined organic layers were dried over sodium sulfate, filtered and concentrated under reduced pressure to afford N-(6-bromoimidazo[1,2-a]pyridin-8-yl)acetamide.

LC/MS (Method C) retention time=0.21 min; [M+H]⁺=255

### Step 3: Preparation of 8-acetamido-N-(4-fluoro-3-methoxy-phenyl)-N-methyl-imidazo(1,2-a]pyridine-6-carboxamide

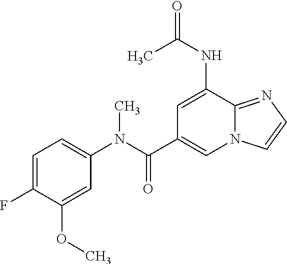 [SMILES: COc1cc(N(C)C(=O)c2cc(NC(C)=O)c3nccn3c2)ccc1F]

Bis(benzonitrile)palladium chloride (0.159 g, 0.394 mmol, 0.10 equiv.) and 4,5-bis(diphenylphosphino)-9,9-dimethylxanthene (0.232 g, 0.394 mmol, 0.10 equiv) were added to a mixture of N-(6-bromoimidazo[1,2-a]pyridin-8-yl)acetamide (1.000 g, 3.936 mmol), 4-fluoro-3-methoxy-N-methyl-aniline (0.641 g, 4.13 mmol, 1.05 equiv.) and triethylamine (0.83 mL, 5.9 mmol, 1.5 equiv.) in toluene (9.8 mL). The vial was flushed with nitrogen, then filled with CO gas and pressurized with CO gas to 10 bar. The vessel was heated to 110° C. for 6 hours. The reaction mixture was filtered through celite and the filtrate was concentrated under reduced pressure. The residue was purified by flash chromatography over silica gel (eluting cyclohexane/ethyl acetate) to afford 8-acetamido-N-(4-fluoro-3-methoxy-phenyl)-N-methyl-imidazo[1,2-a]pyridine-6-carboxamide as a brown solid.

LC/MS (Method C) retention time=0.86 min; [M+H]⁺=357

### Step 4: Preparation of 8-acetamido-N-(4-fluoro-3-methoxy-phenyl)-3-iodo-N-methyl-imidazo[1,2-a]pyridine-6-carboxamide

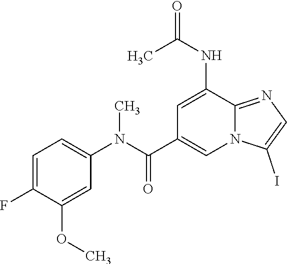 [SMILES: COc1cc(N(C)C(=O)c2cc(NC(C)=O)c3ncc(I)n3c2)ccc1F]

8-acetamido-N-(4-fluoro-3-methoxy-phenyl)-N-methyl-imidazo[1,2-a]pyridine-6-carboxamide (0.460 g, 1.29 mmol) was added to a solution of N-iodosuccinimide (0.326 g, 1.42 mmol, 1.10 equiv.) in acetonitrile (5.2 mL). the mixture was stirred at room temperature for 12 hours. The reaction mixture was then poured in water and extracted with ethyl acetate. The combined organic layers were washed with a solution of sodium thiosulfate, then brine, dried over sodium sulfate, filtered and concentrated under reduced pressure. The crude residue was purified by flash chromatography over silica gel (eluting cyclohexane/ethyl acetate) to afford 8-acetamido-N-(4-fluoro-3-methoxy-phenyl)-3-iodo-N-methyl-imidazo[1,2-a]pyridine-6-carboxamide.

LC/MS (Method C) retention time=1.03 min; [M+H]⁺=483

¹H NMR (400 MHz, CDCl₃) δ ppm 8.40 (br s, 1H), 8.15 (s, 1H), 8.06 (d, J=1.22 Hz, 1H), 7.57 (s, 1H), 6.95 (d, J=10.70, 8.62 Hz, 1H), 6.88 (d, J=7.40, 2.38 Hz, 1H), 6.61-6.68 (m, 1H), 3.87 (s, 3H), 3.49 (s, 3H), 2.22 (s, 3H).

### Step 5: Preparation of methyl N-[5-[8-acetamido-6-((4-fluoro-3-methoxy-phenyl)-methyl-carbamoyl]imidazo[1,2-a]pyridin-3-yl]-2-pyridyl]carbamate (compound X.20)

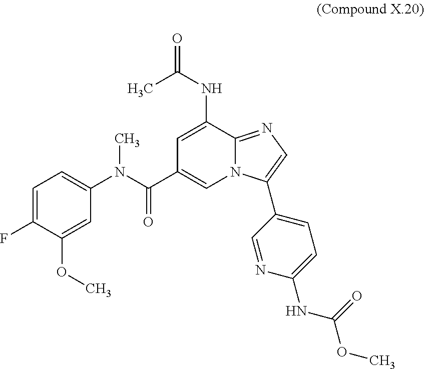 [SMILES: COC(=O)Nc1ccc(-c2cnc3c(NC(C)=O)cc(C(=O)N(C)c4ccc(F)c(OC)c4)cn23)cn1]

8-acetamido-N-(4-fluoro-3-methoxy-phenyl)-3-iodo-N-methyl-imidazo[1,2-a]pyridine-6-carboxamide (0.360 g, 0.747 mmol) and methyl N-[5-(4,4,5,5-tetramethyl-1,3,2-dioxaborolan-2-yl)-2-pyridyl]carbamate (0.311 g, 1.12 mmol, 1.50 equiv.) were dissolved in acetonitrile (5.97 mL). Then a solution of sodium carbonate (0.237 g, 2.24 mmol, 3.00 equiv.) in water (3.0 mL) was added. The reaction mixture was degassed with nitrogen for 15 min. Then chloro(2-dicyclohexylphosphino-2′,4′,6′-triisopropyl-1,1′-biphenyl)[2-(2′-amino-1,1′-biphenyl)]palladium(II) (0.0606 g, 0.0747 mmol, 0.100 equiv.) was added and the reaction mixture was subjected to microwave irradiation up to 100° C. for 1 hour. The reaction mixture was filtered through celite and the filtrate was concentrated under reduced pressure. The crude residue was purified by flash chromatography over neutral alumina (eluting ethyl acetate/methanol), then the desired product was further triturated with ethyl acetate to afford methyl N-[5-[8-acetamido-6-[(4-fluoro-3-methoxy-phenyl)-methyl-carbamoyl]imidazo[1,2-a]pyridin-3-yl]-2-pyridyl]carbamate as a white solid.

LC/MS (Method C) retention time=1.00 min; [M+H]⁺=507

¹H NMR (400 MHz, DMSO-d₆) δ ppm 10.48 (s, 1H), 10.07 (s, 1H), 8.37 (s, 1H), 8.11 (s, 1H), 7.95-8.04 (m, 2H), 7.76 (s, 1H), 7.70 (br d, 1H), 7.28-7.35 (m, 1H), 7.04 (dd, 1H), 6.67-6.74 (m, 1H), 3.73 (s, 6H), 3.35 (s, 3H), 2.20 (s, 3H).

### Example 9: This example illustrates the preparation of methyl N-[5-[6-[(4-fluoro-3-methoxy-phenyl)-methyl-carbamoyl-8-(methoxymethyl)imidazo[1,2-a]pyridin-3-yl]-2-pyridyl]carbamate (compound X.21 of Table T1)

### Step 1: Preparation of methyl 6-bromoimidazo(1,2-a]pyridine-8-carboxylate

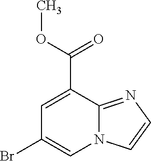

Chloroacetaldehyde (50% in water) (11.0 mL, 86.5 mmol, 2 equiv.) was added to a solution of methyl 2-amino-5-bromo-pyridine-3-carboxylate (10.00 g, 43.3 mmol) in ethanol (173 mL). The resulting solution was stirred at 100° C. for 9 hours and then at 55° C. for 12 hours. The reaction mixture was cooled down and diluted with saturated aqueous sodium bicarbonate solution. The aqueous layer was extracted with ethyl acetate. The combined organic layers were washed with brine, dried over sodium sulfate, filtered and concentrated under reduced pressure to afford methyl 6-bromoimidazo[1,2-a]pyridine-8-carboxylate.

LC/MS (Method C) retention time=0.17 min; [M+H]⁺=255

¹H NMR (400 MHz, CDCl₃) δ ppm 1.40 (s, 1H), 4.03 (s, 3H), 8.23 (d, J=2.00 Hz, 1H), 8.53 (d, J=2.00 Hz, 1H), 8.56 (d, J=1.75 Hz, 1H), 9.72 (d, J=1.75 Hz, 1H).

### Step 2: Preparation of (6-bromoimidazo[1,2-a]pyridin-8-yl)methanol

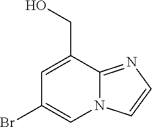 [SMILES: OCc1cc(Br)cn2ccnc12]

To a mixture of calcium chloride (12.0 g, 110 mmol, 10.0 equiv) and sodium borohydride (4.20 g, 110 mmol, 10.0 equiv) in tetrahdrofuran (150 mL) was added a mixture of methyl 6-bromoimidazo[1,2-a]pyridine-8-carboxylate (3.70 g, 11.0 mmol) in tetrahydrofuran at 0° C. Then the mixture was stirred at room temperature for 16 hours. The reaction mixture was cooled down to 0° C., and diluted with saturated aqueous ammonium chloride solution. The aqueous layer was extracted with ethyl acetate and the combined organic layers were washed with brine, dried over sodium sulfate, filtered and concentrated under reduced pressure to afford (6-bromoimidazo[1,2-a]pyridin-8-yl)methanol as an off-white solid.

LC/MS (Method C) retention time=0.17 min; [M+H]⁺=227

### Step 3: Preparation of 6-bromo-8-(methoxymethyl)imidazo[1,2-a]pyridine

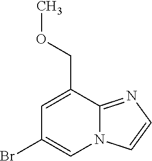

Sodium hydride (60% in oil) (169 mg, 4.21 mmol, 1.50 equiv.) was added to a stirred mixture of (6-bromoimidazo[1,2-a]pyridin-8-yl)methanol (638 mg, 2.81 mmol) in N,N-dimethylformamide (12.8 mL) at 0° C. the mixture was stirred at 0° C. for 30 min, then iodomethane (0.36 mL, 5.6 mmol, 2.0 equiv.) was added dropwise. The mixture was stirred at room temperature for 4 hours. The reaction mixture was cooled down to 0° C. and diluted with saturated aqueous ammonium chloride solution. The aqueous layer was extracted with ethyl acetate and the combined organic layers were washed with brine, dried over sodium sulfate, filtered and concentrated. The residue was purified by flash chromatography over silica gel (eluting cyclohexane/ethyl acetate) to afford 6-bromo-8-(methoxymethyl)imidazo[1,2-a]pyridine as a yellow solid.

LC/MS (Method C) retention time=0.24 min; [M+H]⁺=241

¹H NMR (400 MHz, CDCl₃) δ ppm 3.57 (s, 3H), 4.90 (s, 2H), 7.37 (d, J=1.38 Hz, 1H), 7.59 (d, J=1.00 Hz, 1H), 7.63 8s, 1 h9, 8.25 (s, 1H).

### Step 4: Preparation of N-(4-fluoro-3-methoxy-phenyl)-8-(methoxymethyl)-N-methyl-imidazo[1,2-a]pyridine-6-carboxamide

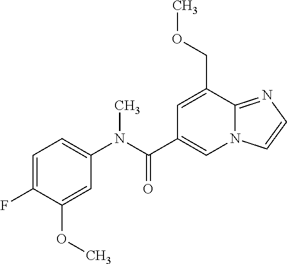 [SMILES: COCc1cc(C(=O)N(C)c2ccc(F)c(OC)c2)cn2ccnc12]

Bis(benzonitrile)palladium chloride (73.7 mg, 0.183 mmol, 0.10 equiv.) and 4,5-bis(diphenylphosphino)-9,9-dimethylxanthene (108 mg, 0.183 mmol, 0.10 equiv) were added to a mixture of 6-bromo-8-(methoxymethyl)imidazo[1,2-a]pyridine (440 mg, 1.825 mmol), 4-fluoro-3-methoxy-N-methyl-aniline (297 mg, 1.92 mmol, 1.05 equiv.) and triethylamine (0.38 mL, 2.7 mmol, 1.5 equiv.) in toluene (4.6 mL). The vial was flushed with nitrogen, then filled with CO gas and pressurized with CO gas to 10 bar. The vessel was heated to 110° C. for 6 hours. The reaction mixture was filtered through celite and the filtrate was concentrated under reduced pressure. The residue was purified by flash chromatography over silica gel (eluting cyclohexane/ethyl acetate) to afford N-(4-fluoro-3-methoxy-phenyl)-8-(methoxymethyl)-N-methyl-imidazo[1,2-a]pyridine-6-carboxamide as a brown solid.

LC/MS (Method C) retention time=0.90 min; [M+H]⁺=344

¹H NMR (400 MHz, CDCl₃) δ ppm 3.32 (s, 3H), 3.49 (s, 3H), 3.81 (s, 3H), 4.71 (s, 2H), 6.60-6.69 (m, 1H), 6.79 (dd, J=7.38, 2.50 Hz, 1H), 6.97 (dd, J=10.76, 8.50 Hz, 1H), 7.04 (s, 1H), 7.55 (d, J=1.13 Hz, 1H), 7.61 (d, J=1.13 Hz, 1H), 8.34 (s, 1H).

### Step 5: Preparation of N-(4-fluoro-3-methoxy-phenyl)-3-iodo-8-(methoxymethyl)-N-methyl-imidazo[1,2-a]pyridine-6-carboxamide

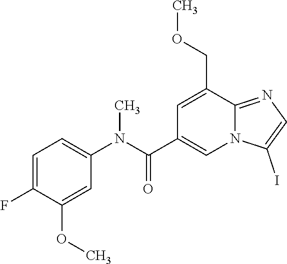 [SMILES: COCc1cc(C(=O)N(C)c2ccc(F)c(OC)c2)cn2c(I)cnc12]

N-(4-fluoro-3-methoxy-phenyl)-8-(methoxymethyl)-N-methyl-imidazo[1,2-a]pyridine-6-carboxamide (0.560 g, 1.63 mmol) was added to a solution of N-iodosuccinimide (0.374 g, 1.63 mmol, 1.00 equiv.) in acetonitrile (6.5 mL). the mixture was stirred at room temperature for one hour. The reaction mixture was then poured in water and extracted with ethyl acetate. The combined organic layers were washed with a solution of sodium thiosulfate, then brine, dried over sodium sulfate, filtered and concentrated under reduced pressure to afford N-(4-fluoro-3-methoxy-phenyl)-3-iodo-8-(methoxymethyl)-N-methyl-imidazo[1,2-a]pyridine-6-carboxamide as a brown solid.

LC/MS (Method C) retention time=1.01 min; [M+H]⁺=470

¹H NMR (400 MHz, CDCl₃) δ ppm 3.36 (s, 3H), 3.51 (s, 3H), 3.83 (s, 3H), 4.74 (s, 2H), 6.62-6.69 (m, 1H), 6.83 (dd, J=7.44, 2.44 Hz, 1H), 6.97-6.99 (m, 1H) 7.27 (s, 1H), 7.66 (s, 1H), 8.15-8.20 (m, 1H).

### Step 6: Preparation of methyl N-[5-[6-T(4-fluoro-3-methoxy-phenyl)-methyl-carbamoyl]-8-(methoxymethyl)imidazo[1,2-a]pyridin-3-yl]-2-pyridyl]carbamate (Compound X.21)

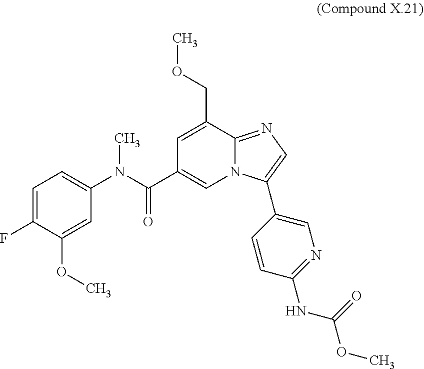 [SMILES: COCc1cc(C(=O)N(C)c2ccc(F)c(OC)c2)cn2c(-c3ccc(NC(=O)OC)nc3)cnc12]

N-(4-fluoro-3-methoxy-phenyl)-3-iodo-8-(methoxymethyl)-N-methyl-imidazo[1,2-a]pyridine-6-carboxamide (100 mg, 0.213 mmol) and methyl N-[5-(4,4,5,5-tetramethyl-1,3,2-dioxaborolan-2-yl)-2-pyridyl]carbamate (88.9 mg, 0.320 mmol, 1.50 equiv.) were dissolved in acetonitrile (1.70 mL). Then a solution of sodium carbonate (67.8 mg, 0.639 mmol, 3.00 equiv.) in water (0.85 mL) was added. The reaction mixture was degassed with argon for 15 min. Then chloro(2-dicyclohexylphosphino-2′,4′,6′-triisopropyl-1,1′-biphenyl)[2-(2′-amino-1,1′-biphenyl)]palladium(II) (17.2 mg, 0.0213 mmol, 0.100 equiv.) was added and the reaction mixture was subjected to microwave irradiation up to 100° C. for 2 hours. The reaction mixture was diluted with water and ethyl acetate. The aqueous layer was extracted with ethyl acetate, the combined organic layers were dried over sodium sulfate, filtered and concentrated under reduced pressure. The crude residue was purified by flash chromatography over silica gel (eluting cyclohexane/ethyl acetate), then the desired product was further triturated with acetonitrile to afford methyl N-[5-[6-[(4-fluoro-3-methoxy-phenyl)-methyl-carbamoyl]-8-(methoxymethyl)imidazo[1,2-a]pyridin-3-yl]-2-pyridyl]carbamate as a white solid.

LC/MS (Method A) retention time=0.77 min; [M+H]⁺=494

¹H NMR (400 MHz, DMSO-d₆) δ ppm 10.45 (s, 1H), 8.38 (d, J=2.2 Hz, 1H), 8.25 (s, 1H), 8.00 (d, J=8.7 Hz, 1H), 7.75 (s, 1H), 7.74 (dd, J=2.2, 8.7 Hz, 1H), 7.29 (dd, J=2.5, 7.6 Hz, 1H), 7.23 (s, 1H), 7.02 (dd, J=8.7, 11.3 Hz, 1H), 6.77 (qd, J=2.2, 8.4 Hz, 1H), 4.70 (s, 2H), 3.74 (d, J=5.4 Hz, 6H), 3.38 (s, 3H), 3.29 (s, 3H).

### Example 10: This example illustrates the preparation of methyl N-[5-[6-[(2-methoxy-4-pyridyl)-methyl-carbamoyl]-8-methyl-imidazo[1,2-a]pyridin-3-yl]-2-pyridyl]carbamate (compound X.22 of Table T1)

### Step 1: Preparation of methyl 8-methylimidazo[1,2-a]pyridine-6-carboxylate

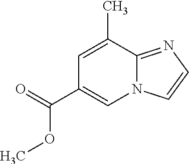

A pressure reactor was charged with 6-bromo-8-methyl-imidazo[1,2-a]pyridine (CAS 217435-65-9) (12.0 g, 56.9 mmol), methanol (200.0 mL), 1,1′-bis(diphenylphosphino)ferrocene-palladium(II)dichloride dichloromethane complex (2.30 g, 2.84 mmol, 0.05 eq) and triethylamine (17.3 g, 171 mmol, 3.00 eq.). The reactor was then flushed with nitrogen, then carbon monoxide, sealed, and pressurized to 2.5 MPa with carbon monoxide. The reaction was stirred at 100° C. for 8 hours. The vessel was then cooled to room temperature and the pressure released. The reaction mixture was concentrated under reduced pressure. The residue was purified by flash chromatography over silica gel (eluting dichloromethane/methanol) to obtain methyl 8-methylimidazo[1,2-a]pyridine-6-carboxylate as a light brown solid.

¹H NMR (400 MHz, CDCl₃) δ: 8.81 (s, 1H), 7.70 (d, J=1.2 Hz, 1H), 7.66 (d, J=1.2 Hz, 1H), 7.54 (s, 1H), 3.95 (s, 3H), 2.65 (s, 3H).

### Step 2: Preparation of methyl 3-bromo-8-methyl-imidazo[1,2-a]pyridine-6-carboxylate

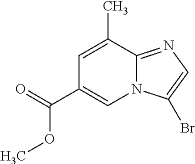

To a stirred solution of methyl 8-methylimidazo[1,2-a]pyridine-6-carboxylate (1.70 g, 8.94 mmol) in dimethylformamide (20 mL) was added at room temperature N-bromosuccinimide (1.91 g, 10.7 mmol, 1.2 eq). The reaction mixture was stirred for 2 hours at room temperature. The reaction mixture was diluted with water. The precipitate was collected by filtration, washed with water and dried under reduced pressure to afford methyl 3-bromo-8-methyl-imidazo[1,2-a]pyridine-6-carboxylate as a brown solid.

LC/MS (Method D) retention time=1.15 min; [M+H]⁺=269

### Step 3: Preparation of 3-bromo-8-methyl-imidazo[1,2-a]pyridine-6-carboxylic acid

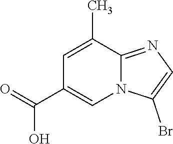

To a stirred solution of methyl 3-bromo-8-methyl-imidazo[1,2-a]pyridine-6-carboxylate (9.00 g, 31.8 mmol) in tetrahydrofuran/water (1:1, 90 mL) was added portionwise at room temperature lithium hydroxide (1.52 g, 63.5 mmol, 2.0 eq). The reaction mixture was diluted with water and extracted with ethyl acetate. The aqueous layer was acidified to pH=2 with 2 M aq. HCl. The precipitate was collected by filtration, washed with water and dried under reduced pressure to afford 3-bromo-8-methyl-imidazo[1,2-a]pyridine-6-carboxylic acid as a brown solid.

LC/MS (Method D) retention time=0.79 min; [M+H]⁺=255

### Step 4: Preparation of 3-bromo-N-(2-methoxy-4-pyridyl)-N,8-dimethyl-imidazo[1,2-a]pyridine-6-carboxamide

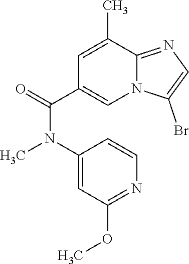 [SMILES: COc1cc(N(C)C(=O)c2cc(C)c3ncc(Br)n3c2)ccn1]

To a solution of 2-methoxy-N-methyl-pyridin-4-amine (0.500 g, 3.62 mmol) and 3-bromo-8-methyl-imidazo[1,2a]pyridine-6-carboxylic acid (1.11 g, 4.34 mmol, 1.2 eq) in pyridine (10.0 mL) was added dropwise phosphoryl trichloride (1.66 g, 10.9 mmol, 3.0 eq). The mixture was stirred at room temperature for 2 hours. The reaction mixture was diluted with water and extracted with ethyl acetate. The combined organic layers were washed with brine, dried over sodium sulfate, filtered, and concentrated under reduced pressure. The residue was purified by flash chromatography over silica gel (eluting petroleum ether/ethyl acetate) to obtain 3-bromo-N-(2-methoxy-4-pyridyl)-N,8-dimethyl-imidazo[1,2-a]pyridine-6-carboxamide as a yellow solid.

LC/MS (Method D) retention time=1.06 min; [M+H]⁺=375

### Step 5: Preparation of methyl N-[5-[6-((2-methoxy-4-pyridyl)-methyl-carbamoyl]-8-methyl-imidazo[1,2-a]pyridin-3-yl]-2-pyridyl]carbamate (Compound X.22)

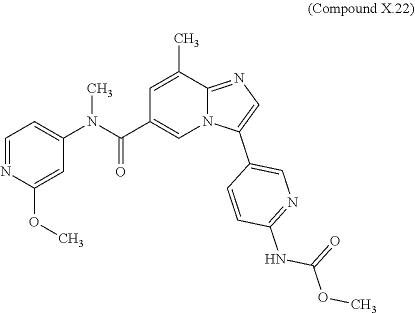 [SMILES: COC(=O)Nc1ccc(-c2cnc3c(C)cc(C(=O)N(C)c4ccnc(OC)c4)cn23)cn1]

3-bromo-N-(2-methoxy-4-pyridyl)-N,8-dimethyl-imidazo[1,2-a]pyridine-6-carboxamide (160 mg, 0.426 mmol) and methyl N-[5-(4,4,5,5-tetramethyl-1,3,2-dioxaborolan-2-yl)-2-pyridyl]carbamate (178 mg, 0.640 mmol, 1.5 eq) were dissolved in dioxane/water (5:1, 5.0 mL) under an atmosphere of nitrogen. Then BrettPhos Pd G3 (CAS 1470372-59-8) (58.0 mg, 0.064 mmol, 0.15 eq) was added, followed by potassium carbonate (118 mg, 0.853 mol, 2.0 eq). The reaction mixture was stirred at 65° C. for 1.5 hour under an atmosphere of nitrogen. The reaction mixture was then concentrated under reduced pressure. The residue was purified by flash chromatography over silica gel (eluting dichloromethane/methanol), then further triturated with acetonitrile to afford methyl N-[5-[6-[(2-methoxy-4-pyridyl)-methyl-carbamoyl]-8-methyl-imidazo[1,2-a]pyridin-3-yl]-2-pyridyl]carbamate as a white solid.

LC/MS (Method D) retention time=0.89 min; [M+H]⁺=447

¹H NMR (400 MHz, DMSO-d₆) δ ppm 10.46 (s, 1H), 8.36 (d, J=2.5 Hz, 1H), 8.20 (d, J=1.7 Hz, 1H), 8.02 (d, J=5.5 Hz, 1H), 7.96 (d, J=8.7 Hz, 1H), 7.78 (d, J=2.0 Hz, 1H), 7.53 (dd, J=8.6, 2.5 Hz, 1H), 7.16-7.09 (m, 1H), 6.87-6.75 (m, 2H), 3.82 (s, 3H), 3.73 (s, 3H), 3.39 (s, 3H), 2.46 (s, 3H).

Example 11: This example illustrates the preparation of methyl N-[5-[6-[(4-cyano-3-methoxy-phenyl)-methyl-carbamoyl]-8-methyl-imidazo[1,2-a]pyridin-3-yl]-2-pyridyl]carbamate (compound X.23 of Table T1)

### Step 1: Preparation of 3-bromo-N-(4-cyano-3-methoxy-phenyl)-8-methyl-imidazo[1,2-a]pyridine-6-carboxamide

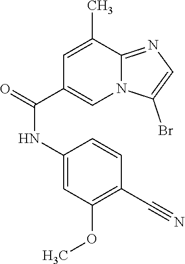 [SMILES: COc1cc(NC(=O)c2cc(C)c3ncc(Br)n3c2)ccc1C#N]

To a solution of 4-amino-2-methoxy-benzonitrile (0.550 g, 3.71 mmol) and 3-bromo-8-methyl-imidazo[1,2a]pyridine-6-carboxylic acid (1.04 g, 4.08 mmol, 1.1 eq) in pyridine (15.0 mL) at 0° C. was added dropwise phosphoryl trichloride (1.71 g, 11.1 mmol, 3.0 eq). The mixture was let to warm up to room temperature while stirring and stirred at room temperature for 2 hours. The reaction mixture was then diluted with water and extracted with ethyl acetate. The combined organic layers were washed with brine, dried over sodium sulfate, filtered, and concentrated under reduced pressure. The residue was purified by flash chromatography over silica gel (eluting petroleum ether/ethyl acetate) to obtain 3-bromo-N-(4-cyano-3-methoxy-phenyl)-8-methyl-imidazo[1,2-a]pyridine-6-carboxamide as a yellow solid.

LC/MS (Method D) retention time=1.51 min; [M+H]⁺=385

### Step 2: Preparation of 3-bromo-N-(4-cyano-3-methoxy-phenyl)-N,8-dimethyl-imidazo[1,2-a]pyridine-6-carboxamide

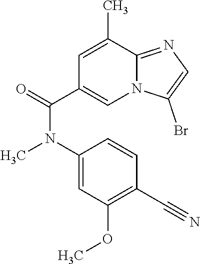 [SMILES: COc1cc(N(C)C(=O)c2cc(C)c3ncc(Br)n3c2)ccc1C#N]

To a solution of 3-bromo-N-(4-cyano-3-methoxy-phenyl)-8-methyl-imidazo[1,2-a]pyridine-6-carboxamide (0.700 g, 1.82 mmol) in dry dimethylformamide (20.0 mL) was added iodomethane (0.464 g, 3.27 mmol, 1.79 eq.) and potassium carbonate (1.26 g, 9.09 mmol, 5.0 eq) at room temperature. The reaction mixture was stirred at 75° C. for 2 hours. The reaction mixture was then diluted with water and extracted with ethyl acetate. The combined organic layers were washed with brine, dried over sodium sulfate, filtered, and concentrated under reduced pressure. The residue was purified by flash chromatography over silica gel (eluting petroleum ether/ethyl acetate) to afford 3-bromo-N-(4-cyano-3-methoxy-phenyl)-N,8-dimethyl-imidazo[1,2-a]pyridine-6-carboxamide as a yellow solid.

LC/MS (Method D) retention time=1.08 min; [M+H]⁺=399

### Step 3: Preparation of methyl N-[5-[6-[(4-cyano-3-methoxy-phenyl)-methyl-carbamoyl]-8-methyl-imidazo[1,2-a]pyridin-3-yl]-2-pyridyl]carbamate (Compound X.23)

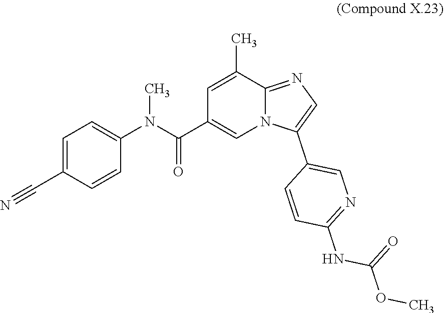 [SMILES: COC(=O)Nc1ccc(-c2cnc3c(C)cc(C(=O)N(C)c4ccc(C#N)cc4)cn23)cn1]

3-bromo-N-(4-cyano-3-methoxy-phenyl)-N,8-dimethyl-imidazo[1,2-a]pyridine-6-carboxamide (380 mg, 0.952 mmol) and methyl N-[5-(4,4,5,5-tetramethyl-1,3,2-dioxaborolan-2-yl)-2-pyridyl]carbamate (397 mg, 1.43 mmol, 1.5 eq) were dissolved in dioxane/water (4:1, 10.0 mL) under an atmosphere of nitrogen. Then BrettPhos Pd G3 (CAS 1470372-59-8) (86.3 mg, 0.0952 mmol, 0.10 eq) was added, followed by potassium carbonate (395 mg, 2.86 mmol, 3.0 eq). The reaction mixture was stirred at 65° C. for 2 hours under an atmosphere of nitrogen. The reaction mixture was then diluted with water and extracted with ethyl acetate. The combined organic layers were washed with brine, dried over sodium sulfate, filtered, and concentrated under reduced pressure. The residue was purified by flash chromatography over silica gel (eluting dichloromethane/methanol) to afford methyl N-[5-[6-[(4-cyano-3-methoxy-phenyl)-methyl-carbamoyl]-8-methyl-imidazo[1,2-a]pyridin-3-yl]-2-pyridyl]carbamate as a yellow solid.

LC/MS (Method D) retention time=1.38 min; [M+H]⁺=471

¹H NMR (400 MHz, DMSO-d₆) δ ppm 10.45 (s, 1H), 8.32 (d, J=1.6 Hz, 1H), 8.16 (s, 1H), 7.98 (d, J=8.7 Hz, 1H), 7.76 (s, 1H), 7.60 (dd, J=8.4, 4.2 Hz, 2H), 7.33 (s, 1H), 7.13 (s, 1H), 6.87 (dd, J=8.3, 1.7 Hz, 1H), 3.81 (s, 3H), 3.74 (s, 3H), 3.42 (s, 3H), 2.45 (s, 3H).

Throughout this description, temperatures are given in degrees Celsius (° C.) and “mp.” means melting point. LC/MS means Liquid Chromatography Mass Spectrometry and the description of the apparatus and the method is as follows:

### The LC/MS Apparatus and Method:

### Method A:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

### Method B:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Method C:

Spectra were recorded on a Mass Spectrometer from Waters (Acquity QDa Mass Spectrometer) equipped with an electrospray source (Polarity: Positive and Negative Polarity Switch), Capillary: 0.8 kV, Cone range: 25 V, Extractor: V (No extractor voltage for QDa detector) Source Temperature: 120° C., Desolvation Temperature: 600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

### Method D:

Equipment: Shimadzu LCMS 2020 Mass Spectrometer; Column: HALO C18 2.7 μm, 3.0 mm×30 mm; Mobile Phase: MeCN (with either 0.05% HCOOH or 0.05% TFA)—Water (with either 0.05% HCOOH or 0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

The below Table T1 gathers for compounds of formula X.01 to X.23 according to formula (I):
LC/MS data, such as retention time (RT), [M+H]*,
the type of method, and/or
melting point (MP).

TABLE T1

| | | | RT | [M + H] | | MP |
|---|---|---|---|---|---|---|
| Entry | Compound name | Molecule | (min) | (measured) | Method | ° C. |
| X.01 | methyl N-[5-[6-[(4-fluoro- 3-methoxy-phenyl)-meth- yl-carbamoyl]imidazo[1,2- a]pyridin-3-yl]-2-pyridyl] carbamate | 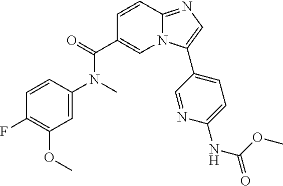 [SMILES: COC(=O)Nc1ccc(-c2cnc3ccc(C(=O)N(C)c4ccc(F)c(OC)c4)cn23)cn1] | 0.87 | 450.2 | B | 208-210 |
| X.02 | methyl N-[5-[6-[(4-fluoro- 3-methoxy-phenyl)-meth- yl-carbamoyl]-8-methyl- imidazo[1,2-a]pyridin-3- yl]-2-pyridyl]carbamate | 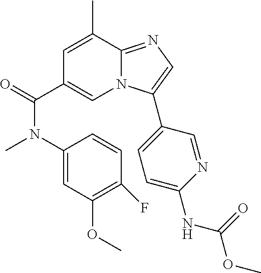 [SMILES: COC(=O)Nc1ccc(-c2cnc3c(C)cc(C(=O)N(C)c4ccc(F)c(OC)c4)cn23)cn1] | 0.92 | 464.5 | B | 160-165 |
| X.03 | methyl N-[5-[6-[(4-fluoro- 3-methoxy-phenyl)-(meth- oxymethyl)carbamoyl]-8- methyl-imidazo[1,2-a]pyr- idin-3-yl]-2-pyridyl] carbamate | 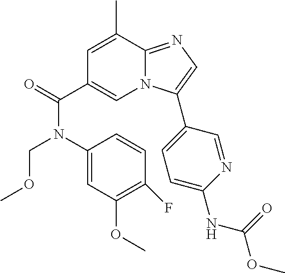 | 0.96 | 494.6 | B | 90-95 |
| X.04 | methyl N-[5-[6-[(4-fluoro- 3-methoxy-phenyl)-(meth- oxymethyl)carbamoyl] imidazo[1,2-a]pyridin-3- yl]-2-pyridyl]carbamate | 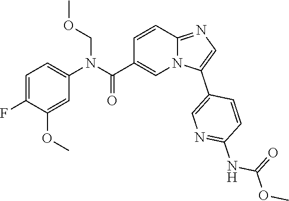 [SMILES: COCN(C(=O)c1ccc2ncc(-c3ccc(NC(=O)OC)nc3)n2c1)c1ccc(F)c(OC)c1] | 0.74 | 480 | A | |
| X.05 | methyl N-[4-[6-[(4-fluoro- 3-methoxy-phenyl)-meth- yl-carbamoyl]imidazo[1,2- a]pyridin-3-yl]phenyl] carbamate | 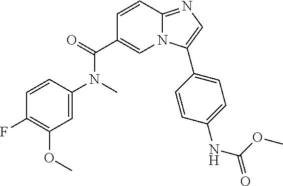 [SMILES: COC(=O)Nc1ccc(-c2cnc3ccc(C(=O)N(C)c4ccc(F)c(OC)c4)cn23)cc1] | 0.70 | 449 | A | 208-210 |
| X.06 | 3-[6-(cyclopropanecarbon- ylamino)-3-pyridyl]-N-(4- fluoro-3-methoxy-phenyl)- N-methyl-imidazo[1,2- a]pyridine-6-carboxamide | 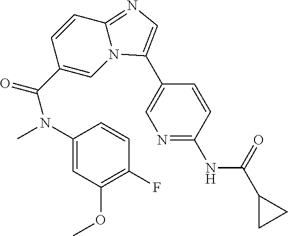 [SMILES: COc1cc(N(C)C(=O)c2ccc3ncc(-c4ccc(NC(=O)C5CC5)nc4)n3c2)ccc1F] | 0.94 | 460 | C | 106-108 |
| X.07 | methyl N-[4-[6-[(4-fluoro- phenyl)-methyl-carbamo- yl]imidazo[1,2-a]pyridin- 3-yl]phenyl]carbamate | 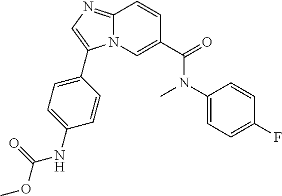 [SMILES: COC(=O)Nc1ccc(-c2cnc3ccc(C(=O)N(C)c4ccc(F)cc4)cn23)cc1] | 0.95 | 419.5 | B | |
| X.08 | methyl N-[4-[6-[(4-chloro- phenyl)-methyl-carbamo- yl]-8-methyl-imidazo[1,2- a]pyridin-3-yl]phenyl] carbamate | 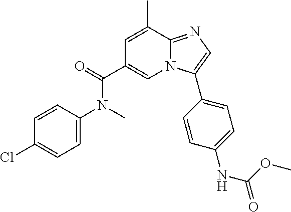 [SMILES: COC(=O)Nc1ccc(-c2cnc3c(C)cc(C(=O)N(C)c4ccc(Cl)cc4)cn23)cc1] | 1.07 | 449.1 | B | 105-110 |
| X.09 | methyl N-[5-[6-[(4-fluoro- 3-methyl-phenyl)-methyl- carbamoyl]imidazo[1,2-a] pyridin-3-yl]-2-pyridyl] carbamate | 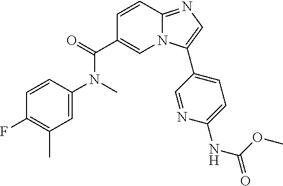 [SMILES: COC(=O)Nc1ccc(-c2cnc3ccc(C(=O)N(C)c4ccc(F)c(C)c4)cn23)cn1] | 0.94 | 434.2 | B | |
| X.10 | methyl N-[5-[6-[(4-cyano- 3-methoxy-phenyl)-methyl- carbamoyl]imidazo[1,2-a] pyridin-3-yl]-2-pyridyl] carbamate | 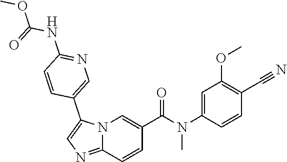 [SMILES: COC(=O)Nc1ccc(-c2cnc3ccc(C(=O)N(C)c4ccc(C#N)c(OC)c4)cn23)cn1] | 0.82 | 457.2 | B | |
| X.11 | methyl N-[5-[6-[(4-chloro- 3-methoxy-phenyl)-methyl- carbamoyl]imidazo[1,2-a] pyridin-3-yl]-2-pyridyl] carbamate | 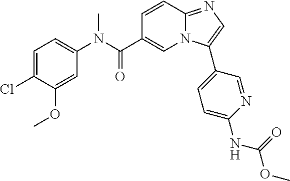 [SMILES: COC(=O)Nc1ccc(-c2cnc3ccc(C(=O)N(C)c4ccc(Cl)c(OC)c4)cn23)cn1] | 0.77 | 466 | A | |
| X.12 | methyl N-[5-[6-[(4-fluoro- phenyl)-methyl-carbamo- yl]-8-methyl-imidazo[1,2- a]pyridin-3-yl]-2-pyridyl] carbamate | 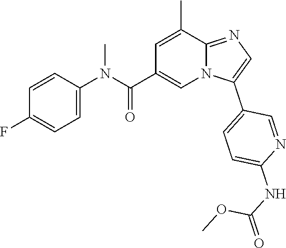 [SMILES: COC(=O)Nc1ccc(-c2cnc3c(C)cc(C(=O)N(C)c4ccc(F)cc4)cn23)cn1] | 0.90 | 434.5 | B | 140-145 |
| X.13 | methyl N-[5-[6-[(4-fluoro- phenyl)-methyl-carbamo- yl]-7-methyl-imidazo[1,2- a]pyridin-3-yl]-2-pyridyl] carbamate | 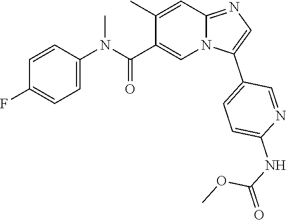 [SMILES: COC(=O)Nc1ccc(-c2cnc3cc(C)c(C(=O)N(C)c4ccc(F)cc4)cn23)cn1] | 0.83 | 434.5 | B | 180-185 |
| X.14 | methyl N-[5-[6-[cyano- methyl-(4-fluoro-3-meth- oxy-phenyl)carbamoyl] imidazo[1,2-a]pyridin-3- yl]-2-pyridyl]carbamate | 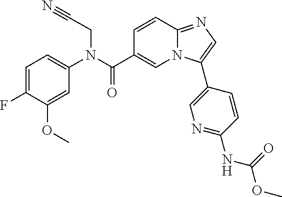 [SMILES: COC(=O)Nc1ccc(-c2cnc3ccc(C(=O)N(CC#N)c4ccc(F)c(OC)c4)cn23)cn1] | 0.71 | 475 | A | |
| X.15 | methyl N-[5-[6-[(4-chloro- 3-methoxy-phenyl)-(cyano- methyl)carbamoyl]imidazo [1,2-a]pyridin-3-yl]-2-pyr- idyl]carbamate | 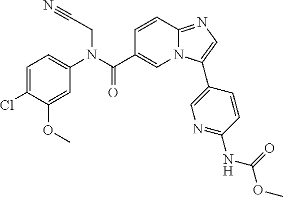 [SMILES: COC(=O)Nc1ccc(-c2cnc3ccc(C(=O)N(CC#N)c4ccc(Cl)c(OC)c4)cn23)cn1] | 0.75 | 491 | A | |
| X.16 | methyl N-[5-[6-[(4-fluoro- 3-methoxy-phenyl)-(meth- ylsulfonylmethyl)carbamo- yl]imidazo[1,2-a]pyridin- 3-yl]-2-pyridyl]carbamate | 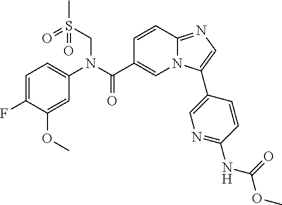 [SMILES: COC(=O)Nc1ccc(-c2cnc3ccc(C(=O)N(CS(C)(=O)=O)c4ccc(F)c(OC)c4)cn23)cn1] | 0.67 | 528 | A | |
| X.17 | N-(4-fluoro-3-methoxy- phenyl)-3-[6-(methoxy- carbamoylamino)-3-pyr- idyl]-N-methyl-imidazo [1,2-a]pyridine-6-carbox- amide | 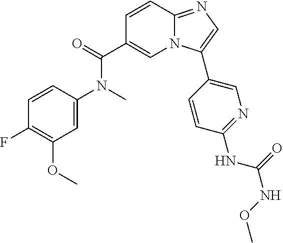 [SMILES: CONC(=O)Nc1ccc(-c2cnc3ccc(C(=O)N(C)c4ccc(F)c(OC)c4)cn23)cn1] | 0.63 | 465 | A | |
| X.18 | methyl N-[5-[8-acetamido- 6-[(4-fluorophenyl)-meth- yl-carbamoyl]imidazo[1,2- a]pyridin-3-yl]-2-pyridyl] carbamate | 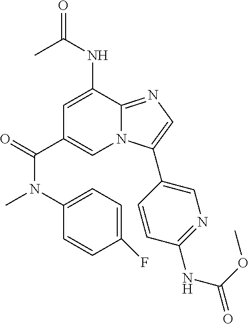 [SMILES: COC(=O)Nc1ccc(-c2cnc3c(NC(C)=O)cc(C(=O)N(C)c4ccc(F)cc4)cn23)cn1] | 0.78 | 477 | A | 218-220 |
| X.19 | methyl N-[5-[6-[ethyl-(4- fluorophenyl)carbamoyl] imidazo[1,2-a]pyridin-3- yl]-2-pyridyl]carbamate | 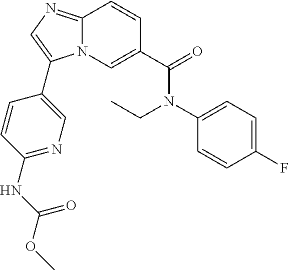 [SMILES: CCN(C(=O)c1ccc2ncc(-c3ccc(NC(=O)OC)nc3)n2c1)c1ccc(F)cc1] | 0.73 | 434 | A | |
| X.20 | methyl N-[5-[8-acetamido- 6-[(4-fluoro-3-methoxy- phenyl)-methyl-carbamo- yl]imidazo[1,2-a]pyridin- 3-yl]-2-pyridyl]carbamate | 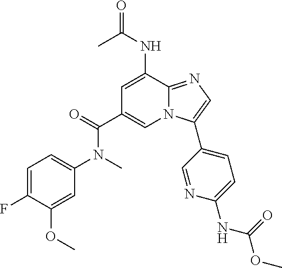 [SMILES: COC(=O)Nc1ccc(-c2cnc3c(NC(C)=O)cc(C(=O)N(C)c4ccc(F)c(OC)c4)cn23)cn1] | 1.00 | 507 | C | 216-218 |
| X.21 | methyl N-[5-[6-[(4-fluoro- 3-methoxy-phenyl)-methyl- carbamoyl]-8-(methoxy- methyl)imidazo[1,2-a] pyridin-3-yl]-2-pyridyl] carbamate | 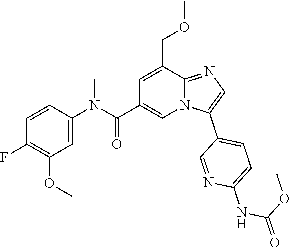 [SMILES: COCc1cc(C(=O)N(C)c2ccc(F)c(OC)c2)cn2c(-c3ccc(NC(=O)OC)nc3)cnc12] | 0.77 | 494 | A | 178-180 |
| X.22 | methyl N-[5-[6-[(2-meth- oxy-4-pyridyl)-methyl- carbamoyl]-8-methyl- imidazo[1,2-a]pyridin-3- yl]-2-pyridyl]carbamate | 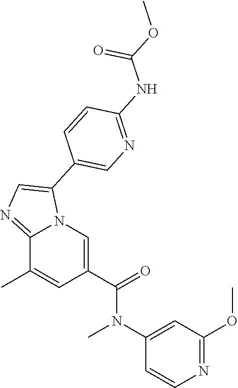 | 0.89 | 447 | D | 167-169 |
| X.23 | methyl N-[5-[6-[(4-cyano- 3-methoxy-phenyl)-methyl- carbamoyl]-8-methyl- imidazo[1,2-a]pyridin-3- yl]-2-pyridyl]carbamate | 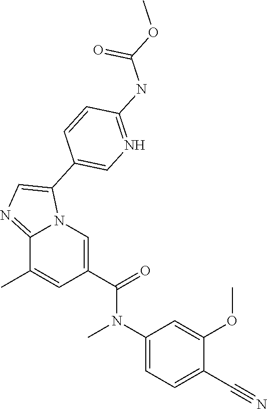 [SMILES: COC(=O)Nc1ccc(-c2cnc3c(C)cc(C(=O)N(C)c4ccc(C#N)c(OC)c4)cn23)cn1] | 1.38 | 471 | D | 197-199 |

### BIOLOGICAL EXAMPLES

### General Examples of Leaf Disk Tests in Well Plates:

Leaf disks or leaf segments of various plant species are cut from plants grown in a greenhouse. The cut leaf disks or segments are placed in multiwell plates (24-well format) onto water agar. The leaf disks are sprayed with a test solution before (preventative) or after (curative) inoculation. Compounds to be tested are prepared as DMSO solutions (max. 10 mg/ml) which are diluted to the appropriate concentration with 0.025% Tween20 just before spraying. The inoculated leaf disks or segments are incubated under defined conditions (temperature, relative humidity, light, etc.) according to the respective test system. A single evaluation of disease level is carried out 3 to 14 days after inoculation, depending on the pathosystem. Percent disease control relative to the untreated check leaf disks or segments is then calculated.

### General Examples of Liquid Culture Tests in Well Plates:

Mycelia fragments or conidia suspensions of a fungus prepared either freshly from liquid cultures of the fungus or from cryogenic storage, are directly mixed into nutrient broth. DMSO solutions of the test compound (max. 10 mg/ml) are diluted with 0.025% Tween20 by a factor of 50 and 10 μl of this solution is pipetted into a microtiter plate (96-well format). The nutrient broth containing the fungal spores/mycelia fragments is then added to give an end concentration of the tested compound. The test plates are incubated in the dark at 24° C. and 96% relative humidity. The inhibition of fungal growth is determined photometrically after 2 to 7 days, depending on the pathosystem, and percent antifungal activity relative to the untreated check is calculated.

### Example A1: Activity Against Phytophthora infestans/Tomato/Leaf Disc Preventative (Late Blight)

Tomato leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 16° C. and 75% rh under a light regime of 24 h darkness followed by 12 h light/12 h darkness in a climate cabinet and the activity of a compound is assessed as percent disease control compared to untreated when an appropriate level of disease damage appears in untreated check leaf disks (5-7 days after application).

The following compounds (from Table T1) gave at least 80% control of Phytophthora infestans at 200 ppm when compared to untreated control under the same conditions, which showed extensive disease development: X.01, X.02, X.03, X.04, X.05, X.06, X.07, X.08, X.09, X.10, X.11, X.12, X.13, X.14, X.15, X.16, X.17, X.18, X.19, X.20, X.21, X.22, and X.23.

### Example A2: Activity Against Plasmopara Viticola/Grape/Leaf Disc Preventative (Late Blight)

Grape vine leaf disks are placed on water agar in multiwell plates (24-well format) and sprayed with the formulated test compound diluted in water. The leaf disks are inoculated with a spore suspension of the fungus 1 day after application. The inoculated leaf disks are incubated at 19° C. and 80% rh under a light regime of 12 h light/12 h darkness in a climate cabinet and the activity of a compound is assessed as percent disease control compared to untreated when an appropriate level of disease damage appears in untreated check leaf disks (6-8 days after application).

The following compounds (from Table T1) gave at least 80% control of Plasmopara viticola at 200 ppm when compared to untreated control under the same conditions, which showed extensive disease development: X.01, X.02, X.03, X.04, X.05, X.06, X.07, X.08, X.09, X.10, X.11, X.12, X.13, X.14, X.15, X.18, X.19, X.20, X.21, X.22, and X.23.

### Example A3: Activity Against Pythium ultimum/Liquid Culture (Seedling Damping Off)

Mycelia fragments and oospores of a newly grown liquid culture of the fungus are directly mixed into nutrient broth (PDB potato dextrose broth). After placing a (DMSO) solution of test compound into a microtiter plate (96-well format), the nutrient broth containing the fungal mycelia/spore mixture is added.

The test plates are incubated at 24° C. and the inhibition of growth is determined photometrically 2-3 days after application.

The following compounds (from Table T1) gave at least 80% control of Pythium ultimum at 20 ppm when compared to untreated control under the same conditions, which showed extensive disease development: X.01, X.02, X.03, X.04, X.05, X.06, X.07, X.08, X.09, X.10, X.11, X.12, X.13, X.14, X.15, X.16, X.17, X.18, X.19, X.20, X.21, X.22, and X.23.

In the below examples, the source and fermentation of Streptomyces chrestomyceticus CBS149411 (used as Component B) are as follows:

### Fermentation of Streptomyces sp.:

Streptomyces sp. Saigon413, isolated in Vietnam before 1961, was deposited at the Westerdijk institute under accession number CBS149411. The deposit was made by Syngenta Ltd., Jealott's Hill Research International Centre, Bracknell, Berkshire, RG42 6EY, UK under the terms of the Budapest Treaty on the International Recognition of the Deposit of Microorganisms for the Purposes of Patent Procedure. The Streptomyces was cultivated in Erlenmeyer flasks with a liquid medium consisting of (g/l) casein hydrolysate 10, glucose 40, K₂HPO₄ 1.25, soytone 2, tryptone, 8 and incubated at 28° C. in an incubator shaking 150 rpm with 25 mm throw for 4 days.

### Large Scale Fermentations:

For large scale production, standard procedures were applied for cultivating Streptomyces chrestomyceticus CBS149411 to high cell density using fed-batch fermentation. After harvesting, the broth was spray dried or freeze dried according to methods known to a person skilled in the art. The final product (TGAI) after spray- or freeze drying had a cell count of 1*10⁵ to 1*10¹³ CFU/g dry mass. TGAI: technical grade active ingredient (unformulated product).

### Example B1: Activity Against Pythium ultimum (Damping Off)

Mycelial fragments of the pathogen, prepared from a fresh liquid culture, were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 48 hrs. The following mixture compositions (A:B) at the reported concentration (in ppm) gave at least 80% disease control in this test.

| Component A | | | Conc. (ppm) |
|---|---|---|---|
| (Compound) | Component B | Ratio A:B | (A:B) |
| X.01 | Picarbutrazox | 1:1 | 6:6 |
| X.01 | Picarbutrazox | 30:1 | 6:0.2 |
| X.01 | Picarbutrazox | 1:10 | 0.6:6 |
| X.01 | Picarbutrazox | 3:1 | 0.6:0.2 |
| X.01 | Cymoxanil | 1:1 | 6:6 |
| X.01 | Cymoxanil | 10:1 | 6:0.6 |
| X.01 | Cymoxanil | 1:10 | 0.6:6 |
| X.01 | Cymoxanil | 1:1 | 0.6:0.6 |
| X.01 | Disodium phosphonate | 1:1 | 6:6 |
| X.01 | Disodium phosphonate | 10:1 | 6:0.6 |
| X.01 | Disodium phosphonate | 1:10 | 0.6:6 |
| X.01 | Disodium phosphonate | 1:1 | 0.6:0.6 |
| X.01 | Difenoconazole | 1:1 | 6:6 |
| X.01 | Difenoconazole | 10:1 | 6:0.6 |
| X.01 | Difenoconazole | 1:10 | 0.6:6 |
| X.01 | Difenoconazole | 1:1 | 0.6:0.6 |
| X.01 | Fludioxonil | 1:1 | 6:6 |
| X.01 | Fludioxonil | 10:1 | 6:0.6 |
| X.01 | Fludioxonil | 1:10 | 0.6:6 |
| X.01 | Fludioxonil | 1:1 | 0.6:0.6 |
| X.01 | Fluazinam | 1:1 | 6:6 |
| X.01 | Fluazinam | 30:1 | 6:0.2 |
| X.01 | Fluazinam | 1:10 | 0.6:6 |
| X.01 | Fluazinam | 3:1 | 0.6:0.2 |
| X.01 | Ametoctradin | 1:1 | 6:6 |
| X.01 | Ametoctradin | 30:1 | 6:0.2 |
| X.01 | Ametoctradin | 1:10 | 0.6:6 |
| X.01 | Ametoctradin | 3:1 | 0.6:0.2 |
| X.01 | Amisulbrom | 1:1 | 6:6 |
| X.01 | Amisulbrom | 30:1 | 6:0.2 |
| X.01 | Amisulbrom | 1:10 | 0.6:6 |
| X.01 | Amisulbrom | 3:1 | 0.6:0.2 |
| X.01 | Cyazofamid | 1:1 | 6:6 |
| X.01 | Cyazofamid | 30:1 | 6:0.2 |
| X.01 | Cyazofamid | 1:10 | 0.6:6 |
| X.01 | Cyazofamid | 3:1 | 0.6:0.2 |
| X.01 | Azoxystrobin | 1:1 | 6:6 |
| X.01 | Azoxystrobin | 10:1 | 6:0.6 |
| X.01 | Azoxystrobin | 1:10 | 0.6:6 |
| X.01 | Azoxystrobin | 1:1 | 0.6:0.6 |
| X.01 | Sedaxane | 1:1 | 6:6 |
| X.01 | Sedaxane | 10:1 | 6:0.6 |
| X.01 | Sedaxane | 1:10 | 0.6:6 |
| X.01 | Sedaxane | 1:1 | 0.6:0.6 |
| X.01 | Pydiflumetofen | 1:1 | 6:6 |
| X.01 | Pydiflumetofen | 10:1 | 6:0.6 |
| X.01 | Pydiflumetofen | 1:10 | 0.6:6 |
| X.01 | Pydiflumetofen | 1:1 | 0.6:0.6 |
| X.01 | Metalaxyl-M | 1:1 | 6:6 |
| X.01 | Metalaxyl-M | 30:1 | 6:0.2 |
| X.01 | Metalaxyl-M | 1:10 | 0.6:6 |
| X.01 | Metalaxyl-M | 3:1 | 0.6:0.2 |
| X.01 | Folpet | 1:1 | 6:6 |
| X.01 | Folpet | 10:1 | 6:0.6 |
| X.01 | Folpet | 1:10 | 0.6:6 |
| X.01 | Folpet | 1:1 | 0.6:0.6 |
| X.01 | Chlorothalonil | 1:1 | 6:6 |
| X.01 | Chlorothalonil | 10:1 | 6:0.6 |
| X.01 | Chlorothalonil | 1:10 | 0.6:6 |
| X.01 | Chlorothalonil | 1:1 | 0.6:0.6 |
| X.01 | Oxathiapiprolin | 1:1 | 6:6 |
| X.01 | Oxathiapiprolin | 30:1 | 6:0.2 |
| X.01 | Oxathiapiprolin | 1:10 | 0.6:6 |
| X.01 | Oxathiapiprolin | 3:1 | 0.6:0.2 |
| X.01 | Fluoxapiprolin | 1:1 | 6:6 |
| X.01 | Fluoxapiprolin | 30:1 | 6:0.2 |
| X.01 | Fluoxapiprolin | 1:10 | 0.6:6 |
| X.01 | Fluoxapiprolin | 3:1 | 0.6:0.2 |
| X.01 | Acibenzolar-S-methyl | 1:1 | 6:6 |
| X.01 | Acibenzolar-S-methyl | 10:1 | 6:0.6 |
| X.01 | Acibenzolar-S-methyl | 1:10 | 0.6:6 |
| X.01 | Acibenzolar-S-methyl | 1:1 | 0.6:0.6 |
| X.01 | Fluopicolide | 1:1 | 6:6 |
| X.01 | Fluopicolide | 30:1 | 6:0.2 |
| X.01 | Fluopicolide | 1:10 | 0.6:6 |
| X.01 | Fluopicolide | 3:1 | 0.6:0.2 |
| X.01 | Zoxamide | 1:1 | 6:6 |
| X.01 | Zoxamide | 30:1 | 6:0.2 |
| X.01 | Zoxamide | 1:10 | 0.6:6 |
| X.01 | Zoxamide | 3:1 | 0.6:0.2 |
| X.01 | Mandipropamid | 1:1 | 6:6 |
| X.01 | Mandipropamid | 30:1 | 6:0.2 |
| X.01 | Mandipropamid | 1:10 | 0.6:6 |
| X.01 | Mandipropamid | 3:1 | 0.6:0.2 |
| X.01 | Cyclobutrifluram | 1:1 | 6:6 |
| X.01 | Cyclobutrifluram | 10:1 | 6:0.6 |
| X.01 | Cyclobutrifluram | 1:10 | 0.6:6 |
| X.01 | Cyclobutrifluram | 1:1 | 0.6:0.6 |
| X.02 | Picarbutrazox | 1:1 | 6:6 |
| X.02 | Picarbutrazox | 30:1 | 6:0.2 |
| X.02 | Picarbutrazox | 1:10 | 0.6:6 |
| X.02 | Picarbutrazox | 3:1 | 0.6:0.2 |
| X.02 | Cymoxanil | 1:1 | 6:6 |
| X.02 | Cymoxanil | 10:1 | 6:0.6 |
| X.02 | Cymoxanil | 1:10 | 0.6:6 |
| X.02 | Cymoxanil | 1:1 | 0.6:0.6 |
| X.02 | Disodium phosphonate | 1:1 | 6:6 |
| X.02 | Disodium phosphonate | 10:1 | 6:0.6 |
| X.02 | Disodium phosphonate | 1:10 | 0.6:6 |
| X.02 | Disodium phosphonate | 1:1 | 0.6:0.6 |
| X.02 | Difenoconazole | 1:1 | 6:6 |
| X.02 | Difenoconazole | 10:1 | 6:0.6 |
| X.02 | Difenoconazole | 1:10 | 0.6:6 |
| X.02 | Difenoconazole | 1:1 | 0.6:0.6 |
| X.02 | Fludioxonil | 1:1 | 6:6 |
| X.02 | Fludioxonil | 10:1 | 6:0.6 |
| X.02 | Fludioxonil | 1:10 | 0.6:6 |
| X.02 | Fludioxonil | 1:1 | 0.6:0.6 |
| X.02 | Fluazinam | 1:1 | 6:6 |
| X.02 | Fluazinam | 30:1 | 6:0.2 |
| X.02 | Fluazinam | 1:10 | 0.6:6 |
| X.02 | Fluazinam | 3:1 | 0.6:0.2 |
| X.02 | Ametoctradin | 1:1 | 6:6 |
| X.02 | Ametoctradin | 30:1 | 6:0.2 |
| X.02 | Ametoctradin | 1:10 | 0.6:6 |
| X.02 | Ametoctradin | 3:1 | 0.6:0.2 |
| X.02 | Amisulbrom | 1:1 | 6:6 |
| X.02 | Amisulbrom | 30:1 | 6:0.2 |
| X.02 | Amisulbrom | 1:10 | 0.6:6 |
| X.02 | Amisulbrom | 3:1 | 0.6:0.2 |
| X.02 | Cyazofamid | 1:1 | 6:6 |
| X.02 | Cyazofamid | 30:1 | 6:0.2 |
| X.02 | Cyazofamid | 1:10 | 0.6:6 |
| X.02 | Cyazofamid | 3:1 | 0.6:0.2 |
| X.02 | Azoxystrobin | 1:1 | 6:6 |
| X.02 | Azoxystrobin | 10:1 | 6:0.6 |
| X.02 | Azoxystrobin | 1:10 | 0.6:6 |
| X.02 | Azoxystrobin | 1:1 | 0.6:0.6 |
| X.02 | Sedaxane | 1:1 | 6:6 |
| X.02 | Sedaxane | 10:1 | 6:0.6 |
| X.02 | Sedaxane | 1:10 | 0.6:6 |
| X.02 | Sedaxane | 1:1 | 0.6:0.6 |
| X.02 | Pydiflumetofen | 1:1 | 6:6 |
| X.02 | Pydiflumetofen | 10:1 | 6:0.6 |
| X.02 | Pydiflumetofen | 1:10 | 0.6:6 |
| X.02 | Pydiflumetofen | 1:1 | 0.6:0.6 |
| X.02 | Metalaxyl-M | 1:1 | 6:6 |
| X.02 | Metalaxyl-M | 30:1 | 6:0.2 |
| X.02 | Metalaxyl-M | 1:10 | 0.6:6 |
| X.02 | Metalaxyl-M | 3:1 | 0.6:0.2 |
| X.02 | Folpet | 1:1 | 6:6 |
| X.02 | Folpet | 10:1 | 6:0.6 |
| X.02 | Folpet | 1:10 | 0.6:6 |
| X.02 | Folpet | 1:1 | 0.6:0.6 |
| X.02 | Chlorothalonil | 1:1 | 6:6 |
| X.02 | Chlorothalonil | 10:1 | 6:0.6 |
| X.02 | Chlorothalonil | 1:10 | 0.6:6 |
| X.02 | Chlorothalonil | 1:1 | 0.6:0.6 |
| X.02 | Oxathiapiprolin | 1:1 | 6:6 |
| X.02 | Oxathiapiprolin | 30:1 | 6:0.2 |
| X.02 | Oxathiapiprolin | 1:10 | 0.6:6 |
| X.02 | Oxathiapiprolin | 3:1 | 0.6:0.2 |
| X.02 | Fluoxapiprolin | 1:1 | 6:6 |
| X.02 | Fluoxapiprolin | 30:1 | 6:0.2 |
| X.02 | Fluoxapiprolin | 1:10 | 0.6:6 |
| X.02 | Fluoxapiprolin | 3:1 | 0.6:0.2 |
| X.02 | Acibenzolar-S-methyl | 1:1 | 6:6 |
| X.02 | Acibenzolar-S-methyl | 10:1 | 6:0.6 |
| X.02 | Acibenzolar-S-methyl | 1:10 | 0.6:6 |
| X.02 | Acibenzolar-S-methyl | 1:1 | 0.6:0.6 |
| X.02 | Fluopicolide | 1:1 | 6:6 |
| X.02 | Fluopicolide | 30:1 | 6:0.2 |
| X.02 | Fluopicolide | 1:10 | 0.6:6 |
| X.02 | Fluopicolide | 3:1 | 0.6:0.2 |
| X.02 | Zoxamide | 1:1 | 6:6 |
| X.02 | Zoxamide | 30:1 | 6:0.2 |
| X.02 | Zoxamide | 1:10 | 0.6:6 |
| X.02 | Zoxamide | 3:1 | 0.6:0.2 |
| X.02 | Mandipropamid | 1:1 | 6:6 |
| X.02 | Mandipropamid | 30:1 | 6:0.2 |
| X.02 | Mandipropamid | 1:10 | 0.6:6 |
| X.02 | Mandipropamid | 3:1 | 0.6:0.2 |
| X.02 | Cyclobutrifluram | 1:1 | 6:6 |
| X.02 | Cyclobutrifluram | 10:1 | 6:0.6 |
| X.02 | Cyclobutrifluram | 1:10 | 0.6:6 |
| X.02 | Cyclobutrifluram | 1:1 | 0.6:0.6 |
| X.03 | Picarbutrazox | 1:1 | 6:6 |
| X.03 | Picarbutrazox | 30:1 | 6:0.2 |
| X.03 | Picarbutrazox | 1:10 | 0.6:6 |
| X.03 | Picarbutrazox | 3:1 | 0.6:0.2 |
| X.03 | Cymoxanil | 1:1 | 6:6 |
| X.03 | Cymoxanil | 10:1 | 6:0.6 |
| X.03 | Cymoxanil | 1:10 | 0.6:6 |
| X.03 | Cymoxanil | 1:1 | 0.6:0.6 |
| X.03 | Disodium phosphonate | 1:1 | 6:6 |
| X.03 | Disodium phosphonate | 10:1 | 6:0.6 |
| X.03 | Disodium phosphonate | 1:10 | 0.6:6 |
| X.03 | Disodium phosphonate | 1:1 | 0.6:0.6 |
| X.03 | Difenoconazole | 1:1 | 6:6 |
| X.03 | Difenoconazole | 10:1 | 6:0.6 |
| X.03 | Difenoconazole | 1:10 | 0.6:6 |
| X.03 | Difenoconazole | 1:1 | 0.6:0.6 |
| X.03 | Fludioxonil | 1:1 | 6:6 |
| X.03 | Fludioxonil | 10:1 | 6:0.6 |
| X.03 | Fludioxonil | 1:10 | 0.6:6 |
| X.03 | Fludioxonil | 1:1 | 0.6:0.6 |
| X.03 | Fluazinam | 1:1 | 6:6 |
| X.03 | Fluazinam | 30:1 | 6:0.2 |
| X.03 | Fluazinam | 1:10 | 0.6:6 |
| X.03 | Fluazinam | 3:1 | 0.6:0.2 |
| X.03 | Ametoctradin | 1:1 | 6:6 |
| X.03 | Ametoctradin | 30:1 | 6:0.2 |
| X.03 | Ametoctradin | 1:10 | 0.6:6 |
| X.03 | Ametoctradin | 3:1 | 0.6:0.2 |
| X.03 | Amisulbrom | 1:1 | 6:6 |
| X.03 | Amisulbrom | 30:1 | 6:0.2 |
| X.03 | Amisulbrom | 1:10 | 0.6:6 |
| X.03 | Amisulbrom | 3:1 | 0.6:0.2 |
| X.03 | Cyazofamid | 1:1 | 6:6 |
| X.03 | Cyazofamid | 30:1 | 6:0.2 |
| X.03 | Cyazofamid | 1:10 | 0.6:6 |
| X.03 | Cyazofamid | 3:1 | 0.6:0.2 |
| X.03 | Azoxystrobin | 1:1 | 6:6 |
| X.03 | Azoxystrobin | 10:1 | 6:0.6 |
| X.03 | Azoxystrobin | 1:10 | 0.6:6 |
| X.03 | Azoxystrobin | 1:1 | 0.6:0.6 |
| X.03 | Sedaxane | 1:1 | 6:6 |
| X.03 | Sedaxane | 10:1 | 6:0.6 |
| X.03 | Sedaxane | 1:10 | 0.6:6 |
| X.03 | Sedaxane | 1:1 | 0.6:0.6 |
| X.03 | Pydiflumetofen | 1:1 | 6:6 |
| X.03 | Pydiflumetofen | 10:1 | 6:0.6 |
| X.03 | Pydiflumetofen | 1:10 | 0.6:6 |
| X.03 | Pydiflumetofen | 1:1 | 0.6:0.6 |
| X.03 | Metalaxyl-M | 1:1 | 6:6 |
| X.03 | Metalaxyl-M | 30:1 | 6:0.2 |
| X.03 | Metalaxyl-M | 1:10 | 0.6:6 |
| X.03 | Metalaxyl-M | 3:1 | 0.6:0.2 |
| X.03 | Folpet | 1:1 | 6:6 |
| X.03 | Folpet | 10:1 | 6:0.6 |
| X.03 | Folpet | 1:10 | 0.6:6 |
| X.03 | Folpet | 1:1 | 0.6:0.6 |
| X.03 | Chlorothalonil | 1:1 | 6:6 |
| X.03 | Chlorothalonil | 10:1 | 6:0.6 |
| X.03 | Chlorothalonil | 1:10 | 0.6:6 |
| X.03 | Chlorothalonil | 1:1 | 0.6:0.6 |
| X.03 | Oxathiapiprolin | 1:1 | 6:6 |
| X.03 | Oxathiapiprolin | 30:1 | 6:0.2 |
| X.03 | Oxathiapiprolin | 1:10 | 0.6:6 |
| X.03 | Oxathiapiprolin | 3:1 | 0.6:0.2 |
| X.03 | Fluoxapiprolin | 1:1 | 6:6 |
| X.03 | Fluoxapiprolin | 30:1 | 6:0.2 |
| X.03 | Fluoxapiprolin | 1:10 | 0.6:6 |
| X.03 | Fluoxapiprolin | 3:1 | 0.6:0.2 |
| X.03 | Acibenzolar-S-methyl | 1:1 | 6:6 |
| X.03 | Acibenzolar-S-methyl | 10:1 | 6:0.6 |
| X.03 | Acibenzolar-S-methyl | 1:10 | 0.6:6 |
| X.03 | Acibenzolar-S-methyl | 1:1 | 0.6:0.6 |
| X.03 | Fluopicolide | 1:1 | 6:6 |
| X.03 | Fluopicolide | 30:1 | 6:0.2 |
| X.03 | Fluopicolide | 1:10 | 0.6:6 |
| X.03 | Fluopicolide | 3:1 | 0.6:0.2 |
| X.03 | Zoxamide | 1:1 | 6:6 |
| X.03 | Zoxamide | 30:1 | 6:0.2 |
| X.03 | Zoxamide | 1:10 | 0.6:6 |
| X.03 | Zoxamide | 3:1 | 0.6:0.2 |
| X.03 | Mandipropamid | 1:1 | 6:6 |
| X.03 | Mandipropamid | 30:1 | 6:0.2 |
| X.03 | Mandipropamid | 1:10 | 0.6:6 |
| X.03 | Mandipropamid | 3:1 | 0.6:0.2 |
| X.03 | Cyclobutrifluram | 1:1 | 6:6 |
| X.03 | Cyclobutrifluram | 10:1 | 6:0.6 |
| X.03 | Cyclobutrifluram | 1:10 | 0.6:6 |
| X.03 | Cyclobutrifluram | 1:1 | 0.6:0.6 |
| X.04 | Picarbutrazox | 1:1 | 6:6 |
| X.04 | Picarbutrazox | 30:1 | 6:0.2 |
| X.04 | Picarbutrazox | 1:10 | 0.6:6 |
| X.04 | Picarbutrazox | 3:1 | 0.6:0.2 |
| X.04 | Cymoxanil | 1:1 | 6:6 |
| X.04 | Cymoxanil | 10:1 | 6:0.6 |
| X.04 | Cymoxanil | 1:10 | 0.6:6 |
| X.04 | Cymoxanil | 1:1 | 0.6:0.6 |
| X.04 | Disodium phosphonate | 1:1 | 6:6 |
| X.04 | Disodium phosphonate | 10:1 | 6:0.6 |
| X.04 | Disodium phosphonate | 1:10 | 0.6:6 |
| X.04 | Disodium phosphonate | 1:1 | 0.6:0.6 |
| X.04 | Difenoconazole | 1:1 | 6:6 |
| X.04 | Difenoconazole | 10:1 | 6:0.6 |
| X.04 | Difenoconazole | 1:10 | 0.6:6 |
| X.04 | Difenoconazole | 1:1 | 0.6:0.6 |
| X.04 | Fludioxonil | 1:1 | 6:6 |
| X.04 | Fludioxonil | 10:1 | 6:0.6 |
| X.04 | Fludioxonil | 1:10 | 0.6:6 |
| X.04 | Fludioxonil | 1:1 | 0.6:0.6 |
| X.04 | Fluazinam | 1:1 | 6:6 |
| X.04 | Fluazinam | 30:1 | 6:0.2 |
| X.04 | Fluazinam | 1:10 | 0.6:6 |
| X.04 | Fluazinam | 3:1 | 0.6:0.2 |
| X.04 | Ametoctradin | 1:1 | 6:6 |
| X.04 | Ametoctradin | 30:1 | 6:0.2 |
| X.04 | Ametoctradin | 1:10 | 0.6:6 |
| X.04 | Ametoctradin | 3:1 | 0.6:0.2 |
| X.04 | Amisulbrom | 1:1 | 6:6 |
| X.04 | Amisulbrom | 30:1 | 6:0.2 |
| X.04 | Amisulbrom | 1:10 | 0.6:6 |
| X.04 | Amisulbrom | 3:1 | 0.6:0.2 |
| X.04 | Cyazofamid | 1:1 | 6:6 |
| X.04 | Cyazofamid | 30:1 | 6:0.2 |
| X.04 | Cyazofamid | 1:10 | 0.6:6 |
| X.04 | Cyazofamid | 3:1 | 0.6:0.2 |
| X.04 | Azoxystrobin | 1:1 | 6:6 |
| X.04 | Azoxystrobin | 10:1 | 6:0.6 |
| X.04 | Azoxystrobin | 1:10 | 0.6:6 |
| X.04 | Azoxystrobin | 1:1 | 0.6:0.6 |
| X.04 | Sedaxane | 1:1 | 6:6 |
| X.04 | Sedaxane | 10:1 | 6:0.6 |
| X.04 | Sedaxane | 1:10 | 0.6:6 |
| X.04 | Sedaxane | 1:1 | 0.6:0.6 |
| X.04 | Pydiflumetofen | 1:1 | 6:6 |
| X.04 | Pydiflumetofen | 10:1 | 6:0.6 |
| X.04 | Pydiflumetofen | 1:10 | 0.6:6 |
| X.04 | Pydiflumetofen | 1:1 | 0.6:0.6 |
| X.04 | Metalaxyl-M | 1:1 | 6:6 |
| X.04 | Metalaxyl-M | 30:1 | 6:0.2 |
| X.04 | Metalaxyl-M | 1:10 | 0.6:6 |
| X.04 | Metalaxyl-M | 3:1 | 0.6:0.2 |
| X.04 | Folpet | 1:1 | 6:6 |
| X.04 | Folpet | 10:1 | 6:0.6 |
| X.04 | Folpet | 1:10 | 0.6:6 |
| X.04 | Folpet | 1:1 | 0.6:0.6 |
| X.04 | Chlorothalonil | 1:1 | 6:6 |
| X.04 | Chlorothalonil | 10:1 | 6:0.6 |
| X.04 | Chlorothalonil | 1:10 | 0.6:6 |
| X.04 | Chlorothalonil | 1:1 | 0.6:0.6 |
| X.04 | Oxathiapiprolin | 1:1 | 6:6 |
| X.04 | Oxathiapiprolin | 30:1 | 6:0.2 |
| X.04 | Oxathiapiprolin | 1:10 | 0.6:6 |
| X.04 | Oxathiapiprolin | 3:1 | 0.6:0.2 |
| X.04 | Fluoxapiprolin | 1:1 | 6:6 |
| X.04 | Fluoxapiprolin | 30:1 | 6:0.2 |
| X.04 | Fluoxapiprolin | 1:10 | 0.6:6 |
| X.04 | Fluoxapiprolin | 3:1 | 0.6:0.2 |
| X.04 | Acibenzolar-S-methyl | 1:1 | 6:6 |
| X.04 | Acibenzolar-S-methyl | 10:1 | 6:0.6 |
| X.04 | Acibenzolar-S-methyl | 1:10 | 0.6:6 |
| X.04 | Acibenzolar-S-methyl | 1:1 | 0.6:0.6 |
| X.04 | Fluopicolide | 1:1 | 6:6 |
| X.04 | Fluopicolide | 30:1 | 6:0.2 |
| X.04 | Fluopicolide | 1:10 | 0.6:6 |
| X.04 | Fluopicolide | 3:1 | 0.6:0.2 |
| X.04 | Zoxamide | 1:1 | 6:6 |
| X.04 | Zoxamide | 30:1 | 6:0.2 |
| X.04 | Zoxamide | 1:10 | 0.6:6 |
| X.04 | Zoxamide | 3:1 | 0.6:0.2 |
| X.04 | Mandipropamid | 1:1 | 6:6 |
| X.04 | Mandipropamid | 30:1 | 6:0.2 |
| X.04 | Mandipropamid | 1:10 | 0.6:6 |
| X.04 | Mandipropamid | 3:1 | 0.6:0.2 |
| X.04 | Cyclobutrifluram | 1:1 | 6:6 |
| X.04 | Cyclobutrifluram | 10:1 | 6:0.6 |
| X.04 | Cyclobutrifluram | 1:10 | 0.6:6 |
| X.04 | Cyclobutrifluram | 1:1 | 0.6:0.6 |
| X.05 | Picarbutrazox | 1:1 | 6:6 |
| X.05 | Picarbutrazox | 30:1 | 6:0.2 |
| X.05 | Picarbutrazox | 1:10 | 0.6:6 |
| X.05 | Picarbutrazox | 3:1 | 0.6:0.2 |
| X.05 | Cymoxanil | 1:1 | 6:6 |
| X.05 | Cymoxanil | 10:1 | 6:0.6 |
| X.05 | Cymoxanil | 1:10 | 0.6:6 |
| X.05 | Cymoxanil | 1:1 | 0.6:0.6 |
| X.05 | Disodium phosphonate | 1:1 | 6:6 |
| X.05 | Disodium phosphonate | 10:1 | 6:0.6 |
| X.05 | Disodium phosphonate | 1:10 | 0.6:6 |
| X.05 | Disodium phosphonate | 1:1 | 0.6:0.6 |
| X.05 | Difenoconazole | 1:1 | 6:6 |
| X.05 | Difenoconazole | 10:1 | 6:0.6 |
| X.05 | Difenoconazole | 1:10 | 0.6:6 |
| K.05 | Difenoconazole | 1:1 | 0.6:0.6 |
| X.05 | Fludioxonil | 1:1 | 6:6 |
| X.05 | Fludioxonil | 10:1 | 6:0.6 |
| X.05 | Fludioxonil | 1:10 | 0.6:6 |
| X.05 | Fludioxonil | 1:1 | 0.6:0.6 |
| X.05 | Fluazinam | 1:1 | 6:6 |
| X.05 | Fluazinam | 30:1 | 6:0.2 |
| X.05 | Fluazinam | 1:10 | 0.6:6 |
| X.05 | Fluazinam | 3:1 | 0.6:0.2 |
| X.05 | Ametoctradin | 1:1 | 6:6 |
| X.05 | Ametoctradin | 30:1 | 6:0.2 |
| X.05 | Ametoctradin | 1:10 | 0.6:6 |
| X.05 | Ametoctradin | 3:1 | 0.6:0.2 |
| X.05 | Amisulbrom | 1:1 | 6:6 |
| X.05 | Amisulbrom | 30:1 | 6:0.2 |
| X.05 | Amisulbrom | 1:10 | 0.6:6 |
| X.05 | Amisulbrom | 3:1 | 0.6:0.2 |
| X.05 | Cyazofamid | 1:1 | 6:6 |
| X.05 | Cyazofamid | 30:1 | 6:0.2 |
| X.05 | Cyazofamid | 1:10 | 0.6:6 |
| X.05 | Cyazofamid | 3:1 | 0.6:0.2 |
| X.05 | Azoxystrobin | 1:1 | 6:6 |
| X.05 | Azoxystrobin | 10:1 | 6:0.6 |
| X.05 | Azoxystrobin | 1:10 | 0.6:6 |
| X.05 | Azoxystrobin | 1:1 | 0.6:0.6 |
| X.05 | Sedaxane | 1:1 | 6:6 |
| X.05 | Sedaxane | 10:1 | 6:0.6 |
| X.05 | Sedaxane | 1:10 | 0.6:6 |
| X.05 | Sedaxane | 1:1 | 0.6:0.6 |
| X.05 | Pydiflumetofen | 1:1 | 6:6 |
| X.05 | Pydiflumetofen | 10:1 | 6:0.6 |
| X.05 | Pydiflumetofen | 1:10 | 0.6:6 |
| X.05 | Pydiflumetofen | 1:1 | 0.6:0.6 |
| X.05 | Metalaxyl-M | 1:1 | 6:6 |
| X.05 | Metalaxyl-M | 30:1 | 6:0.2 |
| X.05 | Metalaxyl-M | 1:10 | 0.6:6 |
| X.05 | Metalaxyl-M | 3:1 | 0.6:0.2 |
| X.05 | Folpet | 1:1 | 6:6 |
| X.05 | Folpet | 10:1 | 6:0.6 |
| X.05 | Folpet | 1:10 | 0.6:6 |
| X.05 | Folpet | 1:1 | 0.6:0.6 |
| X.05 | Chlorothalonil | 1:1 | 6:6 |
| X.05 | Chlorothalonil | 10:1 | 6:0.6 |
| X.05 | Chlorothalonil | 1:10 | 0.6:6 |
| X.05 | Chlorothalonil | 1:1 | 0.6:0.6 |
| X.05 | Oxathiapiprolin | 1:1 | 6:6 |
| X.05 | Oxathiapiprolin | 30:1 | 6:0.2 |
| X.05 | Oxathiapiprolin | 1:10 | 0.6:6 |
| X.05 | Oxathiapiprolin | 3:1 | 0.6:0.2 |
| X.05 | Fluoxapiprolin | 1:1 | 6:6 |
| X.05 | Fluoxapiprolin | 30:1 | 6:0.2 |
| X.05 | Fluoxapiprolin | 1:10 | 0.6:6 |
| X.05 | Fluoxapiprolin | 3:1 | 0.6:0.2 |
| X.05 | Acibenzolar-S-methyl | 1:1 | 6:6 |
| X.05 | Acibenzolar-S-methyl | 10:1 | 6:0.6 |
| X.05 | Acibenzolar-S-methyl | 1:10 | 0.6:6 |
| X.05 | Acibenzolar-S-methyl | 1:1 | 0.6:0.6 |
| X.05 | Fluopicolide | 1:1 | 6:6 |
| X.05 | Fluopicolide | 30:1 | 6:0.2 |
| X.05 | Fluopicolide | 1:10 | 0.6:6 |
| X.05 | Fluopicolide | 3:1 | 0.6:0.2 |
| X.05 | Zoxamide | 1:1 | 6:6 |
| X.05 | Zoxamide | 30:1 | 6:0.2 |
| X.05 | Zoxamide | 1:10 | 0.6:6 |
| X.05 | Zoxamide | 3:1 | 0.6:0.2 |
| X.05 | Mandipropamid | 1:1 | 6:6 |
| X.05 | Mandipropamid | 30:1 | 6:0.2 |
| X.05 | Mandipropamid | 1:10 | 0.6:6 |
| X.05 | Mandipropamid | 3:1 | 0.6:0.2 |
| X.05 | Cyclobutrifluram | 1:1 | 6:6 |
| X.05 | Cyclobutrifluram | 10:1 | 6:0.6 |
| X.05 | Cyclobutrifluram | 1:10 | 0.6:6 |
| X.05 | Cyclobutrifluram | 1:1 | 0.6:0.6 |
| X.06 | Picarbutrazox | 1:1 | 6:6 |
| X.06 | Picarbutrazox | 30:1 | 6:0.2 |
| X.06 | Picarbutrazox | 1:10 | 0.6:6 |
| X.06 | Picarbutrazox | 3:1 | 0.6:0.2 |
| X.06 | Cymoxanil | 1:1 | 6:6 |
| X.06 | Cymoxanil | 10:1 | 6:0.6 |
| X.06 | Cymoxanil | 1:10 | 0.6:6 |
| X.06 | Disodium phosphonate | 1:1 | 6:6 |
| X.06 | Disodium phosphonate | 10:1 | 6:0.6 |
| X.06 | Difenoconazole | 1:1 | 6:6 |
| X.06 | Difenoconazole | 10:1 | 6:0.6 |
| X.06 | Fludioxonil | 1:1 | 6:6 |
| X.06 | Fludioxonil | 10:1 | 6:0.6 |
| X.06 | Fluazinam | 1:1 | 6:6 |
| X.06 | Fluazinam | 30:1 | 6:0.2 |
| X.06 | Ametoctradin | 1:1 | 6:6 |
| X.06 | Ametoctradin | 30:1 | 6:0.2 |
| X.06 | Ametoctradin | 1:10 | 0.6:6 |
| X.06 | Amisulbrom | 1:1 | 6:6 |
| X.06 | Amisulbrom | 30:1 | 6:0.2 |
| X.06 | Cyazofamid | 1:1 | 6:6 |
| X.06 | Cyazofamid | 30:1 | 6:0.2 |
| X.06 | Cyazofamid | 1:10 | 0.6:6 |
| X.06 | Azoxystrobin | 1:1 | 6:6 |
| X.06 | Azoxystrobin | 10:1 | 6:0.6 |
| X.06 | Azoxystrobin | 1:10 | 0.6:6 |
| X.06 | Azoxystrobin | 1:1 | 0.6:0.6 |
| X.06 | Sedaxane | 1:1 | 6:6 |
| X.06 | Sedaxane | 10:1 | 6:0.6 |
| X.06 | Pydiflumetofen | 1:1 | 6:6 |
| X.06 | Pydiflumetofen | 10:1 | 6:0.6 |
| X.06 | Metalaxyl-M | 1:1 | 6:6 |
| X.06 | Metalaxyl-M | 30:1 | 6:0.2 |
| X.06 | Metalaxyl-M | 1:10 | 0.6:6 |
| X.06 | Folpet | 1:1 | 6:6 |
| X.06 | Folpet | 10:1 | 6:0.6 |
| X.06 | Folpet | 1:10 | 0.6:6 |
| X.06 | Chlorothalonil | 1:1 | 6:6 |
| X.06 | Chlorothalonil | 10:1 | 6:0.6 |
| X.06 | Chlorothalonil | 1:10 | 0.6:6 |
| X.06 | Oxathiapiprolin | 1:1 | 6:6 |
| X.06 | Oxathiapiprolin | 30:1 | 6:0.2 |
| X.06 | Oxathiapiprolin | 1:10 | 0.6:6 |
| X.06 | Fluoxapiprolin | 1:1 | 6:6 |
| X.06 | Fluoxapiprolin | 30:1 | 6:0.2 |
| X.06 | Fluoxapiprolin | 1:10 | 0.6:6 |
| X.06 | Acibenzolar-S-methyl | 1:1 | 6:6 |
| X.06 | Acibenzolar-S-methyl | 10:1 | 6:0.6 |
| X.06 | Acibenzolar-S-methyl | 1:10 | 0.6:6 |
| X.06 | Fluopicolide | 1:1 | 6:6 |
| X.06 | Fluopicolide | 30:1 | 6:0.2 |
| X.06 | Zoxamide | 1 : 1 | 6:6 |
| X.06 | Zoxamide | 30:1 | 6:0.2 |
| X.06 | Zoxamide | 1:10 | 0.6:6 |
| X.06 | Mandipropamid | 1:1 | 6:6 |
| X.06 | Mandipropamid | 30:1 | 6:0.2 |
| X.06 | Cyclobutrifluram | 1:1 | 6:6 |
| X.06 | Cyclobutrifluram | 10:1 | 6:0.6 |
| X.21 | Picarbutrazox | 3.3:1 | 20:6 |
| X.21 | Picarbutrazox | 33.3:1 | 20:0.6 |
| X.21 | Picarbutrazox | 1:3 | 2:6 |
| X.21 | Picarbutrazox | 3.3:1 | 2:0.6 |
| X.21 | Cymoxanil | 3.3:1 | 20:6 |
| X.21 | Cymoxanil | 33.3:1 | 20:0.6 |
| X.21 | Cymoxanil | 1:3 | 2:6 |
| X.21 | Cymoxanil | 3.3:1 | 2:0.6 |
| X.21 | Disodium phosphonate | 3.3:1 | 20:6 |
| X.21 | Disodium phosphonate | 33.3:1 | 20:0.6 |
| X.21 | Disodium phosphonate | 1:3 | 2:6 |
| X.21 | Disodium phosphonate | 3.3:1 | 2:0.6 |
| X.21 | Difenoconazole | 3.3:1 | 20:6 |
| X.21 | Difenoconazole | 33.3:1 | 20:0.6 |
| X.21 | Difenoconazole | 1:3 | 2:6 |
| X.21 | Difenoconazole | 3.3:1 | 2:0.6 |
| X.21 | Fludioxonil | 3.3:1 | 20:6 |
| X.21 | Fludioxonil | 33.3:1 | 20:0.6 |
| X.21 | Fludioxonil | 1:3 | 2:6 |
| X.21 | Fludioxonil | 3.3:1 | 2:0.6 |
| X.21 | Fluazinam | 3.3:1 | 20:6 |
| X.21 | Fluazinam | 33.3:1 | 20:0.6 |
| X.21 | Fluazinam | 1:3 | 2:6 |
| X.21 | Fluazinam | 3.3:1 | 2:0.6 |
| X.21 | Azoxystrobin | 3.3:1 | 20:6 |
| X.21 | Azoxystrobin | 33.3:1 | 20:0.6 |
| X.21 | Azoxystrobin | 1:3 | 2:6 |
| X.21 | Azoxystrobin | 3.3:1 | 2:0.6 |
| X.21 | Sedaxane | 3.3:1 | 20:6 |
| X.21 | Sedaxane | 33.3:1 | 20:0.6 |
| X.21 | Sedaxane | 1:3 | 2:6 |
| X.21 | Sedaxane | 3.3:1 | 2:0.6 |
| X.21 | Pydiflumetofen | 3.3:1 | 20:6 |
| X.21 | Pydiflumetofen | 33.3:1 | 20:0.6 |
| X.21 | Pydiflumetofen | 1:3 | 2:6 |
| X.21 | Pydiflumetofen | 3.3:1 | 2:0.6 |
| X.21 | Metalaxyl-M | 3.3:1 | 20:6 |
| X.21 | Metalaxyl-M | 33.3:1 | 20:0.6 |
| X.21 | Metalaxyl-M | 1:3 | 2:6 |
| X.21 | Metalaxyl-M | 3.3:1 | 2:0.6 |
| X.21 | Folpet | 3.3:1 | 20:6 |
| X.21 | Folpet | 33.3:1 | 20:0.6 |
| X.21 | Folpet | 1:3 | 2:6 |
| X.21 | Folpet | 3.3:1 | 2:0.6 |
| X.21 | Oxathiapiprolin | 3.3:1 | 20:6 |
| X.21 | Oxathiapiprolin | 33.3:1 | 20:0.6 |
| X.21 | Oxathiapiprolin | 1:3 | 2:6 |
| X.21 | Oxathiapiprolin | 3.3:1 | 2:0.6 |
| X.21 | Fluoxapiprolin | 3.3:1 | 20:6 |
| X.21 | Fluoxapiprolin | 33.3:1 | 20:0.6 |
| X.21 | Fluoxapiprolin | 1:3 | 2:6 |
| X.21 | Fluoxapiprolin | 3.3:1 | 2:0.6 |
| X.21 | Fluopicolide | 3.3:1 | 20:6 |
| X.21 | Fluopicolide | 33.3:1 | 20:0.6 |
| X.21 | Fluopicolide | 1:3 | 2:6 |
| X.21 | Fluopicolide | 3.3:1 | 2:0.6 |
| X.21 | Zoxamide | 3.3:1 | 20:6 |
| X.21 | Zoxamide | 33.3:1 | 20:0.6 |
| X.21 | Zoxamide | 1:3 | 2:6 |
| X.21 | Zoxamide | 3.3:1 | 2:0.6 |
| X.21 | Mandipropamid | 3.3:1 | 20:6 |
| X.21 | Mandipropamid | 33.3:1 | 20:0.6 |
| X.21 | Mandipropamid | 1:3 | 2:6 |
| X.21 | Mandipropamid | 3.3:1 | 2:0.6 |
| X.21 | Cyclobutrifluram | 3.3:1 | 20:6 |
| X.21 | Cyclobutrifluram | 33.3:1 | 20:0.6 |
| X.21 | Cyclobutrifluram | 1:3 | 2:6 |
| X.21 | Cyclobutrifluram | 3.3:1 | 2:0.6 |
| X.21 | Mefentrifluconazole | 3.3:1 | 20:6 |
| X.21 | Mefentrifluconazole | 33.3:1 | 20:0.6 |
| X.21 | Mefentrifluconazole | 1:3 | 2:6 |
| X.21 | Mefentrifluconazole | 3.3:1 | 2:0.6 |
| X.21 | Florylpicoxamid | 3.3:1 | 20:6 |
| X.21 | Florylpicoxamid | 33.3:1 | 20:0.6 |
| X.21 | Florylpicoxamid | 1:3 | 2:6 |
| X.21 | Florylpicoxamid | 3.3:1 | 2:0.6 |
| X.21 | Streptomyces chrestomyceticus | 3.3:1 | 20:6 |
| | CBS149411 | | |
| X.21 | Streptomyces chrestomyceticus | 33.3:1 | 20:0.6 |
| | CBS149411 | | |
| X.21 | Streptomyces chrestomyceticus | 1:3 | 2:6 |
| | CBS149411 | | |
| X.21 | Streptomyces chrestomyceticus | 3.3:1 | 2:0.6 |
| | CBS149411 | | |
| X.21 | Compound Z | 3.3:1 | 20:6 |
| X.21 | Compound Z | 33.3:1 | 20:0.6 |
| X.21 | Compound Z | 1:3 | 2:6 |
| X.21 | Compound Z | 3.3:1 | 2:0.6 |

### Example B2: Activity Against Phytophthora capsici (Vegetable Blight)

Sporangia of the pathogen, harvested from a fresh culture grown on artificial media were directly mixed into nutrient broth (PDB potato dextrose broth). After placing a (DMSO) solution of the test compounds into a microtiter plate (96-well format) the nutrient broth containing the sporangia was added. The test plates were incubated at 24 00 and the inhibition of growth was determined photometrically after 48 hrs. The following mixture compositions (A:B) at the reported concentration (in ppm) gave at least 80% disease control in this test.

| Component A | | | Conc. (ppm) |
|---|---|---|---|
| (Compound) | Component B | Ratio A:B | (A:B) |
| X.01 | Picarbutrazox | 1:1 | 6:6 |
| X.01 | Picarbutrazox | 30:1 | 6:0.2 |
| X.01 | Picarbutrazox | 1:10 | 0.6:6 |
| X.01 | Picarbutrazox | 3:1 | 0.6:0.2 |
| X.01 | Cymoxanil | 1:1 | 6:6 |
| X.01 | Cymoxanil | 10:1 | 6:0.6 |
| X.01 | Cymoxanil | 1:10 | 0.6:6 |
| X.01 | Cymoxanil | 1:1 | 0.6:0.6 |
| X.01 | Disodium phosphonate | 1:1 | 6:6 |
| X.01 | Disodium phosphonate | 10:1 | 6:0.6 |
| X.01 | Disodium phosphonate | 1:10 | 0.6:6 |
| X.01 | Disodium phosphonate | 1:1 | 0.6:0.6 |
| X.01 | Difenoconazole | 1:1 | 6:6 |
| X.01 | Difenoconazole | 10:1 | 6:0.6 |
| X.01 | Difenoconazole | 1:10 | 0.6:6 |
| X.01 | Difenoconazole | 1:1 | 0.6:0.6 |
| X.01 | Fludioxonil | 1:1 | 6:6 |
| X.01 | Fludioxonil | 10:1 | 6:0.6 |
| X.01 | Fludioxonil | 1:10 | 0.6:6 |
| X.01 | Fludioxonil | 1:1 | 0.6:0.6 |
| X.01 | Fluazinam | 1:1 | 6:6 |
| X.01 | Fluazinam | 30:1 | 6:0.2 |
| X.01 | Fluazinam | 1:10 | 0.6:6 |
| X.01 | Fluazinam | 3:1 | 0.6:0.2 |
| X.01 | Ametoctradin | 1:1 | 6:6 |
| X.01 | Ametoctradin | 30:1 | 6:0.2 |
| X.01 | Ametoctradin | 1:10 | 0.6:6 |
| X.01 | Ametoctradin | 3:1 | 0.6:0.2 |
| X.01 | Amisulbrom | 1:1 | 6:6 |
| X.01 | Amisulbrom | 30:1 | 6:0.2 |
| X.01 | Amisulbrom | 1:10 | 0.6:6 |
| X.01 | Amisulbrom | 3:1 | 0.6:0.2 |
| X.01 | Cyazofamid | 1:1 | 6:6 |
| X.01 | Cyazofamid | 30:1 | 6:0.2 |
| X.01 | Cyazofamid | 1:10 | 0.6:6 |
| X.01 | Cyazofamid | 3:1 | 0.6:0.2 |
| X.01 | Azoxystrobin | 1:1 | 6:6 |
| X.01 | Azoxystrobin | 10:1 | 6:0.6 |
| X.01 | Azoxystrobin | 1:10 | 0.6:6 |
| X.01 | Azoxystrobin | 1:1 | 0.6:0.6 |
| X.01 | Sedaxane | 1:1 | 6:6 |
| X.01 | Sedaxane | 10:1 | 6:0.6 |
| X.01 | Sedaxane | 1:10 | 0.6:6 |
| X.01 | Sedaxane | 1:1 | 0.6:0.6 |
| X.01 | Pydiflumetofen | 1:1 | 6:6 |
| X.01 | Pydiflumetofen | 10:1 | 6:0.6 |
| X.01 | Pydiflumetofen | 1:10 | 0.6:6 |
| X.01 | Pydiflumetofen | 1:1 | 0.6:0.6 |
| X.01 | Metalaxyl-M | 1:1 | 6:6 |
| X.01 | Metalaxyl-M | 30:1 | 6:0.2 |
| X.01 | Metalaxyl-M | 1:10 | 0.6:6 |
| X.01 | Metalaxyl-M | 3:1 | 0.6:0.2 |
| X.01 | Folpet | 1:1 | 6:6 |
| X.01 | Folpet | 10:1 | 6:0.6 |
| X.01 | Folpet | 1:10 | 0.6:6 |
| X.01 | Folpet | 1:1 | 0.6:0.6 |
| X.01 | Chlorothalonil | 1:1 | 6:6 |
| X.01 | Chlorothalonil | 10:1 | 6:0.6 |
| X.01 | Chlorothalonil | 1:10 | 0.6:6 |
| X.01 | Chlorothalonil | 1:1 | 0.6:0.6 |
| X.01 | Oxathiapiprolin | 1:1 | 6:6 |
| X.01 | Oxathiapiprolin | 30:1 | 6:0.2 |
| X.01 | Oxathiapiprolin | 1:10 | 0.6:6 |
| X.01 | Oxathiapiprolin | 3:1 | 0.6:0.2 |
| X.01 | Fluoxapiprolin | 1:1 | 6:6 |
| X.01 | Fluoxapiprolin | 30:1 | 6:0.2 |
| X.01 | Fluoxapiprolin | 1:10 | 0.6:6 |
| X.01 | Fluoxapiprolin | 3:1 | 0.6:0.2 |
| X.01 | Acibenzolar-S-methyl | 1:1 | 6:6 |
| X.01 | Acibenzolar-S-methyl | 10:1 | 6:0.6 |
| X.01 | Acibenzolar-S-methyl | 1:10 | 0.6:6 |
| X.01 | Acibenzolar-S-methyl | 1:1 | 0.6:0.6 |
| X.01 | Fluopicolide | 1:1 | 6:6 |
| X.01 | Fluopicolide | 30:1 | 6:0.2 |
| X.01 | Fluopicolide | 1:10 | 0.6:6 |
| X.01 | Fluopicolide | 3:1 | 0.6:0.2 |
| X.01 | Zoxamide | 1:1 | 6:6 |
| X.01 | Zoxamide | 30:1 | 6:0.2 |
| X.01 | Zoxamide | 1:10 | 0.6:6 |
| X.01 | Zoxamide | 3:1 | 0.6:0.2 |
| X.01 | Mandipropamid | 1:1 | 6:6 |
| X.01 | Mandipropamid | 30:1 | 6:0.2 |
| X.01 | Mandipropamid | 1:10 | 0.6:6 |
| X.01 | Mandipropamid | 3:1 | 0.6:0.2 |
| X.01 | Cyclobutrifluram | 1:1 | 6:6 |
| X.01 | Cyclobutrifluram | 10:1 | 6:0.6 |
| X.01 | Cyclobutrifluram | 1:10 | 0.6:6 |
| X.01 | Cyclobutrifluram | 1:1 | 0.6:0.6 |
| X.02 | Picarbutrazox | 1:1 | 6:6 |
| X.02 | Picarbutrazox | 30:1 | 6:0.2 |
| X.02 | Picarbutrazox | 1:10 | 0.6:6 |
| X.02 | Picarbutrazox | 3:1 | 0.6:0.2 |
| X.02 | Cymoxanil | 1:1 | 6:6 |
| X.02 | Cymoxanil | 10:1 | 6:0.6 |
| X.02 | Cymoxanil | 1:10 | 0.6:6 |
| X.02 | Cymoxanil | 1:1 | 0.6:0.6 |
| X.02 | Disodium phosphonate | 1:1 | 6:6 |
| X.02 | Disodium phosphonate | 10:1 | 6:0.6 |
| X.02 | Disodium phosphonate | 1:10 | 0.6:6 |
| X.02 | Disodium phosphonate | 1:1 | 0.6:0.6 |
| X.02 | Difenoconazole | 1:1 | 6:6 |
| X.02 | Difenoconazole | 10:1 | 6:0.6 |
| X.02 | Difenoconazole | 1:10 | 0.6:6 |
| X.02 | Difenoconazole | 1:1 | 0.6:0.6 |
| X.02 | Fludioxonil | 1:1 | 6:6 |
| X.02 | Fludioxonil | 10:1 | 6:0.6 |
| X.02 | Fludioxonil | 1:10 | 0.6:6 |
| X.02 | Fludioxonil | 1:1 | 0.6:0.6 |
| X.02 | Fluazinam | 1:1 | 6:6 |
| X.02 | Fluazinam | 30:1 | 6:0.2 |
| X.02 | Fluazinam | 1:10 | 0.6:6 |
| X.02 | Fluazinam | 3:1 | 0.6:0.2 |
| X.02 | Ametoctradin | 1:1 | 6:6 |
| X.02 | Ametoctradin | 30:1 | 6:0.2 |
| X.02 | Ametoctradin | 1:10 | 0.6:6 |
| X.02 | Ametoctradin | 3:1 | 0.6:0.2 |
| X.02 | Amisulbrom | 1:1 | 6:6 |
| X.02 | Amisulbrom | 30:1 | 6:0.2 |
| X.02 | Amisulbrom | 1:10 | 0.6:6 |
| X.02 | Amisulbrom | 3:1 | 0.6:0.2 |
| X.02 | Cyazofamid | 1:1 | 6:6 |
| X.02 | Cyazofamid | 30:1 | 6:0.2 |
| X.02 | Cyazofamid | 1:10 | 0.6:6 |
| X.02 | Cyazofamid | 3:1 | 0.6:0.2 |
| X.02 | Azoxystrobin | 1:1 | 6:6 |
| X.02 | Azoxystrobin | 10:1 | 6:0.6 |
| X.02 | Azoxystrobin | 1:10 | 0.6:6 |
| X.02 | Azoxystrobin | 1:1 | 0.6:0.6 |
| X.02 | Sedaxane | 1:1 | 6:6 |
| X.02 | Sedaxane | 10:1 | 6:0.6 |
| X.02 | Sedaxane | 1:10 | 0.6:6 |
| X.02 | Sedaxane | 1:1 | 0.6:0.6 |
| X.02 | Pydiflumetofen | 1:1 | 6:6 |
| X.02 | Pydiflumetofen | 10:1 | 6:0.6 |
| X.02 | Pydiflumetofen | 1:10 | 0.6:6 |
| X.02 | Pydiflumetofen | 1:1 | 0.6:0.6 |
| X.02 | Metalaxyl-M | 1:1 | 6:6 |
| X.02 | Metalaxyl-M | 30:1 | 6:0.2 |
| X.02 | Metalaxyl-M | 1:10 | 0.6:6 |
| X.02 | Metalaxyl-M | 3:1 | 0.6:0.2 |
| X.02 | Folpet | 1:1 | 6:6 |
| X.02 | Folpet | 10:1 | 6:0.6 |
| X.02 | Folpet | 1:10 | 0.6:6 |
| X.02 | Folpet | 1:1 | 0.6:0.6 |
| X.02 | Chlorothalonil | 1:1 | 6:6 |
| X.02 | Chlorothalonil | 10:1 | 6:0.6 |
| X.02 | Chlorothalonil | 1:10 | 0.6:6 |
| X.02 | Chlorothalonil | 1:1 | 0.6:0.6 |
| X.02 | Oxathiapiprolin | 1:1 | 6:6 |
| X.02 | Oxathiapiprolin | 30:1 | 6:0.2 |
| X.02 | Oxathiapiprolin | 1:10 | 0.6:6 |
| X.02 | Oxathiapiprolin | 3:1 | 0.6:0.2 |
| X.02 | Fluoxapiprolin | 1:1 | 6:6 |
| X.02 | Fluoxapiprolin | 30:1 | 6:0.2 |
| X.02 | Fluoxapiprolin | 1:10 | 0.6:6 |
| X.02 | Fluoxapiprolin | 3:1 | 0.6:0.2 |
| X.02 | Acibenzolar-S-methyl | 1:1 | 6:6 |
| X.02 | Acibenzolar-S-methyl | 10:1 | 6:0.6 |
| X.02 | Acibenzolar-S-methyl | 1:10 | 0.6:6 |
| X.02 | Acibenzolar-S-methyl | 1:1 | 0.6:0.6 |
| X.02 | Fluopicolide | 1:1 | 6:6 |
| X.02 | Fluopicolide | 30:1 | 6:0.2 |
| X.02 | Fluopicolide | 1:10 | 0.6:6 |
| X.02 | Fluopicolide | 3:1 | 0.6:0.2 |
| X.02 | Zoxamide | 1:1 | 6:6 |
| X.02 | Zoxamide | 30:1 | 6:0.2 |
| X.02 | Zoxamide | 1:10 | 0.6:6 |
| X.02 | Zoxamide | 3:1 | 0.6:0.2 |
| X.02 | Mandipropamid | 1:1 | 6:6 |
| X.02 | Mandipropamid | 30:1 | 6:0.2 |
| X.02 | Mandipropamid | 1:10 | 0.6:6 |
| X.02 | Mandipropamid | 3:1 | 0.6:0.2 |
| X.02 | Cyclobutrifluram | 1:1 | 6:6 |
| X.02 | Cyclobutrifluram | 10:1 | 6:0.6 |
| X.02 | Cyclobutrifluram | 1:10 | 0.6:6 |
| X.02 | Cyclobutrifluram | 1:1 | 0.6:0.6 |
| X.03 | Picarbutrazox | 1:1 | 6:6 |
| X.03 | Picarbutrazox | 30:1 | 6:0.2 |
| X.03 | Picarbutrazox | 1:10 | 0.6:6 |
| X.03 | Picarbutrazox | 3:1 | 0.6:0.2 |
| X.03 | Cymoxanil | 1:1 | 6:6 |
| X.03 | Cymoxanil | 10:1 | 6:0.6 |
| X.03 | Cymoxanil | 1:10 | 0.6:6 |
| X.03 | Cymoxanil | 1:1 | 0.6:0.6 |
| X.03 | Disodium phosphonate | 1:1 | 6:6 |
| X.03 | Disodium phosphonate | 10:1 | 6:0.6 |
| X.03 | Disodium phosphonate | 1:10 | 0.6:6 |
| X.03 | Disodium phosphonate | 1:1 | 0.6:0.6 |
| X.03 | Difenoconazole | 1:1 | 6:6 |
| X.03 | Difenoconazole | 10:1 | 6:0.6 |
| X.03 | Difenoconazole | 1:10 | 0.6:6 |
| X.03 | Difenoconazole | 1:1 | 0.6:0.6 |
| X.03 | Fludioxonil | 1:1 | 6:6 |
| X.03 | Fludioxonil | 10:1 | 6:0.6 |
| X.03 | Fludioxonil | 1:10 | 0.6:6 |
| X.03 | Fludioxonil | 1:1 | 0.6:0.6 |
| X.03 | Fluazinam | 1:1 | 6:6 |
| X.03 | Fluazinam | 30:1 | 6:0.2 |
| X.03 | Fluazinam | 1:10 | 0.6:6 |
| X.03 | Fluazinam | 3:1 | 0.6:0.2 |
| X.03 | Ametoctradin | 1:1 | 6:6 |
| X.03 | Ametoctradin | 30:1 | 6:0.2 |
| X.03 | Ametoctradin | 1:10 | 0.6:6 |
| X.03 | Ametoctradin | 3:1 | 0.6:0.2 |
| X.03 | Amisulbrom | 1:1 | 6:6 |
| X.03 | Amisulbrom | 30:1 | 6:0.2 |
| X.03 | Amisulbrom | 1:10 | 0.6:6 |
| X.03 | Amisulbrom | 3:1 | 0.6:0.2 |
| X.03 | Cyazofamid | 1:1 | 6:6 |
| X.03 | Cyazofamid | 30:1 | 6:0.2 |
| X.03 | Cyazofamid | 1:10 | 0.6:6 |
| X.03 | Cyazofamid | 3:1 | 0.6:0.2 |
| X.03 | Azoxystrobin | 1:1 | 6:6 |
| X.03 | Azoxystrobin | 10:1 | 6:0.6 |
| X.03 | Azoxystrobin | 1:10 | 0.6:6 |
| X.03 | Azoxystrobin | 1:1 | 0.6:0.6 |
| X.03 | Sedaxane | 1:1 | 6:6 |
| X.03 | Sedaxane | 10:1 | 6:0.6 |
| X.03 | Sedaxane | 1:10 | 0.6:6 |
| X.03 | Sedaxane | 1:1 | 0.6:0.6 |
| X.03 | Pydiflumetofen | 1:1 | 6:6 |
| X.03 | Pydiflumetofen | 10:1 | 6:0.6 |
| X.03 | Pydiflumetofen | 1:10 | 0.6:6 |
| X.03 | Pydiflumetofen | 1:1 | 0.6:0.6 |
| X.03 | Metalaxyl-M | 1:1 | 6:6 |
| X.03 | Metalaxyl-M | 30:1 | 6:0.2 |
| X.03 | Metalaxyl-M | 1:10 | 0.6:6 |
| X.03 | Metalaxyl-M | 3:1 | 0.6:0.2 |
| X.03 | Folpet | 1:1 | 6:6 |
| X.03 | Folpet | 10:1 | 6:0.6 |
| X.03 | Folpet | 1:10 | 0.6:6 |
| X.03 | Folpet | 1:1 | 0.6:0.6 |
| X.03 | Chlorothalonil | 1:1 | 6:6 |
| X.03 | Chlorothalonil | 10:1 | 6:0.6 |
| X.03 | Chlorothalonil | 1:10 | 0.6:6 |
| X.03 | Chlorothalonil | 1:1 | 0.6:0.6 |
| X.03 | Oxathiapiprolin | 1:1 | 6:6 |
| X.03 | Oxathiapiprolin | 30:1 | 6:0.2 |
| X.03 | Oxathiapiprolin | 1:10 | 0.6:6 |
| X.03 | Oxathiapiprolin | 3:1 | 0.6:0.2 |
| X.03 | Fluoxapiprolin | 1:1 | 6:6 |
| X.03 | Fluoxapiprolin | 30:1 | 6:0.2 |
| X.03 | Fluoxapiprolin | 1:10 | 0.6:6 |
| X.03 | Fluoxapiprolin | 3:1 | 0.6:0.2 |
| X.03 | Acibenzolar-S-methyl | 1:1 | 6:6 |
| X.03 | Acibenzolar-S-methyl | 10:1 | 6:0.6 |
| X.03 | Acibenzolar-S-methyl | 1:10 | 0.6:6 |
| X.03 | Acibenzolar-S-methyl | 1:1 | 0.6:0.6 |
| X.03 | Fluopicolide | 1:1 | 6:6 |
| X.03 | Fluopicolide | 30:1 | 6:0.2 |
| X.03 | Fluopicolide | 1:10 | 0.6:6 |
| X.03 | Fluopicolide | 3:1 | 0.6:0.2 |
| X.03 | Zoxamide | 1:1 | 6:6 |
| X.03 | Zoxamide | 30:1 | 6:0.2 |
| X.03 | Zoxamide | 1:10 | 0.6:6 |
| X.03 | Zoxamide | 3:1 | 0.6:0.2 |
| X.03 | Mandipropamid | 1:1 | 6:6 |
| X.03 | Mandipropamid | 30:1 | 6:0.2 |
| X.03 | Mandipropamid | 1:10 | 0.6:6 |
| X.03 | Mandipropamid | 3:1 | 0.6:0.2 |
| X.03 | Cyclobutrifluram | 1:1 | 6:6 |
| X.03 | Cyclobutrifluram | 10:1 | 6:0.6 |
| X.03 | Cyclobutrifluram | 1:10 | 0.6:6 |
| X.03 | Cyclobutrifluram | 1:1 | 0.6:0.6 |
| X.04 | Picarbutrazox | 1:1 | 6:6 |
| X.04 | Picarbutrazox | 30:1 | 6:0.2 |
| X.04 | Picarbutrazox | 1:10 | 0.6:6 |
| X.04 | Picarbutrazox | 3:1 | 0.6:0.2 |
| X.04 | Cymoxanil | 1:1 | 6:6 |
| X.04 | Cymoxanil | 10:1 | 6:0.6 |
| X.04 | Cymoxanil | 1:10 | 0.6:6 |
| X.04 | Cymoxanil | 1:1 | 0.6:0.6 |
| X.04 | Disodium phosphonate | 1:1 | 6:6 |
| X.04 | Disodium phosphonate | 10:1 | 6:0.6 |
| X.04 | Disodium phosphonate | 1:10 | 0.6:6 |
| X.04 | Disodium phosphonate | 1:1 | 0.6:0.6 |
| X.04 | Difenoconazole | 1:1 | 6:6 |
| X.04 | Difenoconazole | 10:1 | 6:0.6 |
| X.04 | Difenoconazole | 1:10 | 0.6:6 |
| X.04 | Difenoconazole | 1:1 | 0.6:0.6 |
| X.04 | Fludioxonil | 1:1 | 6:6 |
| X.04 | Fludioxonil | 10:1 | 6:0.6 |
| X.04 | Fludioxonil | 1:10 | 0.6:6 |
| X.04 | Fludioxonil | 1:1 | 0.6:0.6 |
| X.04 | Fluazinam | 1:1 | 6:6 |
| X.04 | Fluazinam | 30:1 | 6:0.2 |
| X.04 | Fluazinam | 1:10 | 0.6:6 |
| X.04 | Fluazinam | 3:1 | 0.6:0.2 |
| X.04 | Ametoctradin | 1:1 | 6:6 |
| X.04 | Ametoctradin | 30:1 | 6:0.2 |
| X.04 | Ametoctradin | 1:10 | 0.6:6 |
| X.04 | Ametoctradin | 3:1 | 0.6:0.2 |
| X.04 | Amisulbrom | 1:1 | 6:6 |
| X.04 | Amisulbrom | 30:1 | 6:0.2 |
| X.04 | Amisulbrom | 1:10 | 0.6:6 |
| X.04 | Amisulbrom | 3:1 | 0.6:0.2 |
| X.04 | Cyazofamid | 1:1 | 6:6 |
| X.04 | Cyazofamid | 30:1 | 6:0.2 |
| X.04 | Cyazofamid | 1:10 | 0.6:6 |
| X.04 | Cyazofamid | 3:1 | 0.6:0.2 |
| X.04 | Azoxystrobin | 1:1 | 6:6 |
| X.04 | Azoxystrobin | 10:1 | 6:0.6 |
| X.04 | Azoxystrobin | 1:10 | 0.6:6 |
| X.04 | Azoxystrobin | 1:1 | 0.6:0.6 |
| X.04 | Sedaxane | 1:1 | 6:6 |
| X.04 | Sedaxane | 10:1 | 6:0.6 |
| X.04 | Sedaxane | 1:10 | 0.6:6 |
| X.04 | Sedaxane | 1:1 | 0.6:0.6 |
| X.04 | Pydiflumetofen | 1:1 | 6:6 |
| X.04 | Pydiflumetofen | 10:1 | 6:0.6 |
| X.04 | Pydiflumetofen | 1:10 | 0.6:6 |
| X.04 | Pydiflumetofen | 1:1 | 0.6:0.6 |
| X.04 | Metalaxyl-M | 1:1 | 6:6 |
| X.04 | Metalaxyl-M | 30:1 | 6:0.2 |
| X.04 | Metalaxyl-M | 1:10 | 0.6:6 |
| X.04 | Metalaxyl-M | 3:1 | 0.6:0.2 |
| X.04 | Folpet | 1:1 | 6:6 |
| X.04 | Folpet | 10:1 | 6:0.6 |
| X.04 | Folpet | 1:10 | 0.6:6 |
| X.04 | Folpet | 1:1 | 0.6:0.6 |
| X.04 | Chlorothalonil | 1:1 | 6:6 |
| X.04 | Chlorothalonil | 10:1 | 6:0.6 |
| X.04 | Chlorothalonil | 1:10 | 0.6:6 |
| X.04 | Chlorothalonil | 1:1 | 0.6:0.6 |
| X.04 | Oxathiapiprolin | 1:1 | 6:6 |
| X.04 | Oxathiapiprolin | 30:1 | 6:0.2 |
| X.04 | Oxathiapiprolin | 1:10 | 0.6:6 |
| X.04 | Oxathiapiprolin | 3:1 | 0.6:0.2 |
| X.04 | Fluoxapiprolin | 1:1 | 6:6 |
| X.04 | Fluoxapiprolin | 30:1 | 6:0.2 |
| X.04 | Fluoxapiprolin | 1:10 | 0.6:6 |
| X.04 | Fluoxapiprolin | 3:1 | 0.6:0.2 |
| X.04 | Acibenzolar-S-methyl | 1:1 | 6:6 |
| X.04 | Acibenzolar-S-methyl | 10:1 | 6:0.6 |
| X.04 | Acibenzolar-S-methyl | 1:10 | 0.6:6 |
| X.04 | Acibenzolar-S-methyl | 1:1 | 0.6:0.6 |
| X.04 | Fluopicolide | 1:1 | 6:6 |
| X.04 | Fluopicolide | 30:1 | 6:0.2 |
| X.04 | Fluopicolide | 1:10 | 0.6:6 |
| X.04 | Fluopicolide | 3:1 | 0.6:0.2 |
| X.04 | Zoxamide | 1:1 | 6:6 |
| X.04 | Zoxamide | 30:1 | 6:0.2 |
| X.04 | Zoxamide | 1:10 | 0.6:6 |
| X.04 | Zoxamide | 3:1 | 0.6:0.2 |
| X.04 | Mandipropamid | 1:1 | 6:6 |
| X.04 | Mandipropamid | 30:1 | 6:0.2 |
| X.04 | Mandipropamid | 1:10 | 0.6:6 |
| X.04 | Mandipropamid | 3:1 | 0.6:0.2 |
| X.04 | Cyclobutrifluram | 1:1 | 6:6 |
| X.04 | Cyclobutrifluram | 10:1 | 6:0.6 |
| X.04 | Cyclobutrifluram | 1:10 | 0.6:6 |
| X.04 | Cyclobutrifluram | 1:1 | 0.6:0.6 |
| X.05 | Picarbutrazox | 1:1 | 6:6 |
| X.05 | Picarbutrazox | 30:1 | 6:0.2 |
| X.05 | Picarbutrazox | 1:10 | 0.6:6 |
| X.05 | Picarbutrazox | 3:1 | 0.6:0.2 |
| X.05 | Cymoxanil | 1:1 | 6:6 |
| X.05 | Cymoxanil | 10:1 | 6:0.6 |
| X.05 | Cymoxanil | 1:10 | 0.6:6 |
| X.05 | Cymoxanil | 1:1 | 0.6:0.6 |
| X.05 | Disodium phosphonate | 1:1 | 6:6 |
| X.05 | Disodium phosphonate | 10:1 | 6:0.6 |
| X.05 | Disodium phosphonate | 1:10 | 0.6:6 |
| X.05 | Disodium phosphonate | 1:1 | 0.6:0.6 |
| X.05 | Difenoconazole | 1:1 | 6:6 |
| X.05 | Difenoconazole | 10:1 | 6:0.6 |
| X.05 | Difenoconazole | 1:10 | 0.6:6 |
| X.05 | Difenoconazole | 1:1 | 0.6:0.6 |
| X.05 | Fludioxonil | 1:1 | 6:6 |
| X.05 | Fludioxonil | 10:1 | 6:0.6 |
| X.05 | Fludioxonil | 1:10 | 0.6:6 |
| X.05 | Fludioxonil | 1:1 | 0.6:0.6 |
| X.05 | Fluazinam | 1:1 | 6:6 |
| X.05 | Fluazinam | 30:1 | 6:0.2 |
| X.05 | Fluazinam | 1:10 | 0.6:6 |
| X.05 | Fluazinam | 3:1 | 0.6:0.2 |
| X.05 | Ametoctradin | 1:1 | 6:6 |
| X.05 | Ametoctradin | 30:1 | 6:0.2 |
| X.05 | Ametoctradin | 1:10 | 0.6:6 |
| X.05 | Ametoctradin | 3:1 | 0.6:0.2 |
| X.05 | Amisulbrom | 1:1 | 6:6 |
| X.05 | Amisulbrom | 30:1 | 6:0.2 |
| X.05 | Amisulbrom | 1:10 | 0.6:6 |
| X.05 | Amisulbrom | 3:1 | 0.6:0.2 |
| X.05 | Cyazofamid | 1:1 | 6:6 |
| X.05 | Cyazofamid | 30:1 | 6:0.2 |
| X.05 | Cyazofamid | 1:10 | 0.6:6 |
| X.05 | Cyazofamid | 3:1 | 0.6:0.2 |
| X.05 | Azoxystrobin | 1:1 | 6:6 |
| X.05 | Azoxystrobin | 10:1 | 6:0.6 |
| X.05 | Azoxystrobin | 1:10 | 0.6:6 |
| X.05 | Azoxystrobin | 1:1 | 0.6:0.6 |
| X.05 | Sedaxane | 1:1 | 6:6 |
| X.05 | Sedaxane | 10: | 6:0.6 |
| X.05 | Sedaxane | 1:10 | 0.6:6 |
| X.05 | Sedaxane | 1:1 | 0.6:0.6 |
| X.05 | Pydiflumetofen | 1:1 | 6:6 |
| X.05 | Pydiflumetofen | 10:1 | 6:0.6 |
| X.05 | Pydiflumetofen | 1:10 | 0.6:6 |
| X.05 | Pydiflumetofen | 1:1 | 0.6:0.6 |
| X.05 | Metalaxyl-M | 1:1 | 6:6 |
| X.05 | Metalaxyl-M | 30:1 | 6:0.2 |
| X.05 | Metalaxyl-M | 1:10 | 0.6:6 |
| X.05 | Metalaxyl-M | 3:1 | 0.6:0.2 |
| X.05 | Folpet | 1:1 | 6:6 |
| X.05 | Folpet | 10:1 | 6:0.6 |
| X.05 | Folpet | 1:10 | 0.6:6 |
| X.05 | Folpet | 1:1 | 0.6:0.6 |
| X.05 | Chlorothalonil | 1:1 | 6:6 |
| X.05 | Chlorothalonil | 10:1 | 6:0.6 |
| X.05 | Chlorothalonil | 1:10 | 0.6:6 |
| X.05 | Chlorothalonil | 1:1 | 0.6:0.6 |
| X.05 | Oxathiapiprolin | 1:1 | 6:6 |
| X.05 | Oxathiapiprolin | 30:1 | 6:0.2 |
| X.05 | Oxathiapiprolin | 1:10 | 0.6:6 |
| X.05 | Oxathiapiprolin | 3:1 | 0.6:0.2 |
| X.05 | Fluoxapiprolin | 1:1 | 6:6 |
| X.05 | Fluoxapiprolin | 30:1 | 6:0.2 |
| X.05 | Fluoxapiprolin | 1:10 | 0.6:6 |
| X.05 | Fluoxapiprolin | 3:1 | 0.6:0.2 |
| X.05 | Acibenzolar-S-methyl | 1:1 | 6:6 |
| X.05 | Acibenzolar-S-methyl | 10:1 | 6:0.6 |
| X.05 | Acibenzolar-S-methyl | 1:10 | 0.6:6 |
| X.05 | Acibenzolar-S-methyl | 1:1 | 0.6:0.6 |
| X.05 | Fluopicolide | 1:1 | 6:6 |
| X.05 | Fluopicolide | 30:1 | 6:0.2 |
| X.05 | Fluopicolide | 1:10 | 0.6:6 |
| X.05 | Fluopicolide | 3:1 | 0.6:0.2 |
| X.05 | Zoxamide | 1:1 | 6:6 |
| X.05 | Zoxamide | 30:1 | 6:0.2 |
| X.05 | Zoxamide | 1:10 | 0.6:6 |
| X.05 | Zoxamide | 3:1 | 0.6:0.2 |
| X.05 | Mandipropamid | 1:1 | 6:6 |
| K.05 | Mandipropamid | 30:1 | 6:0.2 |
| X.05 | Mandipropamid | 1:10 | 0.6:6 |
| X.05 | Mandipropamid | 3:1 | 0.6:0.2 |
| X.05 | Cyclobutrifluram | 1:1 | 6:6 |
| X.05 | Cyclobutrifluram | 10:1 | 6:0.6 |
| X.05 | Cyclobutrifluram | 1:10 | 0.6:6 |
| X.05 | Cyclobutrifluram | 1:1 | 0.6:0.6 |
| X.06 | Picarbutrazox | 1:1 | 6:6 |
| X.06 | Picarbutrazox | 30:1 | 6:0.2 |
| X.06 | Picarbutrazox | 1:10 | 0.6:6 |
| X.06 | Picarbutrazox | 3:1 | 0.6:0.2 |
| X.06 | Cymoxanil | 1:1 | 6:6 |
| X.06 | Cymoxanil | 10:1 | 6:0.6 |
| X.06 | Cymoxanil | 1:10 | 0.6:6 |
| X.06 | Cymoxanil | 1:1 | 0.6:0.6 |
| X.06 | Disodium phosphonate | 1:1 | 6:6 |
| X.06 | Disodium phosphonate | 10:1 | 6:0.6 |
| X.06 | Disodium phosphonate | 1:10 | 0.6:6 |
| X.06 | Disodium phosphonate | 1:1 | 0.6:0.6 |
| X.06 | Difenoconazole | 1:1 | 6:6 |
| X.06 | Difenoconazole | 10:1 | 6:0.6 |
| X.06 | Difenoconazole | 1:10 | 0.6:6 |
| X.06 | Difenoconazole | 1:1 | 0.6:0.6 |
| X.06 | Fludioxonil | 1:1 | 6:6 |
| X.06 | Fludioxonil | 10:1 | 6:0.6 |
| X.06 | Fludioxonil | 1:10 | 0.6:6 |
| X.06 | Fludioxonil | 1:1 | 0.6:0.6 |
| X.06 | Fluazinam | 1:1 | 6:6 |
| X.06 | Fluazinam | 30:1 | 6:0.2 |
| X.06 | Fluazinam | 1:10 | 0.6:6 |
| X.06 | Fluazinam | 3:1 | 0.6:0.2 |
| X.06 | Ametoctradin | 1:1 | 6:6 |
| X.06 | Ametoctradin | 30:1 | 6:0.2 |
| X.06 | Ametoctradin | 1:10 | 0.6:6 |
| X.06 | Ametoctradin | 3:1 | 0.6:0.2 |
| X.06 | Amisulbrom | 1:1 | 6:6 |
| X.06 | Amisulbrom | 30:1 | 6:0.2 |
| X.06 | Amisulbrom | 1:10 | 0.6:6 |
| X.06 | Amisulbrom | 3:1 | 0.6:0.2 |
| X.06 | Cyazofamid | 1:1 | 6:6 |
| X.06 | Cyazofamid | 30:1 | 6:0.2 |
| X.06 | Cyazofamid | 1:10 | 0.6:6 |
| X.06 | Cyazofamid | 3:1 | 0.6:0.2 |
| X.06 | Azoxystrobin | 1:1 | 6:6 |
| X.06 | Azoxystrobin | 10:1 | 6:0.6 |
| X.06 | Azoxystrobin | 1:10 | 0.6:6 |
| X.06 | Azoxystrobin | 1:1 | 0.6:0.6 |
| X.06 | Sedaxane | 1:1 | 6:6 |
| X.06 | Sedaxane | 10:1 | 6:0.6 |
| X.06 | Sedaxane | 1:10 | 0.6:6 |
| X.06 | Sedaxane | 1:1 | 0.6:0.6 |
| X.06 | Pydiflumetofen | 1:1 | 6:6 |
| X.06 | Pydiflumetofen | 10:1 | 6:0.6 |
| X.06 | Pydiflumetofen | 1:10 | 0.6:6 |
| X.06 | Pydiflumetofen | 1:1 | 0.6:0.6 |
| X.06 | Metalaxyl-M | 1:1 | 6:6 |
| X.06 | Metalaxyl-M | 30:1 | 6:0.2 |
| X.06 | Metalaxyl-M | 1:10 | 0.6:6 |
| X.06 | Folpet | 1:1 | 6:6 |
| X.06 | Folpet | 10:1 | 6:0.6 |
| X.06 | Folpet | 1:10 | 0.6:6 |
| X.06 | Folpet | 1:1 | 0.6:0.6 |
| X.06 | Chlorothalonil | 1:1 | 6:6 |
| X.06 | Chlorothalonil | 10:1 | 6:0.6 |
| X.06 | Chlorothalonil | 1:1 | 0.6:0.6 |
| X.06 | Oxathiapiprolin | 1:1 | 6:6 |
| X.06 | Oxathiapiprolin | 30:1 | 6:0.2 |
| X.06 | Oxathiapiprolin | 1:10 | 0.6:6 |
| X.06 | Oxathiapiprolin | 3:1 | 0.6:0.2 |
| X.06 | Fluoxapiprolin | 1:1 | 6:6 |
| X.06 | Fluoxapiprolin | 30:1 | 6:0.2 |
| X.06 | Fluoxapiprolin | 1:10 | 0.6:6 |
| X.06 | Fluoxapiprolin | 3:1 | 0.6:0.2 |
| X.06 | Acibenzolar-S-methyl | 1:1 | 6:6 |
| X.06 | Acibenzolar-S-methyl | 10:1 | 6:0.6 |
| X.06 | Acibenzolar-S-methyl | 1:10 | 0.6:6 |
| X.06 | Acibenzolar-S-methyl | 1:1 | 0.6:0.6 |
| X.06 | Fluopicolide | 1:1 | 6:6 |
| X.06 | Fluopicolide | 30:1 | 6:0.2 |
| X.06 | Fluopicolide | 1:10 | 0.6:6 |
| X.06 | Fluopicolide | 3:1 | 0.6:0.2 |
| X.06 | Zoxamide | 1:1 | 6:6 |
| X.06 | Zoxamide | 30:1 | 6:0.2 |
| X.06 | Mandipropamid | 1:1 | 6:6 |
| X.06 | Mandipropamid | 30:1 | 6:0.2 |
| X.06 | Mandipropamid | 1:10 | 0.6:6 |
| X.06 | Mandipropamid | 3:1 | 0.6:0.2 |
| X.06 | Cyclobutrifluram | 1:1 | 6:6 |
| X.06 | Cyclobutrifluram | 10:1 | 6:0.6 |
| X.06 | Cyclobutrifluram | 1:10 | 0.6:6 |
| X.06 | Cyclobutrifluram | 1:1 | 0.6:0.6 |

### Example 13: Activity Against Phytophthora infestans (Late Blight of Potato/Tomato)

Sporangia of the pathogen from cryogenic storage were directly mixed into nutrient broth (pea medium). After placing a (DMSO) solution of the test compounds into a microtiter plate (96-well format) the nutrient broth containing the fungal spores was added. The test plates were incubated at 24 00 and the inhibition of growth was determined photometrically after 5 days. The following mixture compositions (A:B) at the reported concentration (in ppm) gave at least 80% disease control in this test.

| | | | Conc. |
|---|---|---|---|
| Component A | | Ratio | (ppm) |
| (Compound) | Component B | A:B | (A:B) |
| X.01 | Picarbutrazox | 1:1 | 6:6 |
| X.01 | Picarbutrazox | 30:1 | 6:0.2 |
| X.01 | Picarbutrazox | 1:10 | 0.6:6 |
| X.01 | Picarbutrazox | 3:1 | 0.6:0.2 |
| X.01 | Cymoxanil | 1:1 | 6:6 |
| X.01 | Cymoxanil | 10:1 | 6:0.6 |
| X.01 | Cymoxanil | 1:10 | 0.6:6 |
| X.01 | Cymoxanil | 1:1 | 0.6:0.6 |
| X.01 | Disodium phosphonate | 1:1 | 6:6 |
| X.01 | Disodium phosphonate | 10:1 | 6:0.6 |
| X.01 | Disodium phosphonate | 1:10 | 0.6:6 |
| X.01 | Disodium phosphonate | 1:1 | 0.6:0.6 |
| X.01 | Difenoconazole | 1:1 | 6:6 |
| X.01 | Difenoconazole | 10:1 | 6:0.6 |
| X.01 | Difenoconazole | 1:10 | 0.6:6 |
| X.01 | Difenoconazole | 1:1 | 0.6:0.6 |
| X.01 | Fludioxonil | 1:1 | 6:6 |
| X.01 | Fludioxonil | 10:1 | 6:0.6 |
| X.01 | Fludioxonil | 1:10 | 0.6:6 |
| X.01 | Fludioxonil | 1:1 | 0.6:0.6 |
| X.01 | Fluazinam | 1:1 | 6:6 |
| X.01 | Fluazinam | 30:1 | 6:0.2 |
| X.01 | Fluazinam | 1:10 | 0.6:6 |
| X.01 | Fluazinam | 3:1 | 0.6:0.2 |
| X.01 | Ametoctradin | 1:1 | 6:6 |
| X.01 | Ametoctradin | 30:1 | 6:0.2 |
| X.01 | Ametoctradin | 1:10 | 0.6:6 |
| X.01 | Ametoctradin | 3:1 | 0.6:0.2 |
| X.01 | Amisulbrom | 1:1 | 6:6 |
| X.01 | Amisulbrom | 30:1 | 6:0.2 |
| X.01 | Amisulbrom | 1:10 | 0.6:6 |
| X.01 | Amisulbrom | 3:1 | 0.6:0.2 |
| X.01 | Cyazofamid | 1:1 | 6:6 |
| X.01 | Cyazofamid | 30:1 | 6:0.2 |
| X.01 | Cyazofamid | 1:10 | 0.6:6 |
| X.01 | Cyazofamid | 3:1 | 0.6:0.2 |
| X.01 | Azoxystrobin | 1:1 | 6:6 |
| X.01 | Azoxystrobin | 10:1 | 6:0.6 |
| X.01 | Azoxystrobin | 1:10 | 0.6:6 |
| X.01 | Azoxystrobin | 1:1 | 0.6:0.6 |
| X.01 | Sedaxane | 1:1 | 6:6 |
| X.01 | Sedaxane | 10:1 | 6:0.6 |
| X.01 | Sedaxane | 1:10 | 0.6:6 |
| X.01 | Sedaxane | 1:1 | 0.6:0.6 |
| X.01 | Pydiflumetofen | 1:1 | 6:6 |
| X.01 | Pydiflumetofen | 10:1 | 6:0.6 |
| X.01 | Pydiflumetofen | 1:10 | 0.6:6 |
| X.01 | Pydiflumetofen | 1:1 | 0.6:0.6 |
| X.01 | Metalaxyl-M | 1:1 | 6:6 |
| X.01 | Metalaxyl-M | 30:1 | 6:0.2 |
| X.01 | Metalaxyl-M | 1:10 | 0.6:6 |
| X.01 | Metalaxyl-M | 3:1 | 0.6:0.2 |
| X.01 | Folpet | 1:1 | 6:6 |
| X.01 | Folpet | 10:1 | 6:0.6 |
| X.01 | Folpet | 1:10 | 0.6:6 |
| X.01 | Folpet | 1:1 | 0.6:0.6 |
| X.01 | Chlorothalonil | 1:1 | 6:6 |
| X.01 | Chlorothalonil | 10:1 | 6:0.6 |
| X.01 | Chlorothalonil | 1:10 | 0.6:6 |
| X.01 | Chlorothalonil | 1:1 | 0.6:0.6 |
| X.01 | Oxathiapiprolin | 1:1 | 6:6 |
| X.01 | Oxathiapiprolin | 30:1 | 6:0.2 |
| X.01 | Oxathiapiprolin | 1:10 | 0.6:6 |
| X.01 | Oxathiapiprolin | 3:1 | 0.6:0.2 |
| X.01 | Fluoxapiprolin | 1:1 | 6:6 |
| X.01 | Fluoxapiprolin | 30:1 | 6:0.2 |
| X.01 | Fluoxapiprolin | 1:10 | 0.6:6 |
| X.01 | Fluoxapiprolin | 3:1 | 0.6:0.2 |
| X.01 | Acibenzolar-S-methyl | 1:1 | 6:6 |
| X.01 | Acibenzolar-S-methyl | 10:1 | 6:0.6 |
| X.01 | Acibenzolar-S-methyl | 1:10 | 0.6:6 |
| X.01 | Acibenzolar-S-methyl | 1:1 | 0.6:0.6 |
| X.01 | Fluopicolide | 1:1 | 6:6 |
| X.01 | Fluopicolide | 30:1 | 6:0.2 |
| X.01 | Fluopicolide | 1:10 | 0.6:6 |
| X.01 | Fluopicolide | 3:1 | 0.6:0.2 |
| X.01 | Zoxamide | 1:1 | 6:6 |
| X.01 | Zoxamide | 30:1 | 6:0.2 |
| X.01 | Zoxamide | 1:10 | 0.6:6 |
| X.01 | Zoxamide | 3:1 | 0.6:0.2 |
| X.01 | Mandipropamid | 1:1 | 6:6 |
| X.01 | Mandipropamid | 30:1 | 6:0.2 |
| X.01 | Mandipropamid | 1:10 | 0.6:6 |
| X.01 | Mandipropamid | 3:1 | 0.6:0.2 |
| X.01 | Cyclobutrifluram | 1:1 | 6:6 |
| X.01 | Cyclobutrifluram | 10:1 | 6:0.6 |
| X.01 | Cyclobutrifluram | 1:10 | 0.6:6 |
| X.01 | Cyclobutrifluram | 1:1 | 0.6:0.6 |
| X.02 | Picarbutrazox | 1:1 | 6:6 |
| X.02 | Picarbutrazox | 30:1 | 6:0.2 |
| X.02 | Picarbutrazox | 1:10 | 0.6:6 |
| X.02 | Picarbutrazox | 3:1 | 0.6:0.2 |
| X.02 | Cymoxanil | 1:1 | 6:6 |
| X.02 | Cymoxanil | 10:1 | 6:0.6 |
| X.02 | Cymoxanil | 1:10 | 0.6:6 |
| X.02 | Cymoxanil | 1:1 | 0.6:0.6 |
| X.02 | Disodium phosphonate | 1:1 | 6:6 |
| X.02 | Disodium phosphonate | 10:1 | 6:0.6 |
| X.02 | Disodium phosphonate | 1:10 | 0.6:6 |
| X.02 | Disodium phosphonate | 1:1 | 0.6:0.6 |
| X.02 | Difenoconazole | 1:1 | 6:6 |
| X.02 | Difenoconazole | 10:1 | 6:0.6 |
| X.02 | Difenoconazole | 1:10 | 0.6:6 |
| X.02 | Difenoconazole | 1:1 | 0.6:0.6 |
| X.02 | Fludioxonil | 1:1 | 6:6 |
| X.02 | Fludioxonil | 10:1 | 6:0.6 |
| X.02 | Fludioxonil | 1:10 | 0.6:6 |
| X.02 | Fludioxonil | 1:1 | 0.6:0.6 |
| X.02 | Fluazinam | 1:1 | 6:6 |
| X.02 | Fluazinam | 30:1 | 6:0.2 |
| X.02 | Fluazinam | 1:10 | 0.6:6 |
| X.02 | Fluazinam | 3:1 | 0.6:0.2 |
| X.02 | Ametoctradin | 1:1 | 6:6 |
| X.02 | Ametoctradin | 30:1 | 6:0.2 |
| X.02 | Ametoctradin | 1:10 | 0.6:6 |
| X.02 | Ametoctradin | 3:1 | 0.6:0.2 |
| X.02 | Amisulbrom | 1:1 | 6:6 |
| X.02 | Amisulbrom | 30:1 | 6:0.2 |
| X.02 | Amisulbrom | 1:10 | 0.6:6 |
| X.02 | Amisulbrom | 3:1 | 0.6:0.2 |
| X.02 | Cyazofamid | 1:1 | 6:6 |
| X.02 | Cyazofamid | 30:1 | 6:0.2 |
| X.02 | Cyazofamid | 1:10 | 0.6:6 |
| X.02 | Cyazofamid | 3:1 | 0.6:0.2 |
| X.02 | Azoxystrobin | 1:1 | 6:6 |
| X.02 | Azoxystrobin | 10:1 | 6:0.6 |
| X.02 | Azoxystrobin | 1:10 | 0.6:6 |
| X.02 | Azoxystrobin | 1:1 | 0.6:0.6 |
| X.02 | Sedaxane | 1:1 | 6:6 |
| X.02 | Sedaxane | 10:1 | 6:0.6 |
| X.02 | Sedaxane | 1:10 | 0.6:6 |
| X.02 | Sedaxane | 1:1 | 0.6:0.6 |
| X.02 | Pydiflumetofen | 1:1 | 6:6 |
| X.02 | Pydiflumetofen | 10:1 | 6:0.6 |
| X.02 | Pydiflumetofen | 1:10 | 0.6:6 |
| X.02 | Pydiflumetofen | 1:1 | 0.6:0.6 |
| X.02 | Metalaxyl-M | 1:1 | 6:6 |
| X.02 | Metalaxyl-M | 30:1 | 6:0.2 |
| X.02 | Metalaxyl-M | 1:10 | 0.6:6 |
| X.02 | Metalaxyl-M | 3:1 | 0.6:0.2 |
| X.02 | Folpet | 1:1 | 6:6 |
| X.02 | Folpet | 10:1 | 6:0.6 |
| X.02 | Folpet | 1:10 | 0.6:6 |
| X.02 | Folpet | 1:1 | 0.6:0.6 |
| X.02 | Chlorothalonil | 1:1 | 6:6 |
| X.02 | Chlorothalonil | 10:1 | 6:0.6 |
| X.02 | Chlorothalonil | 1:10 | 0.6:6 |
| X.02 | Chlorothalonil | 1:1 | 0.6:0.6 |
| X.02 | Oxathiapiprolin | 1:1 | 6:6 |
| X.02 | Oxathiapiprolin | 30:1 | 6:0.2 |
| X.02 | Oxathiapiprolin | 1:10 | 0.6:6 |
| X.02 | Oxathiapiprolin | 3:1 | 0.6:0.2 |
| X.02 | Fluoxapiprolin | 1:1 | 6:6 |
| X.02 | Fluoxapiprolin | 30:1 | 6:0.2 |
| X.02 | Fluoxapiprolin | 1:10 | 0.6:6 |
| X.02 | Fluoxapiprolin | 3:1 | 0.6:0.2 |
| X.02 | Acibenzolar-S-methyl | 1:1 | 6:6 |
| X.02 | Acibenzolar-S-methyl | 10:1 | 6:0.6 |
| X.02 | Acibenzolar-S-methyl | 1:10 | 0.6:6 |
| X.02 | Acibenzolar-S-methyl | 1:1 | 0.6:0.6 |
| X.02 | Fluopicolide | 1:1 | 6:6 |
| X.02 | Fluopicolide | 30:1 | 6:0.2 |
| X.02 | Fluopicolide | 1:10 | 0.6:6 |
| X.02 | Fluopicolide | 3:1 | 0.6:0.2 |
| X.02 | Zoxamide | 1:1 | 6:6 |
| X.02 | Zoxamide | 30:1 | 6:0.2 |
| X.02 | Zoxamide | 1:10 | 0.6:6 |
| X.02 | Zoxamide | 3:1 | 0.6:0.2 |
| X.02 | Mandipropamid | 1:1 | 6:6 |
| X.02 | Mandipropamid | 30:1 | 6:0.2 |
| X.02 | Mandipropamid | 1:10 | 0.6:6 |
| X.02 | Mandipropamid | 3:1 | 0.6:0.2 |
| X.02 | Cyclobutrifluram | 1:1 | 6:6 |
| X.02 | Cyclobutrifluram | 10:1 | 6:0.6 |
| X.02 | Cyclobutrifluram | 1:10 | 0.6:6 |
| X.02 | Cyclobutrifluram | 1:1 | 0.6:0.6 |
| X.03 | Picarbutrazox | 1:1 | 6:6 |
| X.03 | Picarbutrazox | 30:1 | 6:0.2 |
| X.03 | Picarbutrazox | 1:10 | 0.6:6 |
| X.03 | Picarbutrazox | 3:1 | 0.6:0.2 |
| X.03 | Cymoxanil | 1:1 | 6:6 |
| X.03 | Cymoxanil | 10:1 | 6:0.6 |
| X.03 | Cymoxanil | 1:10 | 0.6:6 |
| X.03 | Cymoxanil | 1:1 | 0.6:0.6 |
| X.03 | Disodium phosphonate | 1:1 | 6:6 |
| X.03 | Disodium phosphonate | 10:1 | 6:0.6 |
| X.03 | Disodium phosphonate | 1:10 | 0.6:6 |
| X.03 | Disodium phosphonate | 1:1 | 0.6:0.6 |
| X.03 | Difenoconazole | 1:1 | 6:6 |
| X.03 | Difenoconazole | 10:1 | 6:0.6 |
| X.03 | Difenoconazole | 1:10 | 0.6:6 |
| X.03 | Difenoconazole | 1:1 | 0.6:0.6 |
| X.03 | Fludioxonil | 1:1 | 6:6 |
| X.03 | Fludioxonil | 10:1 | 6:0.6 |
| X.03 | Fludioxonil | 1:10 | 0.6:6 |
| X.03 | Fludioxonil | 1:1 | 0.6:0.6 |
| X.03 | Fluazinam | 1:1 | 6:6 |
| X.03 | Fluazinam | 30:1 | 6:0.2 |
| X.03 | Fluazinam | 1:10 | 0.6:6 |
| X.03 | Fluazinam | 3:1 | 0.6:0.2 |
| X.03 | Ametoctradin | 1:1 | 6:6 |
| X.03 | Ametoctradin | 30:1 | 6:0.2 |
| X.03 | Ametoctradin | 1:10 | 0.6:6 |
| X.03 | Ametoctradin | 3:1 | 0.6:0.2 |
| K.03 | Amisulbrom | 1:1 | 6:6 |
| X.03 | Amisulbrom | 30:1 | 6:0.2 |
| X.03 | Amisulbrom | 1:10 | 0.6:6 |
| X.03 | Amisulbrom | 3:1 | 0.6:0.2 |
| X.03 | Cyazofamid | 1:1 | 6:6 |
| X.03 | Cyazofamid | 30:1 | 6:0.2 |
| X.03 | Cyazofamid | 1:10 | 0.6:6 |
| X.03 | Cyazofamid | 3:1 | 0.6:0.2 |
| X.03 | Azoxystrobin | 1:1 | 6:6 |
| X.03 | Azoxystrobin | 10:1 | 6:0.6 |
| X.03 | Azoxystrobin | 1:10 | 0.6:6 |
| X.03 | Azoxystrobin | 1:1 | 0.6:0.6 |
| X.03 | Sedaxane | 1:1 | 6:6 |
| X.03 | Sedaxane | 10:1 | 6:0.6 |
| X.03 | Sedaxane | 1:10 | 0.6:6 |
| X.03 | Sedaxane | 1:1 | 0.6:0.6 |
| X.03 | Pydiflumetofen | 1:1 | 6:6 |
| X.03 | Pydiflumetofen | 10:1 | 6:0.6 |
| X.03 | Pydiflumetofen | 1:10 | 0.6:6 |
| X.03 | Pydiflumetofen | 1:1 | 0.6:0.6 |
| X.03 | Metalaxyl-M | 1:1 | 6:6 |
| X.03 | Metalaxyl-M | 30:1 | 6:0.2 |
| X.03 | Metalaxyl-M | 1:10 | 0.6:6 |
| X.03 | Metalaxyl-M | 3:1 | 0.6:0.2 |
| X.03 | Folpet | 1:1 | 6:6 |
| X.03 | Folpet | 10:1 | 6:0.6 |
| X.03 | Folpet | 1:10 | 0.6:6 |
| X.03 | Folpet | 1:1 | 0.6:0.6 |
| X.03 | Chlorothalonil | 1:1 | 6:6 |
| X.03 | Chlorothalonil | 10:1 | 6:0.6 |
| X.03 | Chlorothalonil | 1:10 | 0.6:6 |
| X.03 | Chlorothalonil | 1:1 | 0.6:0.6 |
| X.03 | Oxathiapiprolin | 1:1 | 6:6 |
| X.03 | Oxathiapiprolin | 30:1 | 6:0.2 |
| X.03 | Oxathiapiprolin | 1:10 | 0.6:6 |
| X.03 | Oxathiapiprolin | 3:1 | 0.6:0.2 |
| X.03 | Fluoxapiprolin | 1:1 | 6:6 |
| X.03 | Fluoxapiprolin | 30:1 | 6:0.2 |
| X.03 | Fluoxapiprolin | 1:10 | 0.6:6 |
| X.03 | Fluoxapiprolin | 3:1 | 0.6:0.2 |
| X.03 | Acibenzolar-S-methyl | 1:1 | 6:6 |
| X.03 | Acibenzolar-S-methyl | 10:1 | 6:0.6 |
| X.03 | Acibenzolar-S-methyl | 1:10 | 0.6:6 |
| X.03 | Acibenzolar-S-methyl | 1:1 | 0.6:0.6 |
| X.03 | Fluopicolide | 1:1 | 6:6 |
| X.03 | Fluopicolide | 30:1 | 6:0.2 |
| X.03 | Fluopicolide | 1:10 | 0.6:6 |
| X.03 | Fluopicolide | 3:1 | 0.6:0.2 |
| X.03 | Zoxamide | 1:1 | 6:6 |
| X.03 | Zoxamide | 30:1 | 6:0.2 |
| X.03 | Zoxamide | 1:10 | 0.6:6 |
| X.03 | Zoxamide | 3:1 | 0.6:0.2 |
| X.03 | Mandipropamid | 1:1 | 6:6 |
| X.03 | Mandipropamid | 30:1 | 6:0.2 |
| X.03 | Mandipropamid | 1:10 | 0.6:6 |
| X.03 | Mandipropamid | 3:1 | 0.6:0.2 |
| X.03 | Cyclobutrifluram | 1:1 | 6:6 |
| X.03 | Cyclobutrifluram | 10:1 | 6:0.6 |
| X.03 | Cyclobutrifluram | 1:10 | 0.6:6 |
| X.03 | Cyclobutrifluram | 1:1 | 0.6:0.6 |
| X.04 | Picarbutrazox | 1:1 | 6:6 |
| X.04 | Picarbutrazox | 30:1 | 6:0.2 |
| X.04 | Picarbutrazox | 1:10 | 0.6:6 |
| X.04 | Picarbutrazox | 3:1 | 0.6:0.2 |
| X.04 | Cymoxanil | 1:1 | 6:6 |
| X.04 | Cymoxanil | 10:1 | 6:0.6 |
| X.04 | Cymoxanil | 1:10 | 0.6:6 |
| X.04 | Cymoxanil | 1:1 | 0.6:0.6 |
| X.04 | Disodium phosphonate | 1:1 | 6:6 |
| X.04 | Disodium phosphonate | 10:1 | 6:0.6 |
| X.04 | Disodium phosphonate | 1:10 | 0.6:6 |
| X.04 | Disodium phosphonate | 1:1 | 0.6:0.6 |
| X.04 | Difenoconazole | 1:1 | 6:6 |
| X.04 | Difenoconazole | 10:1 | 6:0.6 |
| X.04 | Difenoconazole | 1:10 | 0.6:6 |
| X.04 | Difenoconazole | 1:1 | 0.6:0.6 |
| X.04 | Fludioxonil | 1:1 | 6:6 |
| X.04 | Fludioxonil | 10:1 | 6:0.6 |
| X.04 | Fludioxonil | 1:10 | 0.6:6 |
| X.04 | Fludioxonil | 1:1 | 0.6:0.6 |
| X.04 | Fluazinam | 1:1 | 6:6 |
| X.04 | Fluazinam | 30:1 | 6:0.2 |
| X.04 | Fluazinam | 1:10 | 0.6:6 |
| X.04 | Fluazinam | 3:1 | 0.6:0.2 |
| X.04 | Ametoctradin | 1:1 | 6:6 |
| X.04 | Ametoctradin | 30:1 | 6:0.2 |
| X.04 | Ametoctradin | 1:10 | 0.6:6 |
| X.04 | Ametoctradin | 3:1 | 0.6:0.2 |
| X.04 | Amisulbrom | 1:1 | 6:6 |
| X.04 | Amisulbrom | 30:1 | 6:0.2 |
| X.04 | Amisulbrom | 1:10 | 0.6:6 |
| X.04 | Amisulbrom | 3:1 | 0.6:0.2 |
| X.04 | Cyazofamid | 1:1 | 6:6 |
| X.04 | Cyazofamid | 30:1 | 6:0.2 |
| X.04 | Cyazofamid | 1:10 | 0.6:6 |
| X.04 | Cyazofamid | 3:1 | 0.6:0.2 |
| X.04 | Azoxystrobin | 1:1 | 6:6 |
| X.04 | Azoxystrobin | 10:1 | 6:0.6 |
| X.04 | Azoxystrobin | 1:10 | 0.6:6 |
| X.04 | Azoxystrobin | 1:1 | 0.6:0.6 |
| X.04 | Sedaxane | 1:1 | 6:6 |
| X.04 | Sedaxane | 10:1 | 6:0.6 |
| X.04 | Sedaxane | 1:10 | 0.6:6 |
| X.04 | Sedaxane | 1:1 | 0.6:0.6 |
| X.04 | Pydiflumetofen | 1:1 | 6:6 |
| X.04 | Pydiflumetofen | 10:1 | 6:0.6 |
| X.04 | Pydiflumetofen | 1:10 | 0.6:6 |
| X.04 | Pydiflumetofen | 1:1 | 0.6:0.6 |
| X.04 | Metalaxyl-M | 1:1 | 6:6 |
| X.04 | Metalaxyl-M | 30:1 | 6:0.2 |
| X.04 | Metalaxyl-M | 1:10 | 0.6:6 |
| X.04 | Metalaxyl-M | 3:1 | 0.6:0.2 |
| X.04 | Folpet | 1:1 | 6:6 |
| X.04 | Folpet | 10:1 | 6:0.6 |
| X.04 | Folpet | 1:10 | 0.6:6 |
| X.04 | Folpet | 1:1 | 0.6:0.6 |
| X.04 | Chlorothalonil | 1:1 | 6:6 |
| X.04 | Chlorothalonil | 10:1 | 6:0.6 |
| X.04 | Chlorothalonil | 1:10 | 0.6:6 |
| X.04 | Chlorothalonil | 1:1 | 0.6:0.6 |
| X.04 | Oxathiapiprolin | 1:1 | 6:6 |
| X.04 | Oxathiapiprolin | 30:1 | 6:0.2 |
| X.04 | Oxathiapiprolin | 1:10 | 0.6:6 |
| X.04 | Oxathiapiprolin | 3:1 | 0.6:0.2 |
| X.04 | Fluoxapiprolin | 1:1 | 6:6 |
| X.04 | Fluoxapiprolin | 30:1 | 6:0.2 |
| X.04 | Fluoxapiprolin | 1:10 | 0.6:6 |
| X.04 | Fluoxapiprolin | 3:1 | 0.6:0.2 |
| X.04 | Acibenzolar-S-methyl | 1:1 | 6:6 |
| X.04 | Acibenzolar-S-methyl | 10:1 | 6:0.6 |
| X.04 | Acibenzolar-S-methyl | 1:10 | 0.6:6 |
| X.04 | Acibenzolar-S-methyl | 1:1 | 0.6:0.6 |
| X.04 | Fluopicolide | 1:1 | 6:6 |
| X.04 | Fluopicolide | 30:1 | 6:0.2 |
| X.04 | Fluopicolide | 1:10 | 0.6:6 |
| X.04 | Fluopicolide | 3:1 | 0.6:0.2 |
| X.04 | Zoxamide | 1:1 | 6:6 |
| X.04 | Zoxamide | 30:1 | 6:0.2 |
| X.04 | Zoxamide | 1:10 | 0.6:6 |
| X.04 | Zoxamide | 3:1 | 0.6:0.2 |
| X.04 | Mandipropamid | 1:1 | 6:6 |
| X.04 | Mandipropamid | 30:1 | 6:0.2 |
| X.04 | Mandipropamid | 1:10 | 0.6:6 |
| X.04 | Mandipropamid | 3:1 | 0.6:0.2 |
| X.04 | Cyclobutrifluram | 1:1 | 6:6 |
| X.04 | Cyclobutrifluram | 10:1 | 6:0.6 |
| X.04 | Cyclobutrifluram | 1:10 | 0.6:6 |
| X.04 | Cyclobutrifluram | 1:1 | 0.6:0.6 |
| X.05 | Picarbutrazox | 1:1 | 6:6 |
| X.05 | Picarbutrazox | 30:1 | 6:0.2 |
| X.05 | Picarbutrazox | 1:10 | 0.6:6 |
| X.05 | Picarbutrazox | 3:1 | 0.6:0.2 |
| X.05 | Cymoxanil | 1:1 | 6:6 |
| X.05 | Cymoxanil | 10:1 | 6:0.6 |
| X.05 | Cymoxanil | 1:10 | 0.6:6 |
| X.05 | Cymoxanil | 1:1 | 0.6:0.6 |
| X.05 | Disodium phosphonate | 1:1 | 6:6 |
| X.05 | Disodium phosphonate | 10:1 | 6:0.6 |
| X.05 | Disodium phosphonate | 1:10 | 0.6:6 |
| X.05 | Disodium phosphonate | 1:1 | 0.6:0.6 |
| X.05 | Difenoconazole | 1:1 | 6:6 |
| X.05 | Difenoconazole | 10:1 | 6:0.6 |
| X.05 | Difenoconazole | 1:10 | 0.6:6 |
| X.05 | Difenoconazole | 1:1 | 0.6:0.6 |
| X.05 | Fludioxonil | 1:1 | 6:6 |
| X.05 | Fludioxonil | 10:1 | 6:0.6 |
| X.05 | Fludioxonil | 1:10 | 0.6:6 |
| X.05 | Fludioxonil | 1:1 | 0.6:0.6 |
| X.05 | Fluazinam | 1:1 | 6:6 |
| X.05 | Fluazinam | 30:1 | 6:0.2 |
| X.05 | Fluazinam | 1:10 | 0.6:6 |
| X.05 | Fluazinam | 3:1 | 0.6:0.2 |
| X.05 | Ametoctradin | 1:1 | 6:6 |
| X.05 | Ametoctradin | 30:1 | 6:0.2 |
| X.05 | Ametoctradin | 1:10 | 0.6:6 |
| X.05 | Ametoctradin | 3:1 | 0.6:0.2 |
| X.05 | Amisulbrom | 1:1 | 6:6 |
| X.05 | Amisulbrom | 30:1 | 6:0.2 |
| X.05 | Amisulbrom | 1:10 | 0.6:6 |
| X.05 | Amisulbrom | 3:1 | 0.6:0.2 |
| X.05 | Cyazofamid | 1:1 | 6:6 |
| X.05 | Cyazofamid | 30:1 | 6:0.2 |
| X.05 | Cyazofamid | 1:10 | 0.6:6 |
| X.05 | Cyazofamid | 3:1 | 0.6:0.2 |
| X.05 | Azoxystrobin | 1:1 | 6:6 |
| X.05 | Azoxystrobin | 10:1 | 6:0.6 |
| X.05 | Azoxystrobin | 1:10 | 0.6:6 |
| X.05 | Azoxystrobin | 1:1 | 0.6:0.6 |
| X.05 | Sedaxane | 1:1 | 6:6 |
| X.05 | Sedaxane | 10:1 | 6:0.6 |
| X.05 | Sedaxane | 1:10 | 0.6:6 |
| X.05 | Sedaxane | 1:1 | 0.6:0.6 |
| X.05 | Pydiflumetofen | 1:1 | 6:6 |
| X.05 | Pydiflumetofen | 10:1 | 6:0.6 |
| X.05 | Pydiflumetofen | 1:10 | 0.6:6 |
| X.05 | Pydiflumetofen | 1:1 | 0.6:0.6 |
| X.05 | Metalaxyl-M | 1:1 | 6:6 |
| X.05 | Metalaxyl-M | 30:1 | 6:0.2 |
| X.05 | Metalaxyl-M | 1:10 | 0.6:6 |
| X.05 | Metalaxyl-M | 3:1 | 0.6:0.2 |
| X.05 | Folpet | 1:1 | 6:6 |
| X.05 | Folpet | 10:1 | 6:0.6 |
| X.05 | Folpet | 1:10 | 0.6:6 |
| X.05 | Folpet | 1:1 | 0.6:0.6 |
| X.05 | Chlorothalonil | 1:1 | 6:6 |
| X.05 | Chlorothalonil | 10:1 | 6:0.6 |
| X.05 | Chlorothalonil | 1:10 | 0.6:6 |
| X.05 | Chlorothalonil | 1:1 | 0.6:0.6 |
| X.05 | Oxathiapiprolin | 1:1 | 6:6 |
| X.05 | Oxathiapiprolin | 30:1 | 6:0.2 |
| X.05 | Oxathiapiprolin | 1:10 | 0.6:6 |
| X.05 | Oxathiapiprolin | 3:1 | 0.6:0.2 |
| X.05 | Fluoxapiprolin | 1:1 | 6:6 |
| X.05 | Fluoxapiprolin | 30:1 | 6:0.2 |
| X.05 | Fluoxapiprolin | 1:10 | 0.6:6 |
| X.05 | Fluoxapiprolin | 3:1 | 0.6:0.2 |
| X.05 | Acibenzolar-S-methyl | 1:1 | 6:6 |
| X.05 | Acibenzolar-S-methyl | 10:1 | 6:0.6 |
| X.05 | Acibenzolar-S-methyl | 1:10 | 0.6:6 |
| X.05 | Acibenzolar-S-methyl | 1:1 | 0.6:0.6 |
| X.05 | Fluopicolide | 1:1 | 6:6 |
| X.05 | Fluopicolide | 30:1 | 6:0.2 |
| X.05 | Fluopicolide | 1:10 | 0.6:6 |
| X.05 | Fluopicolide | 3:1 | 0.6:0.2 |
| X.05 | Zoxamide | 1:1 | 6:6 |
| X.05 | Zoxamide | 30:1 | 6:0.2 |
| X.05 | Zoxamide | 1:10 | 0.6:6 |
| X.05 | Zoxamide | 3:1 | 0.6:0.2 |
| X.05 | Mandipropamid | 1:1 | 6:6 |
| X.05 | Mandipropamid | 30:1 | 6:0.2 |
| X.05 | Mandipropamid | 1:10 | 0.6:6 |
| X.05 | Mandipropamid | 3:1 | 0.6:0.2 |
| X.05 | Cyclobutrifluram | 1:1 | 6:6 |
| X.05 | Cyclobutrifluram | 10:1 | 6:0.6 |
| X.05 | Cyclobutrifluram | 1:10 | 0.6:6 |
| X.05 | Cyclobutrifluram | 1:1 | 0.6:0.6 |
| X.06 | Picarbutrazox | 1:1 | 6:6 |
| X.06 | Picarbutrazox | 30:1 | 6:0.2 |
| X.06 | Picarbutrazox | 1:10 | 0.6:6 |
| X.06 | Picarbutrazox | 3:1 | 0.6:0.2 |
| X.06 | Cymoxanil | 1:1 | 6:6 |
| X.06 | Cymoxanil | 10:1 | 6:0.6 |
| X.06 | Cymoxanil | 1:10 | 0.6:6 |
| X.06 | Cymoxanil | 1:1 | 0.6:0.6 |
| X.06 | Disodium phosphonate | 1:1 | 6:6 |
| X.06 | Disodium phosphonate | 10:1 | 6:0.6 |
| X.06 | Disodium phosphonate | 1:10 | 0.6:6 |
| X.06 | Disodium phosphonate | 1:1 | 0.6:0.6 |
| X.06 | Difenoconazole | 1:1 | 6:6 |
| X.06 | Difenoconazole | 10:1 | 6:0.6 |
| X.06 | Difenoconazole | 1:10 | 0.6:6 |
| X.06 | Difenoconazole | 1:1 | 0.6:0.6 |
| X.06 | Fludioxonil | 1:1 | 6:6 |
| X.06 | Fludioxonil | 10:1 | 6:0.6 |
| X.06 | Fludioxonil | 1:10 | 0.6:6 |
| X.06 | Fludioxonil | 1:1 | 0.6:0.6 |
| X.06 | Fluazinam | 1:1 | 6:6 |
| X.06 | Fluazinam | 30:1 | 6:0.2 |
| X.06 | Fluazinam | 1:10 | 0.6:6 |
| X.06 | Fluazinam | 3:1 | 0.6:0.2 |
| X.06 | Ametoctradin | 1:1 | 6:6 |
| X.06 | Ametoctradin | 30:1 | 6:0.2 |
| X.06 | Ametoctradin | 1:10 | 0.6:6 |
| X.06 | Ametoctradin | 3:1 | 0.6:0.2 |
| X.06 | Amisulbrom | 1:1 | 6:6 |
| X.06 | Amisulbrom | 30:1 | 6:0.2 |
| X.06 | Amisulbrom | 1:10 | 0.6:6 |
| X.06 | Amisulbrom | 3:1 | 0.6:0.2 |
| X.06 | Cyazofamid | 1:1 | 6:6 |
| X.06 | Cyazofamid | 30:1 | 6:0.2 |
| X.06 | Cyazofamid | 1:10 | 0.6:6 |
| X.06 | Cyazofamid | 3:1 | 0.6:0.2 |
| X.06 | Azoxystrobin | 1:1 | 6:6 |
| X.06 | Azoxystrobin | 10:1 | 6:0.6 |
| X.06 | Azoxystrobin | 1:10 | 0.6:6 |
| X.06 | Azoxystrobin | 1:1 | 0.6:0.6 |
| X.06 | Sedaxane | 1:1 | 6:6 |
| X.06 | Sedaxane | 10:1 | 6:0.6 |
| X.06 | Sedaxane | 1:10 | 0.6:6 |
| X.06 | Sedaxane | 1:1 | 0.6:0.6 |
| X.06 | Pydiflumetofen | 1:1 | 6:6 |
| X.06 | Pydiflumetofen | 10:1 | 6:0.6 |
| X.06 | Pydiflumetofen | 1:10 | 0.6:6 |
| X.06 | Pydiflumetofen | 1:1 | 0.6:0.6 |
| X.06 | Metalaxyl-M | 1:1 | 6:6 |
| X.06 | Metalaxyl-M | 30:1 | 6:0.2 |
| X.06 | Metalaxyl-M | 1:10 | 0.6:6 |
| X.06 | Metalaxyl-M | 3:1 | 0.6:0.2 |
| X.06 | Folpet | 1:1 | 6:6 |
| X.06 | Folpet | 10:1 | 6:0.6 |
| X.06 | Folpet | 1:10 | 0.6:6 |
| X.06 | Folpet | 1:1 | 0.6:0.6 |
| X.06 | Chlorothalonil | 1:1 | 6:6 |
| X.06 | Chlorothalonil | 10:1 | 6:0.6 |
| X.06 | Chlorothalonil | 1:10 | 0.6:6 |
| X.06 | Chlorothalonil | 1:1 | 0.6:0.6 |
| X.06 | Oxathiapiprolin | 1:1 | 6:6 |
| X.06 | Oxathiapiprolin | 30:1 | 6:0.2 |
| X.06 | Oxathiapiprolin | 1:10 | 0.6:6 |
| X.06 | Oxathiapiprolin | 3:1 | 0.6:0.2 |
| X.06 | Fluoxapiprolin | 1:1 | 6:6 |
| X.06 | Fluoxapiprolin | 30:1 | 6:0.2 |
| X.06 | Fluoxapiprolin | 1:10 | 0.6:6 |
| X.06 | Fluoxapiprolin | 3:1 | 0.6:0.2 |
| X.06 | Acibenzolar-S-methyl | 1:1 | 6:6 |
| X.06 | Acibenzolar-S-methyl | 10:1 | 6:0.6 |
| X.06 | Acibenzolar-S-methyl | 1:10 | 0.6:6 |
| X.06 | Acibenzolar-S-methyl | 1:1 | 0.6:0.6 |
| X.06 | Fluopicolide | 1:1 | 6:6 |
| X.06 | Fluopicolide | 30:1 | 6:0.2 |
| X.06 | Fluopicolide | 1:10 | 0.6:6 |
| X.06 | Fluopicolide | 3:1 | 0.6:0.2 |
| X.06 | Zoxamide | 1:1 | 6:6 |
| X.06 | Zoxamide | 30:1 | 6:0.2 |
| X.06 | Zoxamide | 1:10 | 0.6:6 |
| X.06 | Zoxamide | 3:1 | 0.6:0.2 |
| X.06 | Mandipropamid | 1:1 | 6:6 |
| X.06 | Mandipropamid | 30:1 | 6:0.2 |
| X.06 | Mandipropamid | 1:10 | 0.6:6 |
| X.06 | Mandipropamid | 3:1 | 0.6:0.2 |
| X.06 | Cyclobutrifluram | 1:1 | 6:6 |
| X.06 | Cyclobutrifluram | 10:1 | 6:0.6 |
| X.06 | Cyclobutrifluram | 1:10 | 0.6:6 |
| X.06 | Cyclobutrifluram | 1:1 | 0.6:0.6 |
| X.21 | Picarbutrazox | 3.3:1 | 20:6 |
| X.21 | Picarbutrazox | 33.3:1 | 20:0.6 |
| X.21 | Picarbutrazox | 1:3 | 2:6 |
| X.21 | Picarbutrazox | 3.3:1 | 2:0.6 |
| X.21 | Cymoxanil | 3.3:1 | 20:6 |
| X.21 | Cymoxanil | 33.3:1 | 20:0.6 |
| X.21 | Cymoxanil | 1:3 | 2:6 |
| X.21 | Cymoxanil | 3.3:1 | 2:0.6 |
| X.21 | Disodium phosphonate | 3.3:1 | 20:6 |
| X.21 | Disodium phosphonate | 33.3:1 | 20:0.6 |
| X.21 | Disodium phosphonate | 1:3 | 2:6 |
| X.21 | Disodium phosphonate | 3.3:1 | 2:0.6 |
| X.21 | Difenoconazole | 3.3:1 | 20:6 |
| X.21 | Difenoconazole | 33.3:1 | 20:0.6 |
| X.21 | Difenoconazole | 1:3 | 2:6 |
| X.21 | Difenoconazole | 3.3:1 | 2:0.6 |
| X.21 | Fludioxonil | 3.3:1 | 20:6 |
| X.21 | Fludioxonil | 33.3:1 | 20:0.6 |
| X.21 | Fludioxonil | 1:3 | 2:6 |
| X.21 | Fludioxonil | 3.3:1 | 2:0.6 |
| X.21 | Fluazinam | 3.3:1 | 20:6 |
| X.21 | Fluazinam | 33.3:1 | 20:0.6 |
| X.21 | Fluazinam | 1:3 | 2:6 |
| X.21 | Fluazinam | 3.3:1 | 2:0.6 |
| X.21 | Azoxystrobin | 3.3:1 | 20:6 |
| X.21 | Azoxystrobin | 33.3:1 | 20:0.6 |
| X.21 | Azoxystrobin | 1:3 | 2:6 |
| X.21 | Azoxystrobin | 3.3:1 | 2:0.6 |
| X.21 | Sedaxane | 3.3:1 | 20:6 |
| X.21 | Sedaxane | 33.3:1 | 20:0.6 |
| X.21 | Sedaxane | 1:3 | 2:6 |
| X.21 | Sedaxane | 3.3:1 | 2:0.6 |
| X.21 | Pydiflumetofen | 3.3:1 | 20:6 |
| X.21 | Pydiflumetofen | 33.3:1 | 20:0.6 |
| X.21 | Pydiflumetofen | 1:3 | 2:6 |
| X.21 | Pydiflumetofen | 3.3:1 | 2:0.6 |
| X.21 | Metalaxyl-M | 3.3:1 | 20:6 |
| X.21 | Metalaxyl-M | 33.3:1 | 20:0.6 |
| X.21 | Metalaxyl-M | 1:3 | 2:6 |
| X.21 | Metalaxyl-M | 3.3:1 | 2:0.6 |
| X.21 | Folpet | 3.3:1 | 20:6 |
| X.21 | Folpet | 33.3:1 | 20:0.6 |
| X.21 | Folpet | 1:3 | 2:6 |
| X.21 | Folpet | 3.3:1 | 2:0.6 |
| X.21 | Oxathiapiprolin | 3.3:1 | 20:6 |
| X.21 | Oxathiapiprolin | 33.3:1 | 20:0.6 |
| X.21 | Oxathiapiprolin | 1:3 | 2:6 |
| X.21 | Oxathiapiprolin | 3.3:1 | 2:0.6 |
| X.21 | Fluoxapiprolin | 3.3:1 | 20:6 |
| X.21 | Fluoxapiprolin | 33.3:1 | 20:0.6 |
| X.21 | Fluoxapiprolin | 1:3 | 2:6 |
| X.21 | Fluoxapiprolin | 3.3:1 | 2:0.6 |
| X.21 | Fluopicolide | 3.3:1 | 20:6 |
| X.21 | Fluopicolide | 33.3:1 | 20:0.6 |
| X.21 | Fluopicolide | 1:3 | 2:6 |
| X.21 | Fluopicolide | 3.3:1 | 2:0.6 |
| X.21 | Zoxamide | 3.3:1 | 20:6 |
| X.21 | Zoxamide | 33.3:1 | 20:0.6 |
| X.21 | Zoxamide | 1:3 | 2:6 |
| X.21 | Zoxamide | 3.3:1 | 2:0.6 |
| X.21 | Mandipropamid | 3.3:1 | 20:6 |
| X.21 | Mandipropamid | 33.3:1 | 20:0.6 |
| X.21 | Mandipropamid | 1:3 | 2:6 |
| X.21 | Mandipropamid | 3.3:1 | 2:0.6 |
| X.21 | Cyclobutrifluram | 3.3:1 | 20:6 |
| X.21 | Cyclobutrifluram | 33.3:1 | 20:0.6 |
| X.21 | Cyclobutrifluram | 1:3 | 2:6 |
| X.21 | Cyclobutrifluram | 3.3:1 | 2:0.6 |
| X.21 | Mefentrifluconazole | 3.3:1 | 20:6 |
| X.21 | Mefentrifluconazole | 33.3:1 | 20:0.6 |
| X.21 | Mefentrifluconazole | 1:3 | 2:6 |
| X.21 | Mefentrifluconazole | 3.3:1 | 2:0.6 |
| X.21 | Florylpicoxamid | 3.3:1 | 20:6 |
| X.21 | Florylpicoxamid | 33.3:1 | 20:0.6 |
| X.21 | Florylpicoxamid | 1:3 | 2:6 |
| X.21 | Florylpicoxamid | 3.3:1 | 2:0.6 |
| X.21 | Streptomyces chrestomyceticus | 3.3:1 | 20:6 |
| | CBS149411 | | |
| X.21 | Streptomyces chrestomyceticus | 33.3:1 | 20:0.6 |
| | CBS149411 | | |
| X.21 | Streptomyces chrestomyceticus | 1:3 | 2:6 |
| | CBS149411 | | |
| X.21 | Streptomyces chrestomyceticus | 3.3:1 | 2:0.6 |
| | CBS149411 | | |
| X.21 | Compound Z | 3.3:1 | 20:6 |
| X.21 | Compound Z | 33.3:1 | 20:0.6 |
| X.21 | Compound Z | 1:3 | 2:6 |
| X.21 | Compound Z | 3.3:1 | 2:0.6 |

### Example B4: Activity Against Phytophthora infestans (Late Blight of Potato/Tomato)

Tomato leaf disks are placed on water agar in multiwell plates (24-well format) and sprayed with test solutions. After drying, the leaf disks are inoculated with a spore suspension of the pathogen. After appropriate incubation the activity of a compound is assessed 4 dpi (days after inoculation) as preventive fungicidal activity. The following mixture compositions (A:B) at the reported concentration (in ppm) gave at least 80% disease control in this test.

| Component A | | | Conc. (ppm) |
|---|---|---|---|
| (Compound) | Component B | Ratio A:B | (A:B) |
| X.01 | Picarbutrazox | 1:1 | 200:200 |
| X.01 | Picarbutrazox | 10:1 | 200:20 |
| X.01 | Picarbutrazox | 1:3 | 60:200 |
| X.01 | Picarbutrazox | 3:1 | 60:20 |
| X.01 | Cymoxanil | 1:1 | 200:200 |
| X.01 | Cymoxanil | 10:1 | 200:20 |
| X.01 | Cymoxanil | 1:3 | 60:200 |
| X.01 | Cymoxanil | 3:1 | 60:20 |
| X.01 | Disodium phosphonate | 1:1 | 200:200 |
| X.01 | Disodium phosphonate | 10:1 | 200:20 |
| X.01 | Disodium phosphonate | 1:3 | 60:200 |
| X.01 | Disodium phosphonate | 3:1 | 60:20 |
| X.01 | Difenoconazole | 1:1 | 200:200 |
| X.01 | Difenoconazole | 10:1 | 200:20 |
| X.01 | Difenoconazole | 1:3 | 60:200 |
| X.01 | Difenoconazole | 3:1 | 60:20 |
| X.01 | Fludioxonil | 1:1 | 200:200 |
| X.01 | Fludioxonil | 10:1 | 200:20 |
| X.01 | Fludioxonil | 1:3 | 60:200 |
| X.01 | Fludioxonil | 3:1 | 60:20 |
| X.01 | Fluazinam | 1:1 | 200:200 |
| X.01 | Fluazinam | 10:1 | 200:20 |
| X.01 | Fluazinam | 1:3 | 60:200 |
| X.01 | Fluazinam | 3:1 | 60:20 |
| X.01 | Ametoctradin | 1:1 | 200:200 |
| X.01 | Ametoctradin | 10:1 | 200:20 |
| X.01 | Ametoctradin | 1:3 | 60:200 |
| X.01 | Ametoctradin | 3:1 | 60:20 |
| X.01 | Amisulbrom | 1:1 | 200:200 |
| X.01 | Amisulbrom | 10:1 | 200:20 |
| X.01 | Amisulbrom | 1:3 | 60:200 |
| X.01 | Amisulbrom | 3:1 | 60:20 |
| X.01 | Cyazofamid | 1:1 | 200:200 |
| X.01 | Cyazofamid | 10:1 | 200:20 |
| X.01 | Cyazofamid | 1:3 | 60:200 |
| X.01 | Cyazofamid | 3:1 | 60:20 |
| X.01 | Azoxystrobin | 1:1 | 200:200 |
| X.01 | Azoxystrobin | 10:1 | 200:20 |
| X.01 | Azoxystrobin | 1:3 | 60:200 |
| X.01 | Azoxystrobin | 3:1 | 60:20 |
| X.01 | Sedaxane | 1:1 | 200:200 |
| X.01 | Sedaxane | 10:1 | 200:20 |
| X.01 | Sedaxane | 1:3 | 60:200 |
| X.01 | Sedaxane | 3:1 | 60:20 |
| X.01 | Pydiflumetofen | 1:1 | 200:200 |
| X.01 | Pydiflumetofen | 10:1 | 200:20 |
| X.01 | Pydiflumetofen | 1:3 | 60:200 |
| X.01 | Pydiflumetofen | 3:1 | 60:20 |
| X.01 | Metalaxyl-M | 1:1 | 200:200 |
| X.01 | Metalaxyl-M | 10:1 | 200:20 |
| X.01 | Metalaxyl-M | 1:3 | 60:200 |
| X.01 | Metalaxyl-M | 3:1 | 60:20 |
| X.01 | Folpet | 1:1 | 200:200 |
| X.01 | Folpet | 10:1 | 200:20 |
| X.01 | Folpet | 1:3 | 60:200 |
| X.01 | Folpet | 3:1 | 60:20 |
| X.01 | Chlorothalonil | 1:1 | 200:200 |
| X.01 | Chlorothalonil | 10:1 | 200:20 |
| X.01 | Chlorothalonil | 1:3 | 60:200 |
| X.01 | Chlorothalonil | 3:1 | 60:20 |
| X.01 | Oxathiapiprolin | 10:1 | 200:20 |
| X.01 | Oxathiapiprolin | 100:1 | 200:2 |
| X.01 | Oxathiapiprolin | 3:1 | 60:20 |
| X.01 | Oxathiapiprolin | 30:1 | 60:2 |
| X.01 | Fluoxapiprolin | 10:1 | 200:20 |
| X.01 | Fluoxapiprolin | 100:1 | 200:2 |
| X.01 | Fluoxapiprolin | 3:1 | 60:20 |
| X.01 | Fluoxapiprolin | 30:1 | 60:2 |
| X.01 | Acibenzolar-S-methyl | 1:1 | 200:200 |
| X.01 | Acibenzolar-S-methyl | 10:1 | 200:20 |
| X.01 | Acibenzolar-S-methyl | 1:3 | 60:200 |
| X.01 | Acibenzolar-S-methyl | 3:1 | 60:20 |
| X.01 | Fluopicolide | 1:1 | 200:200 |
| X.01 | Fluopicolide | 10:1 | 200:20 |
| X.01 | Fluopicolide | 1:3 | 60:200 |
| X.01 | Fluopicolide | 3:1 | 60:20 |
| X.01 | Zoxamide | 1:1 | 200:200 |
| X.01 | Zoxamide | 10:1 | 200:20 |
| X.01 | Zoxamide | 1:3 | 60:200 |
| X.01 | Zoxamide | 3:1 | 60:20 |
| X.01 | Mandipropamid | 1:1 | 200:200 |
| X.01 | Mandipropamid | 10:1 | 200:20 |
| X.01 | Mandipropamid | 1:3 | 60:200 |
| X.01 | Mandipropamid | 3:1 | 60:20 |
| X.01 | Cyclobutrifluram | 1:1 | 200:200 |
| X.01 | Cyclobutrifluram | 10:1 | 200:20 |
| X.01 | Cyclobutrifluram | 1:3 | 60:200 |
| X.01 | Cyclobutrifluram | 3:1 | 60:20 |
| X.02 | Picarbutrazox | 1:1 | 200:200 |
| X.02 | Picarbutrazox | 10:1 | 200:20 |
| X.02 | Picarbutrazox | 1:3 | 60:200 |
| X.02 | Picarbutrazox | 3:1 | 60:20 |
| X.02 | Cymoxanil | 1:1 | 200:200 |
| X.02 | Cymoxanil | 10:1 | 200:20 |
| X.02 | Cymoxanil | 1:3 | 60:200 |
| X.02 | Cymoxanil | 3:1 | 60:20 |
| X.02 | Disodium phosphonate | 1:1 | 200:200 |
| X.02 | Disodium phosphonate | 10:1 | 200:20 |
| X.02 | Disodium phosphonate | 1:3 | 60:200 |
| X.02 | Disodium phosphonate | 3:1 | 60:20 |
| X.02 | Difenoconazole | 1:1 | 200:200 |
| X.02 | Difenoconazole | 10:1 | 200:20 |
| X.02 | Difenoconazole | 1:3 | 60:200 |
| X.02 | Difenoconazole | 3:1 | 60:20 |
| X.02 | Fludioxonil | 1:1 | 200:200 |
| X.02 | Fludioxonil | 10:1 | 200:20 |
| X.02 | Fludioxonil | 1:3 | 60:200 |
| X.02 | Fludioxonil | 3:1 | 60:20 |
| X.02 | Fluazinam | 1:1 | 200:200 |
| X.02 | Fluazinam | 10:1 | 200:20 |
| X.02 | Fluazinam | 1:3 | 60:200 |
| X.02 | Fluazinam | 3:1 | 60:20 |
| X.02 | Ametoctradin | 1:1 | 200:200 |
| X.02 | Ametoctradin | 10:1 | 200:20 |
| X.02 | Ametoctradin | 1:3 | 60:200 |
| X.02 | Ametoctradin | 3:1 | 60:20 |
| X.02 | Amisulbrom | 1:1 | 200:200 |
| X.02 | Amisulbrom | 10:1 | 200:20 |
| X.02 | Amisulbrom | 1:3 | 60:200 |
| X.02 | Amisulbrom | 3:1 | 60:20 |
| X.02 | Cyazofamid | 1:1 | 200:200 |
| X.02 | Cyazofamid | 10:1 | 200:20 |
| X.02 | Cyazofamid | 1:3 | 60:200 |
| X.02 | Cyazofamid | 3:1 | 60:20 |
| X.02 | Azoxystrobin | 1:1 | 200:200 |
| X.02 | Azoxystrobin | 10:1 | 200:20 |
| X.02 | Azoxystrobin | 1:3 | 60:200 |
| X.02 | Azoxystrobin | 3:1 | 60:20 |
| X.02 | Sedaxane | 1:1 | 200:200 |
| X.02 | Sedaxane | 10:1 | 200:20 |
| X.02 | Sedaxane | 1:3 | 60:200 |
| X.02 | Sedaxane | 3:1 | 60:20 |
| X.02 | Pydiflumetofen | 1:1 | 200:200 |
| X.02 | Pydiflumetofen | 10:1 | 200:20 |
| X.02 | Pydiflumetofen | 1:3 | 60:200 |
| X.02 | Pydiflumetofen | 3:1 | 60:20 |
| X.02 | Metalaxyl-M | 1:1 | 200:200 |
| X.02 | Metalaxyl-M | 10:1 | 200:20 |
| X.02 | Metalaxyl-M | 1:3 | 60:200 |
| X.02 | Metalaxyl-M | 3:1 | 60:20 |
| X.02 | Folpet | 1:1 | 200:200 |
| X.02 | Folpet | 10:1 | 200:20 |
| X.02 | Folpet | 1:3 | 60:200 |
| X.02 | Folpet | 3:1 | 60:20 |
| X.02 | Chlorothalonil | 1:1 | 200:200 |
| X.02 | Chlorothalonil | 10:1 | 200:20 |
| X.02 | Chlorothalonil | 1:3 | 60:200 |
| X.02 | Chlorothalonil | 3:1 | 60:20 |
| X.02 | Oxathiapiprolin | 10:1 | 200:20 |
| X.02 | Oxathiapiprolin | 100:1 | 200:2 |
| X.02 | Oxathiapiprolin | 3:1 | 60:20 |
| X.02 | Oxathiapiprolin | 30:1 | 60:2 |
| X.02 | Fluoxapiprolin | 10:1 | 200:20 |
| X.02 | Fluoxapiprolin | 100:1 | 200:2 |
| X.02 | Fluoxapiprolin | 3:1 | 60:20 |
| X.02 | Fluoxapiprolin | 30:1 | 60:2 |
| X.02 | Acibenzolar-S-methyl | 1:1 | 200:200 |
| X.02 | Acibenzolar-S-methyl | 10:1 | 200:20 |
| X.02 | Acibenzolar-S-methyl | 1:3 | 60:200 |
| X.02 | Acibenzolar-S-methyl | 3:1 | 60:20 |
| X.02 | Fluopicolide | 1:1 | 200:200 |
| X.02 | Fluopicolide | 10:1 | 200:20 |
| X.02 | Fluopicolide | 1:3 | 60:200 |
| X.02 | Fluopicolide | 3:1 | 60:20 |
| X.02 | Zoxamide | 1:1 | 200:200 |
| X.02 | Zoxamide | 10:1 | 200:20 |
| X.02 | Zoxamide | 1:3 | 60:200 |
| X.02 | Zoxamide | 3:1 | 60:20 |
| X.02 | Mandipropamid | 1:1 | 200:200 |
| X.02 | Mandipropamid | 10:1 | 200:20 |
| X.02 | Mandipropamid | 1:3 | 60:200 |
| X.02 | Mandipropamid | 3:1 | 60:20 |
| X.02 | Cyclobutrifluram | 1:1 | 200:200 |
| X.02 | Cyclobutrifluram | 10:1 | 200:20 |
| X.02 | Cyclobutrifluram | 1:3 | 60:200 |
| X.02 | Cyclobutrifluram | 3:1 | 60:20 |
| X.03 | Picarbutrazox | 1:1 | 200:200 |
| X.03 | Picarbutrazox | 10:1 | 200:20 |
| X.03 | Picarbutrazox | 1:3 | 60:200 |
| X.03 | Picarbutrazox | 3:1 | 60:20 |
| X.03 | Cymoxanil | 1:1 | 200:200 |
| X.03 | Cymoxanil | 10:1 | 200:20 |
| X.03 | Cymoxanil | 1:3 | 60:200 |
| X.03 | Cymoxanil | 3:1 | 60:20 |
| X.03 | Disodium phosphonate | 1:1 | 200:200 |
| X.03 | Disodium phosphonate | 10:1 | 200:20 |
| X.03 | Disodium phosphonate | 1:3 | 60:200 |
| X.03 | Disodium phosphonate | 3:1 | 60:20 |
| X.03 | Difenoconazole | 1:1 | 200:200 |
| X.03 | Difenoconazole | 10:1 | 200:20 |
| X.03 | Difenoconazole | 1:3 | 60:200 |
| X.03 | Difenoconazole | 3:1 | 60:20 |
| X.03 | Fludioxonil | 1:1 | 200:200 |
| X.03 | Fludioxonil | 10:1 | 200:20 |
| X.03 | Fludioxonil | 1:3 | 60:200 |
| X.03 | Fludioxonil | 3:1 | 60:20 |
| X.03 | Fluazinam | 1:1 | 200:200 |
| X.03 | Fluazinam | 10:1 | 200:20 |
| X.03 | Fluazinam | 1:3 | 60:200 |
| X.03 | Fluazinam | 3:1 | 60:20 |
| X.03 | Ametoctradin | 1:1 | 200:200 |
| X.03 | Ametoctradin | 10:1 | 200:20 |
| X.03 | Ametoctradin | 1:3 | 60:200 |
| X.03 | Ametoctradin | 3:1 | 60:20 |
| X.03 | Amisulbrom | 1:1 | 200:200 |
| X.03 | Amisulbrom | 10:1 | 200:20 |
| X.03 | Amisulbrom | 1:3 | 60:200 |
| X.03 | Amisulbrom | 3:1 | 60:20 |
| X.03 | Cyazofamid | 1:1 | 200:200 |
| X.03 | Cyazofamid | 10:1 | 200:20 |
| X.03 | Cyazofamid | 1:3 | 60:200 |
| X.03 | Cyazofamid | 3:1 | 60:20 |
| X.03 | Azoxystrobin | 1:1 | 200:200 |
| X.03 | Azoxystrobin | 10:1 | 200:20 |
| X.03 | Azoxystrobin | 1:3 | 60:200 |
| X.03 | Azoxystrobin | 3:1 | 60:20 |
| K.03 | Sedaxane | 1:1 | 200:200 |
| X.03 | Sedaxane | 10:1 | 200:20 |
| X.03 | Sedaxane | 1:3 | 60:200 |
| X.03 | Sedaxane | 3:1 | 60:20 |
| X.03 | Pydiflumetofen | 1:1 | 200:200 |
| X.03 | Pydiflumetofen | 10:1 | 200:20 |
| X.03 | Pydiflumetofen | 1:3 | 60:200 |
| X.03 | Pydiflumetofen | 3:1 | 60:20 |
| X.03 | Metalaxyl-M | 1:1 | 200:200 |
| X.03 | Metalaxyl-M | 10:1 | 200:20 |
| X.03 | Metalaxyl-M | 1:3 | 60:200 |
| X.03 | Metalaxyl-M | 3:1 | 60:20 |
| X.03 | Folpet | 1:1 | 200:200 |
| X.03 | Folpet | 10:1 | 200:20 |
| X.03 | Folpet | 1:3 | 60:200 |
| X.03 | Folpet | 3:1 | 60:20 |
| X.03 | Chlorothalonil | 1:1 | 200:200 |
| X.03 | Chlorothalonil | 10:1 | 200:20 |
| X.03 | Chlorothalonil | 1:3 | 60:200 |
| X.03 | Chlorothalonil | 3:1 | 60:20 |
| X.03 | Oxathiapiprolin | 10:1 | 200:20 |
| X.03 | Oxathiapiprolin | 100:1 | 200:2 |
| X.03 | Oxathiapiprolin | 3:1 | 60:20 |
| X.03 | Oxathiapiprolin | 30:1 | 60:2 |
| K.03 | Fluoxapiprolin | 10:1 | 200:20 |
| X.03 | Fluoxapiprolin | 100:1 | 200:2 |
| X.03 | Fluoxapiprolin | 3:1 | 60:20 |
| X.03 | Fluoxapiprolin | 30:1 | 60:2 |
| X.03 | Acibenzolar-S-methyl | 1:1 | 200:200 |
| X.03 | Acibenzolar-S-methyl | 10:1 | 200:20 |
| X.03 | Acibenzolar-S-methyl | 1:3 | 60:200 |
| X.03 | Acibenzolar-S-methyl | 3:1 | 60:20 |
| X.03 | Fluopicolide | 1:1 | 200:200 |
| X.03 | Fluopicolide | 10:1 | 200:20 |
| X.03 | Fluopicolide | 1:3 | 60:200 |
| X.03 | Fluopicolide | 3:1 | 60:20 |
| X.03 | Zoxamide | 1:1 | 200:200 |
| X.03 | Zoxamide | 10:1 | 200:20 |
| X.03 | Zoxamide | 1:3 | 60:200 |
| X.03 | Zoxamide | 3:1 | 60:20 |
| X.03 | Mandipropamid | 1:1 | 200:200 |
| X.03 | Mandipropamid | 10:1 | 200:20 |
| X.03 | Mandipropamid | 1:3 | 60:200 |
| X.03 | Mandipropamid | 3:1 | 60:20 |
| X.03 | Cyclobutrifluram | 1:1 | 200:200 |
| X.03 | Cyclobutrifluram | 10:1 | 200:20 |
| X.03 | Cyclobutrifluram | 1:3 | 60:200 |
| X.03 | Cyclobutrifluram | 3:1 | 60:20 |
| X.04 | Picarbutrazox | 1:1 | 200:200 |
| X.04 | Picarbutrazox | 10:1 | 200:20 |
| X.04 | Picarbutrazox | 1:3 | 60:200 |
| X.04 | Picarbutrazox | 3:1 | 60:20 |
| X.04 | Cymoxanil | 1:1 | 200:200 |
| X.04 | Cymoxanil | 10:1 | 200:20 |
| X.04 | Cymoxanil | 1:3 | 60:200 |
| X.04 | Cymoxanil | 3:1 | 60:20 |
| X.04 | Disodium phosphonate | 1:1 | 200:200 |
| X.04 | Disodium phosphonate | 10:1 | 200:20 |
| X.04 | Disodium phosphonate | 1:3 | 60:200 |
| X.04 | Disodium phosphonate | 3:1 | 60:20 |
| X.04 | Difenoconazole | 1:1 | 200:200 |
| X.04 | Difenoconazole | 10:1 | 200:20 |
| X.04 | Difenoconazole | 1:3 | 60:200 |
| X.04 | Difenoconazole | 3:1 | 60:20 |
| X.04 | Fludioxonil | 1:1 | 200:200 |
| X.04 | Fludioxonil | 10:1 | 200:20 |
| X.04 | Fludioxonil | 1:3 | 60:200 |
| X.04 | Fludioxonil | 3:1 | 60:20 |
| X.04 | Fluazinam | 1:1 | 200:200 |
| X.04 | Fluazinam | 10:1 | 200:20 |
| X.04 | Fluazinam | 1:3 | 60:200 |
| X.04 | Fluazinam | 3:1 | 60:20 |
| X.04 | Ametoctradin | 1:1 | 200:200 |
| X.04 | Ametoctradin | 10:1 | 200:20 |
| X.04 | Ametoctradin | 1:3 | 60:200 |
| X.04 | Ametoctradin | 3:1 | 60:20 |
| X.04 | Amisulbrom | 1:1 | 200:200 |
| X.04 | Amisulbrom | 10:1 | 200:20 |
| X.04 | Amisulbrom | 1:3 | 60:200 |
| X.04 | Amisulbrom | 3:1 | 60:20 |
| X.04 | Cyazofamid | 1:1 | 200:200 |
| X.04 | Cyazofamid | 10:1 | 200:20 |
| X.04 | Cyazofamid | 1:3 | 60:200 |
| X.04 | Cyazofamid | 3:1 | 60:20 |
| X.04 | Azoxystrobin | 1:1 | 200:200 |
| X.04 | Azoxystrobin | 10:1 | 200:20 |
| X.04 | Azoxystrobin | 1:3 | 60:200 |
| X.04 | Azoxystrobin | 3:1 | 60:20 |
| X.04 | Sedaxane | 1:1 | 200:200 |
| X.04 | Sedaxane | 10:1 | 200:20 |
| X.04 | Sedaxane | 1:3 | 60:200 |
| X.04 | Sedaxane | 3:1 | 60:20 |
| X.04 | Pydiflumetofen | 1:1 | 200:200 |
| X.04 | Pydiflumetofen | 10:1 | 200:20 |
| X.04 | Pydiflumetofen | 1:3 | 60:200 |
| X.04 | Pydiflumetofen | 3:1 | 60:20 |
| X.04 | Metalaxyl-M | 1:1 | 200:200 |
| X.04 | Metalaxyl-M | 10:1 | 200:20 |
| X.04 | Metalaxyl-M | 1:3 | 60:200 |
| X.04 | Metalaxyl-M | 3:1 | 60:20 |
| X.04 | Folpet | 1:1 | 200:200 |
| X.04 | Folpet | 10:1 | 200:20 |
| X.04 | Folpet | 1:3 | 60:200 |
| X.04 | Folpet | 3:1 | 60:20 |
| X.04 | Chlorothalonil | 1:1 | 200:200 |
| X.04 | Chlorothalonil | 10:1 | 200:20 |
| X.04 | Chlorothalonil | 1:3 | 60:200 |
| X.04 | Chlorothalonil | 3:1 | 60:20 |
| X.04 | Oxathiapiprolin | 10:1 | 200:20 |
| X.04 | Oxathiapiprolin | 100:1 | 200:2 |
| X.04 | Oxathiapiprolin | 3:1 | 60:20 |
| X.04 | Oxathiapiprolin | 30:1 | 60:2 |
| X.04 | Fluoxapiprolin | 10:1 | 200:20 |
| X.04 | Fluoxapiprolin | 100:1 | 200:2 |
| X.04 | Fluoxapiprolin | 3:1 | 60:20 |
| X.04 | Fluoxapiprolin | 30:1 | 60:2 |
| X.04 | Acibenzolar-S-methyl | 1:1 | 200:200 |
| X.04 | Acibenzolar-S-methyl | 10:1 | 200:20 |
| X.04 | Acibenzolar-S-methyl | 1:3 | 60:200 |
| X.04 | Acibenzolar-S-methyl | 3:1 | 60:20 |
| X.04 | Fluopicolide | 1:1 | 200:200 |
| X.04 | Fluopicolide | 10:1 | 200:20 |
| X.04 | Fluopicolide | 1:3 | 60:200 |
| X.04 | Fluopicolide | 3:1 | 60:20 |
| X.04 | Zoxamide | 1:1 | 200:200 |
| X.04 | Zoxamide | 10:1 | 200:20 |
| X.04 | Zoxamide | 1:3 | 60:200 |
| X.04 | Zoxamide | 3:1 | 60:20 |
| X.04 | Mandipropamid | 1:1 | 200:200 |
| X.04 | Mandipropamid | 10:1 | 200:20 |
| X.04 | Mandipropamid | 1:3 | 60:200 |
| X.04 | Mandipropamid | 3:1 | 60:20 |
| X.04 | Cyclobutrifluram | 1:1 | 200:200 |
| X.04 | Cyclobutrifluram | 10: | 200:20 |
| X.04 | Cyclobutrifluram | 1:3 | 60:200 |
| X.04 | Cyclobutrifluram | 3:1 | 60:20 |
| X.05 | Picarbutrazox | 1:1 | 200:200 |
| X.05 | Picarbutrazox | 10:1 | 200:20 |
| X.05 | Picarbutrazox | 1:3 | 60:200 |
| X.05 | Picarbutrazox | 3:1 | 60:20 |
| X.05 | Cymoxanil | 1:1 | 200:200 |
| X.05 | Cymoxanil | 10:1 | 200:20 |
| X.05 | Cymoxanil | 1:3 | 60:200 |
| X.05 | Cymoxanil | 3:1 | 60:20 |
| X.05 | Disodium phosphonate | 1:1 | 200:200 |
| X.05 | Disodium phosphonate | 10:1 | 200:20 |
| X.05 | Disodium phosphonate | 1:3 | 60:200 |
| X.05 | Disodium phosphonate | 3:1 | 60:20 |
| X.05 | Difenoconazole | 1:1 | 200:200 |
| X.05 | Difenoconazole | 10:1 | 200:20 |
| X.05 | Difenoconazole | 1:3 | 60:200 |
| X.05 | Difenoconazole | 3:1 | 60:20 |
| X.05 | Fludioxonil | 1:1 | 200:200 |
| X.05 | Fludioxonil | 10:1 | 200:20 |
| X.05 | Fludioxonil | 1:3 | 60:200 |
| X.05 | Fludioxonil | 3:1 | 60:20 |
| X.05 | Fluazinam | 1:1 | 200:200 |
| X.05 | Fluazinam | 10:1 | 200:20 |
| X.05 | Fluazinam | 1:3 | 60:200 |
| X.05 | Fluazinam | 3:1 | 60:20 |
| X.05 | Ametoctradin | 1:1 | 200:200 |
| X.05 | Ametoctradin | 10:1 | 200:20 |
| X.05 | Ametoctradin | 1:3 | 60:200 |
| X.05 | Ametoctradin | 3:1 | 60:20 |
| X.05 | Amisulbrom | 1:1 | 200:200 |
| K.05 | Amisulbrom | 10:1 | 200:20 |
| X.05 | Amisulbrom | 1:3 | 60:200 |
| X.05 | Amisulbrom | 3:1 | 60:20 |
| X.05 | Cyazofamid | 1:1 | 200:200 |
| X.05 | Cyazofamid | 10:1 | 200:20 |
| X.05 | Cyazofamid | 1:3 | 60:200 |
| X.05 | Cyazofamid | 3:1 | 60:20 |
| X.05 | Azoxystrobin | 1:1 | 200:200 |
| X.05 | Azoxystrobin | 10:1 | 200:20 |
| X.05 | Azoxystrobin | 1:3 | 60:200 |
| X.05 | Azoxystrobin | 3:1 | 60:20 |
| X.05 | Sedaxane | 1:1 | 200:200 |
| K.05 | Sedaxane | 10:1 | 200:20 |
| X.05 | Sedaxane | 1:3 | 60:200 |
| X.05 | Sedaxane | 3:1 | 60:20 |
| X.05 | Pydiflumetofen | 1:1 | 200:200 |
| X.05 | Pydiflumetofen | 10:1 | 200:20 |
| X.05 | Pydiflumetofen | 1:3 | 60:200 |
| X.05 | Pydiflumetofen | 3:1 | 60:20 |
| X.05 | Metalaxyl-M | 1:1 | 200:200 |
| K.05 | Metalaxyl-M | 10:1 | 200:20 |
| X.05 | Metalaxyl-M | 1:3 | 60:200 |
| X.05 | Metalaxyl-M | 3:1 | 60:20 |
| X.05 | Folpet | 1:1 | 200:200 |
| X.05 | Folpet | 10:1 | 200:20 |
| X.05 | Folpet | 1:3 | 60:200 |
| X.05 | Folpet | 3:1 | 60:20 |
| X.05 | Chlorothalonil | 1:1 | 200:200 |
| X.05 | Chlorothalonil | 10:1 | 200:20 |
| X.05 | Chlorothalonil | 1:3 | 60:200 |
| X.05 | Chlorothalonil | 3:1 | 60:20 |
| X.05 | Oxathiapiprolin | 10:1 | 200:20 |
| X.05 | Oxathiapiprolin | 100:1 | 200:2 |
| X.05 | Oxathiapiprolin | 3:1 | 60:20 |
| X.05 | Oxathiapiprolin | 30:1 | 60:2 |
| X.05 | Fluoxapiprolin | 10:1 | 200:20 |
| X.05 | Fluoxapiprolin | 100:1 | 200:2 |
| X.05 | Fluoxapiprolin | 3:1 | 60:20 |
| X.05 | Fluoxapiprolin | 30:1 | 60:2 |
| X.05 | Acibenzolar-S-methyl | 1:1 | 200:200 |
| X.05 | Acibenzolar-S-methyl | 10:1 | 200:20 |
| X.05 | Acibenzolar-S-methyl | 1:3 | 60:200 |
| X.05 | Acibenzolar-S-methyl | 3:1 | 60:20 |
| X.05 | Fluopicolide | 1:1 | 200:200 |
| X.05 | Fluopicolide | 10:1 | 200:20 |
| X.05 | Fluopicolide | 1:3 | 60:200 |
| X.05 | Fluopicolide | 3:1 | 60:20 |
| X.05 | Zoxamide | 1:1 | 200:200 |
| X.05 | Zoxamide | 10:1 | 200:20 |
| X.05 | Zoxamide | 1:3 | 60:200 |
| X.05 | Zoxamide | 3:1 | 60:20 |
| X.05 | Mandipropamid | 1:1 | 200:200 |
| X.05 | Mandipropamid | 10:1 | 200:20 |
| X.05 | Mandipropamid | 1:3 | 60:200 |
| X.05 | Mandipropamid | 3:1 | 60:20 |
| X.05 | Cyclobutrifluram | 1:1 | 200:200 |
| X.05 | Cyclobutrifluram | 10:1 | 200:20 |
| X.05 | Cyclobutrifluram | 1:3 | 60:200 |
| X.05 | Cyclobutrifluram | 3:1 | 60:20 |
| X.06 | Picarbutrazox | 1:1 | 200:200 |
| X.06 | Picarbutrazox | 10:1 | 200:20 |
| X.06 | Picarbutrazox | 1:3 | 60:200 |
| X.06 | Picarbutrazox | 3:1 | 60:20 |
| X.06 | Cymoxanil | 1:1 | 200:200 |
| X.06 | Cymoxanil | 10:1 | 200:20 |
| X.06 | Cymoxanil | 1:3 | 60:200 |
| X.06 | Cymoxanil | 3:1 | 60:20 |
| X.06 | Disodium phosphonate | 1:1 | 200:200 |
| X.06 | Disodium phosphonate | 10:1 | 200:20 |
| X.06 | Disodium phosphonate | 1:3 | 60:200 |
| X.06 | Disodium phosphonate | 3:1 | 60:20 |
| X.06 | Difenoconazole | 1:1 | 200:200 |
| X.06 | Difenoconazole | 10:1 | 200:20 |
| X.06 | Difenoconazole | 1:3 | 60:200 |
| X.06 | Difenoconazole | 3:1 | 60:20 |
| X.06 | Fludioxonil | 1:1 | 200:200 |
| X.06 | Fludioxonil | 10:1 | 200:20 |
| X.06 | Fludioxonil | 1:3 | 60:200 |
| X.06 | Fludioxonil | 3:1 | 60:20 |
| X.06 | Fluazinam | 1:1 | 200:200 |
| X.06 | Fluazinam | 10:1 | 200:20 |
| X.06 | Fluazinam | 1:3 | 60:200 |
| X.06 | Fluazinam | 3:1 | 60:20 |
| X.06 | Ametoctradin | 1:1 | 200:200 |
| X.06 | Ametoctradin | 10:1 | 200:20 |
| X.06 | Ametoctradin | 1:3 | 60:200 |
| X.06 | Ametoctradin | 3:1 | 60:20 |
| X.06 | Amisulbrom | 1:1 | 200:200 |
| X.06 | Amisulbrom | 10:1 | 200:20 |
| X.06 | Amisulbrom | 1:3 | 60:200 |
| X.06 | Amisulbrom | 3:1 | 60:20 |
| X.06 | Cyazofamid | 1:1 | 200:200 |
| X.06 | Cyazofamid | 10:1 | 200:20 |
| X.06 | Cyazofamid | 1:3 | 60:200 |
| X.06 | Cyazofamid | 3:1 | 60:20 |
| X.06 | Azoxystrobin | 1:1 | 200:200 |
| X.06 | Azoxystrobin | 10:1 | 200:20 |
| X.06 | Azoxystrobin | 1:3 | 60:200 |
| X.06 | Azoxystrobin | 3:1 | 60:20 |
| X.06 | Sedaxane | 1:1 | 200:200 |
| X.06 | Sedaxane | 10:1 | 200:20 |
| X.06 | Sedaxane | 1:3 | 60:200 |
| X.06 | Sedaxane | 3:1 | 60:20 |
| X.06 | Pydiflumetofen | 1:1 | 200:200 |
| X.06 | Pydiflumetofen | 10:1 | 200:20 |
| X.06 | Pydiflumetofen | 1:3 | 60:200 |
| X.06 | Pydiflumetofen | 3:1 | 60:20 |
| X.06 | Metalaxyl-M | 1:1 | 200:200 |
| X.06 | Metalaxyl-M | 10:1 | 200:20 |
| X.06 | Metalaxyl-M | 1:3 | 60:200 |
| X.06 | Metalaxyl-M | 3:1 | 60:20 |
| X.06 | Folpet | 1:1 | 200:200 |
| X.06 | Folpet | 10:1 | 200:20 |
| X.06 | Folpet | 1:3 | 60:200 |
| X.06 | Folpet | 3:1 | 60:20 |
| X.06 | Chlorothalonil | 1:1 | 200:200 |
| X.06 | Chlorothalonil | 10:1 | 200:20 |
| X.06 | Chlorothalonil | 1:3 | 60:200 |
| X.06 | Chlorothalonil | 3:1 | 60:20 |
| X.06 | Oxathiapiprolin | 10:1 | 200:20 |
| X.06 | Oxathiapiprolin | 100:1 | 200:2 |
| X.06 | Oxathiapiprolin | 3:1 | 60:20 |
| X.06 | Oxathiapiprolin | 30:1 | 60:2 |
| X.06 | Fluoxapiprolin | 10:1 | 200:20 |
| X.06 | Fluoxapiprolin | 100:1 | 200:2 |
| X.06 | Fluoxapiprolin | 3:1 | 60:20 |
| X.06 | Fluoxapiprolin | 30:1 | 60:2 |
| X.06 | Acibenzolar-S-methyl | 1:1 | 200:200 |
| X.06 | Acibenzolar-S-methyl | 10:1 | 200:20 |
| X.06 | Acibenzolar-S-methyl | 1:3 | 60:200 |
| X.06 | Acibenzolar-S-methyl | 3:1 | 60:20 |
| X.06 | Fluopicolide | 1:1 | 200:200 |
| X.06 | Fluopicolide | 10:1 | 200:20 |
| X.06 | Fluopicolide | 1:3 | 60:200 |
| X.06 | Fluopicolide | 3:1 | 60:20 |
| X.06 | Zoxamide | 1:1 | 200:200 |
| X.06 | Zoxamide | 10:1 | 200:20 |
| X.06 | Zoxamide | 1:3 | 60:200 |
| X.06 | Zoxamide | 3:1 | 60:20 |
| X.06 | Mandipropamid | 1:1 | 200:200 |
| X.06 | Mandipropamid | 10:1 | 200:20 |
| X.06 | Mandipropamid | 1:3 | 60:200 |
| X.06 | Mandipropamid | 3:1 | 60:20 |
| X.06 | Cyclobutrifluram | 1:1 | 200:200 |
| X.06 | Cyclobutrifluram | 10:1 | 200:20 |
| X.06 | Cyclobutrifluram | 1:3 | 60:200 |
| X.06 | Cyclobutrifluram | 3:1 | 60:20 |

### Example B35: Activity Against Plasmopara Viticola (Downy Mildew of Grapevine)

Grape vine leaf disks are placed on agar in multiwell plates (24-well format) and sprayed with test solutions. After drying, the leaf disks are inoculated with a spore suspension of the pathogen. After appropriate incubation the activity of a compound is assessed 7 dpi (days after inoculation) as preventive fungicidal activity. The following mixture compositions (A:B) at the reported concentration (in ppm) gave at least 80% disease control in this test.

| Component A | | Ratio | Conc. (ppm) |
|---|---|---|---|
| (Compound) | Component B | A:B | (A:B) |
| X.01 | Picarbutrazox | 1:1 | 200:200 |
| X.01 | Picarbutrazox | 10:1 | 200:20 |
| X.01 | Picarbutrazox | 1:3 | 60:200 |
| X.01 | Picarbutrazox | 3:1 | 60:20 |
| X.01 | Cymoxanil | 1:1 | 200:200 |
| X.01 | Cymoxanil | 10:1 | 200:20 |
| X.01 | Cymoxanil | 1:3 | 60:200 |
| X.01 | Cymoxanil | 3:1 | 60:20 |
| X.01 | Disodium phosphonate | 1:1 | 200:200 |
| X.01 | Disodium phosphonate | 10:1 | 200:20 |
| X.01 | Disodium phosphonate | 1:3 | 60:200 |
| X.01 | Disodium phosphonate | 3:1 | 60:20 |
| X.01 | Difenoconazole | 1:1 | 200:200 |
| X.01 | Difenoconazole | 10:1 | 200:20 |
| X.01 | Difenoconazole | 1:3 | 60:200 |
| X.01 | Difenoconazole | 3:1 | 60:20 |
| X.01 | Fludioxonil | 1:1 | 200:200 |
| X.01 | Fludioxonil | 10:1 | 200:20 |
| X.01 | Fludioxonil | 1:3 | 60:200 |
| X.01 | Fludioxonil | 3:1 | 60:20 |
| X.01 | Fluazinam | 1:1 | 200:200 |
| X.01 | Fluazinam | 10:1 | 200:20 |
| X.01 | Fluazinam | 1:3 | 60:200 |
| X.01 | Fluazinam | 3:1 | 60:20 |
| X.01 | Ametoctradin | 1:1 | 200:200 |
| X.01 | Ametoctradin | 10:1 | 200:20 |
| X.01 | Ametoctradin | 1:3 | 60:200 |
| X.01 | Ametoctradin | 3:1 | 60:20 |
| X.01 | Amisulbrom | 1:1 | 200:200 |
| X.01 | Amisulbrom | 10:1 | 200:20 |
| X.01 | Amisulbrom | 1:3 | 60:200 |
| X.01 | Amisulbrom | 3:1 | 60:20 |
| X.01 | Cyazofamid | 1:1 | 200:200 |
| X.01 | Cyazofamid | 10:1 | 200:20 |
| X.01 | Cyazofamid | 1:3 | 60:200 |
| X.01 | Cyazofamid | 3:1 | 60:20 |
| X.01 | Azoxystrobin | 1:1 | 200:200 |
| X.01 | Azoxystrobin | 10:1 | 200:20 |
| X.01 | Azoxystrobin | 1:3 | 60:200 |
| X.01 | Azoxystrobin | 3:1 | 60:20 |
| X.01 | Sedaxane | 1:1 | 200:200 |
| X.01 | Sedaxane | 10:1 | 200:20 |
| X.01 | Sedaxane | 1:3 | 60:200 |
| X.01 | Sedaxane | 3:1 | 60:20 |
| X.01 | Pydiflumetofen | 1:1 | 200:200 |
| X.01 | Pydiflumetofen | 10:1 | 200:20 |
| X.01 | Pydiflumetofen | 1:3 | 60:200 |
| X.01 | Pydiflumetofen | 3:1 | 60:20 |
| X.01 | Metalaxyl-M | 1:1 | 200:200 |
| X.01 | Metalaxyl-M | 10:1 | 200:20 |
| X.01 | Metalaxyl-M | 1:3 | 60:200 |
| X.01 | Metalaxyl-M | 3:1 | 60:20 |
| X.01 | Folpet | 1:1 | 200:200 |
| X.01 | Folpet | 10:1 | 200:20 |
| X.01 | Folpet | 1:3 | 60:200 |
| X.01 | Folpet | 3:1 | 60:20 |
| X.01 | Chlorothalonil | 1:1 | 200:200 |
| X.01 | Chlorothalonil | 10:1 | 200:20 |
| X.01 | Chlorothalonil | 1:3 | 60:200 |
| X.01 | Chlorothalonil | 3:1 | 60:20 |
| X.01 | Oxathiapiprolin | 10:1 | 200:20 |
| X.01 | Oxathiapiprolin | 100:1 | 200:2 |
| X.01 | Oxathiapiprolin | 3:1 | 60:20 |
| X.01 | Oxathiapiprolin | 30:1 | 60:2 |
| X.01 | Fluoxapiprolin | 10:1 | 200:20 |
| X.01 | Fluoxapiprolin | 100:1 | 200:2 |
| X.01 | Fluoxapiprolin | 3:1 | 60:20 |
| X.01 | Fluoxapiprolin | 30:1 | 60:2 |
| X.01 | Acibenzolar-S-methyl | 1:1 | 200:200 |
| X.01 | Acibenzolar-S-methyl | 10:1 | 200:20 |
| X.01 | Acibenzolar-S-methyl | 1:3 | 60:200 |
| X.01 | Acibenzolar-S-methyl | 3:1 | 60:20 |
| X.01 | Fluopicolide | 1:1 | 200:200 |
| X.01 | Fluopicolide | 10:1 | 200:20 |
| X.01 | Fluopicolide | 1:3 | 60:200 |
| X.01 | Fluopicolide | 3:1 | 60:20 |
| X.01 | Zoxamide | 1:1 | 200:200 |
| X.01 | Zoxamide | 10:1 | 200:20 |
| X.01 | Zoxamide | 1:3 | 60:200 |
| X.01 | Zoxamide | 3:1 | 60:20 |
| X.01 | Mandipropamid | 1:1 | 200:200 |
| X.01 | Mandipropamid | 10:1 | 200:20 |
| X.01 | Mandipropamid | 1:3 | 60:200 |
| X.01 | Mandipropamid | 3:1 | 60:20 |
| X.01 | Cyclobutrifluram | 1:1 | 200:200 |
| X.01 | Cyclobutrifluram | 10:1 | 200:20 |
| X.01 | Cyclobutrifluram | 1:3 | 60:200 |
| X.01 | Cyclobutrifluram | 3:1 | 60:20 |
| X.02 | Picarbutrazox | 1:1 | 200:200 |
| X.02 | Picarbutrazox | 10:1 | 200:20 |
| X.02 | Picarbutrazox | 1:3 | 60:200 |
| X.02 | Picarbutrazox | 3:1 | 60:20 |
| X.02 | Cymoxanil | 1:1 | 200:200 |
| X.02 | Cymoxanil | 10:1 | 200:20 |
| X.02 | Cymoxanil | 1:3 | 60:200 |
| X.02 | Cymoxanil | 3:1 | 60:20 |
| X.02 | Disodium phosphonate | 1:1 | 200:200 |
| X.02 | Disodium phosphonate | 10:1 | 200:20 |
| X.02 | Disodium phosphonate | 1:3 | 60:200 |
| X.02 | Disodium phosphonate | 3:1 | 60:20 |
| X.02 | Difenoconazole | 1:1 | 200:200 |
| X.02 | Difenoconazole | 10:1 | 200:20 |
| X.02 | Difenoconazole | 1:3 | 60:200 |
| X.02 | Difenoconazole | 3:1 | 60:20 |
| X.02 | Fludioxonil | 1:1 | 200:200 |
| X.02 | Fludioxonil | 10:1 | 200:20 |
| X.02 | Fludioxonil | 1:3 | 60:200 |
| X.02 | Fludioxonil | 3:1 | 60:20 |
| X.02 | Fluazinam | 1:1 | 200:200 |
| X.02 | Fluazinam | 10:1 | 200:20 |
| X.02 | Fluazinam | 1:3 | 60:200 |
| X.02 | Fluazinam | 3:1 | 60:20 |
| X.02 | Ametoctradin | 1:1 | 200:200 |
| X.02 | Ametoctradin | 10:1 | 200:20 |
| X.02 | Ametoctradin | 1:3 | 60:200 |
| X.02 | Ametoctradin | 3:1 | 60:20 |
| X.02 | Amisulbrom | 1:1 | 200:200 |
| X.02 | Amisulbrom | 10:1 | 200:20 |
| X.02 | Amisulbrom | 1:3 | 60:200 |
| X.02 | Amisulbrom | 3:1 | 60:20 |
| X.02 | Cyazofamid | 1:1 | 200:200 |
| X.02 | Cyazofamid | 10:1 | 200:20 |
| X.02 | Cyazofamid | 1:3 | 60:200 |
| X.02 | Cyazofamid | 3:1 | 60:20 |
| X.02 | Azoxystrobin | 1:1 | 200:200 |
| X.02 | Azoxystrobin | 10:1 | 200:20 |
| X.02 | Azoxystrobin | 1:3 | 60:200 |
| X.02 | Azoxystrobin | 3:1 | 60:20 |
| X.02 | Sedaxane | 1:1 | 200:200 |
| X.02 | Sedaxane | 10:1 | 200:20 |
| X.02 | Sedaxane | 1:3 | 60:200 |
| X.02 | Sedaxane | 3:1 | 60:20 |
| X.02 | Pydiflumetofen | 1:1 | 200:200 |
| X.02 | Pydiflumetofen | 10:1 | 200:20 |
| X.02 | Pydiflumetofen | 1:3 | 60:200 |
| X.02 | Pydiflumetofen | 3:1 | 60:20 |
| X.02 | Metalaxyl-M | 1:1 | 200:200 |
| X.02 | Metalaxyl-M | 10:1 | 200:20 |
| X.02 | Metalaxyl-M | 1:3 | 60:200 |
| X.02 | Metalaxyl-M | 3:1 | 60:20 |
| X.02 | Folpet | 1:1 | 200:200 |
| X.02 | Folpet | 10:1 | 200:20 |
| X.02 | Folpet | 1:3 | 60:200 |
| X.02 | Folpet | 3:1 | 60:20 |
| X.02 | Chlorothalonil | 1:1 | 200:200 |
| X.02 | Chlorothalonil | 10:1 | 200:20 |
| X.02 | Chlorothalonil | 1:3 | 60:200 |
| X.02 | Chlorothalonil | 3:1 | 60:20 |
| X.02 | Oxathiapiprolin | 10:1 | 200:20 |
| X.02 | Oxathiapiprolin | 100:1 | 200:2 |
| X.02 | Oxathiapiprolin | 3:1 | 60:20 |
| X.02 | Oxathiapiprolin | 30:1 | 60:2 |
| X.02 | Fluoxapiprolin | 10:1 | 200:20 |
| X.02 | Fluoxapiprolin | 100:1 | 200:2 |
| X.02 | Fluoxapiprolin | 3:1 | 60:20 |
| X.02 | Fluoxapiprolin | 30:1 | 60:2 |
| X.02 | Acibenzolar-S-methyl | 1:1 | 200:200 |
| X.02 | Acibenzolar-S-methyl | 10:1 | 200:20 |
| X.02 | Acibenzolar-S-methyl | 1:3 | 60:200 |
| X.02 | Acibenzolar-S-methyl | 3:1 | 60:20 |
| X.02 | Fluopicolide | 1:1 | 200:200 |
| X.02 | Fluopicolide | 10:1 | 200:20 |
| X.02 | Fluopicolide | 1:3 | 60:200 |
| X.02 | Fluopicolide | 3:1 | 60:20 |
| X.02 | Zoxamide | 1:1 | 200:200 |
| X.02 | Zoxamide | 10:1 | 200:20 |
| X.02 | Zoxamide | 1:3 | 60:200 |
| X.02 | Zoxamide | 3:1 | 60:20 |
| X.02 | Mandipropamid | 1:1 | 200:200 |
| X.02 | Mandipropamid | 10:1 | 200:20 |
| X.02 | Mandipropamid | 1:3 | 60:200 |
| X.02 | Mandipropamid | 3:1 | 60:20 |
| X.02 | Cyclobutrifluram | 1:1 | 200:200 |
| X.02 | Cyclobutrifluram | 10:1 | 200:20 |
| X.02 | Cyclobutrifluram | 1:3 | 60:200 |
| X.03 | Picarbutrazox | 1:1 | 200:200 |
| X.03 | Picarbutrazox | 10:1 | 200:20 |
| X.03 | Picarbutrazox | 1:3 | 60:200 |
| X.03 | Picarbutrazox | 3:1 | 60:20 |
| X.03 | Cymoxanil | 1:1 | 200:200 |
| X.03 | Cymoxanil | 10:1 | 200:20 |
| X.03 | Cymoxanil | 1:3 | 60:200 |
| X.03 | Cymoxanil | 3:1 | 60:20 |
| X.03 | Disodium phosphonate | 1:1 | 200:200 |
| X.03 | Disodium phosphonate | 10:1 | 200:20 |
| X.03 | Disodium phosphonate | 1:3 | 60:200 |
| X.03 | Disodium phosphonate | 3:1 | 60:20 |
| X.03 | Difenoconazole | 1:1 | 200:200 |
| X.03 | Difenoconazole | 10:1 | 200:20 |
| X.03 | Difenoconazole | 1:3 | 60:200 |
| X.03 | Difenoconazole | 3:1 | 60:20 |
| X.03 | Fludioxonil | 1:1 | 200:200 |
| X.03 | Fludioxonil | 10:1 | 200:20 |
| X.03 | Fludioxonil | 1:3 | 60:200 |
| X.03 | Fludioxonil | 3:1 | 60:20 |
| X.03 | Fluazinam | 1:1 | 200:200 |
| X.03 | Fluazinam | 10:1 | 200:20 |
| X.03 | Fluazinam | 1:3 | 60:200 |
| X.03 | Fluazinam | 3:1 | 60:20 |
| X.03 | Ametoctradin | 1:1 | 200:200 |
| X.03 | Ametoctradin | 10:1 | 200:20 |
| X.03 | Ametoctradin | 1:3 | 60:200 |
| X.03 | Ametoctradin | 3:1 | 60:20 |
| X.03 | Amisulbrom | 1:1 | 200:200 |
| X.03 | Amisulbrom | 10:1 | 200:20 |
| X.03 | Amisulbrom | 1:3 | 60:200 |
| X.03 | Amisulbrom | 3:1 | 60:20 |
| X.03 | Cyazofamid | 1:1 | 200:200 |
| X.03 | Cyazofamid | 10:1 | 200:20 |
| X.03 | Cyazofamid | 1:3 | 60:200 |
| X.03 | Cyazofamid | 3:1 | 60:20 |
| X.03 | Azoxystrobin | 1:1 | 200:200 |
| X.03 | Azoxystrobin | 10:1 | 200:20 |
| X.03 | Azoxystrobin | 1:3 | 60:200 |
| X.03 | Azoxystrobin | 3:1 | 60:20 |
| X.03 | Sedaxane | 1:1 | 200:200 |
| X.03 | Sedaxane | 10:1 | 200:20 |
| X.03 | Sedaxane | 1:3 | 60:200 |
| X.03 | Sedaxane | 3:1 | 60:20 |
| X.03 | Pydiflumetofen | 1:1 | 200:200 |
| X.03 | Pydiflumetofen | 10:1 | 200:20 |
| X.03 | Pydiflumetofen | 1:3 | 60:200 |
| X.03 | Pydiflumetofen | 3:1 | 60:20 |
| X.03 | Metalaxyl-M | 1:1 | 200:200 |
| X.03 | Metalaxyl-M | 10:1 | 200:20 |
| X.03 | Metalaxyl-M | 1:3 | 60:200 |
| X.03 | Metalaxyl-M | 3:1 | 60:20 |
| X.03 | Folpet | 1:1 | 200:200 |
| X.03 | Folpet | 10:1 | 200:20 |
| X.03 | Folpet | 1:3 | 60:200 |
| X.03 | Folpet | 3:1 | 60:20 |
| X.03 | Chlorothalonil | 1:1 | 200:200 |
| X.03 | Chlorothalonil | 10:1 | 200:20 |
| X.03 | Chlorothalonil | 1:3 | 60:200 |
| X.03 | Chlorothalonil | 3:1 | 60:20 |
| X.03 | Oxathiapiprolin | 10:1 | 200:20 |
| X.03 | Oxathiapiprolin | 100:1 | 200:2 |
| X.03 | Oxathiapiprolin | 3:1 | 60:20 |
| X.03 | Oxathiapiprolin | 30:1 | 60:2 |
| X.03 | Fluoxapiprolin | 10:1 | 200:20 |
| X.03 | Fluoxapiprolin | 100:1 | 200:2 |
| X.03 | Fluoxapiprolin | 3:1 | 60:20 |
| X.03 | Fluoxapiprolin | 30:1 | 60:2 |
| X.03 | Acibenzolar-S-methyl | 1:1 | 200:200 |
| X.03 | Acibenzolar-S-methyl | 10:1 | 200:20 |
| X.03 | Acibenzolar-S-methyl | 1:3 | 60:200 |
| X.03 | Acibenzolar-S-methyl | 3:1 | 60:20 |
| X.03 | Fluopicolide | 1:1 | 200:200 |
| X.03 | Fluopicolide | 10:1 | 200:20 |
| X.03 | Fluopicolide | 1:3 | 60:200 |
| X.03 | Fluopicolide | 3:1 | 60:20 |
| X.03 | Zoxamide | 1:1 | 200:200 |
| X.03 | Zoxamide | 10:1 | 200:20 |
| X.03 | Zoxamide | 1:3 | 60:200 |
| X.03 | Zoxamide | 3:1 | 60:20 |
| X.03 | Mandipropamid | 1:1 | 200:200 |
| X.03 | Mandipropamid | 10:1 | 200:20 |
| X.03 | Mandipropamid | 1:3 | 60:200 |
| X.03 | Mandipropamid | 3:1 | 60:20 |
| X.03 | Cyclobutrifluram | 1:1 | 200:200 |
| X.03 | Cyclobutrifluram | 10:1 | 200:20 |
| X.03 | Cyclobutrifluram | 1:3 | 60:200 |
| X.03 | Cyclobutrifluram | 3:1 | 60:20 |
| X.04 | Picarbutrazox | 1:1 | 200:200 |
| X.04 | Picarbutrazox | 10:1 | 200:20 |
| X.04 | Picarbutrazox | 1:3 | 60:200 |
| X.04 | Picarbutrazox | 3:1 | 60:20 |
| X.04 | Cymoxanil | 1:1 | 200:200 |
| X.04 | Cymoxanil | 10:1 | 200:20 |
| X.04 | Cymoxanil | 1:3 | 60:200 |
| X.04 | Cymoxanil | 3:1 | 60:20 |
| X.04 | Disodium phosphonate | 1:1 | 200:200 |
| X.04 | Disodium phosphonate | 10:1 | 200:20 |
| X.04 | Disodium phosphonate | 1:3 | 60:200 |
| X.04 | Disodium phosphonate | 3:1 | 60:20 |
| X.04 | Difenoconazole | 1:1 | 200:200 |
| X.04 | Difenoconazole | 10:1 | 200:20 |
| X.04 | Difenoconazole | 1:3 | 60:200 |
| X.04 | Difenoconazole | 3:1 | 60:20 |
| X.04 | Fludioxonil | 1:1 | 200:200 |
| X.04 | Fludioxonil | 10:1 | 200:20 |
| X.04 | Fludioxonil | 1:3 | 60:200 |
| X.04 | Fludioxonil | 3:1 | 60:20 |
| X.04 | Fluazinam | 1:1 | 200:200 |
| X.04 | Fluazinam | 10:1 | 200:20 |
| X.04 | Fluazinam | 1:3 | 60:200 |
| X.04 | Fluazinam | 3:1 | 60:20 |
| X.04 | Ametoctradin | 1:1 | 200:200 |
| X.04 | Ametoctradin | 10:1 | 200:20 |
| X.04 | Ametoctradin | 1:3 | 60:200 |
| X.04 | Ametoctradin | 3:1 | 60:20 |
| X.04 | Amisulbrom | 1:1 | 200:200 |
| X.04 | Amisulbrom | 10:1 | 200:20 |
| X.04 | Amisulbrom | 1:3 | 60:200 |
| X.04 | Amisulbrom | 3:1 | 60:20 |
| X.04 | Cyazofamid | 1:1 | 200:200 |
| X.04 | Cyazofamid | 10:1 | 200:20 |
| X.04 | Cyazofamid | 1:3 | 60:200 |
| X.04 | Cyazofamid | 3:1 | 60:20 |
| X.04 | Azoxystrobin | 1:1 | 200:200 |
| X.04 | Azoxystrobin | 10:1 | 200:20 |
| X.04 | Azoxystrobin | 1:3 | 60:200 |
| X.04 | Azoxystrobin | 3:1 | 60:20 |
| X.04 | Sedaxane | 1:1 | 200:200 |
| X.04 | Sedaxane | 10:1 | 200:20 |
| X.04 | Sedaxane | 1:3 | 60:200 |
| X.04 | Sedaxane | 3:1 | 60:20 |
| X.04 | Pydiflumetofen | 1:1 | 200:200 |
| X.04 | Pydiflumetofen | 10:1 | 200:20 |
| X.04 | Pydiflumetofen | 1:3 | 60:200 |
| X.04 | Pydiflumetofen | 3:1 | 60:20 |
| X.04 | Metalaxyl-M | 1:1 | 200:200 |
| X.04 | Metalaxyl-M | 10:1 | 200:20 |
| X.04 | Metalaxyl-M | 1:3 | 60:200 |
| X.04 | Metalaxyl-M | 3:1 | 60:20 |
| X.04 | Folpet | 1:1 | 200:200 |
| X.04 | Folpet | 10:1 | 200:20 |
| X.04 | Folpet | 1:3 | 60:200 |
| X.04 | Folpet | 3:1 | 60:20 |
| X.04 | Chlorothalonil | 1:1 | 200:200 |
| X.04 | Chlorothalonil | 10:1 | 200:20 |
| X.04 | Chlorothalonil | 1:3 | 60:200 |
| X.04 | Chlorothalonil | 3:1 | 60:20 |
| X.04 | Oxathiapiprolin | 10:1 | 200:20 |
| X.04 | Oxathiapiprolin | 100:1 | 200:2 |
| X.04 | Oxathiapiprolin | 3:1 | 60:20 |
| X.04 | Oxathiapiprolin | 30:1 | 60:2 |
| X.04 | Fluoxapiprolin | 10:1 | 200:20 |
| X.04 | Fluoxapiprolin | 100:1 | 200:2 |
| X.04 | Fluoxapiprolin | 3:1 | 60:20 |
| X.04 | Fluoxapiprolin | 30:1 | 60:2 |
| X.04 | Acibenzolar-S-methyl | 1:1 | 200:200 |
| X.04 | Acibenzolar-S-methyl | 10:1 | 200:20 |
| X.04 | Acibenzolar-S-methyl | 1:3 | 60:200 |
| X.04 | Acibenzolar-S-methyl | 3:1 | 60:20 |
| X.04 | Fluopicolide | 1:1 | 200:200 |
| K.04 | Fluopicolide | 10:1 | 200:20 |
| X.04 | Fluopicolide | 1:3 | 60:200 |
| X.04 | Fluopicolide | 3:1 | 60:20 |
| X.04 | Zoxamide | 1:1 | 200:200 |
| X.04 | Zoxamide | 10:1 | 200:20 |
| X.04 | Zoxamide | 1:3 | 60:200 |
| X.04 | Zoxamide | 3:1 | 60:20 |
| X.04 | Mandipropamid | 1:1 | 200:200 |
| X.04 | Mandipropamid | 10:1 | 200:20 |
| X.04 | Mandipropamid | 1:3 | 60:200 |
| X.04 | Mandipropamid | 3:1 | 60:20 |
| X.04 | Cyclobutrifluram | 1:1 | 200:200 |
| X.04 | Cyclobutrifluram | 10:1 | 200:20 |
| X.04 | Cyclobutrifluram | 1:3 | 60:200 |
| X.04 | Cyclobutrifluram | 3:1 | 60:20 |
| X.05 | Picarbutrazox | 1:1 | 200:200 |
| X.05 | Picarbutrazox | 10:1 | 200:20 |
| X.05 | Picarbutrazox | 1:3 | 60:200 |
| X.05 | Picarbutrazox | 3:1 | 60:20 |
| X.05 | Cymoxanil | 1:1 | 200:200 |
| X.05 | Cymoxanil | 10:1 | 200:20 |
| X.05 | Cymoxanil | 1:3 | 60:200 |
| X.05 | Cymoxanil | 3:1 | 60:20 |
| X.05 | Disodium phosphonate | 1:1 | 200:200 |
| X.05 | Disodium phosphonate | 10:1 | 200:20 |
| X.05 | Disodium phosphonate | 1:3 | 60:200 |
| X.05 | Disodium phosphonate | 3:1 | 60:20 |
| X.05 | Difenoconazole | 1:1 | 200:200 |
| X.05 | Difenoconazole | 10:1 | 200:20 |
| X.05 | Difenoconazole | 1:3 | 60:200 |
| X.05 | Difenoconazole | 3:1 | 60:20 |
| X.05 | Fludioxonil | 1:1 | 200:200 |
| X.05 | Fludioxonil | 10:1 | 200:20 |
| X.05 | Fludioxonil | 1:3 | 60:200 |
| X.05 | Fludioxonil | 3:1 | 60:20 |
| X.05 | Fluazinam | 1:1 | 200:200 |
| X.05 | Fluazinam | 10:1 | 200:20 |
| X.05 | Fluazinam | 1:3 | 60:200 |
| X.05 | Fluazinam | 3:1 | 60:20 |
| X.05 | Ametoctradin | 1:1 | 200:200 |
| X.05 | Ametoctradin | 10:1 | 200:20 |
| X.05 | Ametoctradin | 1:3 | 60:200 |
| X.05 | Ametoctradin | 3:1 | 60:20 |
| X.05 | Amisulbrom | 1:1 | 200:200 |
| X.05 | Amisulbrom | 10:1 | 200:20 |
| X.05 | Amisulbrom | 1:3 | 60:200 |
| X.05 | Amisulbrom | 3:1 | 60:20 |
| X.05 | Cyazofamid | 1:1 | 200:200 |
| X.05 | Cyazofamid | 10:1 | 200:20 |
| X.05 | Cyazofamid | 1:3 | 60:200 |
| X.05 | Cyazofamid | 3:1 | 60:20 |
| X.05 | Azoxystrobin | 1:1 | 200:200 |
| X.05 | Azoxystrobin | 10:1 | 200:20 |
| X.05 | Azoxystrobin | 1:3 | 60:200 |
| X.05 | Azoxystrobin | 3:1 | 60:20 |
| X.05 | Sedaxane | 1:1 | 200:200 |
| X.05 | Sedaxane | 10:1 | 200:20 |
| X.05 | Sedaxane | 1:3 | 60:200 |
| X.05 | Sedaxane | 3:1 | 60:20 |
| X.05 | Pydiflumetofen | 1:1 | 200:200 |
| X.05 | Pydiflumetofen | 10:1 | 200:20 |
| X.05 | Pydiflumetofen | 1:3 | 60:200 |
| X.05 | Pydiflumetofen | 3:1 | 60:20 |
| X.05 | Metalaxyl-M | 1:1 | 200:200 |
| X.05 | Metalaxyl-M | 10:1 | 200:20 |
| X.05 | Metalaxyl-M | 1:3 | 60:200 |
| X.05 | Metalaxyl-M | 3:1 | 60:20 |
| X.05 | Folpet | 1:1 | 200:200 |
| X.05 | Folpet | 10:1 | 200:20 |
| X.05 | Folpet | 1:3 | 60:200 |
| X.05 | Folpet | 3:1 | 60:20 |
| X.05 | Chlorothalonil | 1:1 | 200:200 |
| X.05 | Chlorothalonil | 10:1 | 200:20 |
| X.05 | Chlorothalonil | 1:3 | 60:200 |
| X.05 | Chlorothalonil | 3:1 | 60:20 |
| K.05 | Oxathiapiprolin | 10:1 | 200:20 |
| X.05 | Oxathiapiprolin | 100:1 | 200:2 |
| X.05 | Oxathiapiprolin | 3:1 | 60:20 |
| X.05 | Oxathiapiprolin | 30:1 | 60:2 |
| X.05 | Fluoxapiprolin | 10:1 | 200:20 |
| X.05 | Fluoxapiprolin | 100:1 | 200:2 |
| X.05 | Fluoxapiprolin | 3:1 | 60:20 |
| X.05 | Fluoxapiprolin | 30:1 | 60:2 |
| X.05 | Acibenzolar-S-methyl | 1:1 | 200:200 |
| X.05 | Acibenzolar-S-methyl | 10:1 | 200:20 |
| X.05 | Acibenzolar-S-methyl | 1:3 | 60:200 |
| X.05 | Acibenzolar-S-methyl | 3:1 | 60:20 |
| X.05 | Fluopicolide | 1:1 | 200:200 |
| X.05 | Fluopicolide | 10:1 | 200:20 |
| X.05 | Fluopicolide | 1:3 | 60:200 |
| X.05 | Fluopicolide | 3:1 | 60:20 |
| X.05 | Zoxamide | 1:1 | 200:200 |
| X.05 | Zoxamide | 10:1 | 200:20 |
| X.05 | Zoxamide | 1:3 | 60:200 |
| X.05 | Zoxamide | 3:1 | 60:20 |
| X.05 | Mandipropamid | 1:1 | 200:200 |
| X.05 | Mandipropamid | 10:1 | 200:20 |
| X.05 | Mandipropamid | 1:3 | 60:200 |
| X.05 | Mandipropamid | 3:1 | 60:20 |
| X.05 | Cyclobutrifluram | 1:1 | 200:200 |
| X.05 | Cyclobutrifluram | 10:1 | 200:20 |
| X.05 | Cyclobutrifluram | 1:3 | 60:200 |
| X.05 | Cyclobutrifluram | 3:1 | 60:20 |
| X.06 | Picarbutrazox | 1:1 | 200:200 |
| X.06 | Picarbutrazox | 10:1 | 200:20 |
| X.06 | Picarbutrazox | 1:3 | 60:200 |
| X.06 | Picarbutrazox | 3:1 | 60:20 |
| X.06 | Cymoxanil | 1:1 | 200:200 |
| X.06 | Cymoxanil | 10:1 | 200:20 |
| X.06 | Cymoxanil | 1:3 | 60:200 |
| X.06 | Disodium phosphonate | 1:1 | 200:200 |
| X.06 | Disodium phosphonate | 10:1 | 200:20 |
| X.06 | Difenoconazole | 1:1 | 200:200 |
| X.06 | Difenoconazole | 10:1 | 200:20 |
| X.06 | Difenoconazole | 1:3 | 60:200 |
| X.06 | Fludioxonil | 1:1 | 200:200 |
| X.06 | Fludioxonil | 10:1 | 200:20 |
| X.06 | Fludioxonil | 1:3 | 60:200 |
| X.06 | Fludioxonil | 3:1 | 60:20 |
| X.06 | Fluazinam | 1:1 | 200:200 |
| X.06 | Fluazinam | 10:1 | 200:20 |
| X.06 | Fluazinam | 1:3 | 60:200 |
| X.06 | Fluazinam | 3:1 | 60:20 |
| X.06 | Ametoctradin | 1:1 | 200:200 |
| X.06 | Ametoctradin | 10:1 | 200:20 |
| X.06 | Ametoctradin | 1:3 | 60:200 |
| X.06 | Ametoctradin | 3:1 | 60:20 |
| X.06 | Amisulbrom | 1:1 | 200:200 |
| X.06 | Amisulbrom | 10:1 | 200:20 |
| X.06 | Amisulbrom | 1:3 | 60:200 |
| X.06 | Amisulbrom | 3:1 | 60:20 |
| X.06 | Cyazofamid | 1:1 | 200:200 |
| X.06 | Cyazofamid | 10:1 | 200:20 |
| X.06 | Cyazofamid | 1:3 | 60:200 |
| X.06 | Azoxystrobin | 1:1 | 200:200 |
| X.06 | Azoxystrobin | 10:1 | 200:20 |
| X.06 | Azoxystrobin | 1:3 | 60:200 |
| X.06 | Azoxystrobin | 3:1 | 60:20 |
| X.06 | Sedaxane | 1:1 | 200:200 |
| X.06 | Sedaxane | 10:1 | 200:20 |
| X.06 | Sedaxane | 1:3 | 60:200 |
| X.06 | Pydiflumetofen | 1:1 | 200:200 |
| X.06 | Pydiflumetofen | 10:1 | 200:20 |
| X.06 | Metalaxyl-M | 1:1 | 200:200 |
| X.06 | Metalaxyl-M | 10:1 | 200:20 |
| X.06 | Metalaxyl-M | 1:3 | 60:200 |
| X.06 | Metalaxyl-M | 3:1 | 60:20 |
| X.06 | Folpet | 1:1 | 200:200 |
| X.06 | Folpet | 10:1 | 200:20 |
| X.06 | Chlorothalonil | 1:1 | 200:200 |
| X.06 | Chlorothalonil | 10:1 | 200:20 |
| X.06 | Chlorothalonil | 1:3 | 60:200 |
| X.06 | Oxathiapiprolin | 10:1 | 200:20 |
| X.06 | Oxathiapiprolin | 100:1 | 200:2 |
| X.06 | Oxathiapiprolin | 3:1 | 60:20 |
| X.06 | Oxathiapiprolin | 30:1 | 60:2 |
| X.06 | Fluoxapiprolin | 10:1 | 200:20 |
| X.06 | Fluoxapiprolin | 100:1 | 200:2 |
| X.06 | Fluoxapiprolin | 3:1 | 60:20 |
| X.06 | Fluoxapiprolin | 30:1 | 60:2 |
| X.06 | Acibenzolar-S-methyl | 1:1 | 200:200 |
| X.06 | Acibenzolar-S-methyl | 10:1 | 200:20 |
| X.06 | Fluopicolide | 1:1 | 200:200 |
| X.06 | Fluopicolide | 10:1 | 200:20 |
| X.06 | Fluopicolide | 1:3 | 60:200 |
| X.06 | Fluopicolide | 3:1 | 60:20 |
| X.06 | Zoxamide | 1:1 | 200:200 |
| X.06 | Zoxamide | 10:1 | 200:20 |
| X.06 | Zoxamide | 1:3 | 60:200 |
| X.06 | Zoxamide | 3:1 | 60:20 |
| X.06 | Mandipropamid | 1:1 | 200:200 |
| X.06 | Mandipropamid | 10:1 | 200:20 |
| X.06 | Mandipropamid | 1:3 | 60:200 |
| X.06 | Mandipropamid | 3:1 | 60:20 |
| X.06 | Cyclobutrifluram | 1:1 | 200:200 |
| X.06 | Cyclobutrifluram | 10:1 | 200:20 |
| X.06 | Cyclobutrifluram | 1:3 | 60:200 |
| X.21 | Picarbutrazox | 1:1 | 200:200 |
| X.21 | Picarbutrazox | 10:1 | 200:20 |
| X.21 | Picarbutrazox | 1:3.3 | 60:200 |
| X.21 | Picarbutrazox | 3:1 | 60:20 |
| X.21 | Cymoxanil | 1:1 | 200:200 |
| X.21 | Cymoxanil | 10:1 | 200:20 |
| X.21 | Cymoxanil | 1:3.3 | 60:200 |
| X.21 | Cymoxanil | 3:1 | 60:20 |
| X.21 | Disodium phosphonate | 1:1 | 200:200 |
| X.21 | Disodium phosphonate | 10:1 | 200:20 |
| X.21 | Disodium phosphonate | 1:3.3 | 60:200 |
| X.21 | Disodium phosphonate | 3:1 | 60:20 |
| X.21 | Difenoconazole | 1:1 | 200:200 |
| X.21 | Difenoconazole | 10:1 | 200:20 |
| X.21 | Difenoconazole | 1:3.3 | 60:200 |
| X.21 | Difenoconazole | 3:1 | 60:20 |
| X.21 | Fludioxonil | 1:1 | 200:200 |
| X.21 | Fludioxonil | 10:1 | 200:20 |
| X.21 | Fludioxonil | 1:3.3 | 60:200 |
| X.21 | Fludioxonil | 3:1 | 60:20 |
| X.21 | Fluazinam | 1:1 | 200:200 |
| X.21 | Fluazinam | 10:1 | 200:20 |
| X.21 | Fluazinam | 1:3.3 | 60:200 |
| X.21 | Fluazinam | 3:1 | 60:20 |
| X.21 | Azoxystrobin | 1:1 | 200:200 |
| X.21 | Azoxystrobin | 10:1 | 200:20 |
| X.21 | Azoxystrobin | 1:3.3 | 60:200 |
| X.21 | Azoxystrobin | 3:1 | 60:20 |
| X.21 | Sedaxane | 1:1 | 200:200 |
| X.21 | Sedaxane | 10:1 | 200:20 |
| X.21 | Sedaxane | 1:3.3 | 60:200 |
| X.21 | Sedaxane | 3:1 | 60:20 |
| X.21 | Pydiflumetofen | 1:1 | 200:200 |
| X.21 | Pydiflumetofen | 10:1 | 200:20 |
| X.21 | Pydiflumetofen | 1:3.3 | 60:200 |
| X.21 | Pydiflumetofen | 3:1 | 60:20 |
| X.21 | Metalaxyl-M | 1:1 | 200:200 |
| X.21 | Metalaxyl-M | 10:1 | 200:20 |
| X.21 | Metalaxyl-M | 1:3.3 | 60:200 |
| X.21 | Metalaxyl-M | 3:1 | 60:20 |
| X.21 | Folpet | 1:1 | 200:200 |
| X.21 | Folpet | 10:1 | 200:20 |
| X.21 | Folpet | 1:3.3 | 60:200 |
| X.21 | Folpet | 3:1 | 60:20 |
| X.21 | Oxathiapiprolin | 10:1 | 200:20 |
| X.21 | Oxathiapiprolin | 100:1 | 200:2 |
| X.21 | Oxathiapiprolin | 3:1 | 60:20 |
| X.21 | Oxathiapiprolin | 30:1 | 60:2 |
| X.21 | Fluoxapiprolin | 10:1 | 200:20 |
| X.21 | Fluoxapiprolin | 100:1 | 200:2 |
| X.21 | Fluoxapiprolin | 3:1 | 60:20 |
| X.21 | Fluoxapiprolin | 30:1 | 60:2 |
| X.21 | Fluopicolide | 1:1 | 200:200 |
| X.21 | Fluopicolide | 10:1 | 200:20 |
| X.21 | Fluopicolide | 1:3.3 | 60:200 |
| X.21 | Fluopicolide | 3:1 | 60:20 |
| X.21 | Zoxamide | 1:1 | 200:200 |
| X.21 | Zoxamide | 10:1 | 200:20 |
| X.21 | Zoxamide | 1:3.3 | 60:200 |
| X.21 | Zoxamide | 3:1 | 60:20 |
| X.21 | Mandipropamid | 1:1 | 200:200 |
| X.21 | Mandipropamid | 10:1 | 200:20 |
| X.21 | Mandipropamid | 1:3.3 | 60:200 |
| X.21 | Mandipropamid | 3:1 | 60:20 |
| X.21 | Cyclobutrifluram | 1:1 | 200:200 |
| X.21 | Cyclobutrifluram | 10:1 | 200:20 |
| X.21 | Cyclobutrifluram | 1:3.3 | 60:200 |
| X.21 | Cyclobutrifluram | 3:1 | 60:20 |
| X.21 | Mefentrifluconazole | 1:1 | 200:200 |
| X.21 | Mefentrifluconazole | 10:1 | 200:20 |
| X.21 | Mefentrifluconazole | 1:3.3 | 60:200 |
| X.21 | Mefentrifluconazole | 3:1 | 60:20 |
| X.21 | Florylpicoxamid | 1:1 | 200:200 |
| X.21 | Florylpicoxamid | 10:1 | 200:20 |
| X.21 | Florylpicoxamid | 1:3.3 | 60:200 |
| X.21 | Florylpicoxamid | 3:1 | 60:20 |
| X.21 | Streptomyces chrestomyceticus | 1:1 | 200:200 |
| | CBS149411 | | |
| X.21 | Streptomyces chrestomyceticus | 10:1 | 200:20 |
| | CBS149411 | | |
| X.21 | Streptomyces chrestomyceticus | 1:3.3 | 60:200 |
| | CBS149411 | | |
| X.21 | Streptomyces chrestomyceticus | 3:1 | 60:20 |
| | CBS149411 | | |
| X.21 | Compound Z | 1:1 | 200:200 |
| X.21 | Compound Z | 10:1 | 200:20 |
| X.21 | Compound Z | 1:3.3 | 60:200 |
| X.21 | Compound Z | 3:1 | 60:20 |

1. A fungicidal composition comprising components (A) and (B) as active ingredients, wherein component (A) is a compound of formula (I): 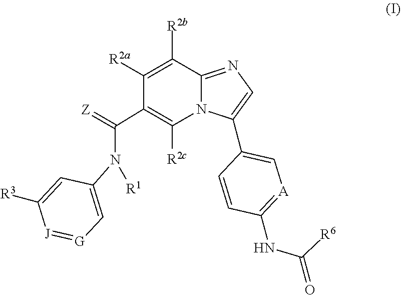
wherein Z is O;
R¹ is selected from C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl, wherein each of the C₁₋₆alkyl, C₁₋₆alkoxy-C₁₋₆alkyl, C₃₋₆cycloalkyl, C₁₋₆alkylsulfonyl-C₁₋₆alkyl, and C₃₋₆cycloalkyl-C₁₋₄alkyl groups is optionally substituted with CN;
R^{2a} is selected from H, C₁₋₆alkyl, C₃₋₆cycloalkyl, and C₁₋₆alkoxy-C₁₋₆alkyl;
R^{2b} is selected from H, CN, C₁₋₆alkyl, C₃₋₆cycloalkyl, C₁₋₆alkoxy-C₁₋₆alkyl, amino, and —NHC(O)C₁₋₆alkyl;
R^{2c} is H;
J is N or CR⁴;
G is CR⁵;
R³ is H;
R⁴ is halogen or CN;
R⁵ is selected from H, C₁₋₆alkyl, C₁₋₆alkoxy, and halogen;
A is CH or N; and
R⁶ is selected from C₁₋₆alkyl, C₁₋₆alkoxy, C₃₋₆cycloalkyl, C₁₋₆alkylamino, C₁₋₆alkoxyamino, and C₁₋₆alkylC₁₋₆alkoxyamino;
or a salt or N-oxide thereof;
and
component (B) is a compound selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide.

2. The fungicidal composition according claim 1, wherein:
R¹ is selected from CH₃, CH₂—CH₃, CH₂—O—CH₃, CH₂—SO₂—CH₃, and CH₂CN;
R^{2a} is selected from H and CH₃;
R^{2b} is selected from H, CH₃, —CH₂OCH₃, NH₂, and —NHC(O)CH₃;
R^{2c} is H;
J is N or CR⁴;
G is CR⁵;
R³ is H;
R⁴ is chloro, fluoro, or CN;
R⁵ is selected from H, CH₃, —OCH₃, and fluoro;
A is CH or N; and
R⁶ is selected from cyclopropyl, —OCH₃, and —NH—O—CH₃.

3. The fungicidal composition according claim 1, wherein the component (A) is a compound selected from:
methyl N-[5-[6-[(4-fluoro-3-methoxy-phenyl)-methyl-carbamoyl]imidazo[1,2-a]pyridin-3-yl]-2-pyridyl]carbamate,
methyl N-[5-[6-[(4-fluoro-3-methoxy-phenyl)-methyl-carbamoyl]-8-methyl-imidazo[1,2-a]pyridin-3-yl]-2-pyridyl]carbamate,
methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate,
methyl N-[5-[6-[(4-fluoro-3-methoxy-phenyl)-(methoxymethyl)carbamoyl]imidazo[1,2-a]pyridin-3-yl]-2-pyridyl]carbamate,
methyl N-[4-[6-[(4-fluoro-3-methoxy-phenyl)-methyl-carbamoyl]imidazo[1,2-a]pyridin-3-yl]phenyl]carbamate, 3-[6-(cyclopropanecarbonylamino)-3-pyridyl]-N-(4-fluoro-3-methoxy-phenyl)-N-methyl-imidazo[1,2-a]pyridine-6-carboxamide,
methyl N-[4-[6-[(4-fluorophenyl)-methyl-carbamoyl]imidazo[1,2-a]pyridin-3-yl]phenyl]carbamate,
methyl N-[4-[6-[(4-chlorophenyl)-methyl-carbamoyl]-8-methyl-imidazo[1,2-a]pyridin-3-yl]phenyl]carbamate,
methyl N-[5-[6-[(4-fluoro-3-methyl-phenyl)-methyl-carbamoyl]imidazo[1,2-a]pyridin-3-yl]-2-pyridyl]carbamate,
methyl N-[5-[6-[(4-cyano-3-methoxy-phenyl)-methyl-carbamoyl]imidazo[1,2-a]pyridin-3-yl]-2-pyridyl]carbamate,
methyl N-[5-[6-[(4-chloro-3-methoxy-phenyl)-methyl-carbamoyl]imidazo[1,2-a]pyridin-3-yl]-2-pyridyl]carbamate,
methyl N-[5-[6-[(4-fluorophenyl)-methyl-carbamoyl]-8-methyl-imidazo[1,2-a]pyridin-3-yl]-2-pyridyl]carbamate,
methyl N-[5-[6-[(4-fluorophenyl)-methyl-carbamoyl]-7-methyl-imidazo[1,2-a]pyridin-3-yl]-2-pyridyl]carbamate,
methyl N-[5-[6-[cyanomethyl-(4-fluoro-3-methoxy-phenyl)carbamoyl]imidazo[1,2-a]pyridin-3-yl]-2-pyridyl]carbamate,
methyl N-[5-[6-[(4-chloro-3-methoxy-phenyl)-(cyanomethyl)carbamoyl]imidazo[1,2-a]pyridin-3-yl]-2-pyridyl]carbamate,
methyl N-[5-[6-[(4-fluoro-3-methoxy-phenyl)-(methylsulfonylmethyl)carbamoyl]imidazo[1,2-a]pyridin-3-yl]-2-pyridyl]carbamate,
N-(4-fluoro-3-methoxy-phenyl)-3-[6-(methoxycarbamoylamino)-3-pyridyl]-N-methyl-imidazo[1,2-a]pyridine-6-carboxamide,
methyl N-[5-[8-acetamido-6-[(4-fluorophenyl)-methyl-carbamoyl]imidazo[1,2-a]pyridin-3-yl]-2-pyridyl]carbamate,
methyl N-[5-[6-[ethyl-(4-fluorophenyl)carbamoyl]imidazo[1,2-a]pyridin-3-yl]-2-pyridyl]carbamate,
methyl N-[5-[8-acetamido-6-[(4-fluoro-3-methoxy-phenyl)-methyl-carbamoyl]imidazo[1,2-a]pyridin-3-yl]-2-pyridyl]carbamate,
methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate,
methyl N-[5-[6-[(2-methoxy-4-pyridyl)-methyl-carbamoyl]-8-methyl-imidazo[1,2-a]pyridin-3-yl]-2-pyridyl]carbamate, and
methyl N-[5-[6-[(4-cyano-3-methoxy-phenyl)-methyl-carbamoyl]-8-methyl-imidazo[1,2-a]pyridin-3-yl]-2-pyridyl]carbamate;
or a salt or N-oxide thereof.

4. The fungicidal composition according to claim 1, wherein the component (A) is:
methyl N-[5-[6-[(4-fluoro-3-methoxy-phenyl)-methyl-carbamoyl]imidazo[1,2-a]pyridin-3-yl]-2-pyridyl]carbamate, or
methyl N-[5-[6-[(4-fluoro-3-methoxy-phenyl)-methyl-carbamoyl]-8-methyl-imidazo[1,2-a]pyridin-3-yl]-2-pyridyl]carbamate, or
methyl N-[5-[6-[(4-fluoro-3-methoxy-phenyl)-(methoxymethyl)carbamoyl]-8-methyl-imidazo[1,2-a]pyridin-3-yl]-2-pyridyl]carbamate, or
methyl N-[5-[6-[(4-fluoro-3-methoxy-phenyl)-(methoxymethyl)carbamoyl]imidazo[1,2-a]pyridin-3-yl]-2-pyridyl]carbamate, or
methyl N-[4-[6-[(4-fluoro-3-methoxy-phenyl)-methyl-carbamoyl]imidazo[1,2-a]pyridin-3-yl]phenyl]carbamate, or 3-[6-(cyclopropanecarbonylamino)-3-pyridyl]-N-(4-fluoro-3-methoxy-phenyl)-N-methyl-imidazo[1,2-a]pyridine-6-carboxamide, or
methyl {N}-[5-[6-[(4-fluoro-3-methoxy-phenyl)-methyl-carbamoyl]-8-(methoxymethyl)imidazo[1,2-a]pyridin-3-yl]-2-pyridyl]carbamate, or
methyl N-[5-[6-[(2-methoxy-4-pyridyl)-methyl-carbamoyl]-8-methyl-imidazo[1,2-a]pyridin-3-yl]-2-pyridyl]carbamate, or
methyl N-[5-[6-[(4-cyano-3-methoxy-phenyl)-methyl-carbamoyl]-8-methyl-imidazo[1,2-a]pyridin-3-yl]-2-pyridyl]carbamate;
or a salt or N-oxide thereof.

5. The fungicidal composition according to claim 1, wherein the component (B) is a compound selected from acibenzolar-S-methyl, ametoctradin, amisulbrom, azoxystrobin, chlorothalonil, cyazofamid, cyclobutrifluram, cymoxanil, difenoconazole, disodium phosphonate, florylpicoxamid, fluazinam, fludioxonil, fluopicolide, fluoxapiprolin, folpet, mandipropamid, mefentrifluconazole, metalaxyl-M, oxathiapiprolin, picarbutrazox, pydiflumetofen, sedaxane, Streptomyces chrestomyceticus, zoxamide, N-[(1R)-1-benzyl-1,3-dimethyl-butyl]-8-fluoro-quinoline-3-carboxamide, and N-[(1S)-1-benzyl-1,3-dimethyl-butyl]-8-fluoro-quinoline-3-carboxamide.

6. The fungicidal composition according to claim 1, wherein the weight ratio of component (A) to component (B) is from 100:1 to 1:100.

7. The fungicidal composition according to claim 1, wherein the weight ratio of component (A) to component (B) is from 20:1 to 1:40.

8. The fungicidal composition according to claim 1, wherein the weight ratio of component (A) to component (B) is from 12:1 to 1:25.

9. The fungicidal composition according to claim 1, wherein the weight ratio of component (A) to component (B) is from 5:1 to 1:15.

10. The fungicidal composition according to claim 1, wherein the weight ratio of component (A) to component (B) is from 2:1 to 1:5.

11. The fungicidal composition according to claim 1, wherein the composition further comprises an additional active ingredient component (C), which is different to component (B), selected from the group consisting of acetamiprid, acibenzolar-S-methyl, ametoctradin, amisulbrom, azoxystrobin, Bacillus amyloliquefaciens, benzovindiflupyr, broflanilid, chlorothalonil, chlothianidin, chlorantraniliprole, Cis-Jasmone, cyantraniliprole, cyazofamid, cyclobutrifluram, cymoxanil, difenoconazole, disodium phosphonate, ethaboxam, fenpicoxamid, florylpicoxamid, fluazinam, fludioxonil, fluindapyr, fluopicolide, fluopyram, fluoxapiprolin, fluoxytioconazole, fluxametamide, fluxapyroxad, folpet, imidaclprid, inpyrfluxam, ipconazole, ipflufenoquin, isocycloseram, isoflucypram, isotianil, mandipropamid, mefentrifulconazole, metalaxyl, metalaxyl-M (mefenoxam), metyltetraprole, oxathiapiprolin, penflufen, phosphite (phosphonate), picarbutrazox, prothioconazole, pydiflumetofen, pyraclostrobin, quinofumerin, sedaxane, silthiofam, Streptomyces chrestomyceticus, tebuconazole, tetraniliprole, thiabendazole, thiamethoxam, tolprocarb, triticonazole, zoxamide, N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, and N-((1S)-1-benzyl-3-chloro-1-methyl-but-3-enyl)-8-fluoro-quinoline-3-carboxamide.

12. The fungicidal composition according to claim 1, wherein the composition further comprises an agriculturally acceptable carrier and, optionally, a surfactant and/or formulation adjuvants.

13. A method of controlling or preventing phytopathogenic diseases, especially phytopathogenic fungi, on useful plants or on propagation material thereof, which comprises applying to the useful plants, the locus thereof or propagation material thereof the fungicidal composition as defined in claim 1.

14. A method according to claim 13, wherein the components (A) and (B) are applied in a sequential manner.

15. A compound of formula (XXI) 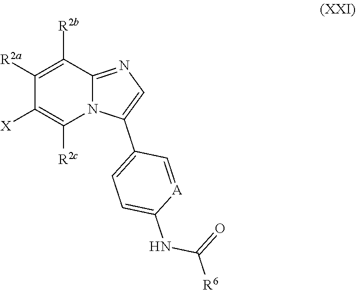
wherein A is CH or N;
R^{2a} is selected from H, C₁₋₆alkyl, C₃₋₆cycloalkyl, and C₁₋₆alkoxy-C₁₋₆alkyl;
R^{2b} is selected from H, CN, C₁₋₆alkyl, C₃₋₆cycloalkyl, C₁₋₆alkoxy-C₁₋₆alkyl, amino, and —NHC(O)C₁₋₆alkyl;
R^{2c} is H;
R⁶ is selected from C₁₋₆alkyl, C₁₋₆alkoxy, C₃₋₆cycloalkyl, C₁₋₆alkylamino, C₁₋₆alkoxyamino, and C₁₋₆alkylC₁₋₆alkoxyamino; and
X is chloro (Cl), bromo (Br), or iodo (I);
or a salt or N-oxide thereof.


SEQUENCE LISTING (1 sequence)

SEQ ID NO: 1 - RNA, 1527 nt, Streptomyces chrestomyceticus
  source 1..1527: mol_type = genomic RNA
acggagagtttgatcctggctcaggacgaacgctggcggcgtgcttaacacatgcaagtcgaacgatgaacttccttcgggaggggattagtggcgaacgggtgagtaacacgtgggcaatctgccctgcactctgggacaagccctggaaacggggtctaataccggatacgacacgggatcgcatgatcttccgtgtggaaagctccggcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaaggtagccgtaccggaaggtgcggctggatcacctccttt